(19) United States
(12) Patent Application Publication
(10) Pub. No.: US 2007/0135340 A1
(43) Pub. Date: Jun. 14, 2007

(54) IGF-1 novel peptides

(76) Inventors: Nadia Rosenthal, Monterotondo (IT); Antonio Musaro, Rome (IT); Nadine Winn, Rome (IT)

Correspondence Address: WENDEROTH, LIND & PONACK, L.L.P., 2033 K STREET N. W., SUITE 800, WASHINGTON, DC, 20006-1021, US

(21) Appl. No.: 11/374,099

(22) Filed: Mar. 14, 2006

Related U.S. Application Data
(63) Continuation-in-part of application No. PCT/IB05/03953, filed on Nov. 29, 2005.

(30) Foreign Application Priority Data
Nov. 29, 2004 (GB) 0426154.1

(51) Int. Cl. A61K 38/30; C07H 21/04; C12P 21/06; C07K 14/65

(52) U.S. Cl. 514/12; 530/399; 435/69.1; 435/320.1; 435/325; 536/23.5

(57) ABSTRACT

The present invention relates to novel polypeptide constructs based on peptides derived from Insulin-like Growth Factor I (IGF-I). The invention also relates to novel uses for IGF-1-derived peptides, particularly for the prevention and treatment of diseases involving regulation of cellular growth or differentiation, regeneration and tissue repair.

The present invention relates to novel polypeptide constructs based on peptides derived from Insulin-like Growth Factor I (IGF-I). The invention also relates to novel uses for IGF-1-derived peptides, particularly for the prevention and treatment of diseases involving regulation of cellular growth or differentiation, regeneration and tissue repair.

All documents mentioned in the text and listed at the end of the description are incorporated herein by reference.

Insulin-like growth factors (IGFs) are members of the highly diverse insulin gene family that includes insulin, IGF-I, IGF-II, relaxin, prothoraciotropic hormone (PTTH), and molluscan insulin-related peptide (1;2;3). The IGFs are circulating, mitogenic peptide hormones that have an important role in stimulating growth, differentiation, metabolism and regeneration both in vitro and in vivo (4;5).

The Insulin-like growth factor-1 (IGF-1) gene gives rise to several isoforms of unprocessed (precursor) IGF-1 which differ by the length of the amino terminal leader (signal) peptide and structure of the carboxy terminal end (E-domain) (discussed in detail below). These unprocessed polypeptides undergo post-translational protease cleavage to remove the leader sequence and the E-domain to yield a 70 amino acid long (mol wt 7,649 D) single chain mature IGF-1 polypeptide with three intrachain disulphide bridges.

The IGF-1 gene gives rise to a heterogeneous pool of mRNA transcripts (Fig 1B). Such heterogeneity of the mRNAs results from several events (or combination of these events): use of alternative transcription start sites located in leader exons (exon 1 and exon 2) (6; 7; 8); alternative post-transcriptional exon splicing (9; 10; 11; 6; 7; 8); and use of different polyadenylation sites (12; 13). These multiple IGF-1 mRNAs transcripts encode different isoforms of precursor IGF-1 peptide (Fig 1C), which undergo post-translational cleavage to release the biologically active mature (70 amino acid long) IGF-1. There is also a degree of heterogeneity in the signal peptides used, that are eventually cleaved during post-translational processing (8). Additionally, alternative splicing of exons at the 3′-end of mRNA precursor introduces further complexity in the variety of IGF-1 transcripts and IGF-1 isoforms translated from these transcripts.

Adding to the confusion in the literature regarding the large array of IGF-I isoforms, IGF-1 functions have also been extensively analysed, with several groups reporting different and contradictory roles of the growth factor in vivo and in vitro. For example, Ito and co-workers showed that IGF-1, lacking class 1 or 2 signal peptides or C-terminal E peptides, increases the transcription of muscle specific genes, and induces a two fold increase of cell size in neonatal rat cardiomyocytes (14) indicating a functional role in regulating cardiac cells hypertrophy. Conversely to this study, cultured neonatal cardiomyocytes treated with an antisense probe to IGF-1 receptor, showed suppressed DNA replication, mitosis and cell proliferation. Moreover, the antisense treatment did not alter the expression of ANF in myocytes or cellular hypertrophy (15).

In vivo, intravenous infusion of IGF-1, lacking class 1 or 2 signal peptides or E peptides, has been shown to induce a significant increase in protein synthesis particularly in the heart, which was not accompanied by significant changes in blood glucose (16). Interestingly, administration of IGF-1 alone or in combination with Growth Hormone (GH) in normal adult rats increased the left ventricular weight compared with placebo-treated rats (17). Transgenic mice generated with the human IGF-1B cDNA showed no striking differences in heart size and cell volume when compared to control mice, but the number of myocytes in the heart was 55% higher in transgenic animals, indicating that IGF-1 overexpression is coupled with myocyte proliferation (18). In addition, these animals do not undergo significant regeneration after injury (P. Anversa, personal communication). In another study, it has been shown that mice overexpressing a truncated form of human IGF-1 (IGF-1A, which does not containing class 1 or 2 signal peptides) under the control of α-skeletal actin promoter induced physiological and then pathological cardiac hypertrophy, associated with a decreased systolic performance and increased fibrosis (19).

Multiple lines of evidence, demonstrating the capacity of a particular insulin-like growth factor-1 isoform (mIgf-1), expressed locally in adult post-mitotic tissues such as skeletal muscle and heart, to recapitulate the regenerative capacity of prenatal/neonatal tissues, have been generated. The mIGF-1 isoform comprises a Class 1 signal peptide, and an Ea extension peptide. Expression of the mIGF-1 isoform as a transgene in animal skeletal and cardiac muscles resolves inflammation, enhances distal cell survival in a paracrine manner, increases chemoattractive mechanisms and mobilizes circulating bone marrow and endogenous progenitor cells to repair tissue damage. In neonatal tissues, this isoform is expressed at high levels but declines soon after birth in extrahepatic tissues and decreases further during ageing. It has been demonstrated that mIgf-1, delivered as a muscle-specific transgene or virus to mouse skeletal muscle, enhances repair of skeletal muscle damage, enhancement of exercise-induced hypertrophy, reversal of age-related atrophy, and prevention of dystrophic muscle degeneration (20; 21, 22; 23). When expressed as a cardiac-specific transgene, mIGF-1 transiently increased cardiac mass during post-natal stages due to sustained increases in protein translational components and heightened expression of physiological but not pathological markers of cardiac growth and hypertrophy. Induction of myocardial infarction produces localised damage, cell death and massive inflammation but mIGF-1 transgenic animals rapidly resolve in complete repair of the injured heart without scar formation and late-onset proliferation near the site of injury. Down-regulation of specific inflammatory cytokines suggests that mIGF-1 improves cardiac regeneration in part by modulation of the inflammatory response. Since supplementary expression of this growth factor does not alter normal heart development or long-term postnatal cardiac form and function, the enhancement of cardiac regeneration and repair by localised expression of mIGF-1 suggests novel and clinically feasible therapeutic strategies.

Taken together, these data implicate mIGF-1 as a powerful enhancer of the regeneration response, mediating the recruitment of bone marrow and other progenitor cells to sites of tissue damage and augmenting local repair mechanisms. However, the precise mechanism of mIGF-1 action has until now proved elusive.

### SUMMARY OF THE INVENTION

The effect of various C terminal extension or E peptides of IGF-I has been studied in a range of cells and organisms. Whereas the current consensus suggests that it is the mature form of IGF-1 that is responsible for the various physiological effects noted for this protein, the applicant has ascribed many of these effects to the E peptides. Until now, the multiple E peptides generated by alternate splicing have been largely ignored as a potential source of functional diversity.

The invention thus involves the generation and systemic or localized delivery of IGF-1 protein isoforms, including small peptides (35-41 aa) encoding the various sequences of the IGF-1 E peptides to damaged or degenerating tissues. The underlying premise of the invention is that IGF-1 E peptides have unique subsets of function encoded in the full length protein, in particular, the regenerative capacity of IGF-1.

There is some confusion in the literature regarding the nomenclature of the various E peptides and consequently the way in which the various isoforms of IGF-1 are referred to. Before the identification of the IGF-1 En isoform, as described herein, two IGF-1 isoforms had been identified in murine muscle. These have been termed IGF-1 Ea and IGF-1 Eb [24]. In comparison, three IGF-1 isoforms have so far been identified in human skeletal muscle. These have been termed IGF-1 Ea, IGF-1 Eb, and IGF-1 Ec [25], [26]. Both the human and murine isoforms that are translated from parts of exons 4 and 6 of their respective IGF-1 genes are termed IGF-1 Ea. The consistency in the nomenclature breaks down at this point, and the human isoform that is translated from parts of exons 4, 5 and 6 is termed IGF-1 Ec, whereas the murine isoform translated from the same exons is termed IGF-1 Eb. Both the murine isoformIGF-1 Eb and the human isoformIGF-1 Ec have been termed the “mechano growth factor” because of the their shared functions and exon structure.

It is appreciated in the art that murine and human IGF-1 Ea are equivalent and murine IGF-1 Eb is equivalent to human IGF-1 Ec. To save confusion we have used terminology throughout this specification corresponding to the murine nomenclature i.e. IGF-1 Ea and Eb. However, it will be clear to the reader skilled in the art that where IGF-1 Eb is referred to, the human equivalent of murine IGF-1 Eb is being referred to, i.e. the peptide referred to in the literature as human IGF-1 Ec.

Accordingly, one aspect of the present invention relates to the use of an IGF-I Ea peptide for the regulation of cellular growth or differentiation. The Ea peptide is shown herein to have an important role in differentiation and regeneration of various cell types. For the first time, the inventors have demonstrated that, rather than the mature 70 amino acid IGF-1 peptide, it is the C terminal 35 amino acid Ea peptide that is responsible for some of its functions. More particularly, the Ea peptide and isoforms of IGF-1 comprising the Ea peptide have been shown to be able to induce muscle hypertrophy, in particular skeletal muscle hypertrophy.

Furthermore, various of the physiologically interesting effects of IGF-1 have been assigned by the inventors to the Eb peptide. More particularly, the Eb peptide and isoforms comprising the Eb peptide have been shown to have an important role in the proliferation of various cell types.

Furthermore, the inventors have surprisingly discovered an IGF-1 isoform found in rodents, containing a third E peptide, termed herein “IGF-1 En peptide”. The IGF-1 En peptide is closely related to the human IGF-1 Eb peptide and is thought to possess a similar functionality.

This discovery has significant ramifications. A number of applications have been suggested for mIgf-1, in the correction of various neuromuscular and cardiovascular pathologies. Now that it is known that the entity responsible for regulating certain cellular growth functions are the E peptides of IGF-1, previously ignored as a potential source of functional diversity, it is possible to tailor more precisely the application of these peptides in a therapeutic context. In particular, the teaching of the invention may be applied to conditions such as those listed below, by administering E peptides, or other recombinant proteins including E peptides, or nucleic acids encoding such peptide or protein entities, to an affected patient.

Traumatic Skeletal Muscle Injury

For example, a subset of myogenic progenitors is enhanced in injured mIGF-1 transgenic muscles, expressing the haematopoietic markers CD45, CD11b, c-Kit and Sca-1. Upon muscle injury, these cells increase also in the bone marrow compartment, revealing an unexpected response to distal trauma. Damaged mIGF-1 transgenic muscles activate novel genes implicated in urodele amphibian regeneration. In regenerating MLC/mIGF-1 transgenic muscles, cell populations expressing stem cell and myeloid markers exhibited accelerated myogenic differentiation. In vitro, primary myoblast cultures from MLC/mIGF-1 muscles readily converted co-cultured bone marrow to a myogenic lineage and incorporate bone marrow cells by fusion to muscle fibres (27). The local changes effected by postmitotic expression of the mIGF-1 transgene are illustrated by the enhanced myogenic differentiation of primary stem cell cultures isolated from regenerating mIGF-1 muscles, which unexpectedly re-entered the cell cycle upon serum stimulation, proliferated readily and differentiated upon serum withdrawal. Increased levels of chemokine receptors in mIGF-1 damaged muscle suggest a mechanism whereby circulating cells, which express the corresponding chemokines, are drawn in larger numbers to target organs expressing mIgf-1.

Muscle Wasting

Metabolic abnormalities in advanced chronic heart failure include functional and morphological decrements in the skeletal musculature that result in progressive muscular atrophy. An experimental model of left ventricular dysfunction was used to detect alterations in the skeletal muscle proteolytic ubiquitin-proteasome pathway, and to assess the potential therapeutic role of supplemental mIGF-1 in attenuating muscle atrophy. Twelve weeks after coronary artery ligation, left ventricular dysfunction and enlargement were observed in wildtype mice and in their transgenic littermates expressing mIGF-1 exclusively in skeletal muscle. Skeletal muscular atrophy in wildtype mice with left ventricular dysfunction was accompanied by an increase in myosin heavy chain ubiquitination, enhanced proteasome activity, and robust induction of Atrogin-1, an ubiquitin-conjugating E3 ligase. In contrast, overexpression of transgenic mIGF-1 prevented muscular atrophy and proteasome activity. The findings suggest that atrophy of the skeletal musculature in mice with left ventricular dysfunction occurs through targeting of specific structural proteins by the ubiquitin-proteasome pathway. The inhibition of muscle atrophy by supplemental mIGF-1 expression provides a promising therapeutic avenue for the prevention of skeletal muscle wasting in chronic heart failure (Schulze et al, manuscript submitted).

Amyotrophic Lateral Sclerosis

Amyotrophic lateral sclerosis (ALS) is a progressive, lethal neuromuscular disease that is associated with the degeneration of motor neurons, leading to atrophy of limb, axial, and respiratory muscles. Although certain inherited forms of ALS have been attributed to acquired toxic properties associated with a dominant mutation in the SOD1 gene, the aetiology of the disease and the cellular targets critical to the degenerative process have remained difficult to define.

It has been shown that muscle-restricted, hypertrophic action of mIGF-1 maintained muscle integrity and enhanced satellite cell activity in a mutant SOD1 transgenic mouse model of ALS, induced calcineurin-mediated regenerative pathways, and reduced components of catabolic activity. mIGF-1 transgene expression also stabilized neuromuscular junctions enhanced both proximal and distal neuronal survival in mutant SOD1 mice, thus delaying the onset and progression of the disease. These studies establish skeletal muscle as a primary target for the dominant action of inherited SOD1 mutations in motor neuronal degradation. The protection afforded by mIGF-1 action in skeletal muscle in conjunction with the teaching of the present invention suggests novel therapeutic strategies to attenuate the neuronal degeneration associated with ALS (Dobrowolny et al., manuscript in preparation). For example, E peptides, or other recombinant proteins including E peptides, or nucleic acids encoding such peptide entities, may be administered to an affected patient.

Myocardial Infarction

When delivered as a transgene restricted to the myocardium under the control of the alpha-MHC promoter (α-MHC) to exclude possible endocrine effects on other tissues, an mIGF-1 gene produced accelerated growth during postnatal heart development, but never exceeded wild-type cardiac size in the adult, with a comparative size by 6 months. mIgf1-induced remodelling was accompanied by increased activation of ERK and JNK signalling at one week after birth, and by increased ANP and BNP transcripts at one and two months. Sustained translational activity was observed during all phases of heart development in mIGF-1 overexpressing heart, independent of AKT activation. Early increased heart size of mIGF-1 transgenic hearts was not due to increased cardiomyocyte proliferation, nor did it lead to pathological conditions, as shown by a comparable electrophysiological function between transgenic and wild-type hearts.

The regenerative capacity of mIGF-1 transgenic hearts was analyzed by direct cardiotoxin injection into the heart of four months old mice. Cardiotoxin produced a reproducible and localized damage of the right and left ventricles 48 hours post-injection, in both wild-type and transgenic hearts, with evident cell death and massive inflammation. In contrast to the progression of scar formation in wild-type hearts, transgenic mIGF-1 overexpression induced complete repair of the injured heart after 1 month, without scar formation and with proper tissue reconstitution. Down-regulation of specific inflammatory cytokines suggested that mIGF-1 induced heart regeneration by lowering the inflammatory response. To assess cardiac hyperplasia, cell cycle was assayed by measuring the nuclear incorporation of bromodeoxyuridine (BrdU), a marker of DNA synthesis, 1 month after cardiotoxin injection.

The mIGF-1 transgene induced a significant percentage of total cells to enter cell cycle in response to cardiotoxin injection compared to wild-type hearts. It was found that cardiac cells re-enter the cell cycle, although cells of diverse lineage were found in the myocardium and in the vessels. The nature of these cells is still under investigation. No differences in proliferative state were found in injured heart 24 hour and 1 week after cardiotoxin injection, indicating that the repair program, following the early activation of regeneration program, is activated as a late step in mIGF-1 transgenic hearts.

One aspect of the invention therefore provides an isolated Ea IGF-1 peptide, as defined herein. The invention also provides methods for the regulation of cellular growth or differentiation, comprising exposing a cell to the Ea IGF-1 peptide in a physiologically effective amount. The invention also provides methods for inducing muscular hypertrophy, comprising exposing a cell to an Ea IGF-1 peptide in a physiologically effective amount. Alternatively, the invention provides methods for inducing skeletal muscle hypertrophy comprising exposing a cell to an Ea IGF-1 peptide in a physiologically effective amount.

The invention also provides methods for increasing the total circulating levels of IGF-1 in an organism, comprising exposing the organism to an Ea IGF-1 peptide in a physiologically effective amount. The organism may be a non-human transgenic animal line, which has been transformed with a construct encoding an Ea IGF-1 peptide.

A further aspect of the invention provides an isolated Eb IGF-1 peptide, as defined herein. The invention also provides methods for the regulation of cellular growth, comprising exposing a cell to the Eb IGF-1 peptide in a physiologically effective amount. The invention also provides methods for inducing cell proliferation, comprising exposing a cell to the Eb IGF-1 peptide in a physiologically effective amount.

A further aspect of the invention provides an isolated En IGF-1 peptide, as defined herein. The invention also provides methods for the regulation of cellular growth, comprising exposing a cell to the En IGF-1 peptide in a physiologically effective amount. The invention also provides methods for inducing cell proliferation, comprising exposing a cell to the En IGF-1 peptide in a physiologically effective amount.

By the “IGF-1 Ea peptide” is meant the 35 amino acid C terminal peptide translated from part of exons 4 and 5 of the IGF-1 gene as part of the IGF-1 propeptide and which is cleaved off during post-translational processing.

By the “IGF-1 Eb peptide” is meant the 40 or 41, depending on the species of origin, amino acid C terminal peptide translated from parts of exons 4, 5 and 6 of the IGF-1 gene as part of the IGF-1 propeptide and which is cleaved off during post-translational processing.

By the “IGF-1 En peptide” is meant the 63 amino acid C terminal peptide translated from parts of exons 4, and 5 of the IGF-1 gene as part of the IGF-1 propeptide and which is cleaved off during post-translational processing.

Where the various peptides of the invention have been cleaved from either a prepropeptide or a propeptide, then the invention also envisages minor variations in the length of these peptides. This minor variation may arise through variations in the position of the cleavage site. For example, if the human Class 1/IGF-1/Ea peptide is cleaved between the class 1 signal molecule and the IGF-1 Ea portion of the molecule, between amino acids ATA|GPE as indicated, then the class 1 signal peptide will have the sequence as recited in SEQ ID NO:14 (the Human Class 1 signal peptide), starting with the amino acid sequence MGK . . . and ending with . . . ATA, whilst the IGF-1 Ea portion will comprise the sequences as recited in SEQ ID NO:30 (the Human mature IGF-1 peptide sequence) contiguous with the sequence recited in SEQ ID NO:22 (the Human Ea peptide), starting with the amino acid sequence GPE . . . and ending with . . . YRM.

However, either portion of the molecule, the class 1 signal peptide or the IGF-1 Ea, may be “n” amino acids shorter or longer than the sequences described above as a result of variations in the position of cleavage. If the molecule is cleaved between amino acids AT|AGPE as indicated, then the class 1 signal peptide molecule will be one amino acid shorter, ending with the amino acid sequence . . . SAT, whilst the IGF-1 Ea portion of the molecule will be one amino acid longer, starting with the amino acid sequence AGPE . . . The same variation can occur with any of the peptides of the invention, between any of the possible cleavage sites. For example, variation may occur between any class of signal molecule and IGF-1 molecule, between any class of E peptide and IGF-1 molecule or between any combinations of signal peptide, IGF-1 peptide and E peptide. “n” may be 1, 2, 3, 4, 5, 6, 7, 8, 9, 10, 15, or 20 amino acids shorter or longer.

Although the Applicant does not wish to be limited or bound by any particular theory, it is postulated herein that the complete IGF-1 prepropreptide (i.e. including signal and E peptides) or other incompletely processed forms of IGF-1 (e.g. in which signal peptides have been processed off the preproprotein but in which the E peptides remain) may perform the same or overlapping functions as the isolated E peptides. For example, in experiments performed herein, showing regeneration of transgenic IGF-1 isoform muscles, none of the over-expressed prepro-variants of IGF-1 were found to be processed to pro-IGF-1Ea/Eb or mature IGF-1 during proliferation. This result suggests that the entire prepro-peptide or a partially processed form, including the E-peptide and potentially also the signal peptide, influenced proliferation of myoblasts in this system. These findings also shed light on the outstanding question of whether the IGF-1 isoforms are active or properly localised without cleavage of the signal or E peptides. The data presented herein strongly indicate that the entire IGF-1 prepropeptide does not necessarily need full processing in order to be active, and raise the possibility that specificity of IGF-1 function might be determined by processing status, or by the presence of isolated E-peptide once the prepropeptide is cleaved.

The data also strongly indicate that different signal peptides may confer unique properties on IGF-1, such as inhibition of IGF-1 secretion, subcellular compartmentalisation and membrane association. This in turn may control protein stability, bioavailability, or potential intracellular functions of unprocessed prepropeptide in cases where IGF-1 gene expression is induced in proliferating cells. The induction of signal peptide cleavage may also provide available propeptides to associate with binding proteins or extracellular matrix. This in turn may control protein stability, bioavailability, or receptor specificity.

The data also suggest that the different E peptides are largely retained on the IGF-1 protein body in differentiated muscle, and confer additional and separate properties on IGF-1 when the molecule is secreted as a propeptide, such as control of IGF-1 secretion, subcellular compartmentalisation, association with membrane, binding proteins, or extracellular matrix and association with receptors. This in turn may control protein stability, bioavailability, or receptor specificity.

Furthermore, it is postulated herein that the various prepropeptide, propeptide and mature IGF-1 molecules display different functions at different stages of processing. For example, the Class 1/IGF-1/Ea prepropeptide molecule may display one or more different function when compared to the IGF-1/Ea propeptide, which in turn may display one or more different functions to either the isolated IGF-1 or isolated Ea peptide molecule. The same may be true for any class of signal peptide in combination with any variant of the E peptide. Accordingly, aspects of the invention that are discussed herein in connection with the E peptides may also be relevant to polypeptides that comprise the E peptide within their sequence. The invention therefore also includes various uses and applications of complete IGF-1 prepropeptides or IGF-1 propeptides.

By “prepropeptide” is meant a peptide, as defined herein, having the formula NH₂—A—B—C—D—COOH. The terms of this formula are given below.

By “propeptide” is meant a peptide, as defined herein, having the formula NH₂—B—C—D—COOH, wherein the optional signal sequence has either been cleaved from the molecule, or was not originally present.

Particular prepropeptides contemplated by the invention include Class1/IGF-1/Ea, Class1/IGF-1/Eb, Class2/IGF-1/Ea and Class2/IGF-1/Eb. Particular propeptides contemplated by the invention include IGF-1/Ea and IGF-1/Eb. All these various constructs form aspects of the present invention.

According to one aspect of the invention, therefore, there are provided methods for the regulation of cellular growth, differentiation or proliferation, comprising exposing a cell to any of the prepropeptides or propeptides, as defined herein, in a physiologically effective amount. More preferably, there are provided methods for the regulation of cellular growth or differentiation, comprising exposing a cell to any of the preferred prepropeptides or propeptides, as defined herein, in a physiologically effective amount.

Additionally, there are provided methods for the regulation of cellular growth or differentiation, comprising exposing a cell to Class1/IGF-1/Ea, Class2/IGF-1/Ea and/or IGF-1Ea in a physiologically effective amount. The invention also provides methods for inducing cell proliferation, comprising exposing a cell to Class1/IGF-1/Eb, Class2/IGF-1/Eb and/or IGF-1/Eb in a physiologically effective amount.

The invention also provides methods for inducing muscular hypertrophy, comprising exposing a cell to any of the preferred prepropeptides or propeptides, as defined herein, in a physiologically effective amount. These methods for inducing muscular hypertrophy may comprise exposing a cell to Class1/IGF-1/Ea, Class2/IGF-1/Ea or IGF-1Ea in a physiologically effective amount. More specifically, the invention provides methods for inducing skeletal muscle hypertrophy comprising exposing a cell to Class1/IGF-1/Ea, Class2/IGF-1/Ea or IGF-1Ea peptide in a physiologically effective amount.

The invention also provides methods for the regulation of cellular growth, comprising exposing a cell to the En IGF-1 prepropeptide in a physiologically effective amount. The invention also provides methods for inducing cell proliferation, comprising exposing a cell to the En IGF-1 prepropeptide in a physiologically effective amount.

The invention also contemplates prepropeptides and propeptides which have been modified to alter their function or stability. In one embodiment of this aspect of the invention, the prepropeptides or propeptides may be modified to increase their half-life in an organism to which it is intended they be administered. As described herein, it is postulated that the E peptide can be cleaved from the core IGF-I molecule by thrombin or a similar protease, and therefore, the invention contemplates stabilising the prepropeptides or propeptides of the invention by modification of the predicted protease cleavage site. Such modifications may result in total removal of the cleavage site or may result in a partial modification to the site which reduces the rate at which the prepropeptide or propeptide is cleaved. Alternatively, such modifications may alter the protease specificity of the site, i.e. the site may be modified from the natural cleavage site to a site for an alternative protease, or alternative class of protease altogether.

Alternatively, another protease cleavage site may be introduced into the prepropeptide or propeptide. Such a site could be tailored to a specific protease. In this scenario, such a prepropeptide or propeptide may then be administered to a patient before, after or sequentially with the specific protease.

In a second embodiment of this aspect of the invention, the prepropeptides or propeptides may be modified to alter their function. For example, the peptides may be altered to increase or decrease their affinity for IGF-1 binding proteins. The peptides may also be modified to have preferential affinity for certain tissue types or injured tissues, or may have altered affinities for different binding proteins that affect their bioavailability or target tissues.

Although the Applicant does not wish to be limited or bound by any particular theory, it is postulated herein that the IGF-1 isoforms with the Class 1 signal peptide are capable of cross-activating expression of specific transcripts emanating from the endogenous IGF-1 gene. According to this aspect of the invention, therefore, there are provided methods for cross-activating expression of specific transcripts emanating from the endogenous IGF-1 gene, comprising exposing a cell to an IGF-1 isoform comprising the Class 1 signal peptide in a physiologically effective amount. For example, MLC/Class 1 IGF-1Ea transgene was found to increase endogenous levels of Class 2 IGF-1Ea, raising the possibility that hypertrophy in these animals is predominately due to the Class 2 IGF-1Ea isoform, which is considered the more potent combination of signal and E peptides for promoting hypertrophy. Similarly, the MLC/Class 1 IGF-1b transgene increased endogenous levels of Class 1 IGF-1Ea. Although the Applicant does not wish to be limited or bound by any particular theory, it is postulated herein that the IGF-1 isoforms activate distinct non-overlapping gene pathways. For example, in experiments performed herein, in particular the Affymetrix transcriptional profiling data, a very clear picture emerges of almost complete lack of overlap in signalling pathways between isoforms. That is to say, each isoform activates a unique set of downstream genetic pathways in the target tissue. According to this aspect of the invention, therefore, the methods of the invention, as described herein, are envisaged to result in similar up- and/or down- regulation profiles as those described in Tables 9-16. For example, where a method of the invention involves the use of a Class 1/IGF-1/Ea isoform, then it is envisaged that administration of a physiologically effective amount of the isoform will result in a similar pattern of gene up- and down-regulation as that described in Tables 9-16 for this particular isoform. The same applies to the other three IGF-1 isoforms. A similar pattern of gene up- and/or down-regulation may result in values within +/−40% of those disclosed in Tables 9-16, preferably +/−35%, 30%, 25%, 20%, 15%, 10%, 8%, 7%, 6%, 5%, 4%, 3%, 2%, 1% or less than within +/−1% of those values disclosed in Tables 9-16.

Preferably, the IGF-1 Ea peptide and IGF-1 Eb peptide (referred to as the Ec peptide) are of human origin.

By “regulation of cellular growth” is meant that the peptide entity is capable of altering, preferably increasing cellular growth. Said cells may be singular or form part of a cellular mass, such as a tissue or organ. In particular, the Ea peptide may cause hypertrophy of muscle tissue or adipose tissue. Examples of muscle tissue will be known to the person of skill in the art and include skeletal (striated), smooth and cardiac muscle tissue.

By “differentiation” is meant that the peptide entity is capable of inducing biochemical and structural changes in an unspecialised cell, thereby causing its form and function to become specialised. Examples of such differentiation include repair of diseased (including cancerous) cells, alteration of the genetic constitution of cells, induction of specific cell types and cell fates, changing the immunological profiles of cells, and inducing particular desired immune functions or properties. The alteration of the property may result in the cell undergoing differentiation towards a more specialised form or function, for example from a stem cell towards an adult cell with a specialised function (for example circulating bone marrow-derived cells such as myeloid progenitors).

By “proliferation” is meant that the peptide entity is capable of inducing an increase in the number of cells. The number of cells increase as a result of cell growth and cell division. Said cells may be singular or form part of a cellular mass, such as a tissue or organ. An increase in proliferation may result in 10% more cells, preferably greater than 20%, 30%, 40%, 50%, 75%, 100%, 200%, 500% or 1000% or more than the original cell number within a specified period of time. Such a period may be 12 hours, 24 hours, 2 days, 7, days, 2 weeks, a month, 3 months, 6 months, or even a year or more.

The peptide can work either as an isolated peptide or as a fusion with another entity. The peptide of the invention will typically be a polypeptide e.g. consisting of between 20 and 500 amino acids. The polypeptide preferably consists of no more than 200 amino acids (e.g. no more than 190, 180, 170, 160, 150, 140, 130, 120, 110, 100, 90, 80, 70, 60 or no more than 50). Details of particular preferred polypeptides for use in accordance with the invention are given below.

The peptides of the invention may be used to regulate cellular growth or differentiation in any cell type, for example, muscle tissue; nervous tissue; adipose tissue; cartilage; bone; hepatic tissue; kidney tissue; hair; or skin.

The peptides of the invention may be used in the prevention and treatment of cellular trauma. The trauma may be to any cell type and caused as a result of any form of trauma. Particular examples include crush injury; surgical damage; muscle tear injury; nerve damage; surgical damage; myocardial infarction; stroke; ischemia; burns; bone fractures or UV damage.

Although the Applicant does not wish to be limited or bound by any particular theory, it is postulated herein that the E peptide can be cleaved from the core IGF-1 molecule by thrombin. The putative cleavage site that separates the variable E peptides, to generate the mature 70 amino acid IGF-1 protein, has been partially characterised. Unexpectedly, this consensus sequence, highly conserved between species, potentially corresponds to a thrombin cleavage site, raising the exciting possibility that the clotting cascade may act as a stimulus for regenerative action of the IGF-1 precursor via the release of E peptides. In this model, the activation of prothrombin to thrombin, as a result of cellular trauma, results in the cleavage of fibrinogen to fibrin and the subsequent formation of blood clots. It is thus postulated that the mature 70 amino acid IGF-I peptide and E peptides circulate in the body fused together as an inactive pro-form of the IGF-1 protein. Once activated as a result of trauma, thrombin then cleaves, and as a consequence activates the various E peptides from the IGF-I proprotein. In this model, this cleavage and activation of the IGF-I E peptides will occur only at sites of localised cellular trauma, providing an activation signal that is economic, in the sense that it is not needlessly wasteful, and localised, in the sense that it is specific to a site where regenerative capacity is required.

As a consequence of the Applicant's theory, the invention also provides for inactive pro-forms of the IGF-1 Ea and Eb peptides which are activatable by thrombin cleavage. Such peptides of the invention may be administered prior to trauma in an attempt to prevent and reduce the damage. For example, in the case of patients at high risk of a condition such as myocardial infarction, peptides may be administered to achieve systemic circulating levels of a proprotein form of an IGF-1 E peptide, thus allowing for a faster response to localised cellular trauma if and when this occurs.

Peptides of the invention may also be administered to patients awaiting surgery. Heightened systemic levels of the IGF-1 Ea pro-form would provide a patient with an enhanced ability to deal with cellular trauma caused by surgery.

Peptides of the invention may also be administered shortly after trauma. An example is provided by the case of a patient having suffered internal injuries. Administration of the peptides of the invention will provide the patient with a heightened ability to deal with such injuries. Internal injuries may be as a result of a physical trauma, such as a vehicle accident, or may be from a pre-existing condition such as a myocardial infarction.

Peptides of the invention may be delivered locally or systemically to enhance the regeneration of injured or degenerating tissue.

Peptides of the invention can also be used to treat external injuries by topical application. One example is provided by a patient suffering wounds to the skin. Such wounds could be caused by a variety of traumas including laceration and burns.

Peptides of the invention can also be topically, co-administered with thrombin. For example, peptides of the invention may be administered during surgical procedures, along with thrombin, to aid with haemostasis.

Peptides of the invention can also be used in the prevention and treatment of muscular atrophy and related conditions. Such muscular atrophy may be as a result of the ageing process (sarcopenia). Muscle weakening and frailty are well documented effects of the ageing process. Peptides of the invention may be administered as a preventative measure, that is, as a regular supplement to slow the muscular atrophy caused by the ageing process. They may also be administered at a later stage to both slow and reverse the age related affects of muscular atrophy.

Such muscular atrophy may be caused by neuromuscular or neurodegenerative disorders. These disorders may be acquired or hereditary and can include Parkinson's Disease, including early onset forms (Autosomal recessive juvenile Parkinson's; ARJP), Lewy body dementias, and general synucleinopathies; Alzheimer's disease, including frontotemporal dementias (FTD), corticobasal degeneration (CBD), progressive supranuclear palsy (PSP), and general tauopathies and amyloidopathies; Amyotrophic Lateral Sclerosis, including adult-onset motor neuron disease; Huntington's disease, including spino-cerebellar ataxias and adult onset trinucleotide repeat disorders.

Such muscular atrophy may be caused by muscular dystrophies; such conditions can include Becker muscular dystrophy, distal muscular dystrophy, Duchenne's muscular dystrophy, limb-girdle muscular dystrophy, myotonic dystrophy and oculopharyngeal muscular dystrophy.

Moreover, said muscular atrophy may be induced by congestive heart failure, cardiomyopathies, atherosclerosis, acute insult including myocarditis or myocardial infarction.

In a further aspect, the peptides of the invention may be used to treat muscular atrophy caused by the disuse of a muscle. This disuse may be caused by a spinal chord injury or through the immobilisation of a limb through injury, for example, resulting from correction of a bone fracture. Said disuse may also be caused as result of a patient suffering from a stroke.

Peptides of the invention may also be used for increasing muscle hypertrophy. Muscle hypertrophy may be increased as an aid to physical therapy. An example is provided by helping a patient gain weight after a severe illness, injury, or continuing infection. Peptides of the invention may also be used to increase muscle hypertrophy in livestock in order to increase yields. Examples of clear commercial relevance include cattle, sheep, pigs and fish. Other examples may be found in sports medicine, or in body-building.

Peptides of the invention may also be used for increasing adipose tissue deposits. Ea peptides, and fusion proteins including these peptides, will be of particular use in this context. Adipose tissue may be increased to help a patient gain weight after a severe illness, injury, or continuing infection. Peptides of the invention may also be used to aid in the treatment of Anorexia nervosa or Bulimia nervosa.

Although the Applicant does not wish to be limited or bound by any particular theory, it is postulated herein that mIGF-1 accelerates the timing of regeneration and reduces the amount of mononucleated infiltrating cells post-injury. The local expression of mIGF-1 improves the regenerative phase increasing the pool of satellite cells and modulating the inflammatory response of injured skeletal muscle. Furthermore, mIGF-1 modulates inflammatory cytokines, such as MCP1, MCP2, MIP-1α, and MIP-1β at early stages, stimulating a qualitative environment for complete functional recovery. It is proposed that mIGF-1 modulates inflammatory cytokines at early stages, stimulating a qualitative environment for a complete functional recovery.

As a consequence of the Applicant's theory, the invention also provides for use of the peptides of the invention for improving the dystrophic environment and/or stimulating the regenerative capacity of stem cells. Ea peptides, and fusion proteins including these peptides, will be of particular use in this context.

A peptide of the invention may have the formula NH₂—A—B—C—D—COOH, wherein: —A— is an optional N-terminus amino acid sequence consisting of a amino acids; —B— is an optional amino acid sequence consisting of b amino acids; —C— is a sequence derived from an IGF-1 Ea or Eb peptide; and —D— is an optional C-terminus amino acid sequence consisting of d amino acids. The positions of entities B and C relative to each other may be reversed in the protein sequence, if necessary.

As defined above, —A— is an optional N-terminus amino acid sequence consisting of a amino acids. The value of a is generally at least 1 (e.g. at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, 100, 150, 200, 250, 300, 350, 400, 450, 500, etc.), but can be zero (i.e. —A— is absent). Examples of typical —A— moieties include leader sequences to direct protein trafficking, or short peptide sequences which facilitate cloning or purification (e.g. histidine tags i.e. Hisₙ where n=3, 4, 5, 6, 7, 8, 9, 10 or more). In some embodiments, moiety —A— is, or terminates at its N-terminus with, a methionine residue. Other examples of —A— moieties include IGF-1 signal peptides, such as the Class 1 IGF-1 signal peptide, consisting of 48 amino acid residues derived from parts of exons 1 and 3 of the IGF-1 gene; the Class 2 IGF-1 signal peptide, consisting of 32 amino acid residues derived from parts of exons 2 and 3 of the IGF-1 gene; and the Class 3 IGF-1 signal peptide, consisting of 22 amino acid residues derived from exon 3 of the IGF-1 gene. Other suitable N-terminus amino acid sequences will be apparent to those skilled in the art.

As defined above, —B— is an optional amino acid sequence consisting of b amino acids. The value of b is generally at least 1 (e.g. at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, 100, 150, 200, 250, 300, 350, 400, 450, 500, etc.), but can be zero (i.e. —B— is absent). One example of a suitable entity for B includes the mature 70 amino acid IGF-1 peptide derived from parts of exons 3 and 4 of the IGF-1 gene. For example, a fusion peptide including the mature IGF-1 form lined either to the Ea or Eb IGF-1 peptide might be used to act as a biologically inactive propeptide that is cleaved when required, thereby to elicit its cell regulating effects.

As defined above, —C— is a sequence derived from an IGF-1 Ea, Eb or En peptide. The function of —C— is to act as a regulator of cellular growth, as set out above. In instances where —C— is the isolated IGF-1 Ea, Eb or En peptide, the peptide will be constitutively active as a regulator of cellular growth. Alternatively, when expressed as a fusion with other peptide entities, by tailoring the identity of the fusion partner, various effects can be achieved. For example, when tethered to the mature 70 amino acid IGF-1 peptide, the fusion will act as a kind of proprotein, which can be administered systemically to the circulation of a patient to provide function when and where this is required. Furthermore, when tethered to the mature 70 amino acid IGF-1 peptide and any one of the classes of signal peptide, the fusion protein will act as a prepropeptide, which in line with the Applicant's theory described above, may perform the same or overlapping functions as the isolated E peptides.

In some embodiments, the amino acid sequence of —C— shares less than x% sequence identity to the b amino acids which are N-terminal of sequence —C— in the specific protein from which —C— is derived. In general, the value of x is 60 or less (e.g. 50, 40, 30, 20, 10 or less).

As defined above, —D—is an optional C-terminus amino acid sequence consisting of d amino acids. The value of d is generally at least 1 (e.g. at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, 100, 150, 200, 250, 300, 350, 400, 450, 500, etc.), but can be zero (i.e. —D— is absent). Examples of typical —D— moieties include sequences to direct protein trafficking, short peptide sequences which facilitate cloning or purification (e.g. comprising histidine tags i.e. Hisₙ where n=3, 4, 5, 6, 7, 8, 9, 10 or more), or sequences which enhance protein stability. Other suitable C-terminus amino acid sequences will be apparent to those skilled in the art. In certain embodiments, the function of —D— is to facilitate expression of the protein in an expression system.

The value of a+d may be 0 or greater (e.g. at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, 100, 150, 200, 250, 300, 350, 400, 450, 500 etc.). It is preferred that the value of a+d is at most 1000 (e.g. at most 900, 800, 700, 600, 500, 450, 400, 350, 300, 250, 200, 190, 180, 170, 160, 150, 140, 130, 120, 110, 100, 90, 80, 70, 60, 50, 40, 30, 25, 20, 19, 18, 17, 16, 15, 14, 13, 12, 11, 10, 9, 8, 7, 6, 5, 4, 3, 2).

Preferably, component —B—C— of the above-noted formula comprises a fusion of the mature 70 amino acid IGF-1 peptide with the IGF-1 Ea, Eb or En peptide (SEQ ID NO:2, SEQ ID NO:4, SEQ ID NO:22, SEQ ID NO:24, SEQ ID NO:26 or SEQ ID NO:28), or is a functional equivalent thereof.

In some polypeptides, the amino acid sequences of the —A—, —B—, —C— and —D— moieties may contain m amino acid substitutions, where m is an integer. The m amino acids are typically substituted by A, C, D, E, F, G, H, I, K, L, M, N, P, Q, R, S, T, V, W, or Y. Each of the m substitutions may be the same or different as the others. The substitution is preferably by G or, more preferably, by A. The substituting amino acid may be an L or a D amino acid but, where the other amino acids all share a single stereo-configuration (i.e. all D or all L), the substituting amino acid preferably also has that stereo-configuration (although, of course, G has no stereoisomers).

The invention also provides a peptide, comprising amino acid sequence —A—B—C—D—, wherein: —A— is an optional methionine residue; —B— is an optional amino acid sequence with at least a% sequence identity to SEQ ID NO:12, SEQ ID NO:30 or SEQ ID NO:32; and —C— is an amino acid sequence with at least b% sequence identity to SEQ ID NO:2, SEQ ID NO:4, SEQ ID NO:22, SEQ ID NO:24, SEQ ID NO:26 or SEQ ID NO:28; —D— is an optional amino acid sequence.

The value of a is 50 or more. The value of b is 50 or more. The value of c is 50 or more. The value of d is 50 or more. The values of a, b, c and d are independent of each other, and typical values are 60, 70, 80, 90, 95, 96, 97, 98, 99 or 100. Preferably, the value of d is 100.

Preferably, the peptide comprises SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30 or SEQ ID NO:32 or is a functional equivalent thereof. More preferably, the peptide consists of SEQ ID NO:2, SEQ ID NO:4, SEQ ID NO:22, SEQ ID NO:24, SEQ ID NO:26 and/or SEQ ID NO:28, or is a functional equivalent thereof.

The present invention also provides truncations of the peptides of the invention. For example, the N-terminus may be truncated by at least 1, 2, 3, 4, 5, 6, 7, 8, 9, 10, 11, 12, 13, 14, 15, 16, 17, 18, 19 20 or more.

Peptides of the invention (including oligopeptides and polypeptides, collectively “peptides”) may be linear, branched or cyclic, but they are preferably linear chains of amino acids. Where cysteine residues are present, peptides of the invention may be linked to other peptides via disulphide bridges. Peptides of the invention may comprise L-amino acids and/or D-amino acids. The inclusion of D-amino acids may be preferred in order to confer resistance to mammalian proteases.

The N-terminus residue of a peptide of the invention may be covalently modified. Suitable covalent groups include, but are not limited to: acetyl (as in Fuzeon™); a hydrophobic group; carbobenzoxyl; dansyl; T-butyloxycarbonyl; amido; 9-fluorenylmethoxy-carbonyl (FMOC); a lipid; a fatty acid; polyethylene; carbohydrate; etc.

Similarly, the C-terminus residue of a peptide may be covalently modified (e.g. carboxamide, as in Fuzeon™, etc.). Suitable covalent groups include, but are not limited to: acetyl; a hydrophobic group; amido; carbobenzoxyl; dansyl; T-butyloxycarbonyl; 9-fluorenylmethoxy-carbonyl (FMOC); a lipid; a fatty acid; polyethylene; carbohydrate; etc.

Peptides of the invention may be produced by various means.

A preferred method for production is biological synthesis, e.g. the peptides may be produced by translation. This may be carried out in vitro or in vivo. Biological methods are in general restricted to the production of peptides based on L-amino acids, but manipulation of translation machinery (e.g. of aminoacyl-tRNA molecules) can be used to allow the introduction of D-amino acids (or of other non-natural amino acids, such as iodotyrosine or methylphenylalanine, azidohomoalanine, etc.) {28}.

Production of peptides by biological means gives peptides with an N-terminus methionine residue. Where the N-terminus of a peptide of the invention is not a methionine then this residue (and any other extraneous residues) will have to be removed e.g. by proteolytic digestion.

The invention also provides a purified nucleic acid molecule which encodes a polypeptide according to any of the above embodiments of the invention.

Preferably, the purified nucleic acid molecule comprises the nucleic acid sequence as recited in SEQ ID NO:1 (encoding the Mouse Ea peptide protein sequence), SEQ ID NO:3 (encoding the Mouse Eb peptide protein sequence), SEQ ID NO:5 (encoding the Mouse Class 1 IGF-1 signal peptide protein sequence), SEQ ID NO:7 (encoding the Mouse Class 2 IGF-1 signal peptide protein sequence), SEQ ID NO:9 (encoding the Mouse Class 3 IGF-1 signal peptide protein sequence), SEQ ID NO:11 (encoding the Mouse mature IGF-1 peptide protein sequence), SEQ ID NO:13 (encoding Human Class 1 IGF-1 signal peptide protein sequence), SEQ ID NO:15 (encoding the Mouse Class 1 IGF-1 signal peptide protein sequence), SEQ ID NO:17 (encoding the Human Class 2 IGF-1 signal peptide protein sequence), SEQ ID NO:19 (encoding the Mouse Class 2 IGF-1 signal peptide protein sequence), SEQ ID NO:21 (encoding the Human Ea peptide protein sequence), SEQ ID NO:23 (encoding the protein sequence referred to in the literature as the Human Ec peptide), SEQ ID NO:25 (encoding the protein sequence referred to in the literature as the Human Eb peptide), SEQ ID NO:27 (encoding the Mouse En peptide protein sequence), SEQ ID NO:29 (encoding the Human mature IGF-1 peptide protein sequence) or SEQ ID NO:31 (encoding the Mouse mature IGF-1 peptide protein sequence) or is a redundant equivalent or fragment of any one of these sequences.

The invention further provides that the purified nucleic acid molecule consists of the nucleic acid sequences as recited in SEQ ID NO:1 (encoding the Mouse Ea peptide protein sequence), SEQ ID NO:3 (encoding the Mouse Eb peptide protein sequence), SEQ ID NO:5 (encoding the Mouse Class 1 IGF-1 signal peptide protein sequence), SEQ ID NO:7 (encoding the Mouse Class 2 IGF-1 signal peptide protein sequence), SEQ ID NO:9 (encoding the Mouse Class 3 IGF-1 signal peptide protein sequence), SEQ ID NO:11 (encoding the Mouse mature IGF-1 peptide protein sequence), SEQ ID NO:13 (encoding Human Class 1 IGF-1 signal peptide protein sequence), SEQ ID NO:15 (encoding the Mouse Class 1 IGF-1 signal peptide protein sequence), SEQ ID NO:17 (encoding the Human Class 2 IGF-1 signal peptide protein sequence), SEQ ID NO:19 (encoding the Mouse Class 2 IGF-1 signal peptide protein sequence), SEQ ID NO:21 (encoding the Human Ea peptide protein sequence), SEQ ID NO:23 (encoding the protein sequence referred to in the literature as the Human Ec peptide), SEQ ID NO:25 (encoding the protein sequence referred to in the literature as the Human Eb peptide), SEQ ID NO:27 (encoding the Mouse En peptide protein sequence), SEQ ID NO:29 (encoding the Human mature IGF-1 peptide protein sequence) or SEQ ID NO:31 (encoding the Mouse mature IGF-1 peptide protein sequence) or is a redundant equivalent or fragment of any one of these sequences.

The nucleic acid may be DNA or RNA (or hybrids thereof), or their analogues, such as those containing modified backbones (e.g. phosphorothioates) or peptide nucleic acids (PNA). It may be single-stranded (e.g. mRNA) or double-stranded, and the invention includes both individual strands of a double-stranded nucleic acid (e.g. for antisense, priming or probing purposes). It may be linear or circular. It may be labelled. It may be attached to a solid support.

Nucleic acid according to the invention can, of course, be prepared in many ways e.g. by chemical synthesis (e.g. phosphoramidite synthesis of DNA) in whole or in part, by nuclease digestion of longer molecules, by ligation of shorter molecules, from genomic or cDNA libraries, by use of polymerases etc.

Accordingly, the present invention also provides vectors (e.g. plasmids) comprising nucleic acid of the invention (e.g. expression vectors and cloning vectors) and host cells (prokaryotic or eukaryotic) transformed with such vectors.

The invention also provides a process for producing a peptide of the invention, comprising the step of culturing a host cell transformed with nucleic acid of the invention under conditions that induce expression of the peptide.

Suitable expression systems for use in the present invention are well known to those of skill in the art and many are described in detail in references 29 and 30.

Generally, any system or vector that is suitable to maintain, propagate or express nucleic acid molecules to produce a peptide in the required host may be used. The appropriate nucleotide sequence may be inserted into an expression system by any of a variety of well-known and routine techniques, such as, for example, those described in 29.

Generally, the encoding gene can be placed under the control of a control element such as a promoter, ribosome binding site (for bacterial expression) and, optionally, an operator, so that the DNA sequence encoding the desired peptide is transcribed into RNA in the transformed host cell.

Examples of suitable expression systems include, for example, chromosomal, episomal and virus-derived systems, including, for example, vectors derived from: bacterial plasmids, bacteriophage, transposons, yeast episomes, insertion elements, yeast chromosomal elements, viruses such as baculoviruses, papova viruses such as SV40, vaccinia viruses, adenoviruses, fowl pox viruses, pseudorabies viruses and retroviruses, or combinations thereof, such as those derived from plasmid and bacteriophage genetic elements, including cosmids and phagemids. Human artificial chromosomes (HACs) may also be employed to deliver larger fragments of DNA than can be contained and expressed in a plasmid.

Particularly suitable expression systems include microorganisms such as bacteria transformed with recombinant bacteriophage, plasmid or cosmid DNA expression vectors; yeast transformed with yeast expression vectors; insect cell systems infected with virus expression vectors (for example, baculovirus); plant cell systems transformed with virus expression vectors (for example, cauliflower mosaic virus, CaMV; tobacco mosaic virus, TMV) or with bacterial expression vectors (for example, Ti or pBR322 plasmids); or animal cell systems. Cell-free translation systems can also be employed to produce the peptides of the invention.

For long-term, high-yield production of a recombinant peptide, stable expression is preferred. For example, cell lines that stably express the peptide of interest may be transformed using expression vectors which may contain viral origins of replication and/or endogenous expression elements and a selectable marker gene on the same or on a separate vector. Following the introduction of the vector, cells may be allowed to grow for 1-2 days in an enriched media before they are switched to selective media. The purpose of the selectable marker is to confer resistance to selection, and its presence allows growth and recovery of cells that successfully express the introduced sequences. Resistant clones of stably transformed cells may be proliferated using tissue culture techniques appropriate to the cell type.

Mammalian cell lines available as hosts for expression are known in the art and include many immortalised cell lines available from the American Type Culture Collection (ATCC) including, but not limited to, Chinese hamster ovary (CHO), HeLa, baby hamster kidney (BHK), monkey kidney (COS), C127, 3T3, BHK, HEK 293, Bowes melanoma and human hepatocellular carcinoma (for example Hep G2) cells and a number of other cell lines.

In the baculovirus system, the materials for baculovirus/insect cell expression systems are commercially available in kit form from, inter alia, Invitrogen, San Diego Calif. (the “MaxBac” kit). These techniques are generally known to those skilled in the art and are described fully in reference 31. Particularly suitable host cells for use in this system include insect cells such as Drosophila S2 and Spodoptera Sf9 cells.

There are many plant cell culture and whole plant genetic expression systems known in the art. Examples of suitable plant cellular genetic expression systems include those described in references 32, 33, 34 and 35. In particular, all plants from which protoplasts can be isolated and cultured to give whole regenerated plants can be utilised, so that whole plants are recovered which contain the transferred gene. Practically all plants can be regenerated from cultured cells or tissues, including but not limited to all major species of sugar cane, sugar beet, cotton, fruit and other trees, legumes and vegetables.

Examples of particularly preferred prokaryotic expression systems include those that use streptococci, staphylococci, E. coli, Streptomyces and Bacillus subtilis as host cells. Examples of particularly suitable fungal expression systems include those that use yeast (for example, S. cerevisiae) and Aspergillus as host cells.

An alternative to biological synthesis for producing peptides of the invention involves in vitro chemical synthesis {36, 37}. Solid-phase peptide synthesis is particularly preferred, such as methods based on t-Boc or Fmoc {38} chemistry. Enzymatic synthesis {39} may also be used in part or in full. Where D-amino acids are included in peptides of the invention it is preferred to use chemical synthesis.

Accordingly, the invention also provides a process for producing a peptide of the invention, comprising the step of synthesising the peptide by chemical means. The peptide may be synthesised in whole or in part by such chemical means.

Peptides of the invention are useful regulators of cellular growth in their own right. However, they may be refined to improve this regulatory activity or to improve pharmacologically important features such as bioavailability, toxicology, metabolism, pharmacokinetics, etc. The peptides may therefore be used as lead compounds for further research and refinement.

The invention provides a pharmaceutical composition comprising (a) a peptide of the invention and (b) a pharmaceutical carrier.

Component (a) is the active ingredient in the composition, and this is present at a therapeutically effective amount e.g. an amount sufficient to inhibit infection. The precise effective amount for a given patient will depend upon their size and health, the nature and extent of infection, and the composition or combination of compositions selected for administration. The effective amount can be determined by routine experimentation and is within the judgement of the clinician. For purposes of the present invention, an effective dose will generally be from about 0.01 mg/kg to about 5 mg/kg, or about 0.01 mg/kg to about 50 mg/kg or about 0.05 mg/kg to about 10 mg/kg. Pharmaceutical compositions based on peptides are well known in the art (e.g. FUZEON™). Peptides may be included in the composition in the form of salts and/or esters.

Carrier (b) can be any substance that does not itself induce the production of antibodies harmful to the patient receiving the composition, and which can be administered without undue toxicity. Suitable carriers can be large, slowly metabolized macromolecules such as proteins, polysaccharides, polylactic acids, polyglycolic acids, polymeric amino acids, amino acid copolymers, and inactive virus particles. Such carriers are well known to those of ordinary skill in the art. Pharmaceutically acceptable carriers can include liquids such as water, saline, glycerol and ethanol. Auxiliary substances, such as wetting or emulsifying agents, pH buffering substances, and the like, can also be present in such vehicles. Liposomes are suitable carriers. A thorough discussion of pharmaceutical carriers is available in reference 40.

The carriers may be liposomes. “Liposome” refers to a generally spherical cluster or aggregate of amphipathic compounds, including lipid compounds, typically in the form of one or more concentric layers, for example, monolayers and/or bilayers. The liposomes may be formulated, for example, from ionic lipids and/or non-ionic lipids. The preparation of suitable liposomes would be well known to those of skill in the art (see, for example, reference 41). The peptide may be incorporated in the liposome in a variety of ways. Generally speaking, the peptide may be incorporated by being associated covalently or non-covalently with one or more of the materials which are included in the liposomes. In a preferred embodiment, the peptide is incorporated in the liposome via non-covalent associations. As known to those skilled in the art, non-covalent association is generally a function of a variety of factors, including, for example, the polarity of the involved molecules and the charge (positive or negative), if any, of the involved molecules, and the like. Non-covalent bonds are preferably selected from the group consisting of ionic interaction, dipole-dipole interaction, hydrogen bonds, hydrophilic interactions, van der Waal's forces, and any combinations thereof. Preferably, the peptide is incorporated in the liposome by means of a transmembrane domain that forms part of the peptide. Preferably, the peptide is incorporated in the liposome such that sequence derived from an HR2 domain is on the outside face of the liposome.

Pharmaceutical compositions of the invention may be prepared in various forms. For example, the compositions may be prepared as injectables, either as liquid solutions or suspensions. Solid forms suitable for solution in, or suspension in, liquid vehicles prior to injection can also be prepared. The composition may be prepared for topical administration e.g. as an ointment, gel, cream or powder. The composition may be prepared for oral administration e.g. as a tablet or capsule, or as a syrup (optionally flavoured). The composition may be prepared for pulmonary administration e.g. as an inhaler, using a fine powder or a spray. The composition may be prepared as a suppository or pessary. The composition may be prepared for nasal, aural or ocular administration e.g. as drops, as a spray, or as a powder (as described in reference 42). The composition may be lyophilised.

The pharmaceutical composition is preferably sterile. It is preferably pyrogen-free. It is preferably buffered e.g. at between pH 6 and pH 8, generally around pH 7.

The invention also provides a delivery device containing a pharmaceutical composition of the invention. The device may be, for example, a syringe or an inhaler.

The invention provides a peptide of the invention for use as a medicament. The invention also provides a method for treating a subject suffering from or at risk of contracting a disease or medical condition, comprising administering to the subject a pharmaceutical composition of the invention. The invention also provides the use of a pharmaceutical composition of the invention in the manufacture of a medicament for treating a subject.

Particular conditions that may be treated by the pharmaceutical compositions of the present invention include traumatic skeletal muscle injury, muscle wasting, amyotrophic lateral sclerosis and myocardial infarction.

The subject is preferably a mammal, more preferably a human. The human may be an adult or a child. A composition intended for children may also be administered to adults e.g. to assess safety, dosage, immunogenicity, etc.

Compositions of the invention will generally be administered directly to a subject. Direct delivery may be accomplished by parenteral injection (e.g. subcutaneously, intraperitoneally, intravenously, intramuscularly, or to the interstitial space of a tissue), or by rectal, oral (e.g. tablet, spray), vaginal, topical, transdermal or transcutaneous, intranasal, pulmonary or other mucosal administration.

Dosage treatment can be a single dose schedule or a multiple dose schedule.

Gene therapy may be employed to effect the endogenous production of the peptide by the relevant cells in the subject. Gene therapy is used to treat permanently the inappropriate production of the peptide by replacing a defective gene with a corrected therapeutic gene.

Gene therapy of the present invention can occur in vivo or ex vivo. Ex vivo gene therapy requires the isolation and purification of patient cells, the introduction of a therapeutic gene and introduction of the genetically altered cells back into the patient. In contrast, in vivo gene therapy does not require isolation and purification of a patient's cells.

Ex vivo gene therapy may also involve the isolation and purification of adult stem cells, the introduction of a gene encoding a peptide of the invention and introduction of the genetically altered adult stem cells into the patient. For example, circulating bone-marrow derived cells may be used to target a tissue restricted gene expression cassette, encoding an E peptide, to damaged, inflamed or degenerating tissues. More specifically myeloid progenitor cells may be used for direct muscle delivery. These cells migrate to sites of injury as a normal component of the inflammation response, where they differentiate into a number of cell types, including macrophages which increase muscle satellite cell proliferation and enhance regeneration (43). Myeloid progenitors have recently been shown to fuse directly with differentiated skeletal muscle fibres and transdifferentiate along a myogenic lineage (44). Endogenous myeloid cell fusion is normally a rare event, however once incorporated into the damaged muscle bed, activation of the E peptide gene in the new fused myeloid nuclei will enhance subsequent recruitment and incorporation of additional circulating cells, thereby augmenting the overall incorporation of the E peptide gene-carrying myeloid cells over time. Repeated administration of engineered myeloid cells carrying the E peptide gene may optimise gene uptake in chronic degenerative conditions.

The therapeutic gene is typically “packaged” for administration to a patient. Gene delivery vehicles may be non-viral, such as liposomes, or replication-deficient viruses, such as adenovirus as described by Berkner (45) or adeno-associated virus (AAV) vectors as described by Muzyczka, (46; 47). For example, a nucleic acid molecule encoding a polypeptide of the invention may be engineered for expression in a replication-defective retroviral vector. This expression construct may then be isolated and introduced into a packaging cell transduced with a retroviral plasmid vector containing RNA encoding the peptide, such that the packaging cell now produces infectious viral particles containing the gene of interest. These producer cells may be administered to a subject for engineering cells in vivo and expression of the peptide in vivo (48).

Another approach is the administration of “naked DNA” in which the therapeutic gene is directly injected into the bloodstream or muscle tissue.

The uses and methods of the invention can be used therapeutically (e.g. for treating an existing infections) or prophylactically (e.g. in a situation where disease is expected and where establishment of disease is to be prevented). Therapeutic use is preferred, and efficacy of treatment can be tested by monitoring the patient after administration of the pharmaceutical composition of the invention, such as by monitoring symptoms.

The invention also includes the use of a peptide according to any of the above-described aspects of the invention, or a functional equivalent thereof, a nucleic acid molecule encoding said peptide or functional equivalent, in the manufacture of a medicament for increasing muscle hypertrophy.

The invention also includes the use of a peptide according to any of the above-described aspects of the invention, or a functional equivalent thereof, a nucleic acid molecule encoding said peptide or functional equivalent, in the manufacture of a medicament for decreasing muscle atrophy.

The invention also includes the use of a peptide according to any of the above-described aspects of the invention, or a functional equivalent thereof, a nucleic acid molecule encoding said peptide or functional equivalent, in the manufacture of a medicament for increasing cell proliferation and/or differentiation, for example, for purposes such as any type of wound healing including tissue damage associated with cardiovascular disorders, skeletal muscle injury, increasing tissue (e.g. muscle) growth following trauma, enhancing repair of skeletal muscle damage, enhancement of exercise-induced hypertrophy, reversal of age-related atrophy and prevention of dystrophic muscle degeneration.

The invention also includes the use of a peptide according to any of the above-described aspects of the invention, or a functional equivalent thereof, a nucleic acid molecule encoding said peptide or functional equivalent, in the manufacture of a medicament for attenuation of neuronal degeneration.

The term “comprising” encompasses “including” as well as “consisting of” e.g. a composition “comprising” X may consist exclusively of X or may include something additional e.g X+Y.

The term “about” in relation to a numerical value x means, for example, x±10%.

The word “substantially” does not exclude “completely” e.g. a composition which is “substantially free” from Y may be completely free from Y. Where necessary, the word “substantially” may be omitted from the definition of the invention.

The term “functional equivalent”, as used herein, refers to a sequence that has an analogous function to the sequence of which it is a functional equivalent. By “analogous function” is meant that the sequences share a common function, for example, in the regulation of cellular growth or differentiation, and, in some embodiments, a common evolutionary origin. In some embodiments, a functionally equivalent sequence may exhibit sequence identity with the sequence of which it is a functional equivalent. Preferably, the sequence identity between the functional equivalent and the sequence of which it is a functional equivalent is at least 50% across the length of the functional equivalent. More preferably, the identity is at least 60% across the length of the functional equivalent. Even more preferably, identity is greater than 70%, 80%, 85%, 90%, 95%, 96%, 97%, 98% or 99% across the length of the functional equivalent. Functional equivalents include mutants of the sequences of which they are functional equivalents, i.e. containing amino acid substitutions, insertions or deletions from said sequence, provided that function is retained. Functional equivalents with improved function compared to the sequences of which they are functional equivalents may be designed through the systematic or directed mutation of specific residues in said sequences. Functional equivalents include sequences containing conservative amino acid substitutions that do not affect the function or activity of the sequence in an adverse manner.

References to a percentage sequence identity between two amino acid sequences means that, when aligned, a percentage of the amino acids are the same in the two sequences. This alignment and the percent homology or sequence identity can be determined using software programs known in the art, for example those described in section 7.7.18 of reference 49. A preferred alignment is determined by the Smith-Waterman homology search algorithm using an affine gap search with a gap open penalty of 12 and a gap extension penalty of 2, BLOSUM matrix of 62. The Smith-Waterman homology search algorithm is disclosed in reference 50.

The use of “NH2” and “COOH” in peptide sequences implies only the direction of the peptide chain from N-terminus to C-terminus, and does not imply that the N-terminus residue must have a free —NH₂ group or that the C-terminus must have a free —COOH group (although nor is such a situation excluded). On the contrary, the N- and C-termini may be covalently modified.

Preferably, the IGF-1 Ea peptide of the invention will be glycosylated. However, it may not be glycosylated or partially glycosylated in some embodiments. For example, when expressed so as to include the Class 1 signal peptide, the Ea peptide is glycosylated. When expressed so as to include the Class 2 signal peptide, the Ea peptide is not glycosylated. To achieve correct glycosylation patterns reflective of the human molecule as it occurs naturally, recombinant molecules may either be produced in mammalian (preferably human) cell culture or may be expressed in bespoke recombinant systems such as yeast that have been modified to co-express the necessary glycosylation enzymes (e.g. Glycofi Inc.).

Although the Applicant does not wish to be limited or bound by any particular theory, it is postulated herein that the distribution pattern of the IGF-1 E peptide can be altered by the class of signal peptide encoded by the nucleic acid encoding a peptide of the invention. For example, when expressed so as to include the Class 1 signal peptide, the IGF-1 E peptide has been shown to act in a paracrine manner. In contrast, when expressed so as to include the Class 2 signal peptide, the IGF-1 E peptide has been shown to act in an endocrine manner. As a consequence of the Applicant's theory, the invention also provides for IGF-1 E peptides, comprising the class 1, class 2 or class 3 signal peptide, specifically designed for either localised or systemic delivery. These signal peptides may be used to direct the delivery of a peptide of the invention in any of the forms described above. For example, situations can be envisaged wherein the desired response, according to any aspect of the invention, can be achieved through the delivery of an E peptide of the invention to a particular cell or tissue type. Alternatively, it may be necessary to deliver an E peptide in conjunction with the mature IGF-1 peptide to achieve the desired response. Further still, a peptide of the invention may be delivered under the control of the various signal peptides, in conjunction with other useful peptides, for example, human growth hormone, optionally linked as a multipeptide unit. Alternatively, it is postulated herein that the fate of the various IGF-1 isoforms may be regulated at a tissue type level. For example, IGF-1 isoforms expressed in skeletal muscle tissue have been shown to act in a paracrine manner. It may therefore be that the cleavage pattern of IGF-1 in skeletal muscle differs from that evident in tissues such as the liver, and this endows the resulting peptides with a different cellular fate or extracellular distribution.

The invention also includes a transgenic animal comprising a nucleic acid encoding a peptide of the invention or a functional equivalent thereof or a fusion protein as defined above. Such transgenic animals may in particular include sheep, pigs, cows, chickens, goats and fish. Particular commercial utilities of such animals may be evident from an increased size, or an increased edible volume, provided by such animals.

Various aspects and embodiments of the present invention will now be described in more detail by way of example. It will be appreciated that modification of detail may be made without departing from the scope of the invention.

### BRIEF DESCRIPTION OF FIGURES

FIG. 1. Muscle Atrophy and Increased Activity of the Proteasome in Mice with Left Ventricular Dysfunction. A, Skeletal muscle atrophy develops in mice with CLVD. B, Increased protein ubiquitination in skeletal muscle of mice with CLVD. C, Activity of the 20S proteasome increases in several atrophying muscles in chronic CLVD (all P<0.05 vs. WT sham) (EDL−M. extensor digitorum longus; SOL−M. soleus; QUA−M. quadriceps; n=6 animals per datapoint).

FIG. 2. Transgenic Overexpression of mIGF-1 Prevents Muscle Atrophy and Increased Proteasome Activity in Left Ventricular Dysfunction. A, Transgenic overexpression of MLC/mIGF-1 prevented the development of muscular atrophy in animals with CLVD. B, Increased protein ubiquitination in skeletal muscle of mice with CLVD is absent in MLC/mIGF-1 mice. C, Overexpression of mIGF-1 blocks enhanced 20S proteasome activity in chronic CLVD (EDL−M. extensor digitorum longus; SOL−M. soleus; QUA−M. quadriceps; n=6 animals per datapoint).

FIG. 3. Expression of atrogin-1/MAFbx. A, Expression of atrogin-1/MAFbx increases in several muscles of animals with CLVD. B, Quantitative real-time PCR revealed increased of atrogin-1/MAFbx expression in the atrophying quadriceps muscle of animals with CLVD (*P<0.05 vs. WT sham; n=6 animals per datapoint) which is prevented by transgenic overexpression of mIgf-1. C, In SOL8 cells, serum starvation for 72 h increases atrogin-1/MAFbx expression which is reduced by serum incubation and stimulation with IGF-1 for 3 h (data from 3 independent experiments).

FIG. 4. Ubiquitin-mediated Proteolysis of MyHC. A, Analysis of MyHC ubiquitination in vitro. While Western analysis shows no differences in overall protein levels between dexamethasone-treated (10 ng/ml for 24 h) cells and controls, ubiquitination of MyHC is robustly enhanced under dexamethasone compared to controls. B, Increased ubiquitination of MyHC in atrophying skeletal muscle 12 weeks after coronary artery ligation and development of CLVD. Expression of the MLC/mIGF-1 transgene prevents the increase in ubiquitination of MyHC in skeletal muscles of animals with CLVD.

FIG. 5. Reduced of Akt Phosphorylation and FOXO Activation in Atrophying Skeletal Muscle is Prevented in MLC/mIGF-1 mice. A, Skeletal muscle of mice with CLVD shows reduced Akt activity compared to controls while overexpression of MLC/mIGF-1 enhances Akt activity without changes in animals with CLVD (n=3 per datapoint). B, Increased activation of FOXO 4 in skeletal muscle of mice with CLVD compared to controls. Transgenic overexpression of mIGF-1 blocks activation of FOXO transcription factors by enhancing FOXO phosphorylation (n=3 per datapoint).

FIG. 6. Characterisation of MHC/mIGF-1 transgenic mice. (A) Schematic representation of the rodent Igfl gene. (B, C) Northern blot analysis of total RNA (10 μg) from different aged wild type and transgenic hearts, using the rat IGF-1 32P-labeled probe. Ethidium Bromide was used to verify equal RNA loading amount and RNA integrity.

FIG. 7. Physiological analysis of mIGF-1 transgenic hearts. (A) Histological analysis of wild-type and transgenic hearts by Hematoxylin and Eosin staining. The relative increase in heart weight/body weight (p-value<0.05) of transgenic hearts is resolved by six months. Values are the average of six independent analyses. (B) RT-PCR of different hypertrophic markers in adult hearts. 0.5 μg of total RNA was used for each single PCR. PCR values were normalised for actin content. (C) Western blot analysis of AKT and S6 ribosomal protein phosphorylation. 50 μg of total cell extracts at different age of postnatal heart development were loaded onto SDS-PAGE gel. Total amount of proteins was normalised for AKT and S6 ribosomal protein.

FIG. 8. Representative electrocardiograms obtained in non-transgenic (NTG) and IGF-1 transgenic (TG) mice in the D2 derivation. The plain arrows indicate the P waves that are amplified in the transgenic mice. The dotted arrows indicate a prolongation and non-homogenous depolarisation of the ventricles in the TG mouse.

FIG. 9. Full cardiac regeneration in mIGF-1 transgenic mice. (A, B and C) Thricrome staining of 4 months old wild-type and transgenic hearts at 48 hours, 1 week and 1 month after CTX injection in left ventricular wall. Comparable results were obtained with similar analyses on six different groups of animals

FIG. 10. Early events characterising mIGF-1 induced regeneration. (A) RT-PCR of inflammatory interleukins IL6 and IL1β 24 hours after cardiotoxin injection in wild-type and transgenic hearts. PCR was normalized by actin content in each sample. (B) Real time PCR of the anti-inflammatory cytokines IL10 and IL4 in transgenic (gray bars) and wild-type (white bars) hearts 24 hour and 1 week after cardiotoxin injection. The results are the average of three independent experiments. (C) Expression of p21WAF1/CIP1 in wild-type and transgenic hearts injected with cardiotoxin. Actin was used to verify equal protein loading amount.

FIG. 11. Cells proliferation accompanies mIGF-1 induced heart regeneration. BrdU was provided ad libidum for 1 month after cardiotoxin injection at 0.1%. Paraffin sections of 10 μm were stained with a biotinalated mouse monoclonal antibody to visualized nuclei that incorporated BrdU in wild type (A) and transgenic (B) hearts. All the sections where the injury was evident were analysed. Cardiac cells (C) and cells of other lineage (D) were observed around the injury.

FIG. 12. mIGF-1 expression delays the progression of the disease and enhances the survival of SOD1_{G93A} mutant mice (a) Western blot analysis for human SOD transgenic protein in wild type (lane 1), MLC/mIGF-1 (lane 2), SOD1_{G93A} (lanes 3) and SOD_{G9C3A}mIGF-1 (lanes 4) transgenic mice. (b) Northern blot analysis for mIGF-1 transgene expression in skeletal muscle of wild type (lane 1), MLC/mIGF-1 (lane 2), SOD1_{G93A} (lanes 3) and SOD_{G93A}mIGF-1 (lanes 4) transgenic mice; in brain of MLC/mIGF-1 (lane 5), SOD_{G93A}mIGF-1 (lanes 6) mice; in spinal cord of MLC/mIGF-1 (lane 5), SOD_{G93A}mIGF-1 (lanes 6) mice. (c) Age of onset of disease symptoms in SOD1_{G93A} and SOD_{G93A}mIGF-1 mice. (d) Analysis of the progression of the disease in SOD1_{G93A} and SOD_{G93A}mIGF-1 mice. (e) Survival analysis of SOD1_{G93A} and SOD_{G93A}mIGF-1 mice.

FIG. 13. mIGF-1 expression attenuates muscle wasting and promotes regenerative pathways in SOD1_{G93A} mice. (a) Muscle histological analysis of wild type (A), MLC/mIGF-1 (B), SOD1_{G93A} (C, E) and SOD_{G93A}mIGF-1 (D, F) mice at different age and stage of disease. (b) Analysis of fiber type size differences in the quadriceps underscores the relative attenuation of muscle atrophy in SOD_{G93A}mIGF-1 compared to SOD1_{G93A} mice. (c) Western blot analysis for molecular markers of muscle regeneration, activated satellite cells, and maturation (Pax 7, desmin, myogenin, and neo-MyHC). Muscle protein lysates were obtained from quadriceps of wild type (lane 1), MLC/mIGF-1 (lane 2), SOD1_{G93A} (lanes 3, 5) and SOD_{G93A}mIGF-1 (lanes 4, 6) transgenic mice at different ages of the clinical disease (lanes 3 and 4 at 28 days of age; lanes 5 and 6 at 123 days of age). Immunoblotting for α-tubulin served as a control for protein loading. (c) Immunofluorescence analysis for MyHC-fast performed on Soleus muscles of wild type, SOD1_{G93A} and SOD_{G93A}mIGF-1 before (80 days) and after symptom onset (123, 138 days of age). Bar, 50 μm. (d) Walk test of SOD1_{G93A} (black circles) and SOD_{G93A}mIGF-1 (white circles) transgenic mice. The expression of mIGF-1 maintained the functional performance of SOD1_{G93A} skeletal muscle.

FIG. 14. Transgenic mIGF-1 expression induces chronic CnA-β1 expression in SOD1_{G93A} mice. (a) Northern blot analysis for the CnA-β1 of wild type (lane 1), MLC/mIGF-1 (lane 2), SOD1_{G93A} (lane 3), and SOD_{G93A}mIGF-1 (lane 4) transgenic mice. Ethidium bromide staining was used to verify equal loading of the RNA sample. (b) Lysates of the same muscle tissues used in FIG. 2b were tested by western blotting using

CNA-β1 specific antibody. (c) Immunofluorescence of 7 μm transverse sections from Quadriceps muscles of SOD1_{G93A} and SOD_{G93A}mIGF-1 at paralysis stage. CnA-β1 shows a nuclear localization. A regenerating fiber is indicated by the presence of central nucleus (red arrow). Nuclei were visualized by Hoechst dye (blue). Bar, 20 μm.

FIG. 15. Maintenance of the neuromuscular junction configuration in SOD1_{G93A}×MLC/mIGF-1 transgenic mice. (a) Immunofluorescent analysis of 7 μm transverse sections from muscles of wild type, MLC/mIgf-1, SOD1_{G93A} and SOD_{G93A}mIGF-1 transgenic mice at 123 days of age. α-bungarotoxin antibody identified diffusion of acetylcholine receptor (AChR) expression in SOD1_{G93A} muscle (yellow arrow); whereas AChR showed a transitory polyinnervation, as indicated by the presence of two clusters in a single fiber (red arrows). Bar, 20 μm. (b) Northern blot of total RNA samples (15 μg) from quadriceps of wild type (lane 1), MLC/mIGF-1 (lane 2), SOD1_{G93A} (lane 3), and SOD_{G93A}mIGF-1 (lanes 4, 5) transgenic muscles at 123 (lanes 1-4) and 150 (lane 5) days of age, hybridized with AChR ₃₂P-labeled probe. Ethidium bromide staining was used to verify equal loading of the RNA sample. (c) Western blot analysis for agrin from quadriceps of wild type (lane 1), MLC/mIGF-1 (lane 2) SOD1_{G93A} (lane 3) and SOD_{G93A}mIGF-1 (lane 4) transgenic muscles. SOD1_{G93A} and SOD_{G93A}mIGF-1 mice were analyzed at comparable end-stage disease.

FIG. 16. Transgenic mIGF-1 expression protects motor neuron from degeneration. (a) Quantification of surviving motoneurons in the ventral spinal cord of wild type (Wt) SOD1_{G93A} (S) and SOD_{G93A}mIGF-1 (S/I) mice at different age. *p<0.01;** p<0.001. (b) Immunofluorescence analysis identified GFAP positive astrocytes in ventral horn of SOD1_{G93A} and SOD_{G93A}mIGF-1 transgenic mice at different ages: A, B 28 days of age; C, D 123 days of age. The intensity of the GFAP signal revealed progressive astrocytosis. Bar, 20 μm. Insert in D shows Western blot analysis for GFAP in the spinal cord of SOD1_{G93A} (lanes 1, 3) and SOD_{G93A}mIGF-1 (lanes 2, 4) mice at 28 (lanes 1,2) and 123 (lanes 3,4) days of age.(c) RT-PCR analysis of TNF-α and β-actin of wild type (lane 1), MLC/mIGF-1 (lane 2), SOD1_{G93A}(lane 3) and SOD_{G93A}mIGF-1 (lane 4) transgenic mice at 123 days of age. Lane 5 shows a negative control consisting of RT-PCR mix without cDNA template. Lane 6 identifies the RNA positive control for TNF-α obtained from spleen.

FIG. 17. A Schematic representation of the IGF-1 gene and the various signal/E peptide isoforms.

FIG. 18. The effect of IGF-1 signal peptides on myoblast differentiation.

FIG. 19. The effect of the IGF-1 E peptides on myoblast growth.

FIG. 20. Schematic representation of the various IGF-1 isoforms tested in vivo and their phenotypic effect.

FIG. 21: Enhanced cardiac regeneration and functions in transgenic mice after myocardial infarction. a) Extent of fibrotic invasion 2 months after LCA. Wild-type (WT) and transgenic (TG) hearts were perfused with 4% paraformaldehyde (PFA) after avertin injection. Hearts in PFA were photograph with a Leica MZ12 stereo microscope. Arrows indicate fibrotic tissue. LA, left atrium; LV, left ventricle; RA, right atrium; RV, right ventricle. Right: trichrome staining of cardiac tissues. b) Functional recovery of mIGF-1 transgenic mice. Eight WT and TG mice were anaesthetized with avertin post-myocardial infarction (1 month and 2 months) and cardiac parameters were analysed with a high-resolution ultrasound system (VisualSonics Inc.). Mean percentage values of fractional shortening (FS, upper panel) and ejection fraction (EF, lower panel) are representative of three readings on each animal and of average among groups. * Shows significant values (p<0.05) between uninjured and injured hearts in WT (yellow square) and TG (red square) animals. § Shows significant values between WT and TG injured hearts. c) Representative echocardiographic recordings of heart function with and without injury in WT (upper panels) and TG hearts (lower panels). The heart function of WT mice 2 months after MI was dramatically impaired, confounding the reading and the recording in all animals tested.

FIG. 22: Enhanced cardiac regeneration in mIGF-1 transgenic mice after CTX injection. Functional recovery of mIGF-1 transgenic mice. Eight WT and TG mice were anaesthetized with avertin post-myocardial infarction (1 month) and cardiac parameters were analysed with high-resolution ultrasound. Left panels: WT and TG heart parameters. Right panels: mean percentages of ejection fraction (EF) and fractional shortening (FS), and mean thickness (millimetres) of the posterior wall are representative of three readings on each animal and of average among each group. LVIDs, left ventricle internal dimension in systole; LVIDd, left ventricle internal dimension in diastole; LV PWs, left ventricle posterior wall in systole; LVPWd, left ventricle posterior wall in diastole. Asterisk indicates significant values decreasing in WT compared to TG hearts (p-value<0.05).

FIG. 23: Late cell proliferation in regenerating mIGF-1 transgenic hearts. a) BrdU was provided ad libitum at 0.1% for up to 1 month after CTX infarction of WT and TG hearts or injected at 100 μg/g each day for 48 hours or 1 week. Paraffin sections of 10 μm were stained with a biotinylated mouse monoclonal antibody to visualize nuclei with BrdU incorporation. 10 sections (10 μm) bordering the injured site were analysed. b) Statistical analysis of BrdU positive cells counted at different time points after CTX injection. 10 sections for each experiment were analysed and the percentage of positive nuclei was calculated based on the amount of total nuclei present in the frame observed. No relative increases in BrdU incorporation were seen at 24 hours or 1 week post-CTX injection in either WT or TG hearts. Asterisk indicates significant relative values in the BrdU positive hearts at 1 month (p-value<0.05). Values are the average of three independent experiments.

FIG. 24: Characteristics of BrdU positive cells in regenerating mIGF-1 transgenic hearts. BrdU was provided ad libitum for up to 1 month at 0.1% after CTX injection of WT or TG hearts. a, b, c) BrdU positive cells were identified in paraffin sections of TG cardiac muscle (10 μm) stained with anti-biotinylated-BrdU antibody and photographed at 100×magnification. Arrows indicate BrdU positive cardiomyocytes (left and middle panels) and cells lining blood vessel (right panel). d) Cardiac myocytes and e) non-muscle cells were isolated from WT and TG hearts IM after CTX injection. Dissociated cell cultures were analysed for BrdU and haematoxylin to visualise proliferating nuclei. The experiment was performed on three hearts each from WT and TG animals. f) Confocal microscopic analysis of BrdU positive cells in TG heart tissue at 100×magnification. Cardiomyocytes were visualised by an anti-myosin antibody. White arrows indicate BrdU positive cardiac myocytes; non-cardiomyocyte cells are indicated by red arrows.

FIG. 25: FIG. 1. mIGF-1 enhances the activation of satellite cells. Immunofluorescent analysis of 7 um transverse sections from wild type and MLC/mIGF-1 injured muscles. Desmin antibody identified a pronounced activation of satellite cells in MLC/mIGF-1 muscle.

FIG. 26: mIGF-1 accelerates muscle regeneration. Scheme of the different phases characterising muscle regeneration in wild type and MLC/mIGF-1 transgenic mice.

FIG. 27: mIGF-1 expression negatively modulates inflammatory response during muscle regeneration. FACS analysis of molecular markers of inflammatory cells (CD11b, Gr1, CD45) in wild type and MLC/mIGF-1 transgenic injured muscle.

FIG. 28: Muscle architectures is rapidly restored in mIGF-1 injured muscle. Histological analysis of wild type and MLC/mIGF-1 transgenic muscle after 5 and 15 days post-injury.

FIG. 29: mIGF-1 improves stem cell-mediated muscle regeneration in dystrophic muscle. Histological analysis of mdx and mdx/mIGF-1 dystrophic muscle after stem cell transplantation. Brown fibers revealed that the transplanted MLC/hAP bone marrow stem cells massively participate to muscle regeneration and repair in mdx/mIGF-1 dystrophic mice.

FIG. 30 Analysis of data relating to the identification of the En isoform.

FIG. 31 Expression analysis of transiently transfected L6E9 cells. A Pattern of luciferase activity in control transfected L6E9 cells. B RT-PCR analysis on 1 μg of total RNA isolated from differently transfected cells at all time points. C Luciferase activity in IGF-1 isoform transfected L6E9 cells. Luciferase assay was performed on 51 μl of 1:500 dilutions of protein lysates prepared for every time point. Measurements were done for 10 seconds. D Western analysis of IGF-1 isoform expression. 50 μg of protein were loaded for each sample. Molecular weights (kDa) of prepro IGF-1 isoforms are indicated above the appropriate band.

FIG. 32—Morphology of IGF-1 isoform transfected L6E9 cells. Pictures were taken 24 hours (A) and 72 hours (B) after shift to differentiation medium. A After 1 day in DM, cells transfected with the Class 1 IGF-1Ea isoform showed the enhanced elongation of cells (indicated by black arrows), while cells transfected with the other 5 isoforms were less efficient in starting the differentiative response. B At day 3 of differentiation cultures transfected with Class 1 IGF-1Ea and Class 2 IGF-1Ea showed enlarged fibers, while 22-IGF-1Ea as well as the three IGF-1 isoforms containing the Eb-peptide did not show an effect on fiber size.

FIG. 33 Effects of IGF-1 isoforms during growth. 50 μg of every protein sample were used for Western blot analysis. A Histone H3 phosphorylation was lower in Class 1 IGF-1Ea and Class 2 IGF-1Ea cultures, while Eb-containing IGF-1 isoforms and the 22-IGF-1Ea showed increased phosphorylation . B Activation level of MAP-kinases JNK1, Erk1 and 2 and p38. Class 2 IGF-1Ea cultures showed lower levels of phsopho-JNK1 and Class 1 IGf-1Ea cells up-regulated phosphorylation of ERk1 and 2. The other isoforms did not induce changes in MAP-kinase phosphorylation.

FIG. 34 Effects of IGF-1 isoforms during differentiation. 50 μg of every sample were used for Western analysis. A upper panel: Induction of myogenin by IGF-1 isoforms. Lower panel: Induction of MEF2C by IGF-1 isoforms. B upper panel: Activation of Akt in IGF-1 isoform transfected cells. Lower panel: phosphorylation of S6 ribosomal protein in IGF-1 isoform expressing cells.

FIG. 35: Northern blot analysis of transgene expression of founder (Fo) lines. 10 μg of total RNA from female and male three-months-old quadriceps muscles were used for Northern analysis and probed with a SV40-specific probe A MLC/Class 1 IGF-1Ea founder. A Lanes 1 and 2 are female; lanes 3 and 4 are male samples. B MLC/Class 2 IGF-1Ea. Lanes 1, 2, 5, 6, 9, 10, 13, and 14 are female, lanes 3, 4, 7, 8, 11, 12, 15, and 16 are male samples. C MLC/Class 1 IGF-1Eb founders A and B. Lanes 1, 2, 5, and 6 are female; lanes 3, 4, 7, and 8 are male samples. D MLC/Class 2 IGF-1Eb founders A-D. Lanes 3, 4, 7, 8, 11, and 12 are female; lanes 1, 2, 5, 6, 9, 10, and 13 are male samples. E MLC/22-IGF-1Ea founder A. Lane 1 is female; lanes 2 and 3 are male samples. F MLC/22-IGF-1Eb founders A-C. Lanes 1, 6, and 7 are female; lanes 2, 3, 4, 5, and 8 are male samples.

FIG. 36: Northern comparison of selected transgenic founder line expression levels. 10 μg of total RNA form quadriceps muscle of male mice were used and probed with a SV40-specific probe. A Northern comparison of all selected founder lines for each IGF-1 isoform transgenic line. B Density of IGF-1 isoform transgene bands detected with Northern analysis (A)

FIG. 37—figure legend missing

FIG. 38 Comparison of selected lines to MLC/mIGF-1 transgenic line. 10 μg of total quadriceps RNA from male one-month-old animals was used for Northern analysis and probed with a SV40-specific probe. A Comparison of selected founder lines to MLC/mIGF-1 . B Density of IGF-1 isoform transgene bands detected with Northern analysis (A).

FIG. 39 Fold over-expression of IGF-1 isoform transgenes. The level of IGF-1 isoform over-expression was compared by quantitative RT-PCR. A Fold over-expression of IGF-1 isoforms in relation to WT total IGF-1 level. B Fold over-expression of isoforms with the wt expression level of the same isoform as a reference. For a better overview the graph was prepared without MLC/Class 2 IGF-1Eb. C Fold over-expression over wt levels of the same isoform with MLC/Class 2 IGF-1Eb

FIG. 40 Protein expression of IGF-1 isoform transgenes. 50 μg of protein from male quadriceps muscle from one-month (A) and six-months-old mice (C) were used for Western analysis with an antibody to detect all IGF-1 isoforms. Band density was determined using the Radames software for one-month-old (B) and six-month-old samples (D).

FIG. 41 Expression of endogenous isoforms. Quantitative RT-PCR was used to assess if the over-expression of IGF-1 isoforms had an effect on the expression of endogenous isoforms. The transgenic line analyzed is indicated on top of the graph. A Results for line MLC/mIGF-1 show significant up-regulation of endogenous Class 2 IGF-1Ea transcripts (p=0.04), while other isoforms were not affected. B Line MLC/Class 1 IGF-1Eb samples showed significantly more Class 1 IGF-1Ea transcripts (p=0.03), but no changes for other isoforms. No effect on endogenous isoform expression was seen in lines MLC/Class 2 IGF-1Ea and Class 2 IGF-1Eb (C and D). The values of each isoform were always compared to the WT levels of the same isoform.

FIG. 42 Northern analysis of endogenous IGF-1 isoform expression in IGF-1 transgenic mice. 10 μg of total RNA from three-month-old male quadriceps muscle from WT and transgenic mice were analyzed with the specific probes indicated. A MLC/mIGF-1 samples probed with an exon 2-specific probe showed mild induction of exon 2-containing transcripts specifically in the transgenic skeletal muscle groups. B MLC/mIGF-1 samples probed hybridized with an exon 5-specific probe revealed no changes. C No changes in MLC/Class 2 IGF-1Ea samples probed with the 186 exon 1-specific probe. D MLC/Class 2 IGF-1Ea derived samples screened with the exon 5-specific probe were also unchanged. E MLC/Class 2 IGF-1Eb samples analyzed with an exon 2-specific probe were negative. F MLC/Class 2 IGF-1Eb samples probed with the 186 exon 1-specific probe showed similar levels to WT samples.

FIG. 43 Effect of IGF-1 isoform over-expression on body weight. Number of animals is indicated at the bottom of the columns. All measurements were performed on male animals. A MLC/mIGF-1 animals show significantly elevated body weight by 1 (p=0.01) and 2-3 months of age (p=0.0001). B MLC/Class 2 IGF-1Ea mice are significantly heavier at all ages analyzed (p=0.01 for all). C MLC/Class 1 IGF-1Eb and MLC/Class 2 IGF-1Eb (D) do not have an effect on body weight.

FIG. 44 Effect of IGF-1 isoform over-expression on the weight of visceral organs. Tissue weights were taken from male transgenic mice (1, 3, and 6 months of age) and their negative littermates. The number of animals is indicated in every diagram. All organ values were normalized for bodyweight. A MLC/mIGF-1 animals did not show any effect on the weight of visceral organs. Only the weight of the EDP was significantly increased at 3 (p=0.006) and 6 months of age (p=0.03). MLC/Class 2 IGF-1Ea (B), MLC/Class 1 IGF-1Eb (C), and MLC/Class 2 IGF-1Eb animals (D) did not show an effect of the weight of distal organs.

FIG. 45 Effect of IGF-1 isoform over-expression on total circulating IGF-1 levels. All blood samples were taken from male transgenic mice and age matched WT animals (the number of animals is indicated on the bottom of the columns). A At 3 month of age MLC/mIGF-1 and MLC/Class 2 IGF-1Ea showed significantly elevated IGF-1 levels in the serum (p=0.02 and p=0.005 respectively), while serum levels of MLC/Class 1 IGF-1Eb and MLC/Class 2 IGF-1Eb levels were unchanged. B By the age of six months, only MLC/Class 2 IGF-1Ea levels were significantly increased (p=0.0001) and MLC/mIGF-1 total circulating IGF-1 levels were still elevated (7.4%). MLC/Class 1 IGF-1Ea and MLC/Class 2 IGF-1Eb showed no change.

FIG. 46 Skeletal muscle weights of IGF-1 isoform transgenic lines. One-, three-, and six-months-old male mice were used for skeletal muscle weight measurements. The number of mice is indicated on the top right corner of every graph. All values were normalized for bodyweight. A MLC/mIGF-1 fast skeletal muscle groups showed a significant weight increase (p<0.0002 for all) at three and six months, soleus weight was not changed. B MLC/Class 2 IGF-1Ea fast muscle groups were increased in weight at three months and six months of age (p<0.007 for all), while values at one months were elevated, but apart from gastrocnemius not significant. The weight of the slow soleus muscle was not influenced. C MLC/Class 1 IGF-1Eb one-month-old samples did not show any changes, while the weight of quad and TA showed a significant increase (p<0.03) at three months. By the age of six months, all fast muscles but the EDL were significantly increased in weight (p=0.01). D Line MLC/Class 2 IGF-1Eb samples showed elevated skeletal muscle weight at one and six months of age, while three-month-old samples were unchanged. None of the values reached significance.

FIG. 47 Histological analysis of MLC/Class 2IGF-1 Ea muscles. Six-month-old transgenic mice were compared to littermates of the same sex (n=4 for both). A Representative pictures of EDL and TA sections stained with NADH-TR (upper panel). Identity of fast IIB fibers was confirmed with antibodies against type IIB myosin and laminin (lower panel). B CSA analysis of EDL and TA fast, intermediate, and slow fibers. C Size distribution of different fiber types of the TA muscle. D Fiber type composition of the EDL muscle. E Analysis of the total number of fibers in the EDL muscle.

FIG. 48 Histological analysis of MLC/Class 1 IGF-1Eb muscles. Six-months-old mice were compared to littermates of the same sex (n=4 for both). A Representative pictures of EDL and TA sections stained with NADH-TR. B CSA analysis of EDL and TA fast, intermediate, and slow fibers. C Size distribution of different fiber types of the TA muscle. D Total number of fibers of the EDL muscle. E Whole muscle CSA of TA, EDL, and soleus. F Fiber type composition of the EDL muscle.

FIG. 49 Histological analysis of MLC/Class 2IGF-1 Eb muscles. Six-month-old transgenic mice were compared to littermates of the same sex (n=4 for both). A Representative pictures of EDL and TA sections stained with NADH-TR (upper panel). Identity of fast IIB fibers was confirmed with antibodies against type IIB myosin and laminin (lower panel). B CSA analysis of EDL and TA fast, intermediate, and slow fibers. C Size distribution of different fiber types of the TA muscle. D Fiber type composition of the EDL muscle. E Analysis of the total number of fibers in the EDL muscle.

FIG. 50 Functional analyses of IGF-1 isoform transgenic muscles (1). Two and a half-months-old animals of male sex were analyzed for EDL and soleus electrophysiological properties. MLC/mIGF-1 animals were analyzed at six month of age and are therefore shown separately. The number of animals from each line is indicated at the bottom of each column. A EDL single twitch force generation (F_{twitch}) is shown in the left panel and was only significant for MLC/Class 2 IGF-1Ea (p=0.01). Soleus F_{twitch} is shown in the left panel and was significantly increased in MLC/Class 1 IGF-1Eb (p=0.04). Data for MLC/mIGF-1 was not available. B EDL contraction speed (Fᵣₑₛₚ) is shown in the left panel and showed a significant change for MLC/Class 2 IGF-1Ea (p=0.04), while soleus Fᵣₑₛₚ shown in the right panel, was unchanged.

FIG. 51 Functional analyses of IGF-1 isoform transgenic muscles (2). Two and a half-months-old animals of male sex were analyzed for EDL and soleus electrophysiological properties. MLC/mIGF-1 animals were analyzed at six month of age and are therefore shown separately. The number of animals from each line is indicated at the bottom of each column. A EDL tetanic force (Fₘₐₓ) is shown in the left panel and was significant for MLC/Class 2 IGF-1Ea (p=0.003) and MLC/mIGF-1 (p=0.01). Soleus Fₘₐₓ is shown in the right panel and was unchanged in all transgenic lines. B The time the EDL muscle needed to half Fₘₐₓ (T_{fatigue}) is shown in the left panel and was not significantly changed in any of the samples. Results for the soleus muscle are shown in the right panel and were unchanged as well. C The specific force F_{spec} was calculated by dividing Fₘₐₓ by the weight of each analyzed muscle. Results for EDL (left panel) and soleus (right panel) were not significantly influenced by the presence of different IGF-1 isoform transgenes.

FIG. 52 mRNA expression and activation of IGF-1R. A Northern blot analysis of IGF-1R expression. 10 μg of total RNA from one-months-old male quadriceps muscle from WT and transgenic mice were used and the IGF-1R mRNA was detected with an IGF-1R-specific probe. Levels of expression did not show any changes induced by any of the transgenes. B Immunoprecipitation of the β-subunit of the IGF-1R. 1.3 μg of protein per sample was immunoprecipitated with an IGF-1Rβ-specific antibody. Western blot analysis was performed with an anti-phosphotyrosine antibody to detect the phosphorylation of the intracellular IGF-1R β-subunit. All transgenes activated the IGF-1R. C Density graph for phospho-IGF-1R-specific bands. Activation of IGF-1R was strongest in Class 2 IGF-1 transgenes.

FIG. 53 Kinetworks Phospho-site screen. The entire processing of samples was performed by the Kinetworks service: Transgenic quadriceps samples from two one-month-old animals per genotype have been sent for analysis together with one negative littermate from each line (total n=4). A Results for members of the Akt pathway. No significant changes were observed. B Screening results for members of the S6-kinase family. Significant changes were only seen for specific residues of S6K p85. C Members of the MAP-kinase pathway were not significantly influenced by IGF-1 isoform over-expression. D Phosphorylation level of IKKa and β. MLC/Class 1 IGF-1Eb significantly increased IKKa-phosphorylation (p=0.01).

FIG. 54 Histological analysis of CTX-injected skeletal muscle. Three-month-old male WT and transgenic mice (n=4 for each genotype and time point) were subjected to CTX injury. Samples were taken at two, five, and ten days after injury. 8 μm transverse sections of the TA muscle were cut and stained with trichrome staining. The mid region of the injury was identified for each injury and pictures were taken at a 20× magnification. Representative pictures of samples for two days after injection are shown in the upper panel, five day samples are represented in the middle panel, and the lower panel shows the muscle morphology ten days after CTX injection.

FIG. 55 Expression of endogenous IGF-1 isoforms in WT CTX-injected muscle. Three-months-old male quadriceps RNA from WT and transgenic mice (n=3 for all) was used for quantitative RT-PCR analysis of CTX-injury induced changes in endogenous IGF-1 isoform expression. A Class 1 IGF-1Ea RNA was significantly induced at day five (p=0.003) and day ten (p=0.001) after CTX-injection. B Class 2 IGF-1Ea RNA showed a similar trend as seen for Class 1 IGF-1Ea RNA, but was only significantly increased after ten days of regeneration (p=0.005). C Class 1 IGF-1Eb did not show significant changes in response to injury, but a tendencious increase was noted. D Class 2 IGF-1Eb was transiently and significantly induced at day five after injury (p=0.02).

FIG. 56 Influence of IGF-1 isoform transgenes on endogenous isoform expression after CTX-injury. Three-months-old male quadriceps RNA from WT and transgenic mice (n=3 for all) was used for quantitative RT-PCR analysis of injury-induced changes in endogenous IGF-1 isoform expression in the background of IGF-1 isoform over-expression. All values were compared to WT values of the same time point to evaluate if IGF-1 isoform over-expression influenced the normal WT expression pattern of IGF-1 isoforms after injury. A Analysis of endogenous Class 1 IGF-1Ea molecules revealed significant changes at day five after injury for Lines MLC/Class 1 IGF-1Eb (p=0.001) and MLC/Class 2 IGF-1Eb (p=0.04). B Endogenous Class 2 IGF-1Ea expression was not influenced in MLC/mIGF-1 and MLC/Class 1 IGF-1Eb animals after injury. C Endogenous levels of Class 1 IGF-1 Ea were not significantly changed. D Endogenous Class 2 IGF-1Eb mRNA was also not altered in the background of any of the transgenes.

FIG. 57 Distribution of regulated genes by cellular localization. The genes that were regulated (either up- or down-regulated) in each IGF1-isoform transgenic were pooled and classified by cellular localization according to their Gene Ontology annotations (available at www.Affymetrix.com). The values in each pie chart therefore represent the total number of regulated genes scored as belonging to each compartment. A MLC/Class 1 IGF-1Ea B MLC/Class 1 IGF-1Eb C MLC/Class 2 IGF-1Ea D MLC/Class 2 IGF-1Eb

### EXAMPLES

### Example 1

Transgenic Overexpression of IGF-1 Inhibits Ubiquitin-Mediated Muscle Atrophy

The present study was undertaken to investigate whether activation of the ubiquitin-proteasome pathway contributes to muscle atrophy in the syndrome of chronic heart failure. Since progressive muscle atrophy in advanced stages of chronic heart failure correlates with low serum levels and reduced local expression of IGF-1 (51,52), it is hypothesised that expression of an MLC/mIGF-1 transgene encoding a locally acting isoform of IGF-1 normally induced in response to muscle damage (53), could prevent the development of heart failure-associated muscle atrophy and concomitant activation of the proteasome.

Here it is shown that muscle activation of the ubiquitin-proteasome pathway in the setting of chronic left ventricular dysfunction is accompanied by selective induction of the muscle-specific ubiquitin ligase atrogin-1/MAFbx (for Muscle Atrophy F-box). Activation of Foxo transcription factors occurs in the skeletal muscle in chronic left ventricular dysfunction. Transgenic supplementation of the mIGF-1 isoform prevents muscle atrophy and activation of the proteasome. Further, overexpression of mIGF-1 specifically inhibited activation of Foxo4, the most abundant of these factors in skeletal muscle, and blocked expression of atrogin-1/M4Fbx. These studies establish a role for ubiquitin-mediated proteolytic degradation in muscle atrophy accompanying chronic left ventricular dysfunction and highlight the potential therapeutic value of supplementing the local mIGF-1 isoform in the treatment of progressive muscle wasting.

Methods

Animal Model Myocardial infarction was induced under anaesthesia in 8-12 weeks old male FVB mice (wildtype, WT) and in an FVB transgenic mouse line (MLC/mIgf-1) with skeletal muscle-restricted expression of the Exon1-Ea isoform of the rat IGF-1 gene (53). Progressive cardiac dysfunction was induced by ligation of the left coronary artery while sham-operated animals underwent the same procedure without ligation of the coronary artery. All mice underwent echocardiography after 2 and 12 weeks and were sacrificed after 12 weeks by injection of pentobarbital. The investigation conformed to the Guide for the Care and Use of Laboratory Animals published by the US National Institute of Health (NIH Publication No. 85-23, revised 1996) and was approved by the Harvard Medical School Standing Committee on Animals.

Histology Organs were removed, fixed in 4% paraformaldehyde and embedded in paraffin for further histological analysis. Tissue samples were cut in 5 μm thick sections and stained using hematoxylin and eosin. Morphological analysis of muscle fiber cross-sectional area was performed on tissue scans using ImagePro software (ImagePro Plus 4.5, Media Cybernetics).

Cell culture SOL8 cells (American Tissue Culture Collection) were cultured in DMEM containing 20% fetal calf serum, 100 U/ml penicillin and 100 μg/ml streptomycin. Cells were incubated with tumor necrosis factor-α (TNFα 20 ng/ml), interleukin-1β (IL-1 β, 50 ng/ml), insulin-like growth factor-1 (IGF-1, 10 ng/ml) and dexamethasone (Dexa, 10 ng/ml).

Real-Time PCR Total RNA (100 ng) was assessed by real-time PCR (LightCycler, Roche) using primers for atrogin-1/M4Fbx (sense: 5′-GAC TGG ACT TCT CGA CTG CC-3′and antisense: 5′-TCA GCC TCT GCA TGA TGT TC-3′) and β-tubulin (sense: 5′-CTG GGC TAA AGG CCA C-3′and antisense: 5′-AGA CAC TTT GGG CGA G-3′). The expression was normalized to expression levels of β-tubulin.

Western Analysis Protein levels were analyzed by Western analysis using specific monoclonal antibodies for the detection of myosin heavy chain (MF20, Developmental Studies Hybridoma Bank) or ubiquitin (P4D1, Santa Cruz Biotechnology). Polyclonal antibodies against phospho-Akt, total Akt, phospho-Foxo 1, 3 and 4 and total Foxo were from Cell Signaling. After incubation with HRP-conjugated secondary antibody, specific bands were visualized by enzymatic chemiluminescence (Perkin Elmer).

Immunoprecipitation 30 μl of protein A sepharose beads were incubated with 1 μg anti-ubiquitin antibody. 300 μg of total protein from the soluble fraction of the muscle lysates were incubated with antibody-bead complexes for two hours rotating at 4° C. Beads were centrifuged and washed three times with 0.5 ml lysis buffer and one time with 0.5 ml ice-cold PBS. The beads were resuspended in SDS sample buffer, incubated at 95° C. for five minutes, centrifuged, and the supernatant electrophoresed through a SDS-PAGE system. After transfer to a PVDF membrane, further immunoblotting was performed.

Proteasome Activity Protein lysates were incubated at 37° C. with proteasome assay buffer containing 10 μmol/ml SLLVT-AMC (Calbiochem) as substrate for the chymotrypsin-like activity of the proteasome. Fluorescence of free 7-AMC as a measure of proteasome activity was assessed in intervals over 60 min on a temperature-controlled fluorescence reader (Perkin Elmer).

Statistical Analysis All experiments were performed at least three times and data are expressed as mean±SEM. Data were analyzed by Student's t-test. One-way ANOVA with post-hoc analysis was used for analysis of data sets of more than two groups. A p-value of less than 0.05 was considered statistically significant.

Results

Assessment of Animals with Chronic Left Ventricular Dysfunction and Controls

Thirty-nine WT and 34 MLC/mIGF-1 transgenic mice were included in the study (Table 1). The animals were randomly assigned to a group undergoing ligation of the left coronary artery or sham surgery. Perioperative mortality and total mortality over the study period was comparable between WT and MLC/mIGF-1 (p=NS). Echocardiographic assessment demonstrated the expected development of progressive left ventricular enlargement and systolic dysfunction following left coronary artery ligation. Baseline differences in left ventricular dimensions between WT and MLC/mIGF-1 mice persisted throughout the study with comparable increases in wall thickness of the non-infarcted ventricular wall and ventricular volume (Table 2).

TABLE 1
Morphometric Assessment

| | WT | | MLC/mIgf-1 | |
|---|---|---|---|---|
| Weight (g) | Sham | MI | Sham | MI |
| Total Body | 25.9 ± 2.0 | 26.1 ± 2.2 | 34.5 ± 5.9‡§ | 33.3 ± 3.8‡§ |
| (8-10 wks) | | | | |
| (20-22 wks) | 29.6 ± 2.8 | 32.9 ± 2.5 | 38.1 ± 8.9* | 40.1 ± 7.8† |
| Heart | 0.16 ± 0.02 | 0.21 ± 0.05 | 0.18 ± 0.04 | 0.25 ± 0.1 |
| Heart/Total | 5.42 ± 0.66 | 6.46 ± 1.65* | 4.82 ± 1.22 | 6.42 ± 3.65 |
| (×10⁻³) | | | | |
| EDL (×10⁻³) | 63 ± 9 | 54 ± 11* | 69 ± 17 | 68 ± 12† |
| SOL (×10⁻³) | 154 ± 15 | 138 ± 13* | 178 ± 32* | 172 ± 23§ |
| QUA (×10⁻³) | 186 ± 13 | 171 ± 17* | 214 ± 31* | 208 ± 29§ |

(Data are presented as mean ± SD; WT—wildtype; MI—myocardial infarction; EDL - M. extensor digitorum longus; SOL - M. soleus; QUA - M. quadriceps; *p < 0.05 vs. WT sham; †p < 0.05 vs. WT MI; ‡p < 0.001 vs. WT sham; § p < 0.001 vs. WT MI)

TABLE 2
Echocardiographic Assessment of Left Ventricular Function

| | WT | | MLC/mIGF-1 | |
|---|---|---|---|---|
| | Sham | MI | Sham | MI |
| AWT (mm) | 1.25 ± 0.11 | 1.28 ± 0.08 | 1.37 ± 0.13* | 1.39 ± 0.13 |
| PWT (mm) | 1.13 ± 0.14 | 1.20 ± 0.14 | 1.25 ± 0.15 | 1.27 ± 0.14 |
| LVEDD (mm) | 2.70 ± 0.16 | 3.19 ± 0.59* | 2.87 ± 0.30 | 3.19 ± 0.46 |
| LVESD (mm) | 1.08 ± 0.26 | 1.66 ± 0.50 | 1.21 ± 0.16 | 1.61 ± 0.37† |
| LV Volume (cm³) | 0.03 ± 0.01 | 0.047 ± 0.03‡ | 0.032 ± 0.01 | 0.049 ± 0.02† |
| FS (%) | 57.8 ± 2.5 | 48.5 ± 7.5‡ | 57.5 ± 5.6 | 48.6 ± 5.3§ |

(Data are presented as mean ± SD; WT—wildtype; MI—myocardial infarction; AWT—anterior wall thickness; PWT—posterior wall thickness; LVEDD—left ventricular end-diastolic diameter; LVESD—left ventricular end-systolic diameter; FS—fractional shortening; *p < 0.05 vs. WT sham; †p < 0.05 vs. mIgf-1^{+/+} sham; ‡p < 0.001 vs. WT sham; §p < 0.01 vs. mIgf-1^{+/+} sham)

Skeletal Muscle Atrophy and Activation of the Ubiquitin-Proteasome-Pathway in Chronic Left Ventricular Dysfunction

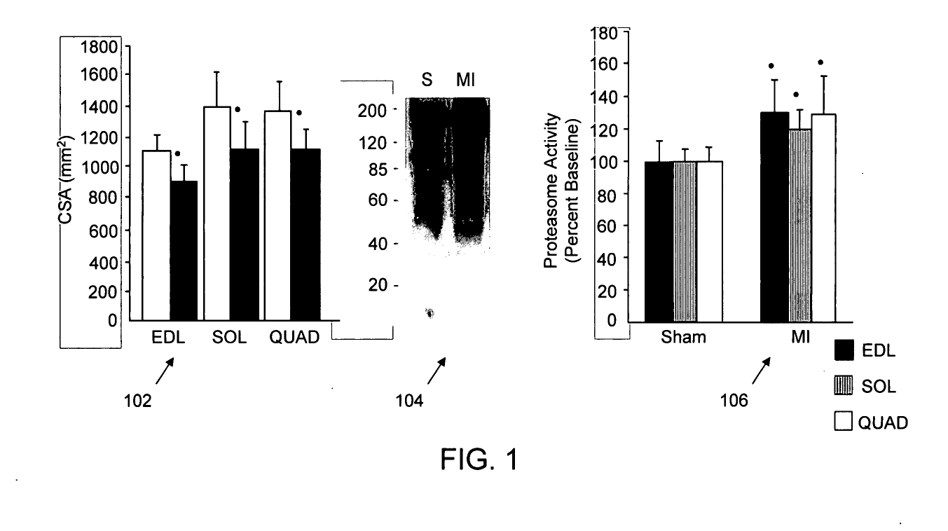

Assessment of skeletal muscle revealed a reduction in muscle fiber cross-sectional area in WT animals with CLVD (FIG. 1A). This reduction was paralleled by a decrease in isolated muscle weight (Table 1). Next the skeletal muscle activation of the proteolytic ubiquitin-proteasome pathway in WT and MLC/mIGF-1 transgenic animals was assessed. Immunoprecipitation of total ubiquitinated proteins followed by anti-ubiquitin immunoblotting revealed an increase in ubiquitinated substrates in soluble fractions of atrophying muscles (FIG. 1B). The activity of the 20S proteasome was measured by assessment of the chymotrypsin-like proteolytic activity. WT animals with CLVD and muscle atrophy displayed an increase in 20S proteasome activity in skeletal muscle by 40% (p<0.05 vs. controls) (FIG. 1C).

Transgenic Overexpression of mIGF-1 Prevents Activation of the Ubiquitin-Proteasome-Pathway and Muscle Atrophy in Chronic Left Ventricular Dysfunction

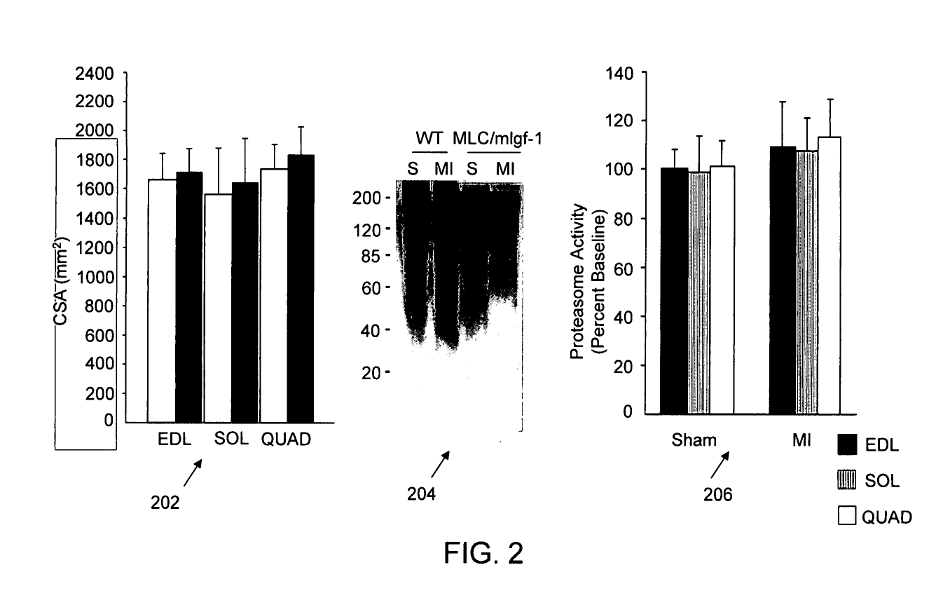

In MLC/mIGF-1 mice, muscle fiber cross-sectional area was increased at baseline due to the anabolic function of locally overexpressed mIGF-1 (54). In contrast to WT animals, MLC/mIGF-J mice with CLVD suffered no reduction of muscle fiber cross-sectional area (FIG. 2A). Further, transgenic overexpression of mIGF-1 prevented the increase in total muscle protein ubiquitination following myocardial infarction (FIG. 2B). Finally, the increase in proteasome activity was prevented in skeletal muscle of MLC/mIGF-1 transgenic mice with CLVD (FIG. 2C).

Expression of the Muscle-specific Ubiquitin Ligase atrogin-1/MAFbx in Skeletal Muscle of Animals with Chronic Left Ventricular Dysfunction

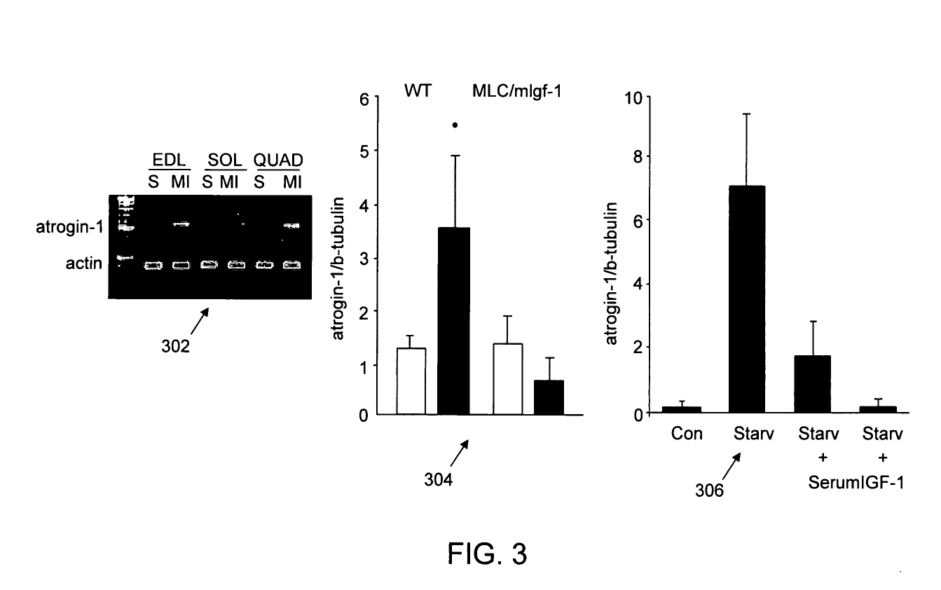

E3-ligases are ubiquitin-protein conjugating enzymes with a crucial role in the molecular cascade of protein ubiquitination, which marks them for rapid proteolytic degradation (Bodine et al. 2001; Gomes et al. 2001). The expression of atrogin-1/MAFbx in skeletal muscle of mice with CLVD was determined and showed a robust expression of atrogin-1/MAFbx in several muscles following myocardial infarction (FIG. 3A). Transcripts of atrogin-1/MAFbx were strongly induced in atrophying muscles of animals with CLVD (3.48±1.38 vs. 1.21±0.32 arbitrary units; p<0.01 vs. WT sham). In MLC/mIGF-1 transgenic muscle, expression of atrogin-1/MAFbx remained at basal levels following the development of CLVD (FIG. 3B).

The regulation of atrogin-1/MAFbx in the setting of muscle atrophy was confirmed in SOL8 cells starved for 72 h. Intriguingly, this effect was reduced by stimulation with 20% serum for 3 h and completely normalized by stimulation with IGF-1 for 3 h (FIG. 3C).

Polyubiquitination of Myosin Heavy Chain in Muscle Atrophy

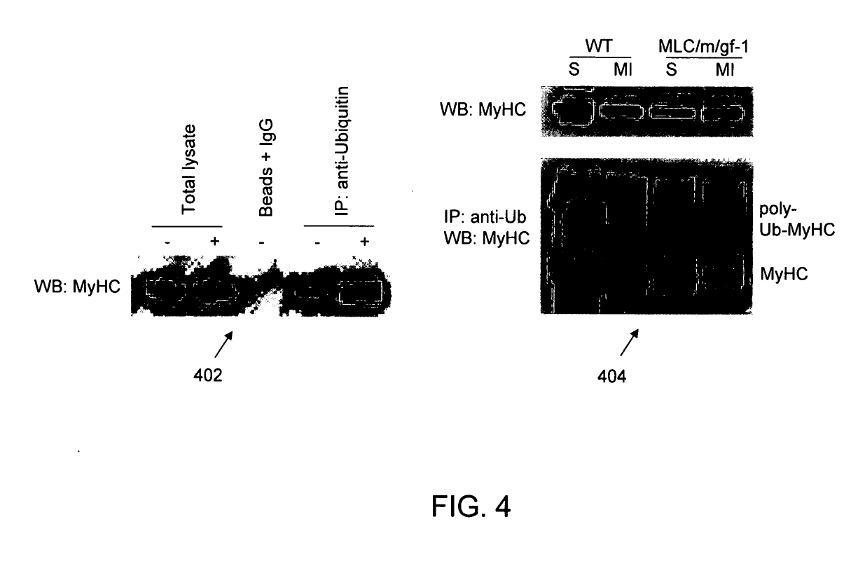

To assess whether increased ubiquitination of myosin heavy chain (MyHC), a known target of ubiquitination, (Solomon and Goldberg 1996; Acharyya et al. 2004) could be induced in vitro, SOL8 myogenic cell cultures were incubated with different inducers of ubiquitination. Dexamethasone induced total protein ubiquitination after 24 h and analysis of total ubiquitinated proteins followed by immunoblotting revealed a strong increase in MyHC ubiquitination (FIG. 4A) which was also seen after serum withdrawal and TNFα and IL-1β incubation (data not shown).

To determine whether MyHC was a also target of ubiquitin-mediated proteolytic degradation in vivo, similar analyses were performed on atrophied skeletal muscles of mice with CLVD and found an increased fraction of ubiquitinated MyHC in WT animals with chronic CLVD (FIG. 4B). In contrast, expression of the MLC/mIGF-1 transgene blocked the increase in MyHC ubiquitination in skeletal muscle of animals with chronic CLVD.

Activation of Foxo Transcription Factors in Muscle Atrophy is Inhibited by mIgf-1

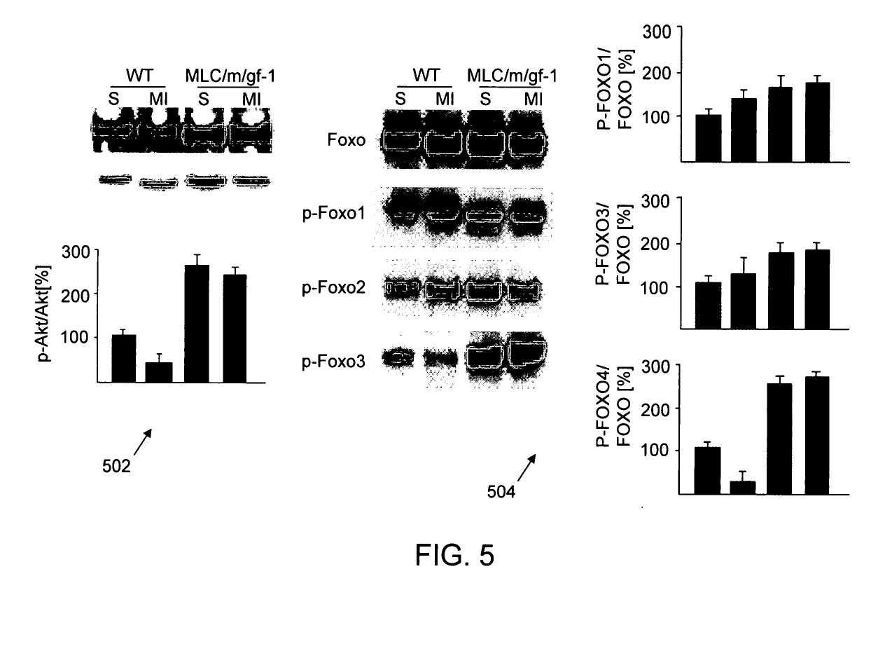

Reduced activity of the P13 Kinase/Akt pathway leading to enhanced activation of its downstream target, Foxo transcription factors, and expression of atrogin-1/MAFbx has been associated with muscle atrophy (55). Reduced Akt activity in muscle of WT mice with CLVD was prevented by mIGF-1 transgene expression (FIG. 5A). Activation of Foxo transcription factors indicated by reduced phosphorylation in muscle of WT mice with CLVD was also abrogated by mIGF-1 transgene expression, which specifically enhanced Foxo4 phosphorylation in muscle from mice with CLVD (FIG. 5B).

Discussion

In this study, an animal model of chronic left ventricular dysfunction (CLVD) was employed to investigate the ubiquitin-proteasome pathway in skeletal muscle atrophy and to explore potential therapeutic avenues for its prevention. An activation of the ubiquitin-proteasome pathway in atrophying skeletal muscle accompanied by Foxo activation and expression of the atrophy related ubiquitin-protein ligase atrogin-1/MAFbx is demonstrated. These changes are prevented by transgenic overexpression of mIgf-1.

Consideration of IGF-1 isoforms is critical in the interpretation of current studies on the effects of supplementary growth factors. Exogenously administered IGF-1 induces muscle hypertrophy through autocrine and paracrine mechanisms (56) and muscle-specific overexpression of a circulating IGF-1 isoform results in profound muscle growth mediated through increased protein synthesis and DNA accretion (57). Overexpression of the mIGF-1 isoform counters the decline in muscle mass in senescence (53) and in mdx mice (58). Gene transfer of mIGF-1 under the control of muscle-specific regulatory elements prevents age-related loss of skeletal muscle mass and function even when administered at senescence (20). Additionally, mIGF-1 may act as a potent regenerative agent, as increased stem cell recruitment to sites of muscle injury was observed in mice expressing the MLC/mIGF-1 transgene. When isolated from MLC/mIGF-1 muscles, these progenitor cells exhibit accelerated myogenic differentiation and induce muscle-specific markers in co-cultured bone marrow cells (Musaro et al. 2004). Therefore, it is likely that locally produced mIGF-1 counteracts atrophy through signal transduction pathways that may be distinct from those activated by circulating IGF-1.

### Example 2

Full Regeneration of the Mammalian Heart

Methods

Generation of α-MyHC/mIGF-1 Transzenic Mice

We generated transgenic mice (FVB) with a rat mIGF-1 cDNA driven by the mouse α-MyHC promoter (59). Transgenic mice were generated by standard methods and selected by PCR using tail digests. Transgenic animals were maintained as heterozygotes. The animals were housed in a temperature-controlled (22° C.) room with a 12:12 hour light-dark cycle. All the analyses were performed on male mice.

RNA Preparation and Northern Blot Analysis

Total RNA from wild type (WT) and mIGF-1 transgenic (TG) hearts was obtained by RNATRIZOL extraction (Gibco-BRL). RNA (10 μg) was analyzed on 1.3% agarose gels and hybridized as described (60).

Histological Analysis

Mice at different ages were anesthetized before cervical dislocation, and hearts were perfused with 4% paraformaldehyde (PFA) as previously described (61), then excised and embedded in paraffin. Paraffin sections (10 μm) were stained with haematoxylin and eosin and analyzed morphologically. Connective tissue was visualized by using Masson's Trichrome stain as described by Manufacture (Sigma). Cell size was analyzed by measuring the size of single nuclei cells in a 40× magnification. 10 sections (10 μm) from WT and TG hearts were used for cell measurement. Cells were measured in the left ventricle. Statistical analysis was performed as described below.

Cardiac injury

3-4 months-old WT and TG mice were anesthetized by Avertin injection (0.1 ml/10 g of a 2.5% solution). The tongue was retracted and a tracheal cannula (1.3×1 mm, OD×ID, Harvard Apparatus) was inserted into the trachea. The cannula was attached to the mouse ventilator (Model 687, Harvard Apparatus) via the Y-shaped connector. Ventilation was performed with a tidal volume of 200 μl and a respiratory rate of 120/min. The chest cavity was opened in the left fourth intercostal space. The heart was exposed and 25 μl of CTX 10 μM (Latoxan) were injected in the heart wall of the left ventricle or the left descending artery was ligated. The chest cavity, muscle, and skin were then closed by a 6-0 silk suture (Ethicon).

LCA was performed on avertin anesthetized mice as described above. Ventilation was performed with a tidal volume of 300 μl and a respiratory rate of 120/min. The chest cavity was opened in the left fourth intercostal space and the left coronary artery (LCA) was ligated with a 8.0 no absorbable suture (ethicon) below the left atrium to produce a 40% infarct size. The chest cavity, muscle, and skin were then closed by a 6-0 silk suture (Ethicon). Mice were kept under ventilation until they were completely awake from anesthetic.

Echocardiography

Eight 13 and 23 week-old males from WT and TG lines were weighed and lightly anaesthetized with pentobarbital (30 mg/kg i.p.) to allow analysis of cardiac anatomy and function on a Sonos 5500 (Hewlett Packard) with a 15 MHz linear transducer (15L6) (Philips Ultrasound, USA). The images were stored in a digital format on a magnetic optical disk for review and analysis. The left hemithorax was shaved and an ultrasound transmission gel was applied to the precordium. The heart was first imaged in the two-dimensional mode (2D) in the parasternal long-axis view to obtain the aortic root dimensions. The aortic flow velocity and the heart rate (HR) were measured with pulsed-wave Doppler on the same section. The sample volume cursor was placed in the aortic root and the transducer angled slightly, which allowed aortic flow parallel to the interrogation beam so that maximum aortic flow velocity was obtained easily. The cardiac output (CO) was calculated from the following equation: CO=0.785×D2×VTI×HR where D is the internal diameter of the aortic root and VTI is the velocity-time integral of the Doppler aortic spectrum. Then the pulsed Doppler window was placed between the tip of the mitral valve leaflets to record the mitral inflow velocities. The maximal speed of the early (E) and late (A) mitral filling were measured as well as the mean deceleration time of the E wave (DT) and the duration of the A wave (Adur). By placing the Doppler between aortic flow and mitral valve, the isovolumetric relaxation (IVRT) time was measured. Left ventricular cross sectional internal diameters in end-diastole (LVEDD) and in end-systole (LVESD) were obtained by an M-mode analysis of a 2D-short axis view at the papillary muscle level. The ejection and shortening fractions were calculated. From this view, the diastolic septum (S) and posterior wall (PW) thicknesses were measured. The left ventricular mass (LVM) was calculated with the following formula: LVM=1.055×[(S+PW+LVEDD)3−(LVEDD)3]. All the measurements were performed on, at least three beats, according to the guidelines of the American Society of Echocardiography.

Ultrasound Analysis of Inured Hearts

Eight 13 week-old males from WT and TG lines were analyzed by echocardiography one month after CTX injection in the left ventricle wall or after 1 month and 2 months after LCA ligation. The mice were weighed and lightly anaesthetized by Avertin injection (0.1 ml/10 g of a 2.5% solution). Cardiac anatomy and function were measured with a Vevo 660 (VisualSonics) Ultrasound, and by the use of a 630 RMV (real-time-micro-visualization) scanhead (Visualsonics). The analysis was very sensitive due to the high-resolution images that the VisualSonics Ultrasound can acquire. The left hemithorax was shaved and an ultrasound transmission gel (Parkers Laboratories Inc.) was applied to the precordium. The heart was imaged in the two-dimensional mode (2D) in the parasternal short-axis view to obtain left ventricular cross sectional internal diameters in end-diastole (LVEDD) and in end-systole (LVESD) by an M-mode analysis. The ejection and shortening fractions were calculated. From this view, the posterior wall (PW) thicknesses was also measured. Movie recordings of left ventricle motion were analysed in B-mode and in parastemal short-axis (PSA). 300 different frames covering cycles of ventricular contraction and distension (systole and diastole) were recorded.

Immunohistochemistry and BrdU Analysis

BrdU (Sigma) was administered ad libitum at 0.1% in the drinking water or injected intraperitoneally at 100 μg/g. Hearts were perfused with 4% PFA and embedded in paraffin. Sections were stained with anti-BrdU (BD-Pharmingen) as prescribed by the manufacturer. Positive nuclei were quantified by counting all nuclei and BrdU positive nuclei in 10 sections (10 μm) of WT and TG hearts bordering and covering the CTX injured side. Statistical analysis was performed as described below. Immunofluorescence was performed on frozen sections (10 Elm) of WT and TG hearts 1 month after CTX injection. BrdU was analyzed with a mouse anti-BrdU purchased from Amersham Biosciences, and cardiac muscle cells were stained with an anti-myosin antibody from Sigma (M7648). Nuclei were visualized by Hoechst dye (Sigma). Images were processed with a Leica DM RHC fluorescent microscope and a DC500 Digital Camera.

Isolation of Cardiac Cells

Adult mouse cardiomyocytes were isolated and cultured following the instructions of www.signaling-gateway.org.

Western Blot Analysis

Hearts from WT and TG mice were excised and excess blood was removed by washing in PBS 1X. Hearts were lysed in buffer containing 20 mM Tris-HCl (pH 8.0), 150 mM NaCl, 5 mM MgCI2, 10% glycerol, 1% Triton, 0.5% NP40, supplemented with 1 mM proteases and phosphatase inhibitor cocktail. 50 μg of proteins were loaded onto SDSPAGE gel and blotted on PVDF membrane. Phospho-Akt (Pharmingen) and Phospho-S6 (Cell Signaling) were used at a concentration of 1:500 and 1:1000 respectively in 5% BSA. Mouse monoclonal p21 antibody was purchased form Santa Cruz and used at a concentration of 1:250 in 5% milk. The blots were normalized for Akt (Transduction Laboratories), S6 ribosomal protein (Cell Signaling) and actin (goat polyclonal, Santa Cruz).

Real Time PCR and Reverse Transcriptase PCR

1 μg of RNA was used to set up the reaction of reverse transcription as prescribed by Manufacture (Promega). Real time PCR was performed using 10 μl of the Syber Green Dynamo™ Master Mix (Finnzymes, Espoo, Finland), along with 1 μl of cDNA and 0.75 mM of each primer in a total reaction volume of 20 μl. Duplicated samples were incubated at 95° for 3 min, followed by 45 cycles of amplification (95°, 10 sec; 56°, 20 sec; 72°, 30 sec). Results for each cytokine were normalized to ubiquitin ligase expression.

Primers
IL1β,

| | |
|---|---|
| forward | 5′-acatcaacaagagcttgacccaggc-3′ |
| reverse | 5′-agctcatatggtccgacagcacga-3′; |

IL6,

| | |
|---|---|
| forward | 5′-aggataccactcccaacagacgtg-3′ |
| reverse | 5′-gtagctatggtactccagaagacc-3′; |

IL10

| | |
|---|---|
| forward | 5′-ccaagccttatcggaaatg-3′ |
| reverse | 5′-tggccttgtagacacc-3′; |

IL4

| | |
|---|---|
| forward | 5′-catcggcattttgaa-3′ |
| reverse | 5′-cgtttggcacatccatctcc-3′; |

GAPDH

| | |
|---|---|
| forward | 5′-tgggtgtgaaccacgaa-3′ |
| reverse | 5′-acagctttccagaggg-3′; |

ANP

| | |
|---|---|
| forward | 5′-atgggctccttctccatcaccctg-3′ |
| reverse | 5′-tcggtaccggaagctgttgcagcc-3′; |

BNP

| | |
|---|---|
| forward | 5′-atggatctcctgaaggtgctgtcc-3′ |
| reverse | 5′-gcgttacagcccaaacgactgacg-3′; |

β-myosin heavy chain

| | |
|---|---|
| forward | 5′-ctgagcagaagcgcaatgcagagtcgg-3′ |
| reverse | 5′-ctcctcattcaggcccttggcaccaatg-3′; |

α-skeletal actin

| | |
|---|---|
| | 5′-atgtgcgacgaagacgagaccacc-3′ |
| reverse | 5′-gccacatacatggcaggcacgttg-3′; |

Glucose transporter 1 (Glut1)

| | |
|---|---|
| forward | 5′-gatcccagcagcaagaaggtgacg-3′ |
| reverse | 5′-tggagaagcccataagcacagcag-3′; |

β-actin

| | |
|---|---|
| forward | 5′-taaaacgcagctcagtaacagtccg-3′ |
| reverse | 5′-tggaatcctgtggcatccatgaaac-3′. |

Statistics

All comparisons between WT and TG mice were performed by means of paired Student's t tests. A significant difference was considered when p<0.05, setted as a double side value.

Results

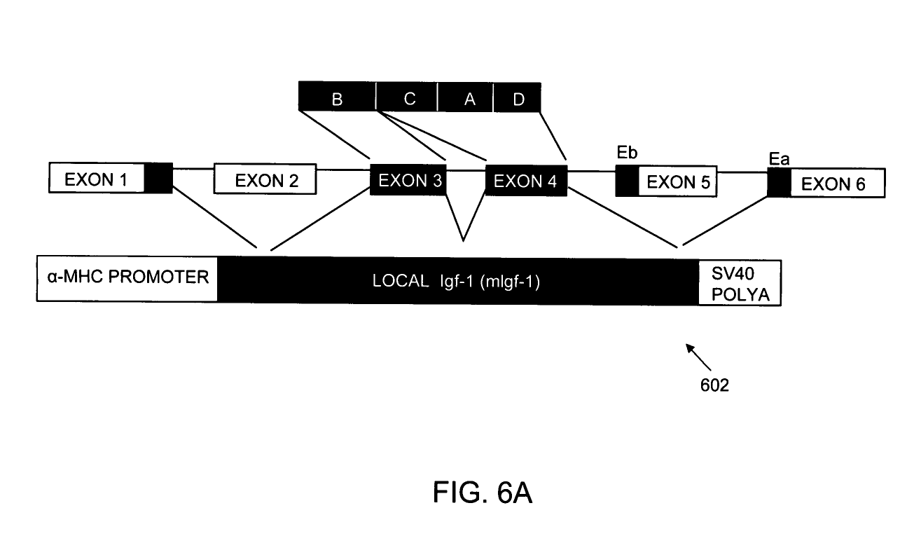

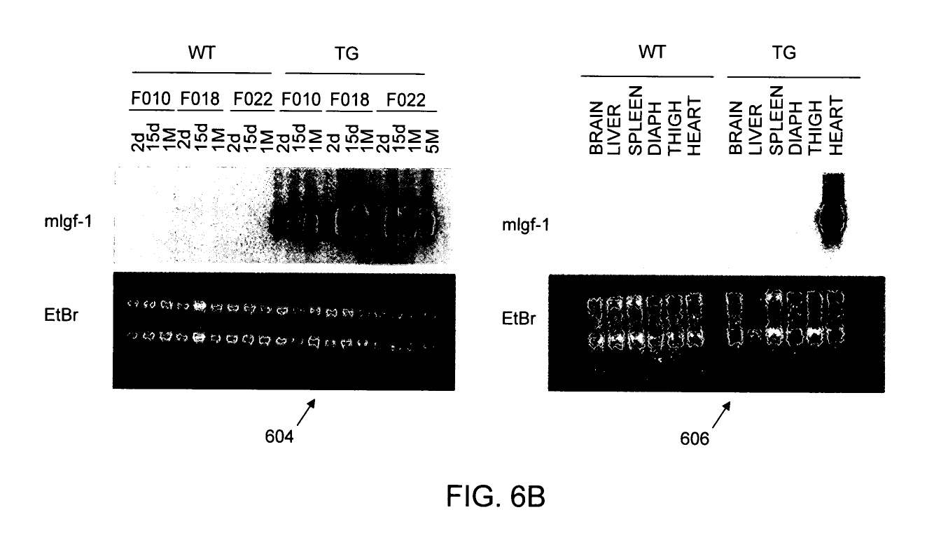

In the present study the ability of a locally acting IGF-1 isoform, mIGF-1, to regenerate the injured heart (62) was tested. Transgenic mice with a rat mIGF-1 cDNA driven by the mouse α-MHC promoter to restrict expression of mIGF-1 to the mouse myocardium and exclude possible endocrine effects on other tissues were generated (FIG. 6A). Transgenic mice developed normally with no perturbation in reproduction and breeding. Initial characterisation of three α-MHC/mIGF-1 transgenic lines with variable transgene expression levels revealed similar cardiac phenotypes. Cardiac restricted mIGF-1 transcript expression levels increased with age in all founders tested and reached a steady-level at two months (FIG. 6B). Expression of the transgene in adult mice was restricted to the heart (FIG. 6C), and endogenous levels of IGF-1 were undetectable in other tissues using the rat probe. A single transgenic line was selected for further analysis (F018).

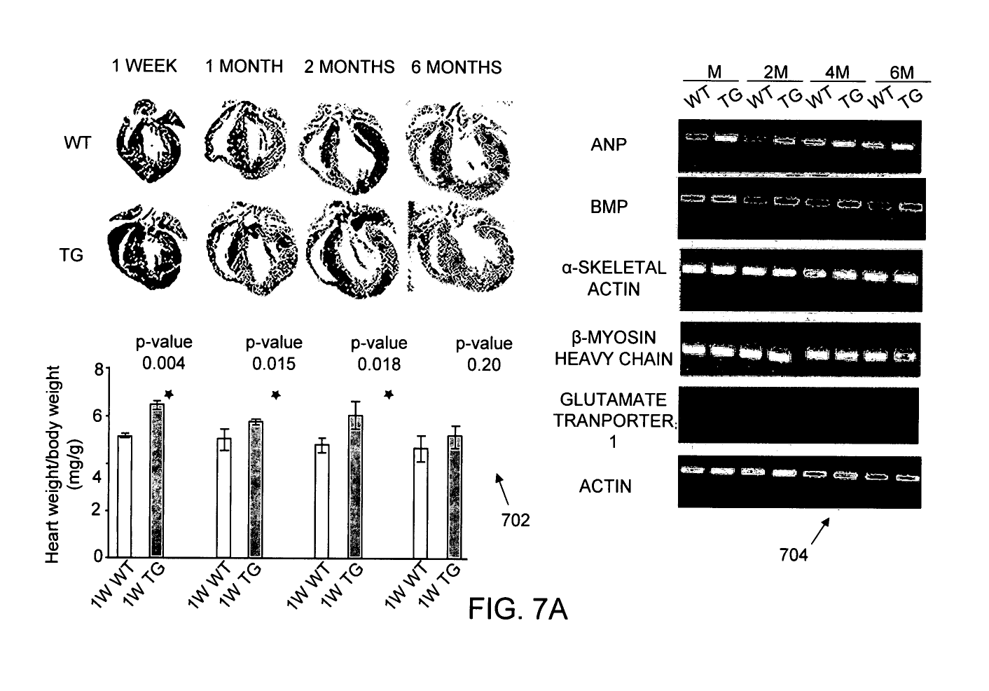

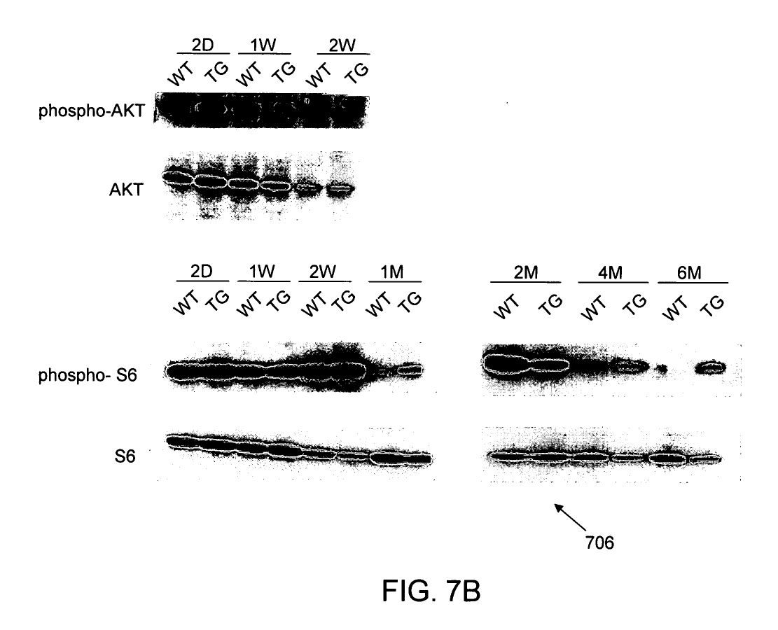

Postnatal transgenic mIGF-1 hearts displayed accelerated cardiomyocyte hypertrophy, precociously attaining wild-type adult heart size (FIG. 7A). Cardiac hypertrophy was related to higher expression levels of ANP at 1 and 2 months, without any further significative change (FIG. 7B). Other markers underlining cardiac hypertrophy, such as BNP, α-skeletal actin, β-myosin heavy chain, and glutamate transporter 1, were not affected (FIG. 7B).

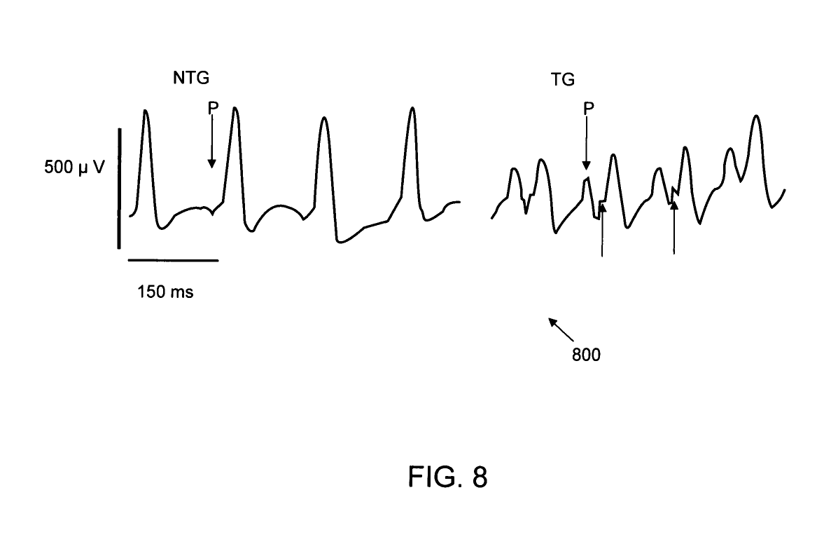

Measurement of cardiac function by echocardiography and electrocardiography (FIG. 8, Table 3) showed that mIGF-1 induces a 20% concentric left ventricular hypertrophy, confirming the histological analysis (Table 3). In transgenic male mice, echocardiography identified a small but significant decrease in cardiac contractility, demonstrated by a 13% decrease in ejection fraction and fractional shortening. Nevertheless, the high level of contractility preserved the resting cardiac output despite compromised diastolic function, identified by the 21% decrease of the E/A ratio and the prolongation of the A wave duration (+14%). These two abnormalities were not linked to prolongation either of the isovolumetric relaxation time or of the mean deceleration time of the E wave. Thus, although systolic and diastolic components of the cardiac function were affected, the hearts were not dilated and cardiac output and blood pressure were maintained normal and not reduced during development (Table 3).

TABLE 3

| Echo- | 13 week-old | | 23 week-old | |
|---|---|---|---|---|
| cardiography | NTG (n = 8) | TG (n = 8) | NTG (n = 8) | TG (n = 8) |
| LVM (mg) | 98 ± 5 | 119 ± 6* | 101 ± 3 | 118 ± 4* |
| LWM/BW | 3.1 ± 0.1 | 3.8 ± 0.2* | 2.9 ± 0.1 | 3.3 ± 0.2 |
| (mg/g) | | | | |
| S (mm) | 0.73 ± 0.01 | 0.82 ± 0.03* | 0.74 ± 0.02 | 0.84 ± 0.02* |
| PW (mm) | 0.59 ± 0.02 | 0.70 ± 0.02* | 0.64 ± 0.02 | 0.70 ± 0.01* |
| LVEDD (mm) | 4.17 ± 0.11 | 4.19 ± 0.08 | 4.10 ± 0.06 | 4.13 ± 0.13 |
| LVESD (mm) | 2.76 ± 0.10 | 3.03 ± 0.07* | 2.64 ± 0.06 | 2.82 ± 0.15 |
| FS (%) | 34 ± 1 | 27 ± 1* | 35 ± 1 | 32 ± 2 |
| EF (%) | 69 ± 2 | 60 ± 2* | 71 ± 2 | 66 ± 3 |
| CO (ml/min) | 26 ± 1 | 28 ± 2 | 38 ± 3 | 31 ± 3 |
| E/A | 1.61 ± 0.08 | 1.27 ± 0.11* | 1.49 ± 0.08 | 1.21 ± 0.07* |
| Adur (ms) | 35 ± 1 | 40 ± 1* | 37 ± 1 | 41 ± 2* |
| DT (ms) | 41 ± 1.7 | 43 ± 0.8 | 42 ± 2.1 | 46 ± 1.1* |
| IVRT (ms) | 21 ± 1 | 24 ± 1 | 25 ± 2 | 26 ± 2 |
| RV/LV | nd | nd | 0.25 ± 0.02 | 0.3 ± 0.01* |

NTG: non transgenic,
TG: transgenic,
SAP: systolic arterial pressure,
HR: heart rate,
PR: PR interval,
QT: QT interval,
LVM: left ventricular mass,
BW: body weight,
S: septal thickness,
PW: posterior wall thickness,
LVEDD: left ventricular end-diastolic diameter,
LVESD: left ventricular end-systolic diameter,
FS: fractional shortening,
EF: ejection fraction,
CO: cardiac output,
E/A: maximal speed of early on late mitral filling ratio,
Adur: duration of the mitral A wave,
DT: mean deceleration time of the E wave,
IVRT: isovolumetric relaxation time and RV/LV: right to left ventricular diameters ratio.
Nd: not determined.
All results are expressed as means ± sem. *P < 0.05 (paired Student's t tests for comparisons).

In skeletal muscle cell lines, it has been reported that muscle growth and hypertrophy are mediated by activation of the AKT/mTOR pathway, leading to up-regulation of the translational machinery (63), and that IGF-1 induces skeletal myotube hypertrophy by the PI(3)K/AKT/mTOR pathway. Compelling evidences coming from lower organisms such as Drosophila Melanogaster, showed that loss or inhibition of either PI(3)K, mTOR or p70S6K resulted in decrease of cell size (64). Conversely, overexpression of the insulin receptor substrate IRS-1 or Akt or p70S6K was sufficient to cause hypertrophy of cells in which they were expressed (65, 66). This growth effect appears to be functionally conserved in mammals, as p70S6K knockout mice have reduced body size and cell growth (67).

In order to elucidate the signaling regulated by mIGF-1 overexpression in the heart, a phosphoprotein screen has been performed on wild-type and transgenic heart lysates by Kinetworks analysis (Kinexus Bioinformatic Corp.). The analysis showed that AKT pathway carrying to S6 phosphorylation is not affected (Table 4). mTOR and p70 S6 kinase phosphorylation levels were not changed (33 CPM for control wild-type vs 26 CPM for transgenic hearts) or detected respectively in transgenic mice compared to wild-type littermates (Table 4). mIGF-1 over-expression maintained a sustained S6 ribosomal protein phosphorylation during all ages analyzed, whereas the activity of the protein in wild-type hearts displayed a more modulated regulation, with a strong activation at two months and a complete decreased phosphorylation at four and six months (FIG. 7C). The sustained activation of S6 ribosomal protein observed in mIGF-1 overexpressing hearts suggested that a continuous need of ex novo protein synthesis is required to maintain the sudden growth and remodeling of the transgenic heart. However, in mammalian cells the precise pathway connecting PI(3)K to the activation of S6 and consequently of the translational machinery is a matter of some dispute (64, 68). It is interesting to notice that, independently from AKT, PDKI has been found to directly phosphorylate p70S6K (69), indicating that AKT has a dispensable role for signaling to p70S6K. In C. elegans, PDK1 and the two PKB isoforms, Akt-1 and -2, as well as the serum-glucocorticoid kinase SGK, function in an insulin/IGF-1 receptor-mediated signalling pathway to regulate metabolism, development and longevity (70, 71). Moreover, mammalian cells lacking PDK1 fail to activate downstream targets in response to IGF-121. PDK1 is considered an alternate member of the AGC kinase family and requires phosphorylation at S241 to be catalytically active (72). Strong activation of PDK1 was observed in mIGF-1 transgenic hearts (Table 3). Importantly, the mIGF-1 transgene hyperphosphorylated PDK1 at S241, the critical activation loop serine present in other AGC kinases, whereas fully processed IGF-1 strongly phosphorylates S39623, further indicating that a novel signalling cascade, independent of Akt and p7OS6K, is activated downstream of the mIGF-1 isoform to increase protein synthesis and growth. Interestingly, the data showed that the phosphorylation state of AKT is not affected, besides a slight postnatal increase, and it is not regulated in parallel to activation of S6 ribosomal protein (FIG. 7C), whereas a strong activation of the downstream mediator of P13K, PDK1, has been observed (241CPM in wild-type hearts vs 507 in transgenic hearts) (Table 4).

TABLE 4
Phosphoprotein screen of 31 phopshoproteins in wild-type and
transgenic heart tissues by Kinetworks analysis (Kinexus
Bioinformatic Corp.). The trace quantity of each band is
defined as CPM and is measured under its intensity profile curve.
Each value is normalized by the amount of protein in each sample.
Each lane corresponds to a specific protein and the Tyrosine or
Serine position phosphorylated is indicated. Transgenic increase
or decrease of band intensity compared to wild-type (control)
is expressed in percentage.

| | | WT | TG | | |
|---|---|---|---|---|---|
| | | Heart | Heart | | |
| | | Norm | Norm | WT | TG |
| | | CPM | CPM | Heart % | Heart % |
| 90 kDa Ribosomal S6 Kinases (S380) | RSK1/2 Lane 7 | | | | |
| 90 kDa Ribosomal S6 Kinases (T573) | RSK1/2 Lane 5 | | | | |
| AMP-activated protein kinase alpha | AMPKa Lane 9 | | | | |
| (T172) | | | | | |
| Bone marrow X (Eph-like) kinase | BMX (Etk) Lane 5 | | | | |
| (Y40) | | | | | |
| Bruton's tyrosine kinase (Y223) | Btk Lane 15 | | | | |
| Calcium/calmodulin-dependent kinase | CaMK2 Lane 15 | | | | |
| II (T286) | | | | | |
| Cyclin-dependent kinase 1 (T161) | CDK1 Lane 19 | | | | |
| Cyclin-dependent kinase 1 (Y15) | CDK1 Lane 16 | | | | |
| | 4E-BP1 (16) | | | | |
| elF4E binding protein (S65) (16) | Lane 6 | | | | |
| | 4E-BP1 (17) | | | | |
| elF4E binding protein (S65) (17) | Lane 6 | | | | |
| | 4E-BP1 (18) | | | | |
| elF4E binding protein (S65) (18) | Lane 6 | | | | |
| Extracellular signal-regulated kinase 1 | ERK1 Lane 14 | 99 | 123 | Control | 24% |
| (T202/Y204) | | | | | |
| Extracellular signal-regulated kinase 2 | ERK2 Lane 14 | 53 | 62 | Control | 17% |
| (T185/Y187) | | | | | |
| Glycogen synthase kinase-3 alpha (S21) | GSK3a Lane 2 | | | | |
| Glycogen synthase kinase-3 beta (S9) | GSK3b Lane 2 | | | | |
| I-kappa-B kinase alpha (S180) | IKKa Lane 9 | | | | |
| I-kappa-B kinase beta (S181) | IKKb Lane 9 | 49 | 43 | Control | −12% |
| Lyn (Y507) (44) | Lyn (44) Lane 20 | 214 | 184 | Control | −14% |
| Lyn (Y507) (46) | Lyn (46) Lane 20 | 108 | 50 | Control | −54% |
| MAP kinase activated protein kinase 2 | MAPKAPK2 | | | | |
| (T334) | Lane 17 | | | | |
| MAP kinase interacting kinase 1 | Mnk1 Lane 4 | | | | |
| (T197/202) | | | | | |
| MAPK/Erk kinase 1/2 (S217/221) | MEK1/2 Lane 12 | 74 | 25 | Control | −66% |
| MKK3/6(1) (S189/S207) | MKK3/6 Lane 10 | | | | |
| MKK6(2) (S207) | MKK6 Lane 10 | 69 | 85 | Control | 23% |
| p38 MAPK (T180/Y182) | p38a MAPK | | | | |
| | Lane 4 | | | | |
| p70 S6 kinase (T389) | S6Ka p70 Lane | | | | |
| | 19 | | | | |
| p70 S6 kinase (T421/T424) | S6Ka p70 Lane | | | | |
| | 17 | | | | |
| p85 S6 kinase 2 (T412) | S6K2 p85 Lane | | | | |
| | 19 | | | | |
| p85 S6 kinase 2 (T444/S447) | S6K2 p85 Lane | | | | |
| | 17 | | | | |
| Phosphoinositide-dependent protein | PDK1 Lane 13 | 241 | 507 | Control | 110% |
| kinase 1 (S241) | | | | | |
| PKC-related kinase 1 (T778) | PRK1 Lane 13 | 488 | 555 | Control | 14% |
| PKC-related kinase 2 (T816) | PRK2 Lane 13 | 54 | 93 | Control | 72% |
| Protein kinase B (T308) | PKBa (Akt1) | | | | |
| | Lane 11 | | | | |
| Protein kinase C alpha/beta (T638) | PKCa/b Lane 6 | 115 | 104 | Control | −10% |
| Protein kinase C delta (T505) | PKCd Lane 14 | 113 | 129 | Control | 14% |
| Protein kinase C zeta (T410)/lambda | PKCz/l Lane 3 | | | | |
| (T403) | | | | | |
| Protein kinase D (Protein kinase mu) | PKCm/PKD | 60 | 17 | Control | −72% |
| (S916) | Lane 3 | | | | |
| Protein kinase theta (T538) | PKCt Lane 10 | | | | |
| Raf (S259) (60) | Raf1 (60) Lane 8 | 127 | 124 | Control | −2% |
| Raf (S259) (70) | Raf1 (70) Lane 8 | 114 | 98 | Control | −14% |
| Retinoblastoma Protein (S780) | Rb Lane 18 | 271 | 209 | Control | −23% |
| Retinoblastoma Protein (S807/S811) | Rb Lane 7 | | | | |
| The mammalian target of Rapamycin | mTOR Lane 13 | 33 | 21 | Control | −36% |
| (S2448) | | | | | |
| Type1 protein phosphatase alpha | PP1a Lane 8 | | | | |
| (T320) | | | | | |
| Zap70 (Y319)/Syk (Y352) | Zap70/Syk Lane | | | | |
| | 20 | | | | |

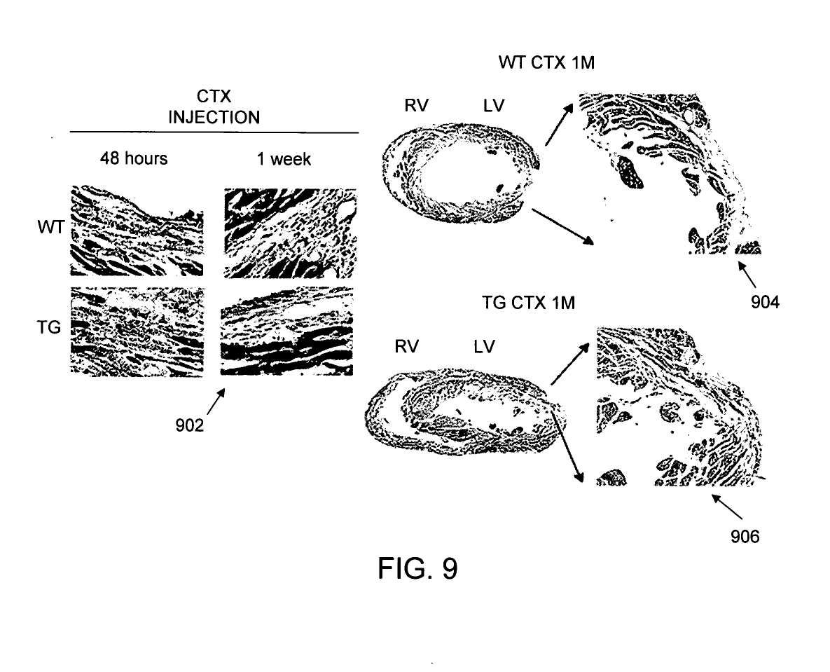

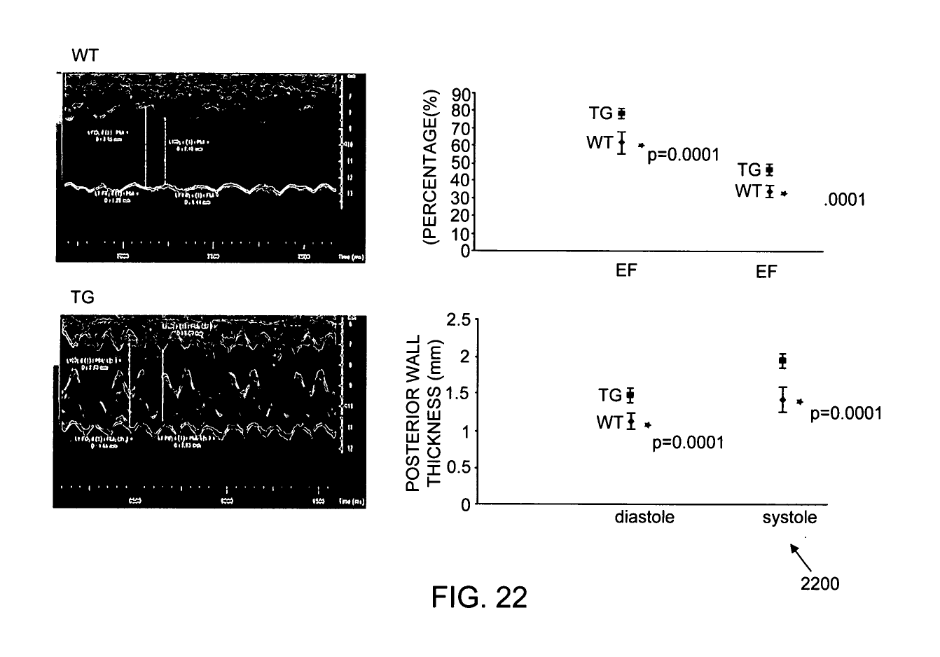

The regenerative capacity of mIGF-1 was analyzed by direct cardiotoxin (CTX) injection into the ventricles of four months old mice. The CTX model was chosen over other models of myocardial lesion such as LAD ligation because it produces a well-delineated transmural lesion, and reduces the risk of ventricular fibrillation. CTX injection in both wild-type and transgenic mIGF-1 hearts produced a reproducible and localized infarction with evident cell death and marked inflammation (FIG. 9A). In contrast to the characteristic progression of scar formation in wild-type hearts (FIG. 9B), mIGF-1 overexpression induced repair of the injured tissue after one month, without scar formation (FIG. 9C). Functional recovery after CTX-induced infarcts of wild-type and mIGF-1 transgenic hearts was analysed with high-resolution echocardiography after one month. mIGF-1 transgenic hearts showed integrity of the posterior wall and normal echocardiographic profiles compared to wild-type hearts (FIG. 22). Measurement of posterior wall thickness showed a significant decrease in the wild-type hearts in both diastolic and systolic parameters compared to transgenic hearts (diastole-WT 1.13+/−0.12 mm, diastole-TG 1.48+/−0.10 mm; systole-WT 1.42+/−0.17 mm, systole-TG 1.95+/−0.09 mm). Mean values of ejection fraction (EF) and fractional shortening (FS) were impaired in wild-type hearts when compared to transgenic hearts (EF 61%+/−7% compared to 78%+/−3%; FS 34%+/−4% compared to 47%+/−3%) (FIG. 22), indicating that mIGF-1 induced both morphological and functional regeneration.

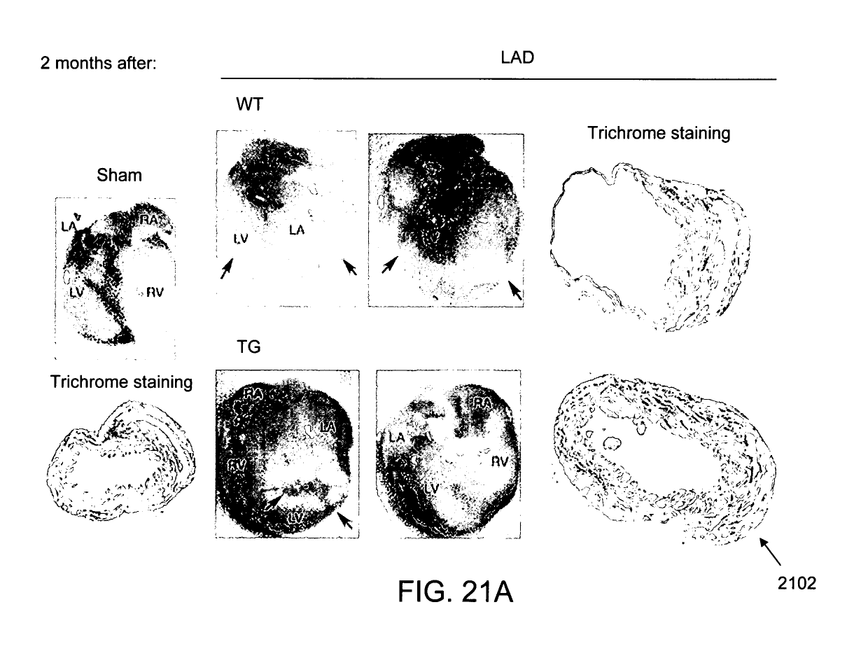

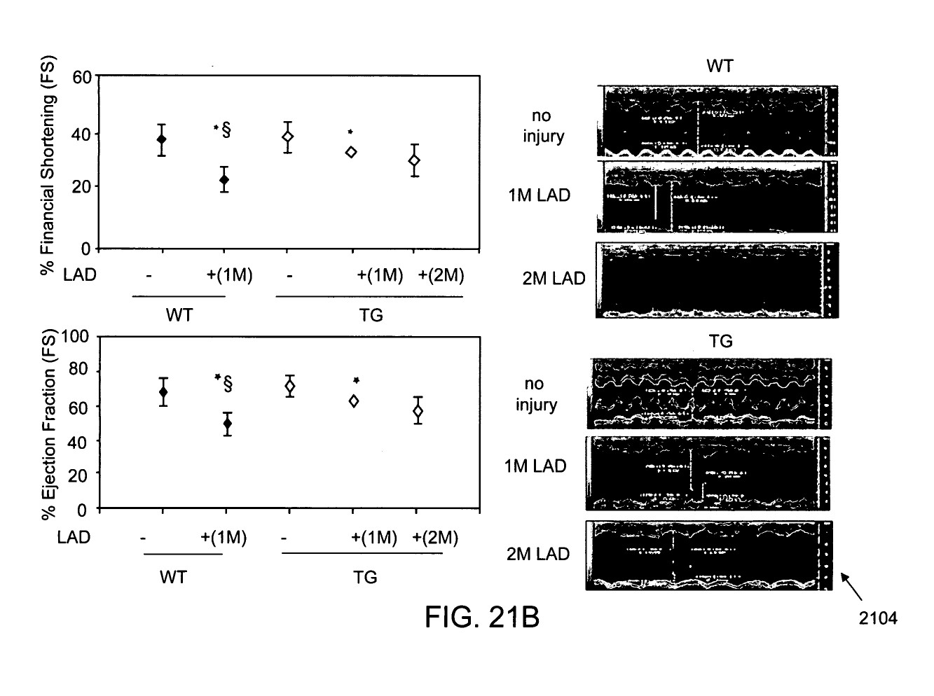

The regenerative capacity of mIGF-1 transgenic hearts was also analyzed by ligation of the left coronary artery (LCA) of four month-old mice. In wild-type mice, LCA induced infarcts characterised by progressive and extended fibrotic tissue formation (FIG. 21a, upper panel), accompanied by functional impairment after 1 month that worsened after 2 months (FIG. 21b and c). Percentage mean values of fractional shortening (FS) and ejection fraction (EF), as measured by high-resolution echocardiography, were significantly decreased compared to sham operated mice (Table 9). In contrast, infarcted mIGF-1 transgenic hearts showed a moderate but significant decrease in the percentage of ejection fraction (EF) and fractional shortening (FS) after 1 month, with no significant changes after 2 months compared to mIGF-1 transgenic sham operated mice and wild-type ligated mice (FIG. 21b, c and Table 9). The mIGF-1-mediated blockade of the normal progressive impairment in infarcted heart function was accompanied by reduced scar formation (FIG. 21a, lower panel). Recovery of cardiac function as well as morphological restoration of infarcted mIGF-1 transgenic hearts was confirmed by normal left ventricular motion in systolic and diastolic phases compared to mIGF-1 transgenic uninjured hearts. In contrast, wild-type hearts presented chamber enlargement and a significant decrease in wall motility near the infarct.

TABLE 9
Cardiac functional parameters in wild-type (WT) and mIGF-1 transgenic
(TG) mice. Ejection fraction (EF) and fractional shortening (FS) were measured in
WT and TG mice with and without MI after 1 and 2 months. Anhestesized mice were
analysed with a visualsonic ultrasound and each value is the average of three
different readings on the same animal from eight male mice in each group. The heart
function of WT mice 2 months after MI was dramatically impaired, confounding the
reading and the recording in all animals tested (data not shown). St. Deviation,
standard deviation; wti, wild-type injured mice; tgi, transgenic injured mice.
Significant values are calculated with the student t-test setting p < 0.05 as a double
side value.

| Measurement | | WT | WT 1M LCA | TG | TG 1M LCA | TG 2M LCA |
|---|---|---|---|---|---|---|
| % EF | Average | 68.1 | 48.7 | 70.8 | 62.7 | 57.47 |
| | St. Deviation | 7.7 | 6.5 | 6.0 | 1.3 | 7.1 |

t-test P < 0.05 wt vs wti 0.031 wti vs tgi 0.014 tg vs tgi 0.015 tgi 1M vs tgi 2M 0.37

| | | | | | | |
|---|---|---|---|---|---|---|
| % FS | Average | 37.7 | 24.0 | 39.1 | 33.6 | 30.68 |
| | St. Deviation | 5.6 | 4.4 | 5.0 | 1.1 | 5.45 |

t-test P < 0.05 wt vs wti 0.046 wti vs tgi 0.014 tg vs tgi 0.039 tgi 1M vs tgi 2M 0.49

The early event characterizing myocardial necrosis comprises Complement activation, free radicals generation, chemokines upregulation, and cytokines cascade (73). IL8, IL6 and C5a are released in the ischemic myocardium and may have a crucial role in neutrophil recruitment (73). Intriguingly, it has been shown that cytokines inhibiting the inflammatory response, such as IL10, could have an important role in suppressing injury and blocking scar formation (74).

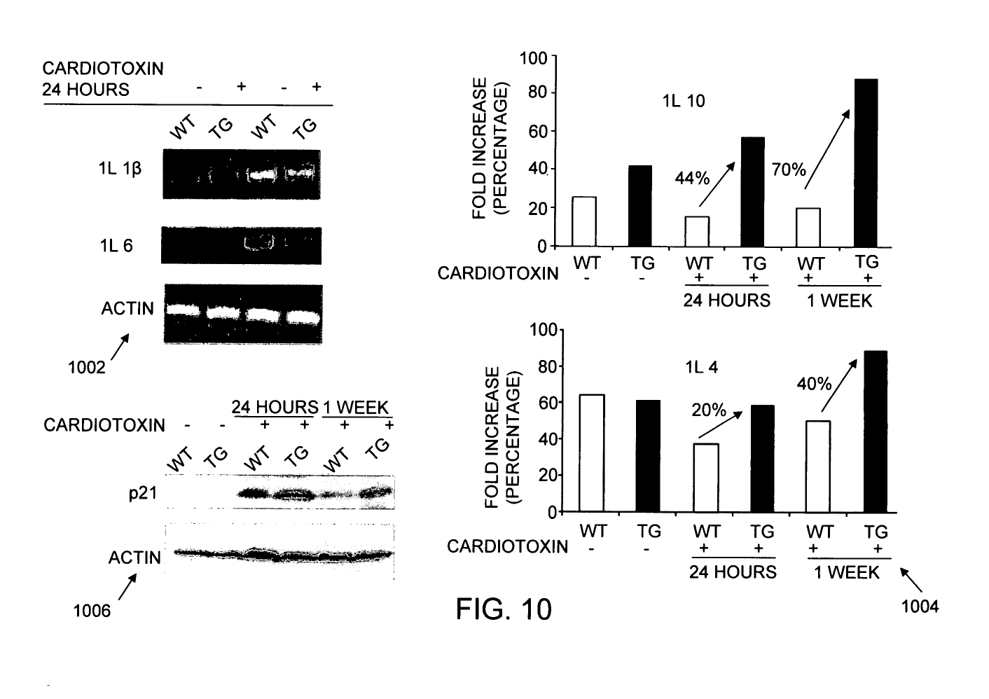

The early mechanisms leading to transgenic heart healing could involve decrease in pro-inflammatory cytokines and an increase in anti-inflammatory cytokines. Real time PCR and reverse transcriptase PCR (RT-PCR) in wild-type and transgenic hearts after 24 hours and one week from injury, showed that the pro-inflammatory IL6 was down-regulated after 24 hours from cardiotoxin injection in transgenic heart, whereas wild-type hearts showed increasing mRNA levels of IL6 (FIG. 10A). IL1β was not affected by cardiotoxin injection in wild-type and transgenic injured hearts (FIG. 10A), indicating that certain cytokines have a specific role in the heart in response to cardiotoxin injury.

The analysis of the anti-inflammatory cytokine IL10 by real time PCR showed 44% increase in transgenic hearts after 24 hour from injury, and to a greater extent at one week (77% compared to wild-type), whereas the level of the cytokine is lower in wild-type injured heart compared to uninjured tissue (FIG. 10B). IL4 was also upregulated in transgenic hearts 1 week after injury, but to a lower extent than IL10 (FIG. 10B).

Interestingly, the CDKs inhibitor p21^{WAF1/CIP1} is upregulated in response to injury in both wild-type and transgenic hearts, but overexpression of mIGF-1 lead to an extended up-regulation of p21 after injury (FIG. 10C). p21 has been implicated in many cellular responses leading to differentiation of several tissues and in the blockage of cell cycle progression (75). Recent compelling evidence showed that IGF-1 activates p21 and that p21 is important for IGF-1-mediated cell survival upon UV irradiation (76). Moreover, an intriguing study showed that spontaneous production of IL6 in rheumatoid arthritis, which is associated with high inflammation of the joints, is suppressed by p21 expression (77,78). The increased p21 expression after injury opens a novel and so far unexpected role for this cdk inhibitor in the heart.

Although regeneration has been presented as an evolutionary variable (79) illustrated by the robust proliferative capacity of the injured heart in other vertebrate such as newt and zebrafish (80,81), recent work has revealed a capacity for excellent regeneration in certain mammalian tissues, like embryonic or fetal skin (82). Compelling evidences of cardiac renewal occurring throughout life in the myocardium has been extensively proved as part of cardiac homeostasis (83,84), although the complete regenerative program in case of extended injury is precluded in mammalian heart by fibrotic tissue formation and consequently cardiac function impairment. This evidence indicates clearly that normally the regenerative program is limited in mammalian heart due to missing signaling present in the lower vertebrates such as newt and zebrafish.

Analysis suggested that the process of regenerative growth and the formation of new myocardial tissue involve modulation of the inflammatory response and changes in cytokines signaling. The myocardial tissue restoration observed in mIGF-1 overexpressing hearts could result from cardiac cell proliferation. To assess cardiac hyperplasia, cell cycle was assayed by measuring the nuclear incorporation of bromodeoxyuridine (BrdU), a marker of DNA synthesis, 1 month after cardiotoxin injection.

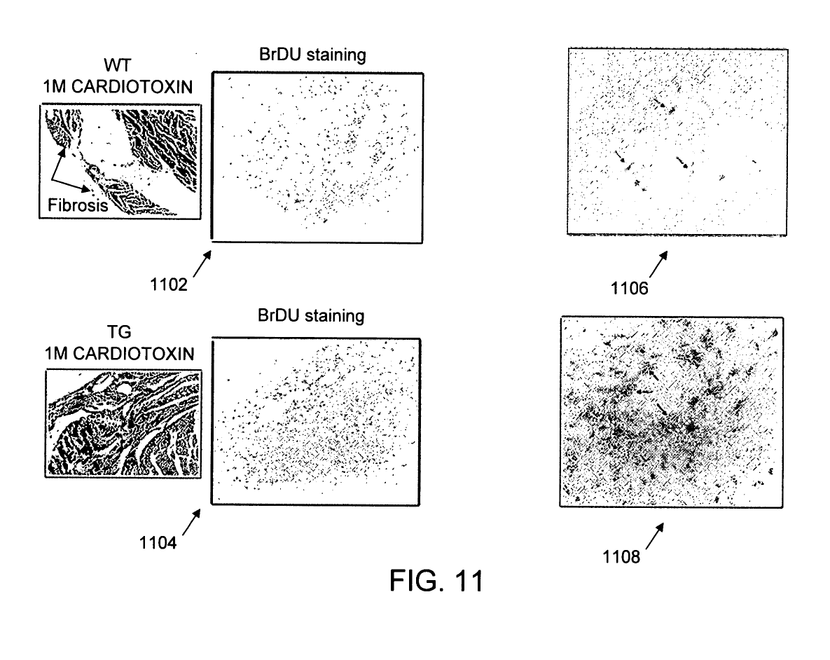

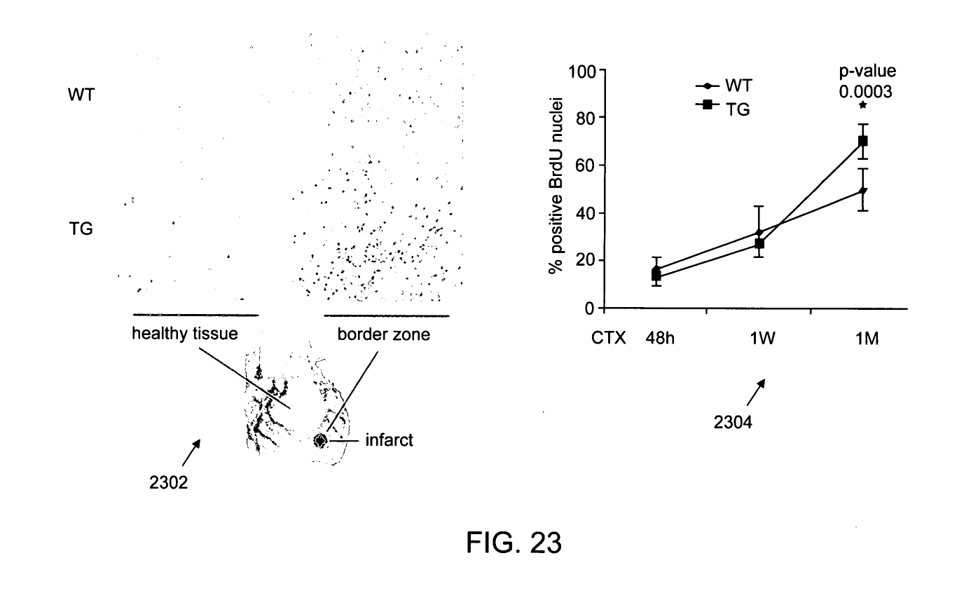

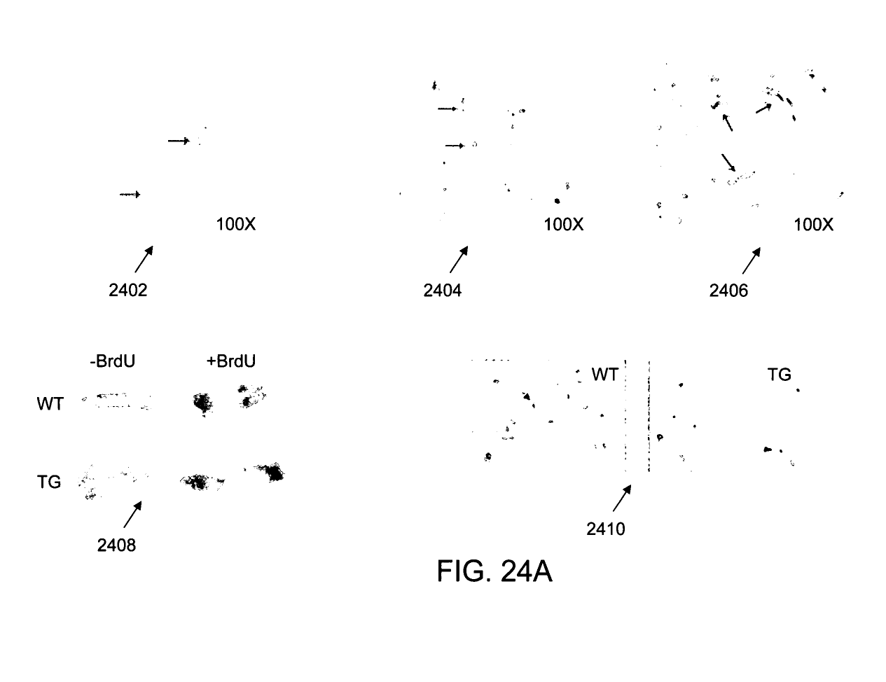

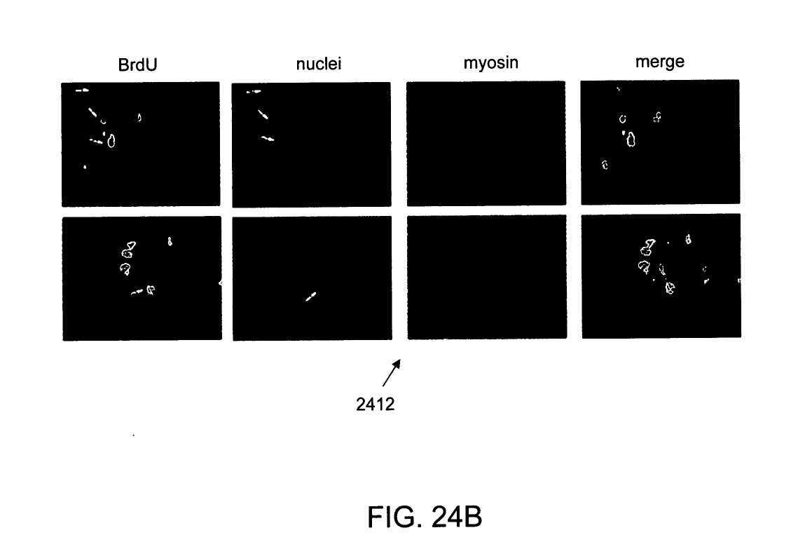

mIGF-1 induced a significant percentage of total cells to enter the cell cycle in response to cardiotoxin injection (FIG. 11B) compared to wild-type hearts (FIG. 11A). It was found that cardiac cells re-enter the cell cycle (FIG. 11C), although cells of diverse lineage were found in the myocardium and in the vessels as shown in FIG. 11D. The nature of these cells is still under investigation. No differences in BrdU incorporation were found in injured hearts 48 hours (13%+/−4% transgenic vs 16%+/−5% wild-type) and 1 week (27%+/−5% transgenic vs 32%+/−11% wild-type) after CTX injection (FIG. 23b). At 1 month after infarct induction, however, 14%+/−0.8% of total cells in the border zone of the mIGF-1 hearts were BrdU positive compared to wild-type hearts (FIG. 23a, b). Frequent incorporation of BrdU in cardiomyocyte nuclei was seen in both cardiac tissue and individual cells after injury (FIG. 24a, b, d, f), although abundant non-muscle cells of diverse morphologies were also labelled in the vessels and surrounding myocardial tissue of mIGF-1 transgenic hearts (FIG. 24c, e, f).

No differences in proliferative state were found in injured heart 24 hour and 1 week after cardiotoxin injection (data not shown), indicating that the regeneration program, following the early activated repair program, is activated as a late step in mIGF-1 overexpressing hearts.

The regenerative properties of the mIGF-1 isoform have been previously documented in skeletal muscle (85,86). In contrast to skeletal muscle, which can regenerate following injury, the mammalian heart has limited restorative capacity. Since supplementary mIGF-1 enables full myocardial regeneration following injury without altering normal heart development or long-term postnatal tissue form and function, it forms the basis of clinically feasible therapeutic strategies to bypass the normal restrictions on mammalian cardiac regeneration.

Despite shifts in signalling pathways accompanied by modest effects on morphology and haemodynamic parameters, continuous expression of mIGF-1 throughout postnatal life did not produce a significant perturbation of normal heart physiology. In contrast to previous studies with other IGF-1 transgenes (19), the hypertrophic growth of the mIGF-1 transgenic hearts did not progress to a pathological phenotype. In response to injury, the cardiac regeneration program induced by mIGF-1 followed a sequential course, involving early resolution of inflammation at the site of injury to prevent scar formation and to make way for the subsequent tissue replacement that restores form and function. Clearly mIGF-1 transgenic hearts are better prepared to contain damage by repressing pro-inflammatory molecules and increasing expression of anti-inflammatory cytokines such as IL4 and IL-10. The mIGF-1 transgene also activates p21, which is important for IGF-1-mediated cell survival upon UV irradiation (76). Prolonging the initial induction of p21 in damaged cardiac tissue enhances DNA repair and genome stability, without precluding cell replacement (87). In addition, expression of p21 suppresses production of IL6 in rheumatoid arthritis, associated with high inflammation of the joints (78). Modulating expression of these important downstream effectors of inflammation may be an important role of the intracellular signalling cascades set in motion by mIGF-1, which provide a conducive environment for cell replacement and tissue restoration.

The delayed cell proliferative response seen in regenerating mIGF-1 transgenic hearts stands in contrast to the effects of direct myocardial injection of fully processed IGF-1 protein, which rapidly induced the appearance of small new myocytes within the infarct at 1 to 2 days after coronary ligation (88). In that system the maximum benefit required a dual stimulation by IGF-1 injection together with the chemotactic effects of co-injected Hepatocyte Growth Factor, pointing either to a different mode of action employed by an expressed transgene product, or to a qualitative difference in the action of the mIGF-1 isoform itself. Elucidation of the roles played by native signal and E peptides in enhancing the regenerative response in mIGF-1 transgenic hearts, without the requirement for additional growth factors, will inform the design of clinically feasible therapeutic strategies to counteract the normal fibrotic tissue formation and consequent cardiac functional impairment in heart disease.

### Example 3

Muscle Expression of a Local IGF-1 Isoform Protects Motor Neurons in an ALS Mouse Model

To assess the effects of supplemental IGF-1 directly on atrophic SOD1 skeletal muscle, a transgenic mouse expressing a full-length precursor of the localized IGF-1 isoform (mIgf-1) that is normally induced transiently in response to muscle damage, but does not enter the circulation (89;90) was exploited. Muscle-restricted mIGF-1 transgene (MLC/mIgf-1) exerts its effects in an autocrine or paracrine manner, circumventing the adverse side effects of systemic rIGF-1 administration. Expression of the MLC/mIGF-1 cassette, delivered either as an inherited transgene or somatically on an AAV vector, induces muscle hypertrophy and strength, and preserves regenerative capacity in senescent and dystrophic mice (20;89;91) through enhanced stem cell recruitment (92). In the present study skeletal muscle is established as a primary target in inherited forms of ALS by showing that localised expression of the co-inherited MLC/mIGF-1 transgene exclusively in the skeletal musculature of SOD1G93A mice counteracted the symptoms of ALS, induced satellite cell activity and markers of regeneration, stabilized neuromuscular junctions and led to a reduction in astrocytosis in the SOD1G93A spinal cord. These observations offer novel approaches to the attenuation of motor neuronal degradation and underscore the importance of IGF-1 isoform selection when designing therapeutic strategies to treat ALS.

Results and Discussion

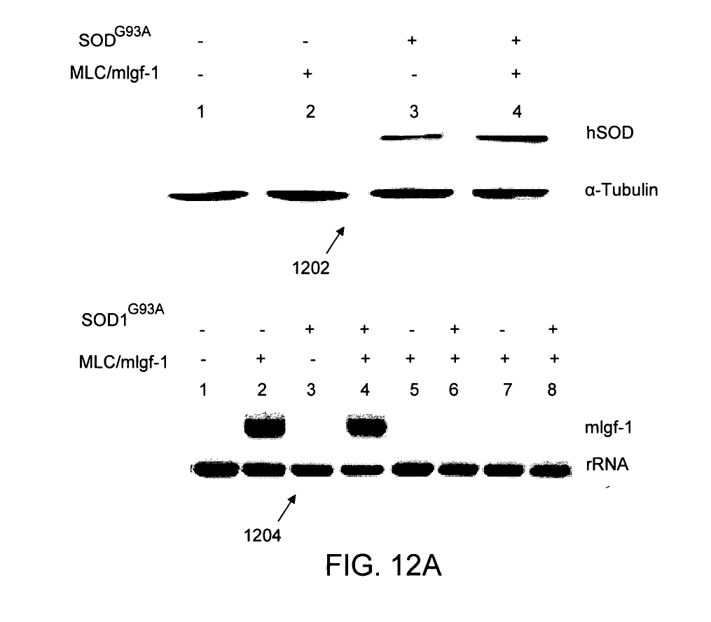

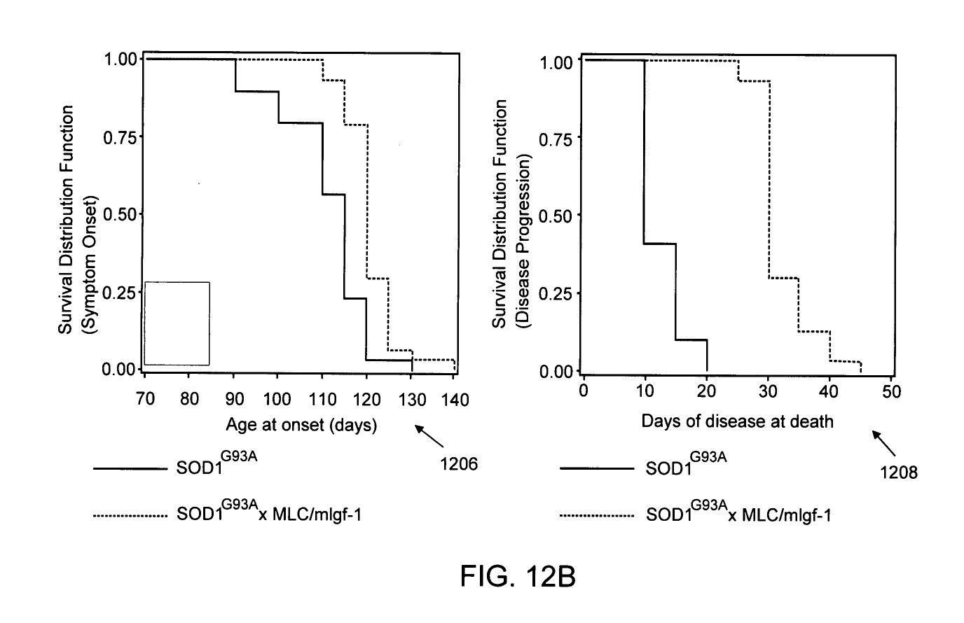

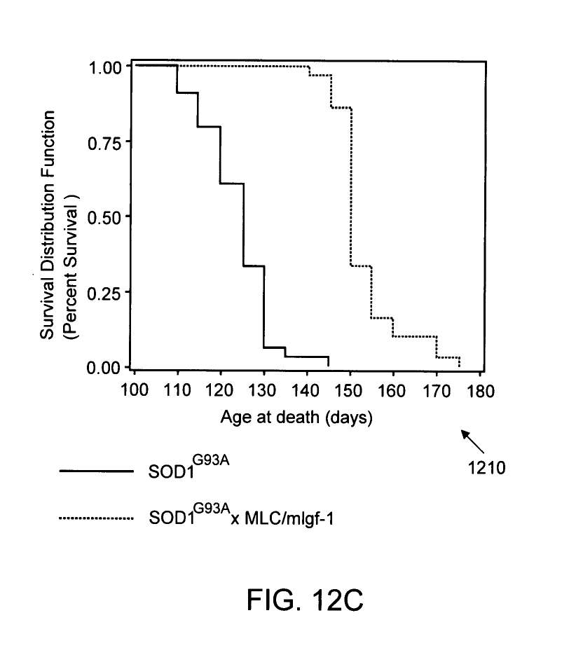

Muscle-specific mIGF-1 delays the progression of the disease and prolongs the life span of SOD1G93A mice. To evaluate the effects of mIGF-1 on the SOD1G93A neurodegenerative phenotype, double transgenic SOD1G93A and MLC/mIGF-1 transgenic mice were compared to their SOD1G93A littermates. The SODG93A and SOD1G93A×MLC/mIGF-1 (SODG93AmIgf-1) transgenic mice were selected for high copy number of the SODG93A allele and for same expression level of the human transgenic protein (FIG. 12a). Notably, mIGF-1 transgene was selectively expressed in skeletal muscle of both MLC/mIGF-1 and SODG93AmIGF-1 transgenic mice (FIG. 12b, lanes 2, 4), whereas it was not expressed in the brain and spinal cord of these mice (FIG. 12b, lane 5-8), not even in skeletal muscle of wild type and SODG93A mice (FIG. 12b, lanes 1, 3). At 111 (1.76 STDERRmean) days of age, disease onset was observed in the mutant SOD1G93A transgenic mice (n=30) (FIG. 12c). Notably, the SOD1G93A mice died within 10 days (0.62 STDERRmean) of clinical disease onset (FIG. 12d), with a maximal life-span of 145 days (FIGS. 12e). By contrast, localized expression of mIGF-1 delayed the onset (FIG. 12c) and in particular the progression (FIG. 12d) of disease, increasing the survival of SODG93AmIGF-1 mice (n =30) by approximately 30 days to a maximal lifespan of 175 days (FIG. 12e). Comparative analysis revealed that the difference between SOD1G93A and SODG93AmIGF-1 were significantly relevant for onset (_{χ}2 LR=18.67, P<0.0001), progression (_{χ}2 LR=67.07, P<0.0001), and survival ((_{χ}2LR=63.24, P<0.0001).

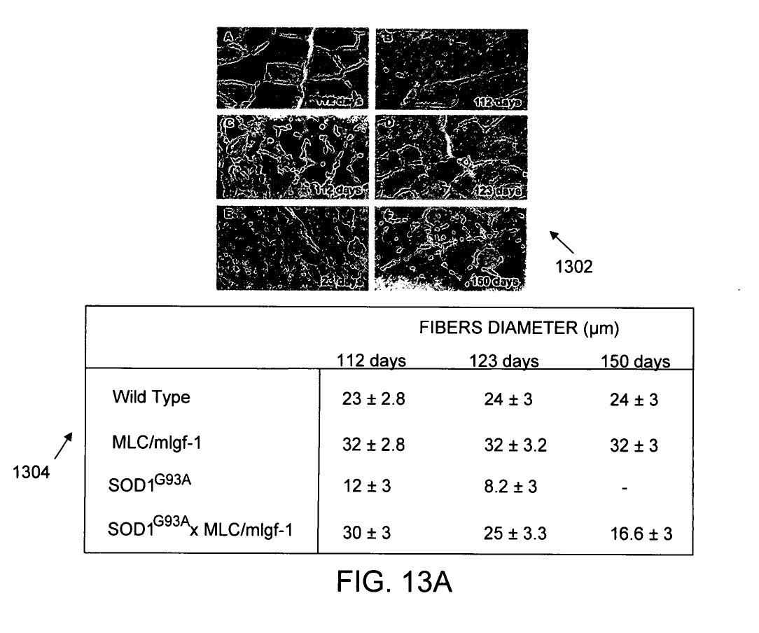

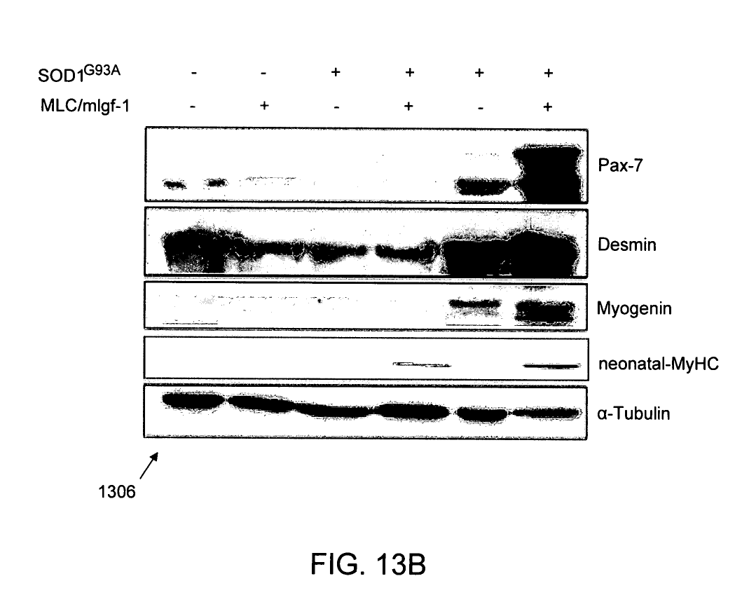

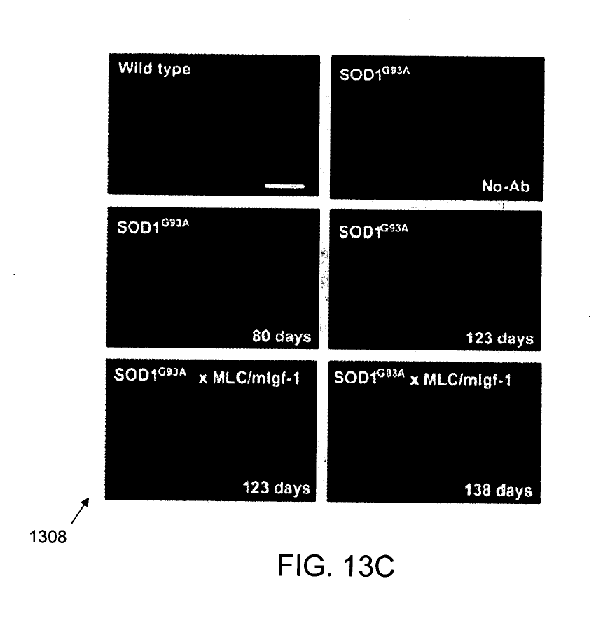

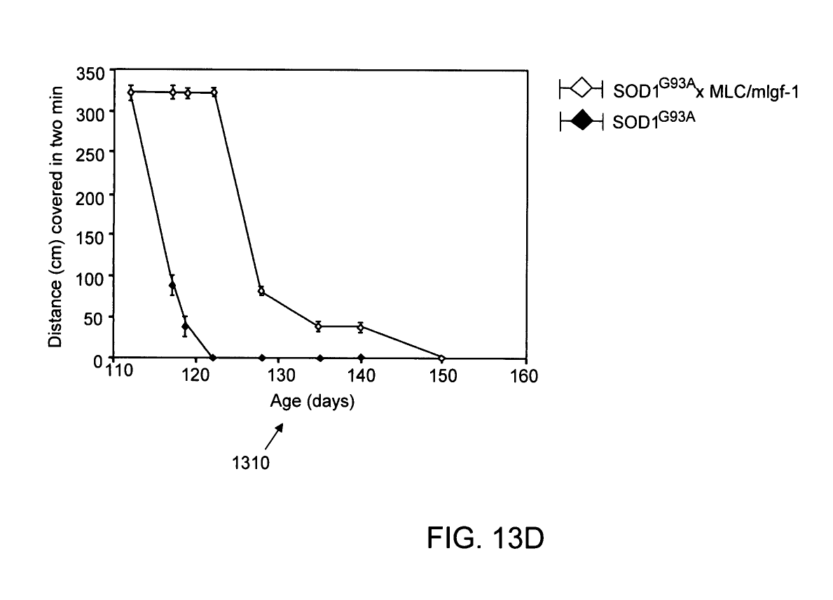

mIGF-1 expression attenuate muscle atrophy, increasing satellite cell activation in SOD1G93A mice. SOD1G93A (n=7) and SODG93AmIGF-1 (n=7) transgenic mice were analysed at different stages of disease. At 123 days, motor neuronal degeneration of SOD1G93A mice was accompanied by severe muscle atrophy (FIG. 13a, panel E), and complete muscle paralysis (hereafter this stage is indicated as paralysis stage). In contrast, at the same age, SODG93AmIGF-1 transgenic mice did not show dramatic and evident signs of muscle disease, but only difficulty in extending the limbs when suspended (hereafter this stage is indicated as symptom onset). Moreover, muscle atrophy was substantially attenuated in SODG93AmIGF-1 offspring even after onset of denervation and paralysis stage (153 days of age) (FIG. 13a, panel F; FIG. 13c). In addition, markers of satellite cell activity, such as Pax-7 and desmin, were increased to varying extents in affected SODG93A mice (FIG. 13c), whereas hallmarks of satellite cell activity and fiber maturation, including centralized nuclei (FIG. 13a yellow arrows), Pax-7 isoforms, desmin, myogenin, and neonatal MyHC expression were present exclusively in the SODG93AmIGF-1 muscles at all stages of disease, including at paralysis stage (FIG. 13c and data not shown), suggesting that satellite cells activation and maturation contribute to the maintenance of muscle phenotype induced by mIGF-1 expression (93). Motor neurons are known to regulate the properties of the myofibers they innervate by selective activation of fiber-specific-gene expression. Immunohistochemical analysis (FIG. 13d) revealed that fiber type composition was completely altered in SOD1G93A soleus muscle even prior to overt disease, with a shift in fiber type, increasing fast fibers. In contrast, the heterogeneity of muscle fibers was maintained for a more extended period in SODG93AmIGF-1 transgenic mice, which showed shift in fiber type composition only at later stage of disease (138 days) (FIG. 13d). In contrast at paralysis stage (153 days) there was not significant difference in fiber type composition between SOD1G93A and SODG93AmIGF-1 transgenic mice (data not shown). The alteration in the heterogeneity of muscle fibers of SOD1G93A mice indicate an alteration in motor neuron activity even prior to overt disease and confirm the hypothesis that the delaying in the progression and severity of ALS diseases, by mIGF-1 expression, may depend on the maintenance of muscle integrity. The functional integrity of the muscles in the SODG93AmIGF-1 transgenic mice was confirmed by a walk test (FIG. 13e) performed at different ages. At 112 days, SOD1G93A mice (n=7) showed symptom onset without evident alterations in fuinctional parameter. The condition of SOD1G93A mice rapidly deteriorated at 115 days, as shown by the shortening of their stride (FIG. 13e). In contrast, the pathological sign of disease were delayed in SODG93AmIGF-1 transgenic mice (n=7), as shown by the capacity of the mice to walk 30±7 cm further when analysed at same age of SOD1G93A mice and by their ability to move for a more extended period of time (FIG. 13e). These data suggest that promotion of muscle satellite cell activity and hypertrophy through mIGF-1 can be considered as an alternate therapeutic approach to counteract muscle wasting associated with ALS disease.

An activated calcineurin isoform is induced in SOD1G93A x MLC/mIGF-1 muscle. It has been reported that mutant SOD1 interferes directly with the protein phosphatase calcineurin-A (CnA) activity, supporting a role for calcineurin-regulated biochemical pathways in the pathogenesis of ALS (94). In addition, in skeletal muscle CnA has been implicated in myocyte hypertrophy (95) and fiber type conversion (96), inducing a signaling cascade that plays an important role in tissue remodeling after injury (97). In a study of selective CnA subunit isoform expression, it was recently established that the alternatively spliced variant CnA-β1 is up-regulated in regenerating skeletal muscle fibers (ms in preparation).

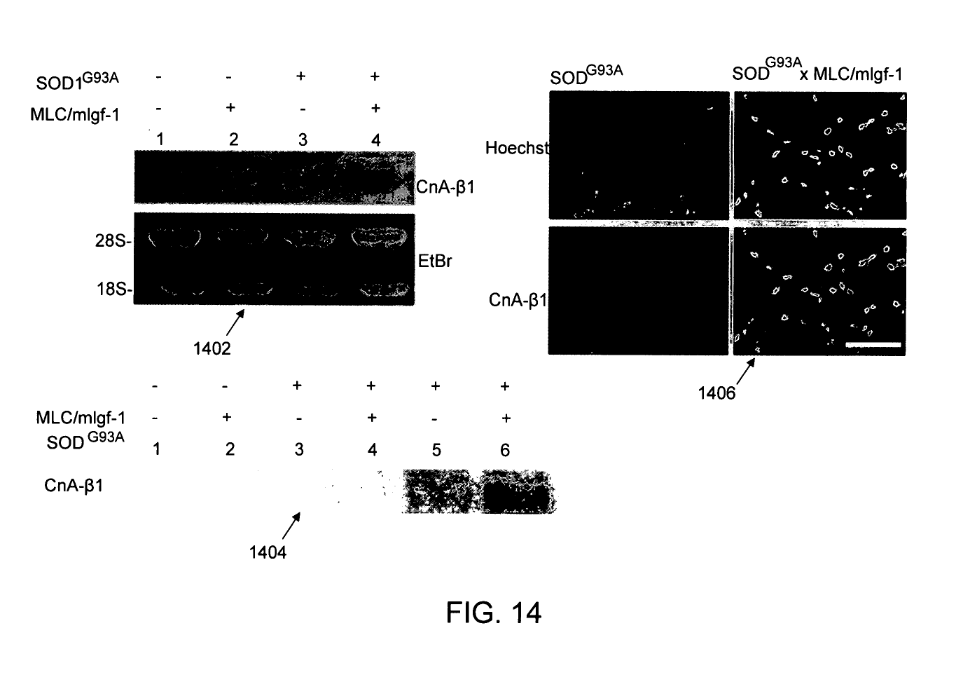

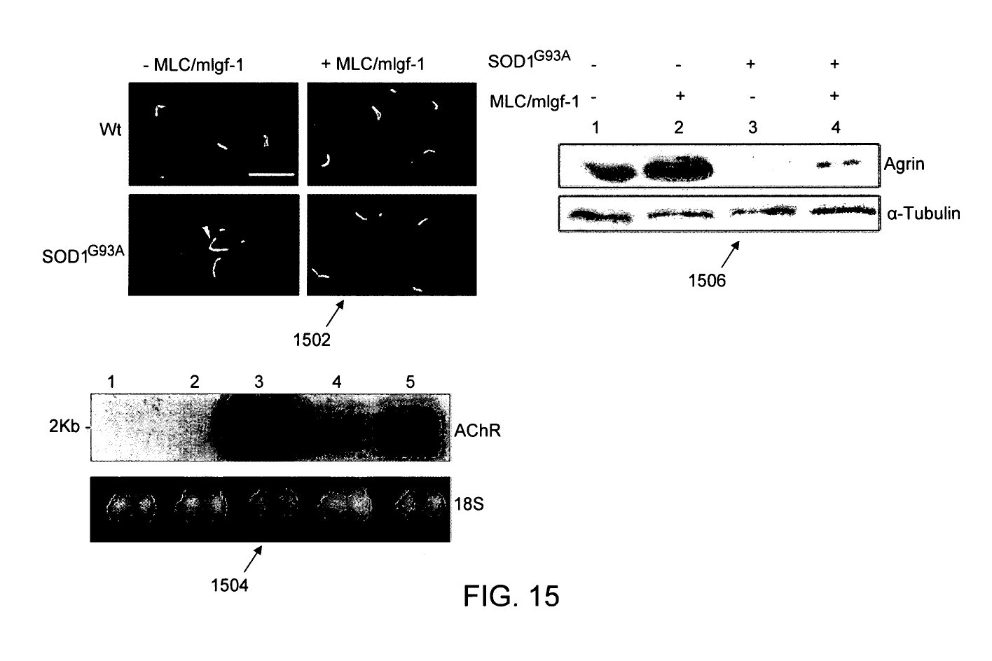

In this study it was verified whether the activation of satellite cells and the maintenance of muscle phenotype involved the induction of CnA-β1 expression. Although the normally low levels of CnA-β1 expression were not raised in SOD1G93A muscles (FIG. 14a lane 3; FIG. 14b lane 5, FIG. 14c), or in uninjured wild type and MLC/mIGF-1 muscle (FIG. 14a and 14b, lane 1 and 2), SODG93AmIGF-1 regenerating muscle dramatically increased CnA-β1 transcripts (63%±5%) (FIG. 14a lane 4) and nuclear protein (52%±7%) (FIG. 3b lane 6, FIG. 3c) after onset of clinical symptoms and its expression remained at high levels even at end stage of disease. CnA-β1 represents a potential molecular player underlying the prolongation of muscle integrity even after denervation.

Preservation of Neuromuscular Junctions in SODG93AmIGF-1 Mice

Alterations in motor neuronal activity typical of denervated muscle and motor neuron diseases also affects the configuration of neuromuscular junctions in SOD1G93A mice, characterised by the diffusion of acetylcholine receptor (AChR) postsynaptic clusters (FIG. 15a, yellow arrow). At 123 days, SOD1G93A paralyzed muscle showed 65%±0.19σ of diffuse AChR expression, whereas AChR cluster aggregates (FIG. 15a, red arrows) were preserved in muscles of age matched SODG93AmIGF-1 mice, which showed only 3.3%±0.4a of diffuse AChR expression. At comparable end-stage disease, SODG93AmIGF-1 muscle displayed only 30%±0.20σ of diffuse AchR expression. These results were confirmed by Northern blot analysis (FIG. 15b); high AchR expression levels in SOD1G93A muscle were reduced in SODG93AmIGF-1 mice at all stages observed.

Densitometric analysis (n=6) revealed that AChR mRNA expression in SOD1G93A paralysed muscle (123 days) was 56% +5% higher than that observed in age matched SODG93AmIGF-1 mice; whereas the increase in mRNA expression in SOD1G93A mice was of 27%±6% when SOD1G93A and SODG93AmIGF-1 mice where analyzed at comparable end-stage disease. This suggests that mIGF-1 delays the progression of the disease, stabilizing the innervation of muscle fibers. The localization of AChR clusters at the endplate requires the expression of agrin, a large proteoglycan in the synaptic cleft that plays an important role in the maintenance of the molecular architecture of the postsynaptic membrane (98). Agrin expression showed a dramatic down-regulation in paralyzed SOD1G93A muscle compared to SODG93AmIGF-1 muscle (FIG. 15c) analyzed at comparable end-stage disease, further underscoring a role for local expression of IGF-1 in the maintenance of muscle innervation. Muscle-restricted mIGF-1 prolongs motor neuronal function in SOD1G93Amice.

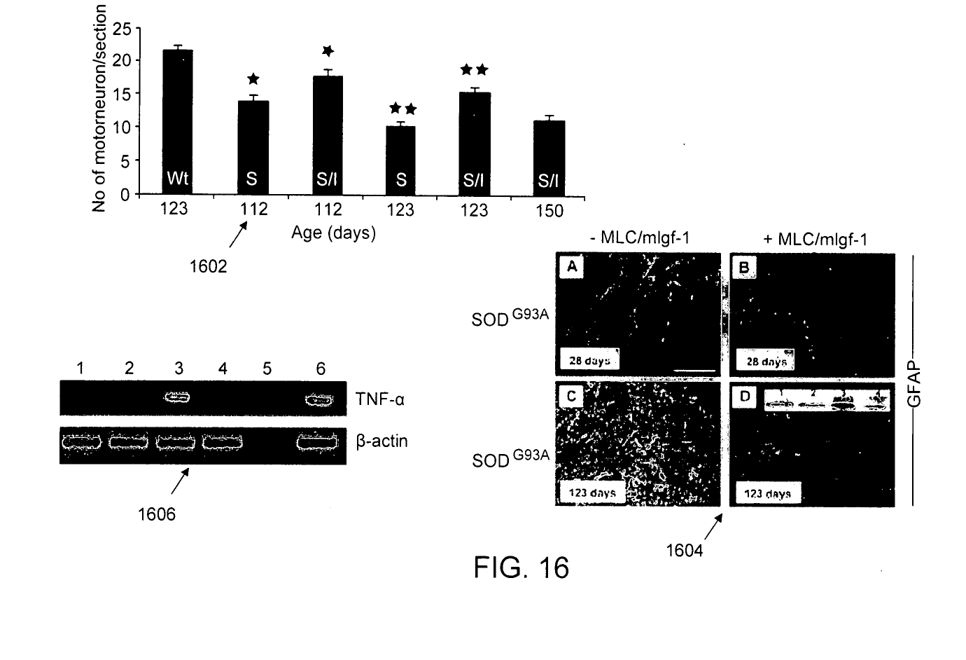

The extent to which muscle-restricted mIGF-1 expression preserves the motorneuron during the progression of ALS disease was then determined. Histological analysis of the ventral spinal cord revealed that SOD1G93A mice (n=7) presented a progressive reduction in the number of motor neuron from clinical onset to end stage disease. Specifically, SOD1G93A mice showed a reduction of 37% and 55% in the number of motorneurons at clinical onset (112 days) and at end stage disease (123 days) respectively (FIG. 16a). In contrast mIGF-1 expression induced motomeuron survival in SODG93AmIGF-1 mice (n=7) at all age and stage observed with significant differences at 112 and 123 days of age (FIG. 16a). One of the prominent markers of motor neuron dysfunction in ALS mice is the activation of astrocytes and microglia, leading to motor weakness and neural loss (99). Comparable patterns of GFAP immunoreactivity were found in spinal cords of SOD1G93A (n=6) and SODG93AmIGF-1 (n=6) transgenic mice before the symptom onset (28 days) (FIG. 16a panels A-B, panel D-insert lanes 1,2). However, at paralysis stage (123 days) the spinal cord of SOD1G93A mice demonstrated a marked increase in astroglial activation (FIG. 16b panel C), with an increase in GFAP expression of about 35%±3% (FIG. 16b panel D-insert, lane 3) compared with the GFAP expression levels displayed in the spinal cord of age matched SODG93AmIGF-1 transgenic mice (FIG. 16b panel D and insert lane 4). At comparable end stage disease, there were no significant differences in GFAP expression between SOD1G93A and SODG93AmIGF-1 mice, although SOD1G93A mice continued to have a 13% more GFAP expression as compared to SODG93AmIGF-1 mice (data not shown).

The activation of astroglia can be correlated with the expression of certain cytokines, such as TNF-α, which enhances the response to inflammatory states and contributes to the progression of neurological dysfunction in SOD1G93A mice (100). While TNF-α expression was normally undetectable in the CNS of healthy mice (FIG. 16c, lanes 1, 2), it was accumulated in the spinal cord of SOD1G93A mice at paralysis stage (123 days) (FIG. 16c lane 3). In contrast, TNF-α expression was not apparent in the spinal cord of SODG93AmIGF-1 transgenic mice (FIG. 16c lane 4). This suggests that MLC/mIGF-1 hypertrophic muscle functions as a protective tissue for the CNS, modulating reactive astrocytosis and inflammatory cytokines that normally exacerbate the pathogenesis of ALS disease.

These results suggest that ALS is a “multi-systemic” disease in which the alteration in structural, physiological and metabolic parameters in different cell types (muscle, motorneurons, glia) may act synergistically to exacerbate the disease and evidences a functional cross-talk between neuronal and not neuronal cells (101).

Therefore, the present study serves to refocus therapeutic strategies to attenuate motor neuronal degradation towards skeletal muscle. It remains to be determnined whether the dramatic prolongation of CNS tissue integrity in SODG93AmIGF-1 mice derives from the direct retrograde transport of transgenic mIgf-1, or indirect action either through distal activation endogenous IGF-1 expression, or through other trophic factors secreted by SODG93AmIGF-1 muscle.

In a previous retrograde transport of the AAV-IGF-1 vector from muscle to motor neuron was deemed necessary to achieve the therapeutic effects, since the same gene delivered to the muscle by a lentiviral vector, which cannot retrotransport, proved ineffective. The results of the present study suggest other explanations. An undefined IGF-1 cDNA used by Kaspar et al (102) may have encoded the more prevalent circulating gene product, which once secreted from the muscle, is not associated with extracellular matrix and disperses in the circulation (90). In contrast, the mIGF-1 isoform used in the present and previous studies (20;89;91;92) does not enter the circulation, but accumulates in the tissue of synthesis where its autocrine/paracrine function is concentrated. Thus, the importance of IGF-1 isoform choice in designing therapeutic strategies cannot be overstressed, since their diverse biological activities lead to radically different outcomes (90).

Whatever the mode of action, the feasibility of localized synthesis of the mIGF-1 isoform to activate survival mechanisms in distal damaged tissues represents a powerful approach to counteract the degeneration of both muscle and motor neuron in ALS disease.

Material and Methods

Mice

SODG93A transgenic mice (Jackson Laboratory) express the human mutant SOD1G93A allele containing the Gly93—>Ala (G93A) substitution, driven by its endogenous human promoter (103). The SOD1G93A B6J mice were crossed with MLC/mIGF-1 FVB mice (89) for 7 different generations to obtain SODG93AmIGF-1 B6J inbred transgenic mice. The animals were housed in a temperature-controlled (22° C.) room with a 12:12 h light-dark cycle.

Walk Test

The walk test was performed in a scaled ramp, accordingly to the method reported by Gurney et al (103). The mice were allowed to explore the cage for 1 minute and then they were left to walk for 2 minutes. The hind feet of the mice were painted with ink and the track left by the mice were recorded on a paper tape. The test was performed in horizontal, laminar flow hood to maintain barrier conditions.

Histological and Immunofluorescence Analysis

Muscle tissue was embedded in TBS-tissue freezing medium and frozen in nitrogen-cooled isopentane. For histological analysis, 7 gm tissue cryosections were fixed in 4% paraformaldehyde and stained with hematoxylin/eosin. For immunofluorescence analysis, 7 μm tissue sections were fixed with 4% paraformaldehyde, washed in PBS with 1% BSA and 0.2% Triton X, pre-incubated 1 hr in 10% goat serum at R.T. and incubated overnight with primary antibodies: neonatal Myosin Heavy Chain (neo-MyHC), MyHC-slo), MyHC-fas), Alexa Fluor™ 488 conjugated α Bungarotoxi), CnA-β), GFA). Nuclei were visualized by Hoechst staining. Stained cells were observed under an inverted microscope (model Axioskop 2 plus; Carl Zeiss Microimaging, Inc) using 20× or 40× lenses, and images were processed using Axiovision 3.1).

RNA Preparation and Northern Analysis.

RNA was extracted from muscles by RNA-TRIZOL-kit (Gibco BRL). Total RNA was separated in 1.3% agarose gel and hybridized as previously described (Musarò and Rosenthal, 1999).

RT-PCR Analysis

RNA from spinal cord of wildtype, MLC/mlgf-1, SODG93A and SODG93AmIGF-1 transgenic mice were used in RT-PCR assay. The following oligonucleotides were used: TNF-α sense 5′CCCAGACCCTCACACACTCAGAT3′ and anti sense 5′TTGTCCCTTGAAGAGAACCTG3′; β-Actin sense 5′GTGGGCCGCTCTAGGCACAA3′ and anti sense 5′CTCTTTGATGTCACGCACGATTTC3′.

Protein Extraction and Western Blot Analysis

Protein extraction was performed in lysis buffer (50 mM Tris-HCl pH 7.4, 1% w/v Triton×100, 0.25% Sodium Deoxycholate, 150 mM Sodium Chloride, 1 mM Phenylmethylsulfonyl Fluoride, 1 μg/ml Aprotinin, 1 μg /ml Leupeptin, 1 μg/ml Pepstatin, 1 mM Sodium Orthovanadate, 1 mM Sodium Fluoride). Equal amounts of protein from each muscle lysate were separated in SDS polyacrylamide gel and transferred onto a Hybond C Extra nitrocellulose membrane. Filters were blotted with antibodies against human-SOD, myogenin, desmin; neo-MyHC, Agrin.

### Example 4

Comparison of Signal Peptide and E Peptide Functions

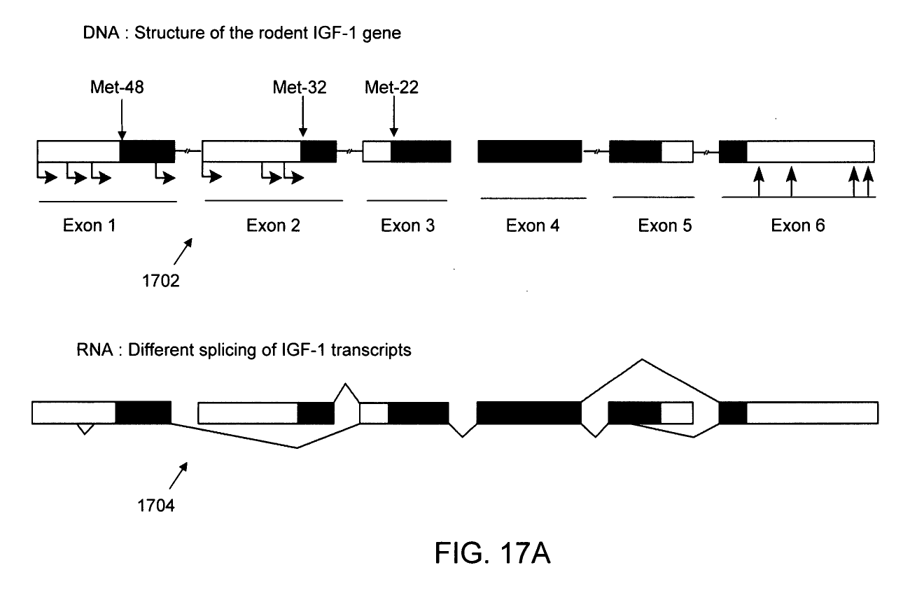

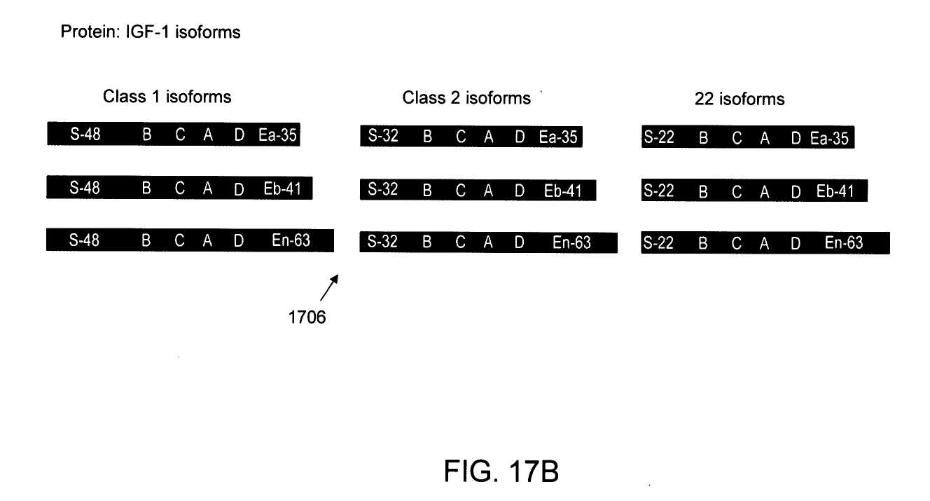

To assess the various effects of the different signal peptides and E peptides a series of studies were carried out involving direct comparisons of the IGF-1 isoforms in various biological systems. FIG. 17 shows a schematic representation of the IGF-1 gene and the various signal/E peptide isoforms.

Table 5 shows the usage of the various IGF-1 isoforms, in different muscle types, in wildtype mice.

TABLE 5
Normal mouse IGF-1 isoform usage.

| | Wildtype | Exercised | Aged muscle (only | Dystrophic |
|---|---|---|---|---|
| Construct | muscle | muscle | in diaphragm) | muscle |
| Class 1 IGF- | + | ++ | + | ++ |
| 1A | | | | |
| Class 1 IGF- | − | − | + | + |
| 1B | | | | |
| Class 2 IGF- | − | − | + | − |
| 1A | | | | |
| Class 2 IGF- | − | − | + | ++ |
| 1B | | | | |

IGF-1 Signal Peptides Control Myoblast Differentiation

To assess the effect of the IGF-1 signal peptides on myoblast differentiation kinetics, L6 proliferating mononucleated myoblast cultures were transfected with MLC/IGF-1A contained in a muscle specific expression vector. IGF-1A was expressed in conjunction with either the class 1, 2, or 3 signal peptide.

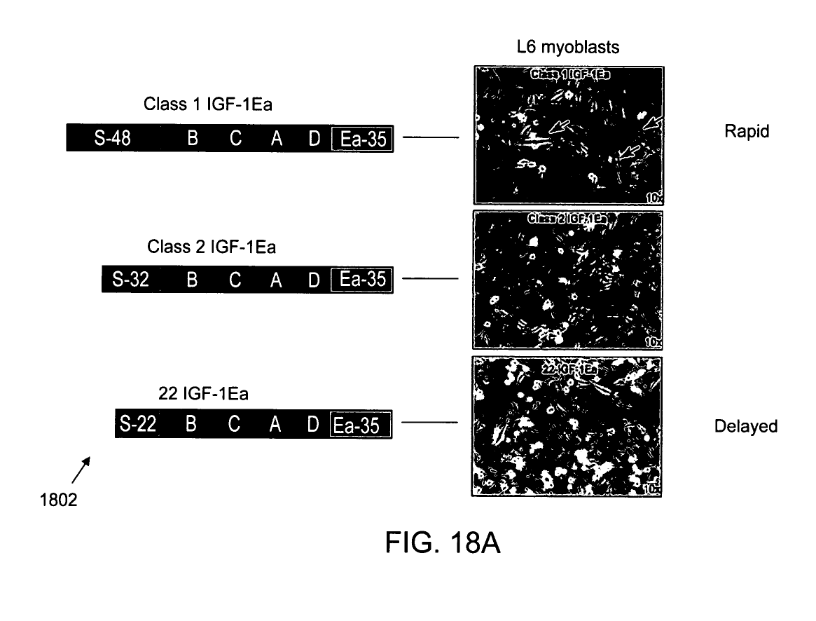

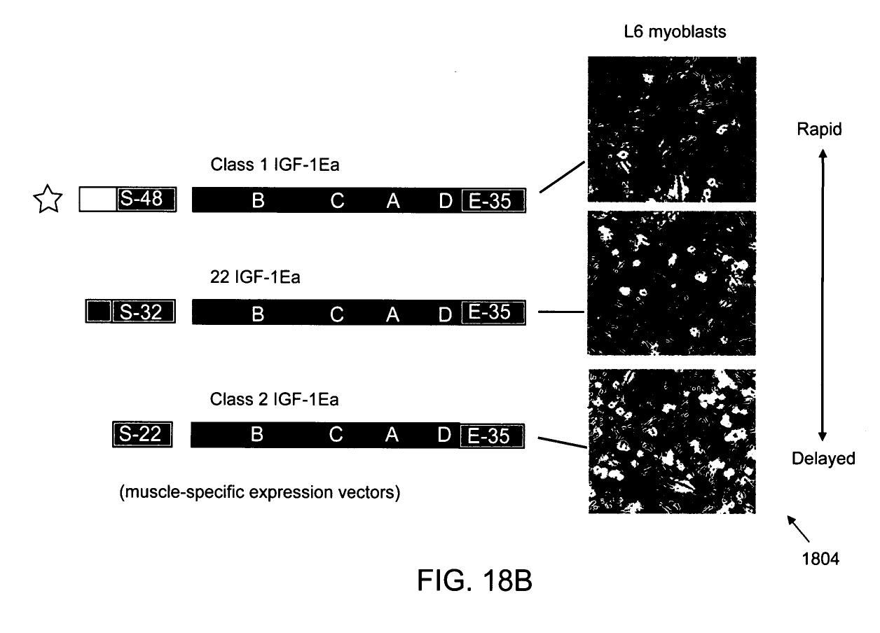

FIG. 18 shows the effect of each of the constructs on myoblast differentiation. Class 1 IGF-1-1A causes L6 myoblasts to undergo rapid differentiation. Class 2 IGF-1A has little effect on L6 differentiation. Class 3 IGF-1A (Δ IGF-1A) causes delayed differentiation in L6 myoblasts.

These results suggest that the fate, and subsequent function of the IGF-1 peptide, is controlled by the various signal peptides.

Different Phenotypes Associated with E Peptides

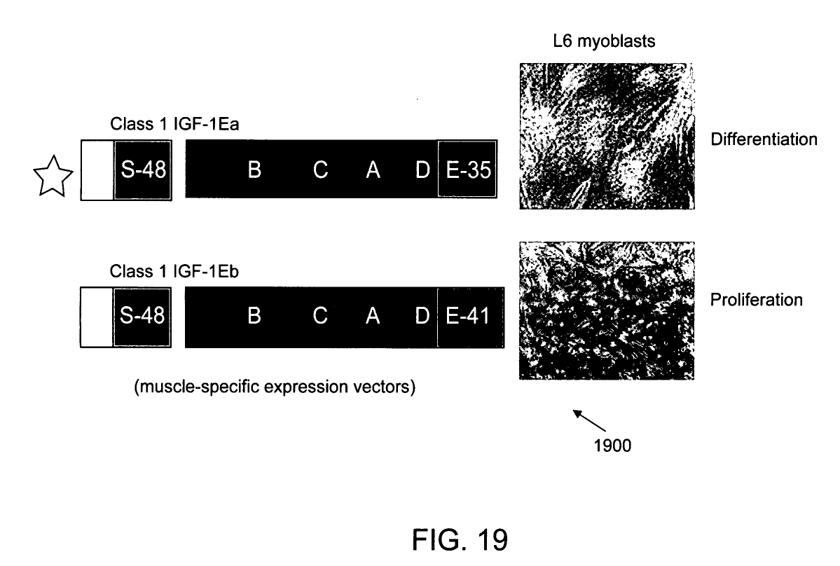

To asses the effect of the IGF-1 E peptides on myoblast growth, L6 proliferating mononucleated myoblast cultures were transfected with MLC/Class 1 IGF-1A or MLC/Class 1 IGF-1B contained in a post-mitotic expression vector. FIG. 19 shows the differing effect of the two constructs. Class 1 IGF-1A induces cellular differentiation in L6 myoblasts. In contrast, the Class 1 IGF-1B construct induces cellular proliferation.

These results suggest that the IGF-1 E peptides are responsible for controlling the different functions of the IGF-1 peptide.

Testing IGF-1 Isoform Function in Vivo.

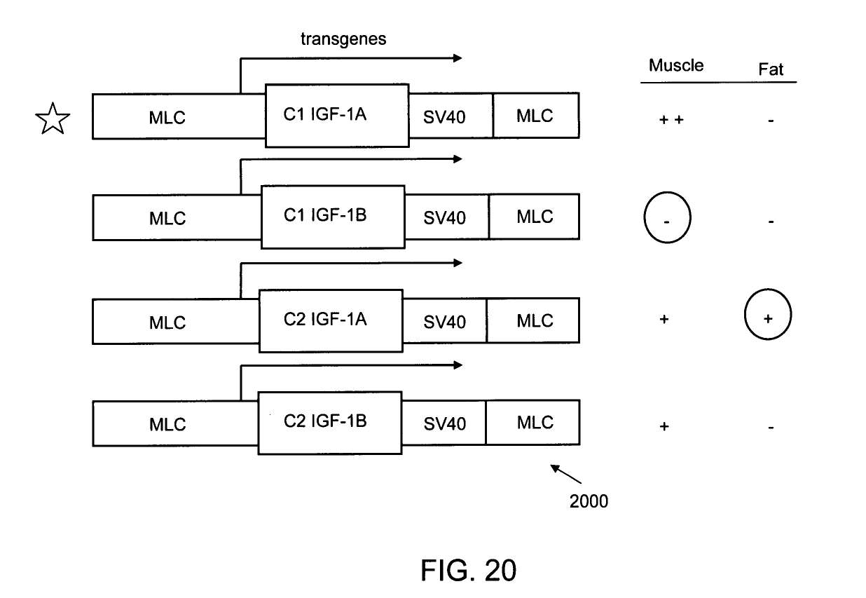

To assess the effects of the varying IGF-1 signal peptides in conjunction with the E peptides in vivo, transgenic mice were engineered following the methodology detailed in the previous examples. A total of six constructs were generated, representing the major IGF-1 isoforms (FIG. 20).

Transgenic mice expressing the Class 1 IGF-1A (mIGF-1) isoform show a hypertrophic response in skeletal muscle and a consequential increase in muscle mass. In comparison, transgenic mice expressing the Class 1 IGF-1B isoform show no increase in muscle hypertrophy. Tables 6-8 show phosphoproteins that are up-regulated, down-regulated or unaltered in Class 1 IGF-1A transgenic mouse muscle.

TABLE 6
Phosphoproteins up-regulated in Class 1 IGF-1A transgenic
mouse muscle

| | % |
|---|---|
| Description | change |
| AMP-activated protein kinase alpha (T172) | 162 |
| Bruton's tyrosine kinase (Y223) | 24 |
| Cyclin-dependent kinase 1 (Y15) | 9 |
| Etk (BMX) (Y40) | 17 |
| Raf (S259) (60) | 16 |
| Phosphoinositide-dependent protein kinase | 53 |
| 1 (S241) | |
| mTOR (S2448) | 112 |
| p70 S6 kinase (T421/T424) | 254 |
| Retinoblastoma Protein (S780) | 36 |

TABLE 7
Phosphoproteins down-regulated in Class 1
IGF-1A transgenic mouse muscle

| | % |
|---|---|
| Description | change |
| Protein kinase C alpha/beta (T638) | −4 |
| Protein kinase C delta (T505) | −70 |
| Protein kinase theta (T538) | −8 |
| Protein kinase D (Protein kinase mu) | −37 |
| (S916) | |
| PKC-related kinase 1 (T778) | −29 |
| PKC-related kinase 2 (T816) | −50 |
| Raf (S259) (70) | −10 |
| Protein kinase B (T308) (Akt) | −4 |
| Glycogen synthase kinase-3 alpha | −50 |
| (S21) | |
| Glycogen synthase kinase-3 beta | −18 |
| (S9) | |
| I-kappa-B kinase beta (S181) | −18 |
| Lyn (Y507) (44) | −5 |
| Lyn (Y507) (46) | −13 |
| MAPK/Erk kinase 1/2 (S217/221) | −60 |
| MKK3/6(1) (S189/S207) | −50 |

TABLE 8
Phosphoproteins unaltered in Class 1 IGF-1A transgenic
mouse muscle

| | % |
|---|---|
| Description | change |
| eIF4E binding protein (S65) (16) | 0 |
| eIF4E binding protein (S65) (17) | 0 |
| eIF4E binding protein (S65) (18) | 0 |
| CaMKII (T286) | 0 |
| Cyclin-dependent kinase 1 (T161) | 0 |
| I-kappaB kinase alpha (S180) | 0 |
| MAP kinase activated protein kinase 2 | 0 |
| (T334) | |
| MKK6 (2) (S207) | 0 |
| MAP kinase interacting kinase 1 | 0 |
| (T197/202) | |
| p38 MAPK (T180/Y182) | 0 |
| p70 S6 kinase (T389) | 0 |
| p85 S6 kinase 2 (T412) | 0 |
| p85 S6 kinase 2 (T444/S447) | 0 |
| 90 kDa Ribosomal S6 Kinases (S380) | 0 |

Transgenic mice expressing the Class 2 IGF-1A isoform exhibit a mildly hypertrophic phenotype in conjunction with a significant increase in adipose tissue. In comparison, transgenic mice expressing the Class 2 IGF-1B isoform exhibit a mildly hypertrophic phenotype with no increase in adipose tissue.

Several inferences can be drawn from these results:
1. Of the local (Class 1) isoforms, the Ea peptide-containing isoform has the most dramatic effect on local tissue. Since the two locally acting isoforms differ only by their E-peptide, a specific role for the Ea peptide can be predicted.
2. Of the circulating (Class 2) isoforms, the Ea peptide-containing isoform has the most dramatic anabolic effect on distal tissues, which implies that it travels to those tissues with the Ea peptide still attached to the IGF-1 peptide. From this it appears that adipose tissue may be the most sensitive to circulating IGF-1A.

From these inferences it can be concluded that the Ea and Eb peptides have different effects on regulation and differentiation of cells, illustrated here with effects on both muscle and adipose tissue.

### Example 5

The Differential Role of IGF-1 Isoforms in Skeletal Muscle

In order to gain a greater understanding of the differential role of the IGF-1 isoforms and the specific functions of both the various signal peptides and E peptides, the studies described in example 4 were continued in more detail.

Two main approaches were taken to attempt to understand the function of different IGF-1 isoforms. Initially four IGF-1 isoforms were transiently over-expressed in a doxocycline-inducible manner in L6E9 cells, a myogenic cell line that doesn't express endogenous IGF-1. To further elucidate the in vivo effects of IGF-1 isoform, six transgenic mouse lines, each over-expressing one of the IGF-1 isoforms in skeletal muscle were generated and analyzed for their effect on the skeletal muscle phenotype. Another tool for understanding IGF-1 function has been the generation of IGF-1 inducible transgenic mice. Selected isoforms were cloned into a doxocycline-inducible vector and transgenic animals were generated and crossed with a skeletal muscle-specific inducer mouse to achieve timed IGF-1 transgene expression.

Testing IGF-1 Isoform Function in vitro

To test the function of IGF-1 isoforms in vitro, the “Tet-on” system was applied (104)(105), in which the gene encoding a modified tetracycline repressor protein (reverse tetracycline transactivator (rtTA)) is expressed via a minimal human CMV promoter, while the different IGF-1 isoforms are under the control of a rtTA responsive target promoter.

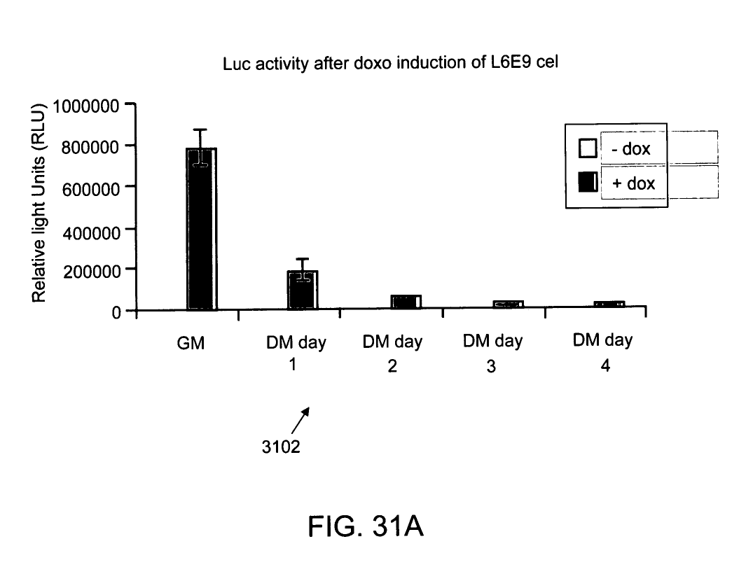

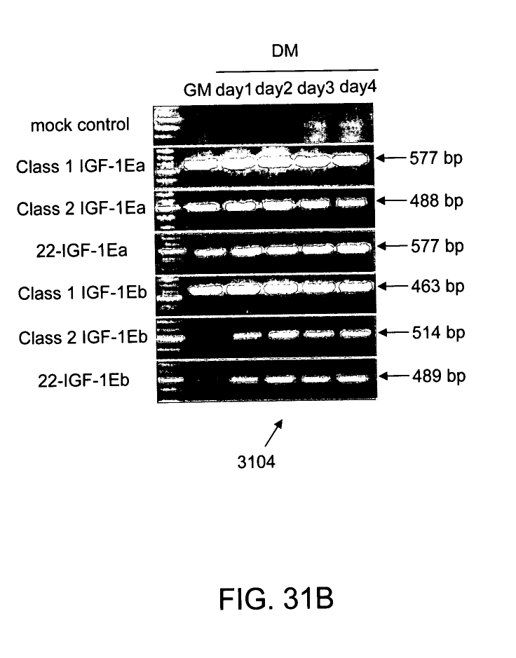

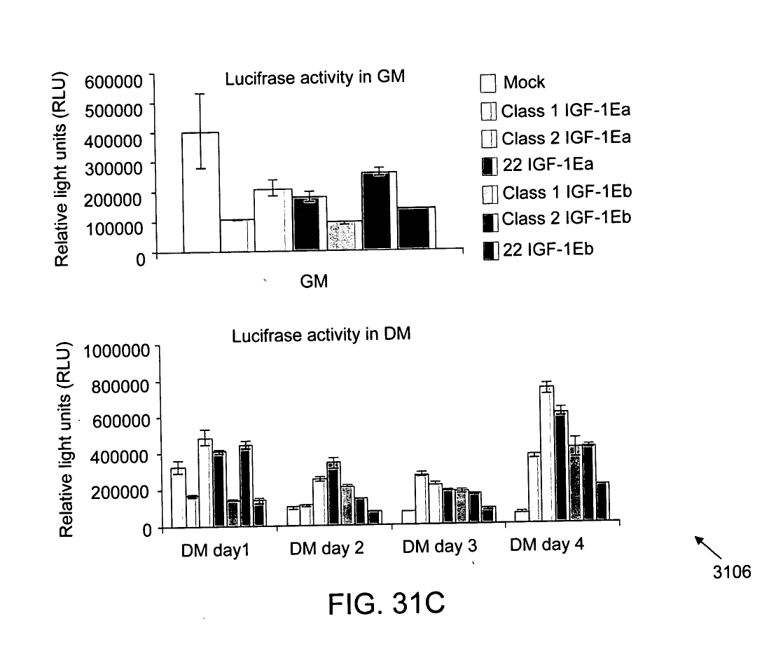

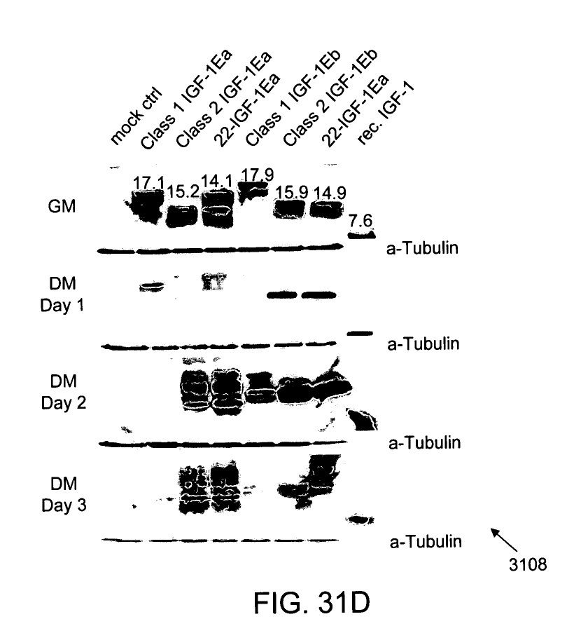

In an initial test experiment, cells were double-transfected with the original pBi-2 vector (only coding for luciferase) and the original pUHrtTA 62-1 to determine the induction efficiency of this inducible system. Results shown in FIG. 31A demonstrate the functionality of the system. Highest induction was achieved in growth medium (GM), where luciferase activity was increased 3152-fold. Throughout the four days of differentiation, luciferase activity decreased but was still sufficiently induced (464-fold at day 1, 138-fold at day 2, 70-fold at day 3, and 64-fold at day 4). The decrease of luciferase signal in the subsequent days is most likely due to a dilution effect of the transfected plasmids. Since no genome-integration appears in transient transfections, the plasmids are not equally inherited by daughter cells and therefore become diluted with ongoing cell replication in GM.

The cell line of choice was the L6E9 cell line, a subdlone of the parental rat neonatal myogenic line, which does not express endogenous IGF-1 but expresses the IGF-1 receptor. Therefore this cell line is a good system for analyzing the effect of single IGF-1 isoforms on myoblast proliferation and differentiation. The system applied in the present work drives transcript expression from an inducible hCMV promoter, which allows the analysis of the IGF-1 isoform actions at all stages of development.

In order to test the effect of inducible IGF-1 isoforms on L6E9 proliferation and differentiation, L6E9 cells were double-transfected with the plasmids encoding the different inducible IGF-1 isoforms and luciferase driven by the inducible tetO/hCMV Pₘᵢₙ₋₁ and Pₘᵢₙ₋₂ promoter (pBi/IGF-1 isoform X) on the one hand, and the rtTA-encoding inducer plasmid encoding the rtTA protein under control of the human CMV promoter (pUHrtTA) on the other hand. Control cells were transfected with the empty pBi-2 vector and pUHrtTA (mock control). Induction of luciferase and IGF-1 isoform expression was achieved by administration of the tetracycline derivate doxocycline (1 μg/ml) to the medium 5 hours after transfection. Cells were kept in growth medium (GM) for one day after transfection and then shifted to differentiation medium (DM) for four days. Presence of IGF-1 was confirmed by RT-PCR and Western blot throughout growth and the differentiation process.

Further confirmation of IGF-1 transgene expression was achieved by monitoring luciferase expression throughout the experiment, which also allowed for a comparison if IGF-1 isoform expression levels. Results shown in FIG. 31C show that luciferase activity was strongest shortly after doxicycline induction in GM (4012-fold in mock controls, 1072-fold in Class 1 IGF-1Ea cultures, 2068-fold in Class 2 IGF-1Ea, 1823-fold in 22-IGF-1Ea, 903-fold in Class 1 IGF-1Eb, 2589-fold in Class 2 IGF-1Eb, and 1435-fold in 22-IGF-1Eb transfected cells). Due to differences in transfection efficiency, a high variation has to be taken into account, but all of the differently transfected cultures continued to demonstrate a sufficiently high level of IGF-1 transgene-induction, even though total values decreased after shift to DM (ranging from 139-fold to 489-fold at day one, from 78-fold to 346-fold at day two, from 74-fold to 277-fold at day three, and from 59-fold to 746-fold at day four). However, induction levels at all time points were considered sufficient to study IGF-1 isoform induced differences.

Results obtained by Western blot analysis show that in comparison to the mock control, where IGF-1 protein was not detectable all transfected cultures over-expressed IGF-1 protein at all time points surveyed (FIG. 31D). Interestingly IGF-1 protein was detected in unprocessed form during growth (GM) and appeared to be processes only upon shift to DM, shown by the appearance of multiple bands corresponding to the different processed forms of IGF-1 (with signal- and E-peptide, without signal-, but with E-peptide, and in case of Ea-peptides containing isoforms, two additional bands are detected, most likely representing glycosylated versions of these isoforms). Notably, at no stage of proliferation and differentiation were the IGF-1 isoforms processed to the mature form of IGF-1 (7.6 kDa), which was loaded as a positive control.

In summary, doxicycline-induced expression of IGF-1 isoforms in double-transfected L6E9 cells was confirmed by measuring luciferase activity, RT-PCR, and Western analysis and was considered sufficient to further analyze the phenotype of the IGF-1 transfected cells.

Morphology of IGF-1 Isoform Transfected Cells

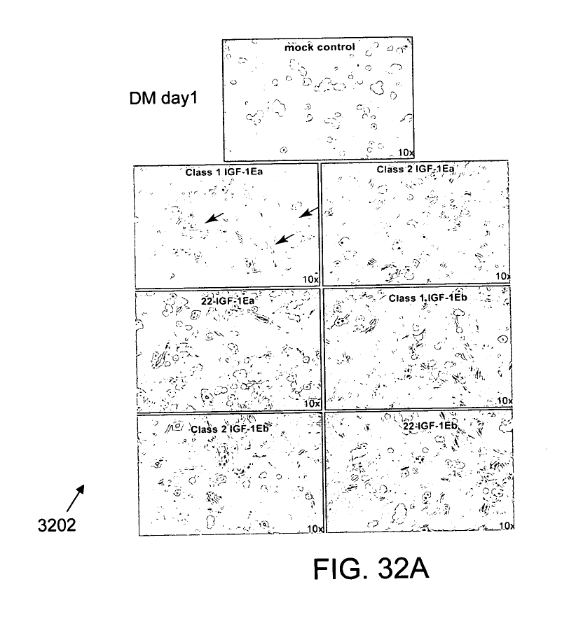

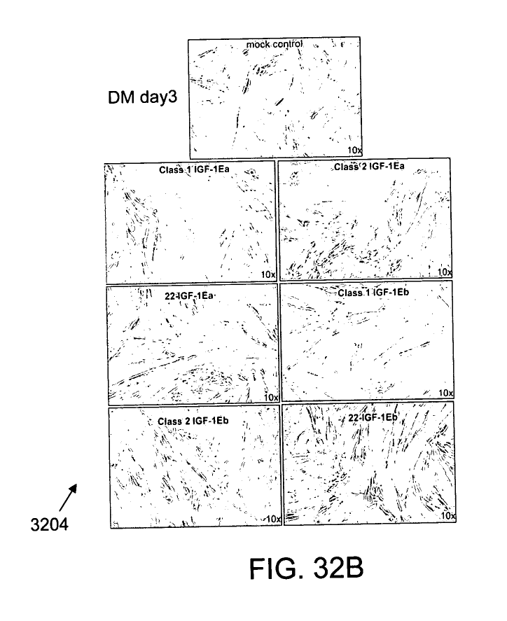

Morphology of IGF-1 isoform transfected cells was observed during all stages of proliferation and differentiation (FIG. 32). In GM, cells did not show any differences in morphology (data not shown). Upon shift to DM, cell morphology was documented after 24 hours (day one), 48 hours (day two), and 72 hours (day three). At day one of differentiation, cells started to elongate as first sign of differentiation. However, in comparison to the mock control cells, cells transfected with Class 1 IGF-1Ea showed an enhanced number of elongated cells, while cells transfected with the other five IGF-1 isoforms had less effect on the differentiation process, indicated by the overall appearance of only few elongated fibers (FIG. 32A). Most importantly, cultures transfected with IGF-1 isoforms containing the Eb-peptide appeared to be more confluent at day one in DM, suggesting a delay in cell-cycle exit.

At day two of differentiation, all the differently transfected cultures displayed elongated myocytes. At this point cells transfected with Class 1 IGF-1Ea already displayed small myofibers, indicating that myocytes started to fuse, again pointing towards an enhancement of the differentiation process. Three days after shift to DM, myofibers were formed in all transfected cell cultures (FIG. 32). Control transfected cells showed very poor fusion and fibers were thin and small. Cultures transfected with IGF-1 isoforms containing the Ea-peptide, over all Class 1 IGF-1Ea and Class 2 IGF-1Ea, showed the formation of large myofibers, while fibers in cultures transfected with 22-IGF-1Ea and the three Eb-containing IGF-1 isoforms were rather thin and more comparable to the size of mock control fibers.

Taken together, transient transfection of L6E9 cells with six different IGF-1 isoforms resulted in an enhanced differentiation of Class 1 IGF-1Ea-transfected cells that culminated in the formation of hypertrophied myofibers. Cells transfected with Class 2 IGF-1Ea showed normal differentiation kinetics but nevertheless showed an increased size of differentiated fibers after day three of differentiation. 22-IGF-1Ea-transfected cells instead showed fibers that were comparable to control fibers, indicating that this IGF-1 isoform does not induce a hypertrophic response. L6E9 cultures transfected with the Eb-peptide containing isoforms of IGF-1 showed a delay in the differentiation process and the formation of normally sized muscle fibers, suggesting that IGF-1 isoforms containing the Eb-peptide do not play a role in mediating IGF-1 induced hypertrophy.

Effects of IGF-1 Isoforms on Myoblast Proliferation

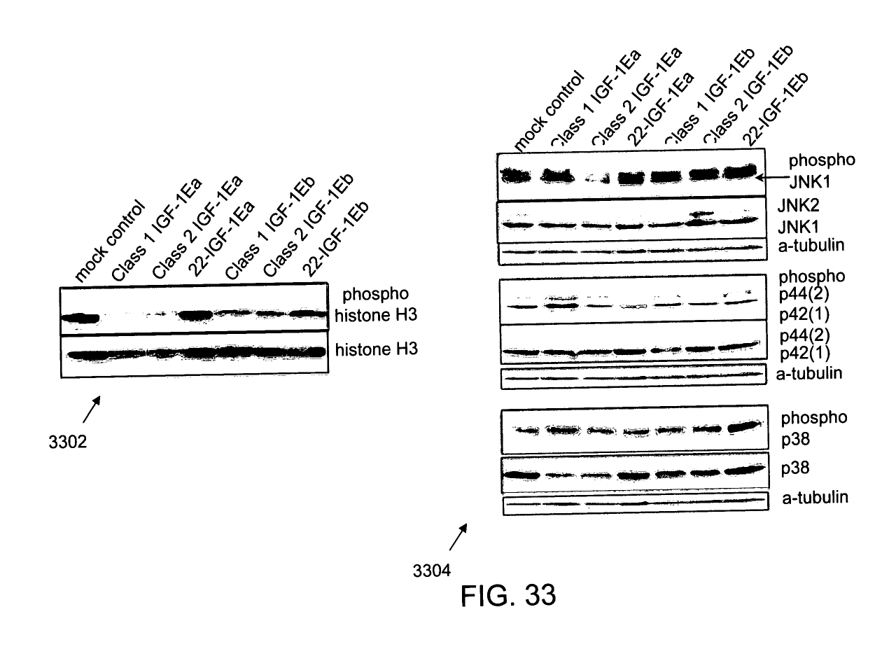

To compare the proliferative state of IGF-1 isoform transfectants during growth, levels of phosphorylated histone H3 were analyzed (FIG. 33A). Phosphorylation of histone H3 is tightly correlated with chromosome condensation during mitosis and meiosis and serves as a marker for cells undergoing mitosis. The mock control as well as the 22-IGF-1Ea samples were slightly over-loaded as shown by total levels of H3 protein and therefore showed a stronger signal for phospho H3. Taking this in account, transfection with Class 1 IGF-1Ea and Class 2 IGF-1Ea samples resulted in decreased amounts of phosphorylated H3, while samples isolated from cultures over-expressing 22-IGF-1Ea, Class 1 IGF-1Eb, Class 2 IGF-1Eb, and 22-IGF-1Eb showed slightly higher levels of histone H3 phosphorylation when compared to mock controls. These findings suggest that expression of Class 1 IGF-1Ea and Class 2 IGF-1Ea had weaker effects on proliferation of L6E9 cells, while isoforms containing the Eb-peptide as well as 22-IGF-1Ea might stimulate proliferation.

One of the major pathways that has been implicated in mediating the proliferative response to IGF-1 is the MAP-kinase pathway. A cascade of events can ultimately lead to phosphorylation-mediated activation of the MAP-kinases SAPK/JNK1, ERK 1 and 2, and p38. To determine eventual differences in activation of these kinases in response to the different IGF-1 isoforms, their level of phosphorylation was analyzed by Western blot (Phosphorylation of SAPK/JNK1 was unchanged in samples from all transfected cultures but Class 2 IGF-1Ea, where levels were slightly decreased. Phosphorylation levels of phospho-Erkl and 2 were up-regulated in Class 2 IGF-1Ea samples, while samples from cultures transfected with the other five isoforms did not show any differences. Finally analysis of p38 phosphorylation showed no major differences among the IGF-1 isoform transfected samples and the mock control.

When compared for their proliferative status during growth, Class 1 IGF-1A and Class 2 IGF-1A showed lower levels of phosphorylated Histone H3 in comparison to mock transfected cells, indicating that these two isoforms may have weaker effects on proliferation then the other four IGF-1 isoforms, whilst the level of phospho-H3 was slightly increased in samples form 22-IGF-1Ea, Class 1 IGF-1Eb, Class 2 IGF-1Eb, and 22-IGF-1Eb transfected cells.

Screening for involvement of different MAP kinases showed subtle differences in the activation by the various IGF-1 isoforms Class 1 IGF-1Ea and the Class 2IGF-1Ea isoforms showed decreased phospho-JNK1 levels in Class 2 IGF-1Ea cultures and increased phospho-Erk1 and 2 levels in Class 1 IGF-1Ea cultures.

Effects of IGF-1 Isoforms on Myoblast Maturation and Differentiation

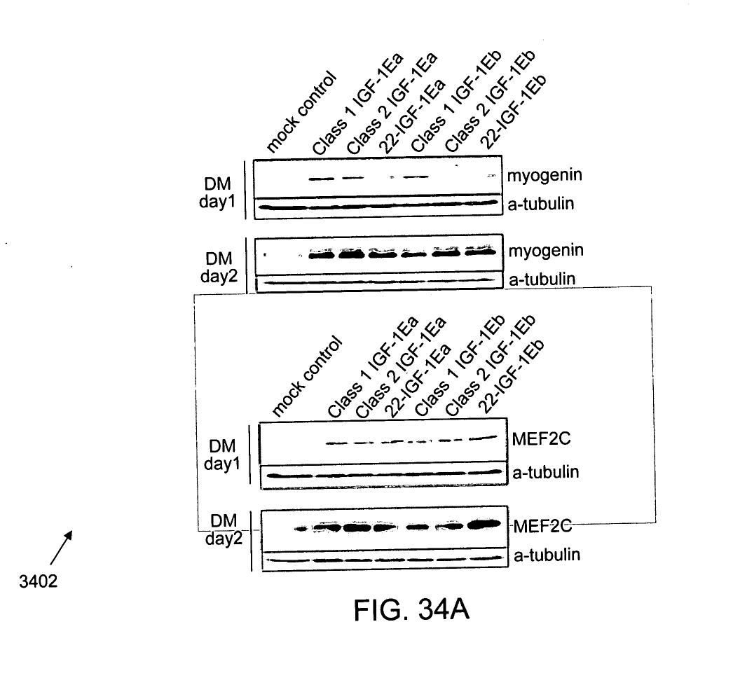

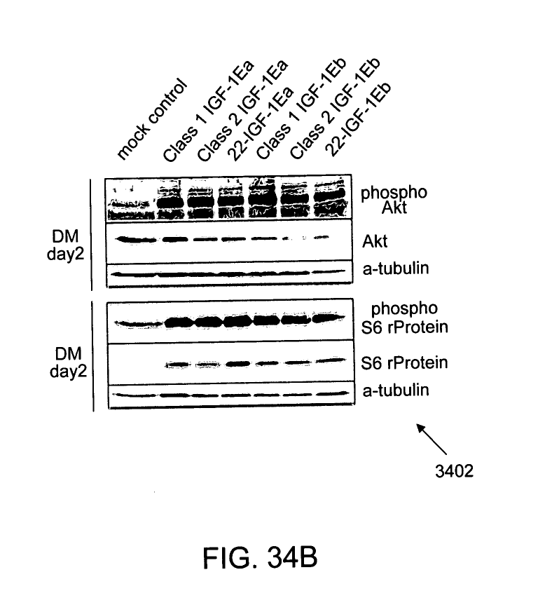

To monitor effects of IGF-1 isoform expression on the differentiation process, cells were analyzed for the necessary expression of the myogenic determination factor (MDF) myogenin, as well as the myocyte enhancing factor (MEF) MEF2C (FIG. 34A upper panel). While not detectable in GM, myogenin was induced in all transfected cultures after 24 hours in DM. Induction was stronger in all IGF-1 transfected cells compared to the mock control. However, subtle differences in the degree of induction in response to different IGF-1 isoforms were noted. Class 1 IGF-1Ea showed the strongest up-regulation of myogenin protein, confirming the enhanced elongation of cells transfected with this isoform. Class 2 IGF-1Ea and Class 1 IGF-1Eb also showed increased up-regulation of myogenin when compared to the other IGF-1 isoforms, while 22-IGF-1Ea, Class 2 IGF-1 Eb and 22-IGF-1Eb showed rather mild induction of myogenin. However, after two days of differentiation all IGF-1 isoform transfected cells showed similar levels of myogenin, which were still higher in comparison to the mock control. To further confirm normal progression of differentiation, protein levels of MEF2C were analyzed (FIG. 34A lower panel). In comparison to the control cells, where MEF2C is not detectable at day one, all IGF-1 isoform transfected cells similarly induced MEF2C. With ongoing differentiation, levels further increased at day two, when also the control cells start expressing low levels of MEF2C.

To efficiently promote growth, the level of protein translation must increase. One of the pathways implicated in IGF-1 mediated stimulation of growth is the PI(3)-kinase pathway. To investigate effects of IGF-1 isoform expression on induction of the PI(3)-kinase pathway, two downstream targets of the P13 kinase pathway, Akt and S6 ribosomal protein, were analyzed.

Activation of the downstream target Akt was analyzed (FIG. 34B upper panel). In comparison to the control cells, which activated Akt mildly, induction was much stronger in cells transfected with the IGF-1 isoforms. Among the different IGF-1 isoforms there was no difference in Akt activation, indicating that the six IGF-1 isoforms uniformly are capable of activating Akt.

One of the downstream effectors of the PI(3)-kinase/Akt pathway is the p70 S6-kinase (p70S⁶K), which phosphorylates the S6 ribosomal protein upon activation. Phosphorylation of the S6 ribosomal protein in turn is directly correlated with an increase in translation. To investigate if IGF-1 isoforms can lead to phosphorylation of S6 ribosomal protein similarly, levels of phospho-S6 ribosomal protein were determined (FIG. 34B lower panel). As seen for Akt activation, all isoforms up-regulated induction of phosphorylation equally when compared to control cells. This further suggests that all of the isoforms similarly stimulate the PI(3)-kinase pathway.

In summary, all IGF-1 isoforms uniformly induced expression of myogenin and MEF2C, even though subtle differences were seen for myogenin induction, where Class 1 IGF-1Ea showed the strongest induction, further implicating this isoform in enhancement of the differentiation process. Cells transfected with this isoform were the first to stop proliferating upon the shift to differentiation medium (DM), started to fuse at day 2 in DM, and showed a stronger up-regulation of myogenic markers, like myogenin. Activation of the PI(3)-kinase pathway, determined by phosphorylation of the downstream S6 ribosomal protein, showed uniform up-regulation compared to the controls, but no differences among the different IGF-1 isoforms. Analysis of the differentiation kinetics in all four transfectants points towards a prominent role of Class 1 IGF-1A (=mIGF-1) in accelerating the differentiation process.

Testing IGF-1 Isoform Function in Vivo

To gain further insight into the role of different IGF-1 isoforms in skeletal muscle in vivo, six transgenic mouse lines over-expressing one of the six IGF-1 isoforms were generated.

Skeletal muscle-specificity is achieved by driving the transgene from the skeletal muscle-specific myosin light chain (MLC) ⅓ promoter and enhancer (FIG. 20). The MLC ⅓ promoter is exclusively activated in mouse skeletal muscle starting at E9.5 and persisting into adulthood, thereby providing post-mitotic and skeletal muscle-specific transgene expression throughout the life of the transgenic animal.

Generation of Transgenic Mouse Lines

All expression constructs generated were sequenced before injection to ensure integrity of every IGF-1 isoform sequence. Vectors were digested to completion with EcoRI to linearize the expression cassette and eliminate plasmid sequences. All injections have been performed by the transgenic service of the EMBL-Monterotondo.

One positive founder was born for the transgenic lines MLC/Class 1 IGF-1Ea (founder A) and MLC/22-IGF-1Ea (founder A). For the Line MLC/Class 2 IGF-1Ea (founders A-D) and MLC/Class 2 IGF-1Eb (founders A-D) four positive founders were obtained, three were generated for MLC/22-IGF-1Eb (founders A-C), and injection of MLC/Class 1 IGF-1Eb yielded two positive founder lines (founders A and B). Each positive founder was mated to wildtype (WT) mice and female and male offspring for each transgenic founder of every line were analyzed for transgene expression.

Analysis of Transgenic Founders

Offspring of all generated founders of each transgenic line (1-4 founders per line) were analyzed for transgene expression by Northern Blot. Where possible, 2 founders of each line have been selected due to high and comparable transgene expression.

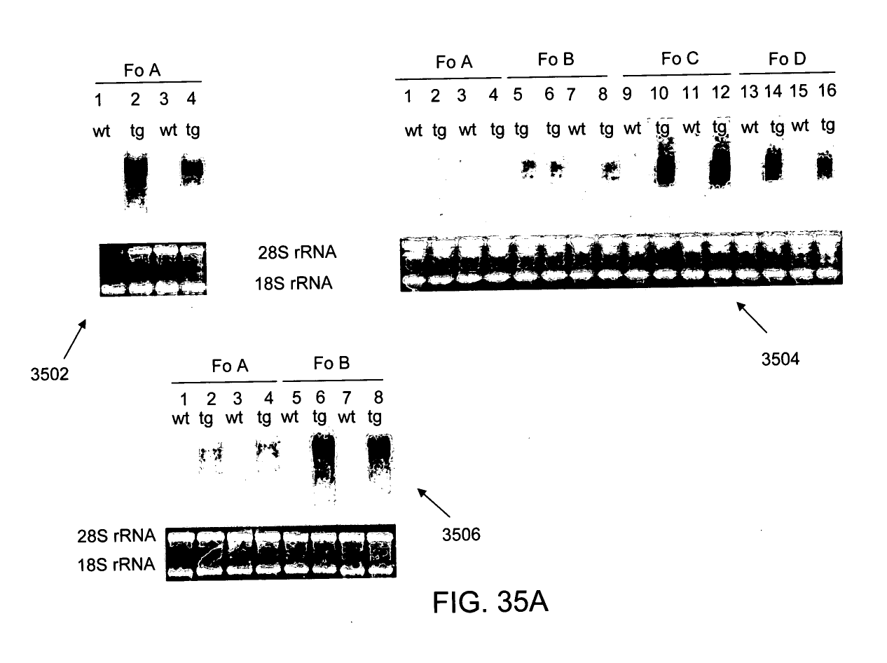

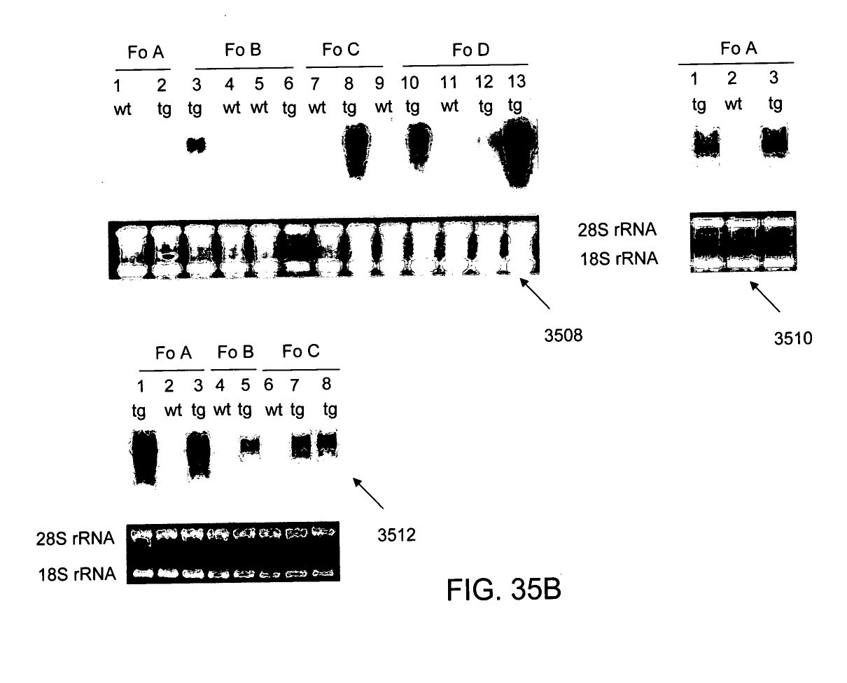

Since the transgenic IGF-1 isoforms are of mouse origin, a probe specific for the most 5′-end of the SV40 poly (A) signal was used to exclusively detect the transgenic mRNAs. Total RNA was isolated from three-months-old male and female quadriceps muscles. For line MLC/Cass 1 IGF-1Ea only one founder was available for expression analysis (FIG. 35A) and therefore further analysis was performed on MLC/Class 1 IGF-1Ea founder A. Line MLC/Class 2 IGF-1Ea founders C and D showed the highest transgene expression, while founders A and B showed moderate to low expression (FIG. 35B). Due to their high transgene expression level, founders C and D were kept for further analysis. The two founders obtained for line MLC/Class 1 IGF-1B expressed moderate (founder A) and high (founder B) levels of the transgene (FIG. 35C) and both founders were further analyzed.

Among founders A to D of line MLC/Class 2 IGF-1Eb founders C and D showed the strongest transgene expression, while founder B expresses low levels of the transgene and founder A showed only traces of a positive signal (FIG. 35D). Founder C and D were therefore chosen for further analysis. The single founder A for line MLC/22-IGF-1Ea showed moderate to high expression of the transgene (FIG. 35E) and was further analyzed. The three founders obtained for line MLC/22-IGF-1Eb showed high (founder A) and moderate (founders B and C) transgene expression (FIG. 35F). Founder A was kept due to highest transgene expression and since founder C gave a slightly stronger signal than founder B, it was kept as well.

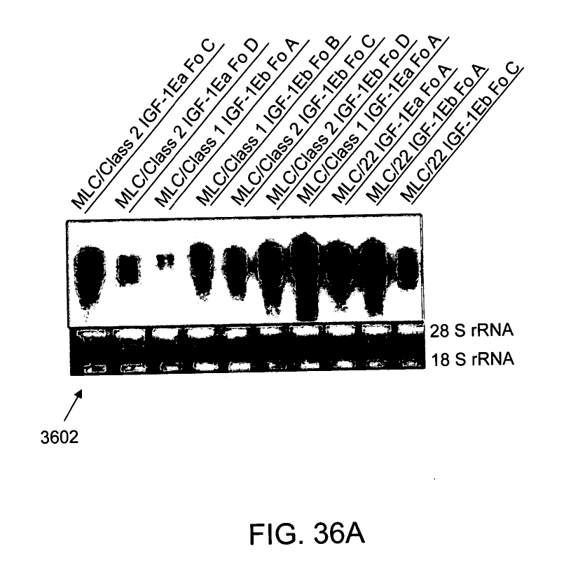

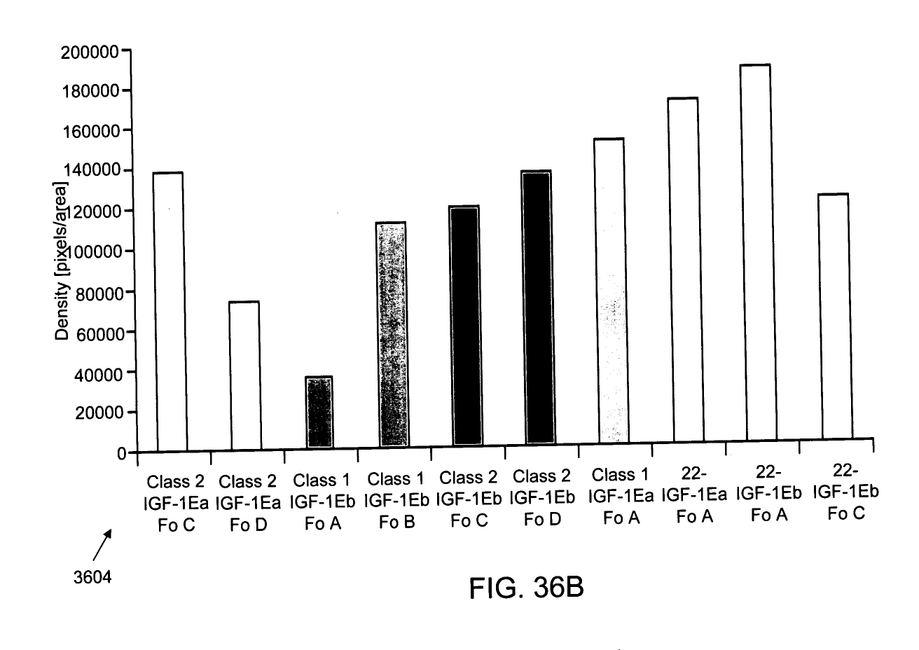

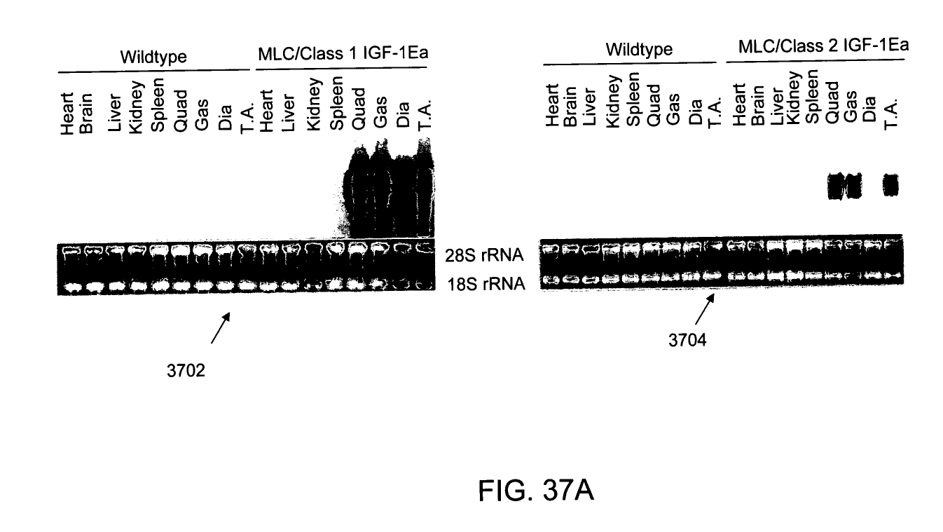

For a better comparison of the transgene expression level among the different founders of each line, total RNA isolated from three-month-old male quadriceps muscles of each selected founder was analyzed by Northern blot using the previously described SV40-specific probe (FIG. 36A). The results were analyzed by using the Radames software to screen the density (pixel/area) of the bands detected by Northern blot (FIG. 36B). Highest expression was detected for line MLC/22-IGF-1Ea founder A (172476 pixel/area) and MLC/22-IGF-1Eb founder A (188542 pixel/area), followed by comparably high expression in lines MLC/Class 2 IGF-1Ea founder C (140161 pixel/area), MLC/Class 1 IGF-1Eb founder B (112511 pixel/area), founders C (120636 pixel/area) and D (137135 pixel/area) of line MLC/Class 2 IGF-1Eb and MLC/22-IGF-1Eb founder C (123382 pixel/area). Expression of MLC/Class 2 IGF-1Ea founder D and MLC/Class 1 IGF-1Eb founder A was low compared to the other founders (30918 and 36675 pixel/area respectively). Based on this comparison, primary founders were selected for each line to perform further analysis. Primary founders of each line were: Founder A for line MLC/Class 1 IGF-1Ea, founder C for MLC/Class 2 IGF-1Ea, founder A for line MLC/22-IGF-1Ea, founder B for line MLC/Class 1 IGF-1Eb, founder D for line MLC/Class 2 IGF-1Eb and founder A for line MLC/22-IGF-1Eb.

Skeletal Muscle-Specific Transzene Expression of Transzenic Lines

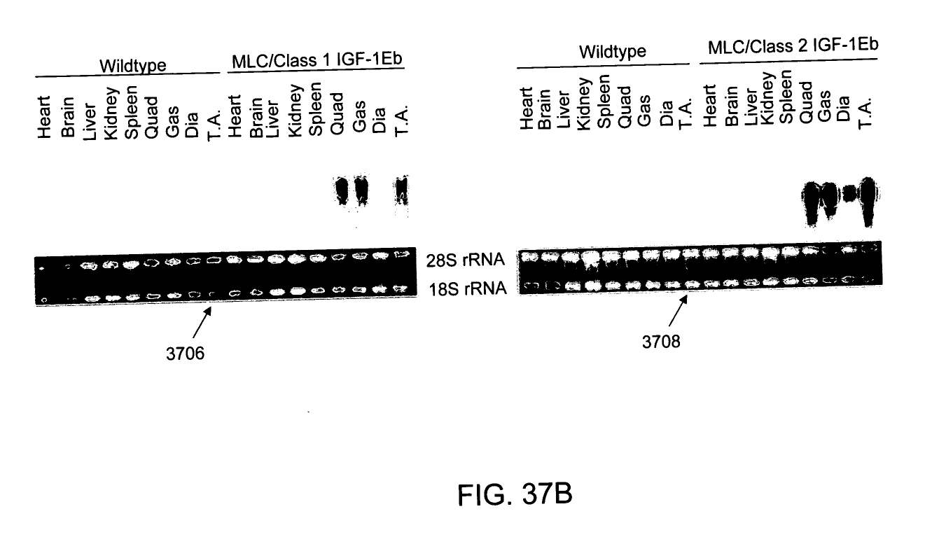

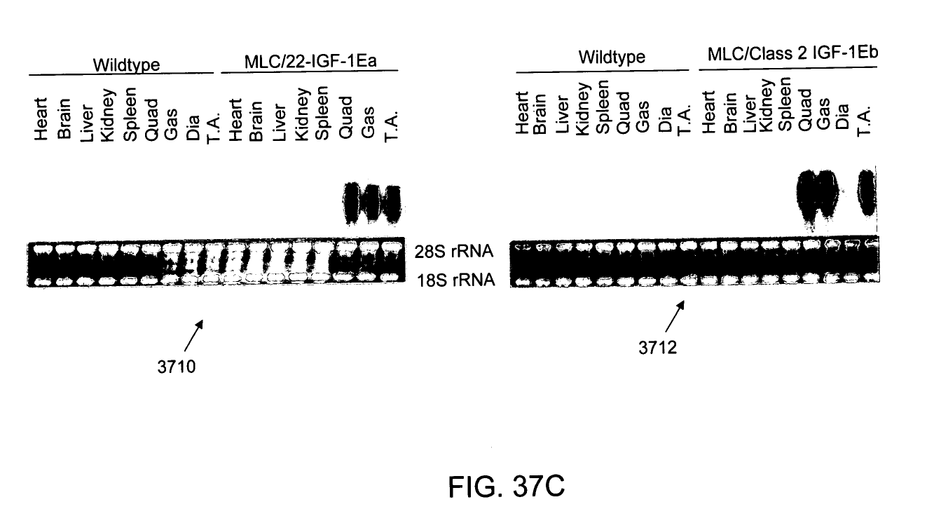

The next crucial step in the analysis of the transgenic founder lines was to determine if the IGF-1 transgenes were expressed in a skeletal muscle-specific manner. For this purpose total RNA of distal organs (heart, brain, liver, kidney, and spleen), as well as different skeletal muscle groups (quadriceps, gastrocnemius, diaphragm, and tibialis anterior (TA)) of male and female wildtype and transgenic three-month-old mice of each selected founder was isolated and analyzed by Northern blot using the previously described SV40-specific probe. FIG. 37A-F shows the results for the highest expressing founder of all six transgenic lines that were selected based on the expression analysis. mRNA and protein analysis of individual muscle groups revealed high to moderate transgene expression levels, depending on the muscle fiber distribution of the examined muscle. No transgene expression was detected in any non-skeletal muscle tissue, such as heart, brain, liver, kidneys, or spleen. Analysis of endogenous IGF-1 isoform expression by Northern Blot and reverse transcription PCR in response to over-expression of a given IGF-1 isoform showed no alterations between WT and transgenic littermates.

Results obtained for the second selected founders were similar. No signal was detectable in any WT tissue and non-muscle transgenic tissue. Specific bands were exclusively found in the skeletal muscle groups of all transgenic lines, confirming expected expression of the transgenic IGF-1 isoforms. Due to the expression pattern of the myosin light chain promoter, transgene expression is highest in the fast IIB fibers, although lower levels are also expressed in fast 2X and 2A fibers. Therefore fast muscles, like the quadriceps, gastrocnemius and TA, showed higher IGF-1 isoform transgene expression than mixed or slow muscles, such as the diaphragm or the soleus that does not express IIB fibers and consequently only expressed the transgene at low levels.

For deeper phenotypic analysis of the IGF-1 isoform transgenic mouse lines, priority was given to the founder that expressed highest levels of the transgene and to the four isoforms mainly expressed in WT animals, namely MLC/Class 1 IGF-1Ea, MLC/Class 2 IGF-1Ea, MLC/Class 1 IGF-1Eb, MLC/Class 2 IGF-1Eb.

During preliminary analysis it became clear though that the MLC/Class 1 IGF-1Ea line was problematic. The mice were aggressive and didn't breed well, which led to loss of this line. Since no second founder was available, the previously well described MLC/mIGF-1 line, generated by Antonio Musaro (Musaro, McCullagh et al. 2001) was used for further comparative work. This line carries the same transgene as used for generating the MLC/Class 1 IGF-1Ea line (Class 1 IGF-1Ea), but of rat and not of mouse origin, and contains the same MLC ⅓-derived regulatory sequences to drive transgene expression. From now on this line is referred to as MLC/mIGF-1.

Comparison of Expression Levels to MLC/mIGF-1 Transgenic Line

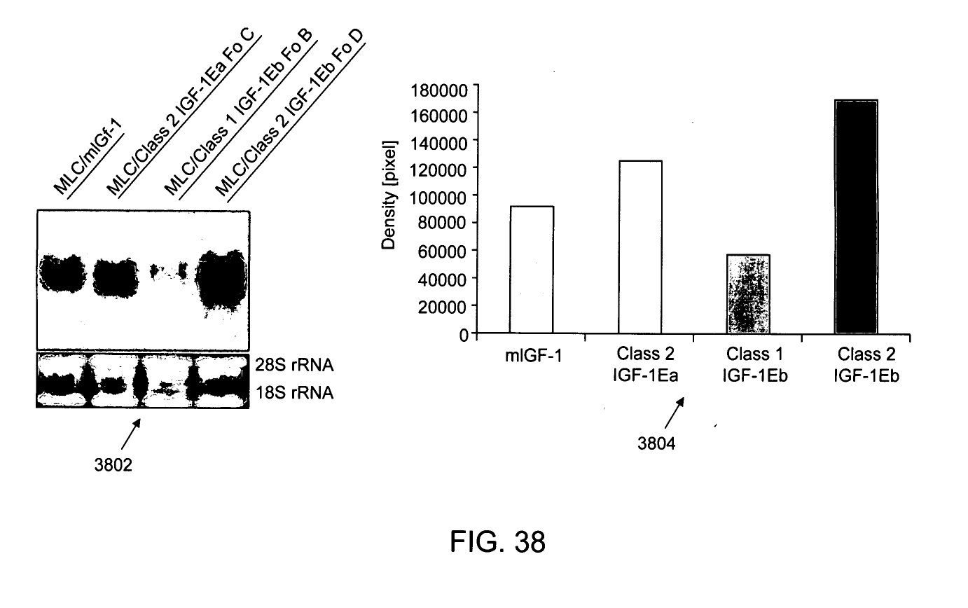

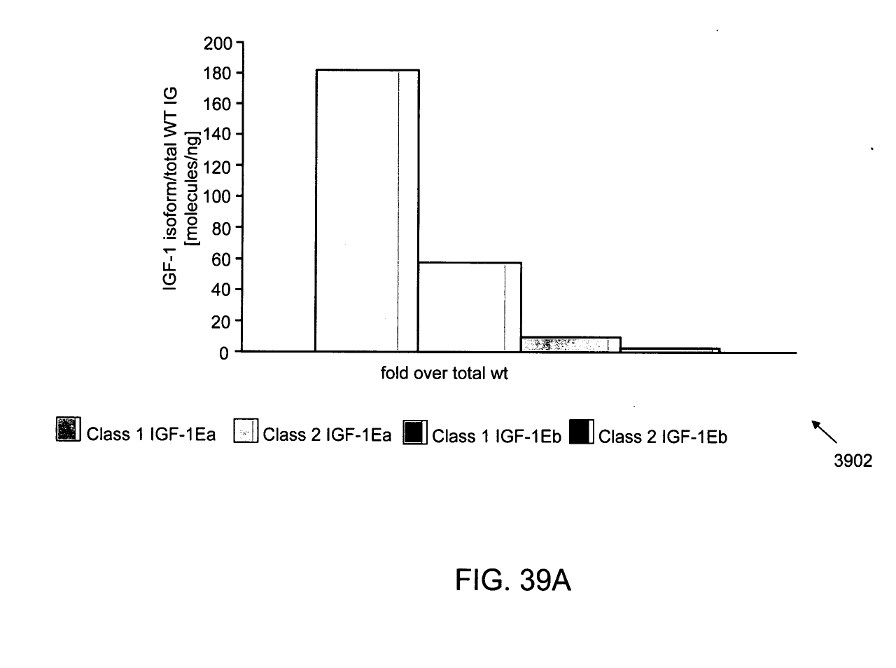

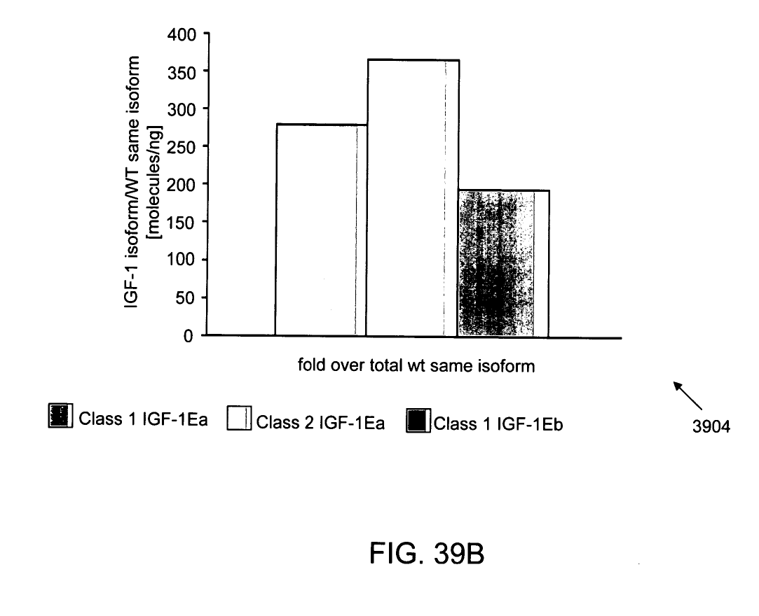

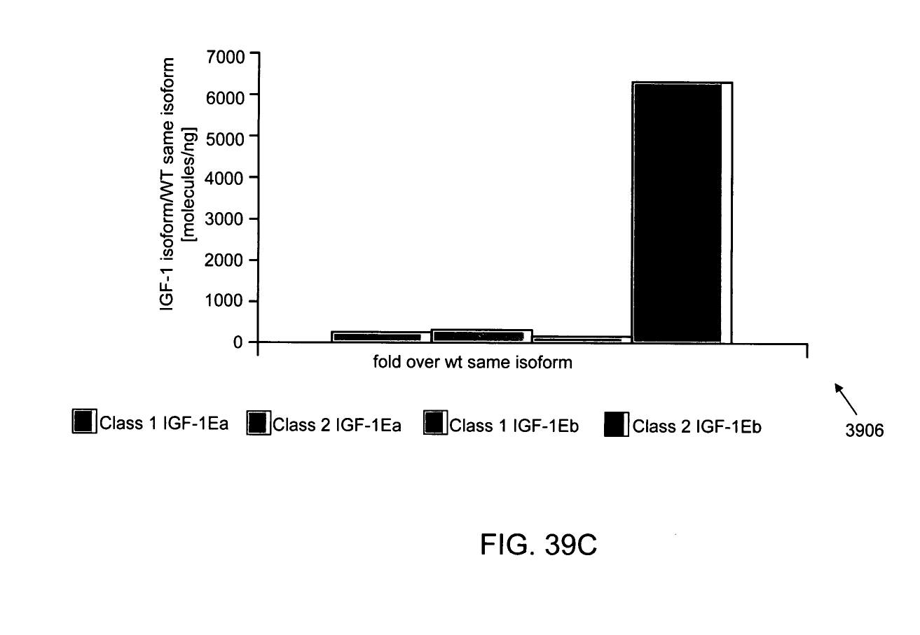

For further work including the MLC/mIGF-1 transgenic line, the expression level of each of the selected founders for every line chosen had to be compared again. Final comparison was done by Northern blot, quantitative real time RT-PCR, and Western blot (FIGS. 38 and 39). Results obtained by Northern blot experiments using of 10 ↑g of total RNA form one-months-old quadriceps probed with the previously described SV40 probe (FIG. 38A) were analyzed with the Radames software to determine the density of bands detected by Northern analysis (FIG. 38B). This analysis revealed highest expression for MLC/Class 2 IGF-1Ea (125627 pixel/area) and MLC/Class 2 IGF-1Eb (171052 pixel/area), followed by MLC/mIGF-1 (92482 pixel/area). Lowest transgene expression levels were detected for line MLC/Class 1 IGF-1Eb (57838 pixel/area).

For quantification in relation to WT total IGF-1 levels and specific IGF-1 isoform levels, quantitative real time RT-PCR was performed on RNA isolated from three-months-old quadriceps muscle of 3 WT mice and 3 transgenic mice of each line. To determine total IGF-1 levels in WT quadriceps muscle, the WT values obtained by quantitative RT-PCR for every isoform were added (3301.2 molecules/ng). Values for the specific isoform over-expressed were then related to this value to gain the total fold-increase over the WT (FIG. 39A). With this type of analysis MLC/mIGF-1 animals (181-fold) and MLC/Class 2 IGF-1Ea (52-fold) showed the highest increase, while MLC/Class 1 IGF-1Eb (8-fold) and MLC/Class 2 IGF-1 Eb (2-fold) instead, showed a rather mild increase. However, if values obtained for the different over-expressed IGF-1 isoforms were related to the WT expression level of the corresponding endogenous isoform, the level of induction was more comparable (FIG. 39B). Line MLC/mIGF-1 showed a 227-fold increase, MLC/Class 1 IGF-1Eb a 190-fold increase, and MLC/Class 2 IGF-1Ea displayed a 364-fold increase. Since the Class 2 IGF-1Eb isoform was not detectable in WT quadriceps muscles, the fold-increase over the WT was much higher for this transgene (6280-fold) (FIG. 39C). The discrepancies in the expression level of the IGF-1 transgenes observed with the different methods, are most likely due to differences in expression level between different animals analyzed and might also affected by different sensitivities of the methods applied.

To relate the mRNA expression level to the protein levels detected in the transgenic muscles, as well as to confirm proper protein expression of the transgenic IGF-1 isoforms,

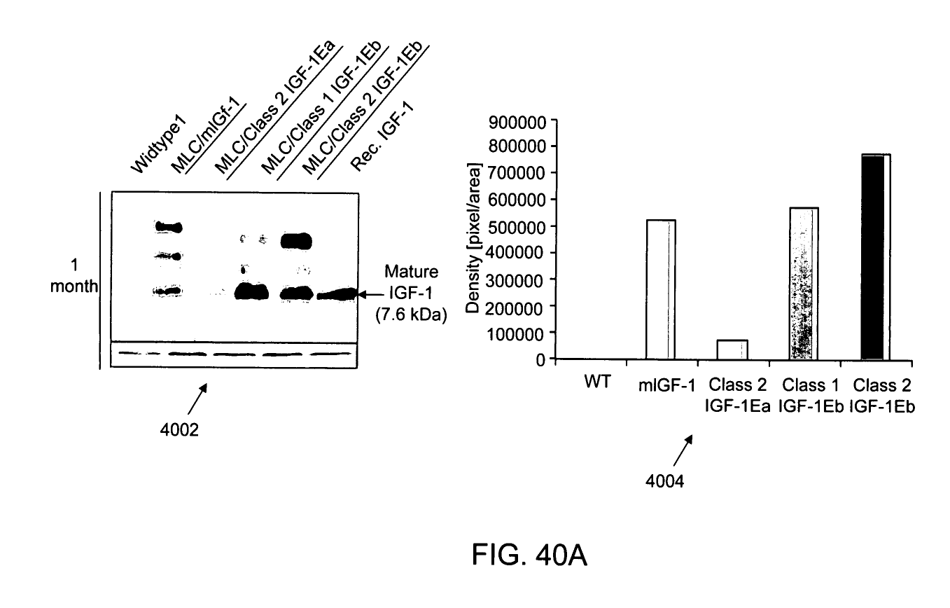

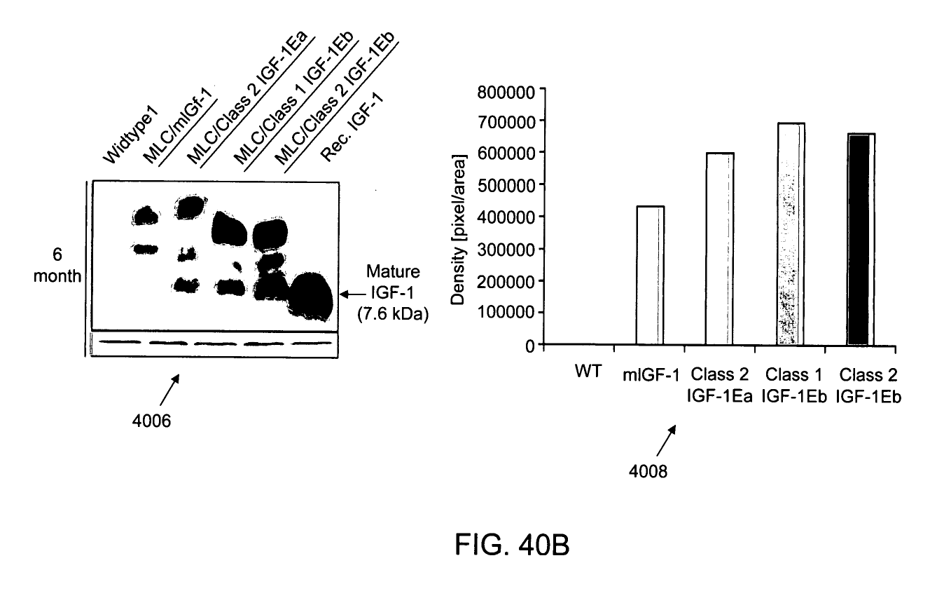

Western blot analysis was done on quadriceps protein lysates of one-month-old and six-months-old WT and transgenic animals for each IGF-1 isoform (FIG. 40). The antibody used for both experiments was directed against the mature IGF-1 sequence and therefore detected all the differently processed forms of IGF-1, represented by the multiple bands detected. For isoforms containing the Ea-peptide two additional bands appeared, most likely reflecting glycosylated forms of those IGF-1 variants. Due to detection of multiple bands, quantification was performed by combining the densities of each detected band for every sample. These values are not absolutely accurate, since the Radames software used for detection and quantification of the single bands, did not detect bands of very low intensity. However, this analysis is still accurate enough to allow the determination of expression levels.

At one month of age, no IGF-1 was detected in the WT sample, while MLC/mIGF-1 (521705 pixel/area), MLC/Class 1 IGF-1Eb (579348 pixel/area), and MLC/Class 2 IGF-1Eb (773249 pixel/area) showed comparable high levels of IGF-1 protein expression. The signal detected for MLC/Class 2 IGF-1Ea (75805 pixel/area) instead, was quite low (FIG. 40A and B). However, the different IGF-1 isoform transgenic lines showed comparable levels of IGF-1 protein expression at six months of age (FIG. 40C and D), while no IGF-1 was detectable in the WT sample. These results confirm that all IGF-1 isoform transgenic lines express the IGF-1 protein to a comparable extent. Variations seen in one-month and six-months-old animals most likely reflect normally occurring differences in expression between different animals of the same line, since these differences were also seen by analysis of transgenic mRNA. Notably, the quite dramatic differences seen in the fold-increase of transgenic mRNA in reference to total WT levels in the different mouse lines are not reflected in the IGF-1 protein expression of these animals.

In summary, comparison of IGF-1 isoform transgene expression on the transcriptional level revealed variable differences among the lines, depending on which method was used. However, despite these differences, expression of IGF-1 protein determined by quantification of results obtained by Western blot analysis showed comparable expression of the different IGF-1 isoforms.

Influence of IGF-1 Isoforms on Endogenous IGF-1 Isoform Expression

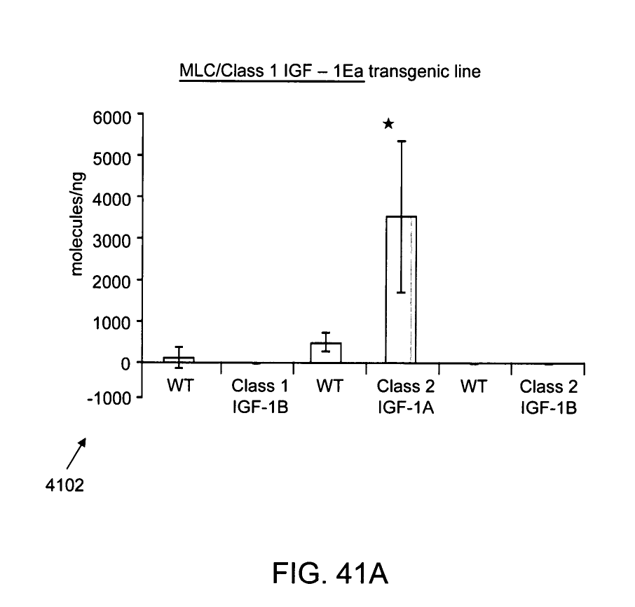

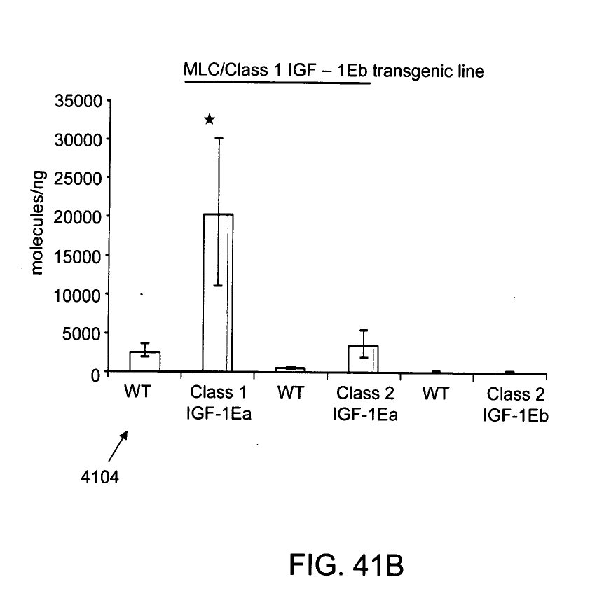

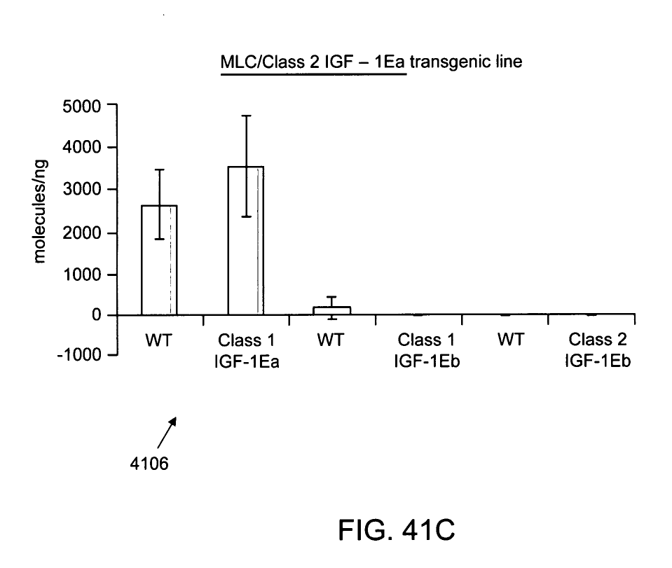

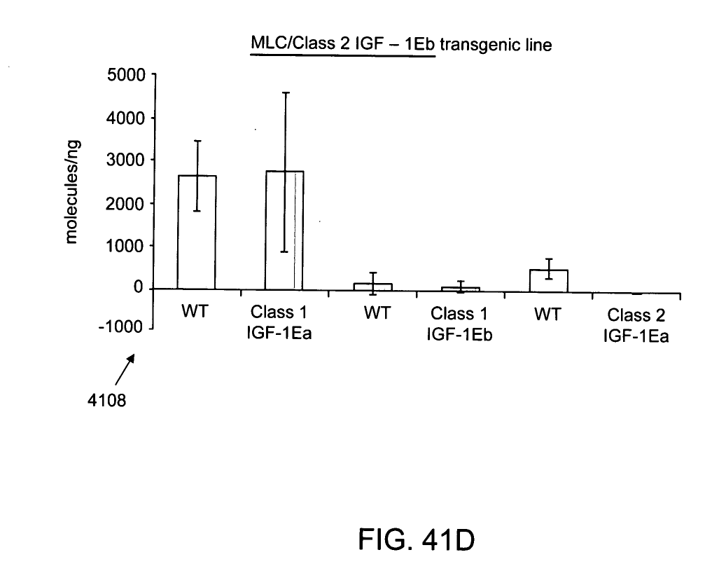

To investigate if the over-expression of IGF-1 isoforms influence the endogenous IGF-1 isoform expression levels, primers were designed for quantitative RT-PCR, allowing amplification of the full-length isoforms, thereby providing reliable evidence for endogenous IGF-1 isoform expression levels. The screening for endogenous IGF-1 isoform expression in each transgenic mouse line was carried out on total RNA from three-months-old quadriceps muscle, isolated from three WT and three transgenic mice from each line. While MLC/Class 2 IGF-1Ea and MLC/Class 2 IGF-1Eb did not have any influence on the expression of endogenous IGF-1 isoforms (FIG. 41C and D), the over-expression of rat Class 1 IGF-1Ea in MLC/mIGF-1 animals up-regulated endogenous Class 2 IGF-1Ea 6.7-fold (p=0.04) without affecting other endogenous IGF-1 isoforms (FIG. 41A). MLC/Class 1 IGF-1Eb transgenic samples showed a 7.7-fold induction of endogenous Class 1 IGF-1Ea (p=0.03), while other IGF-1 isoforms were unchanged (FIG. 41B). The values of each isoform were always compared to the wt expression levels of the corresponding isoform.

To confirm these results, Northern blot analysis was performed with probes designed to bind to specific sequences of the differentially spliced exons. Transgenic lines over-expressing Class 1 isoforms (derived from exon 1) were analyzed with a probe directed against exon 2 sequences, while lines over-expressing Class 2 isoform (derived from exon 2) were analyzed with a probe recognizing the 186 bp 5′-UTR exon 1 sequence, which is subject to a splicing event in 20% of the total IGF-1 mRNA in the liver. By choosing the probe in this region, the majority of endogenous exon 1-derived class 1 transcripts were detected. Over-expressed isoforms containing the Ea-peptide (exon 4-6 splice variant) were analyzed with a probe derived from exon 5 (giving rise to the specific part of the Eb-encoding sequence). Generation of a probe allowing the detection of the exon 4-6 sequence was not possible, since the sequences giving rise to the Ea-peptide are also contained in the exon 4-5-6 splice variant giving rise to the Eb-peptide.

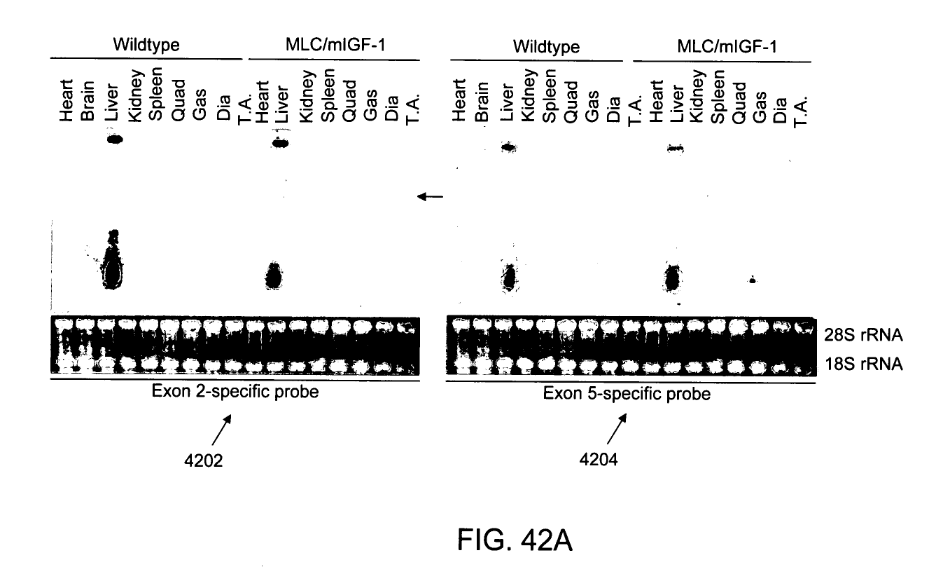

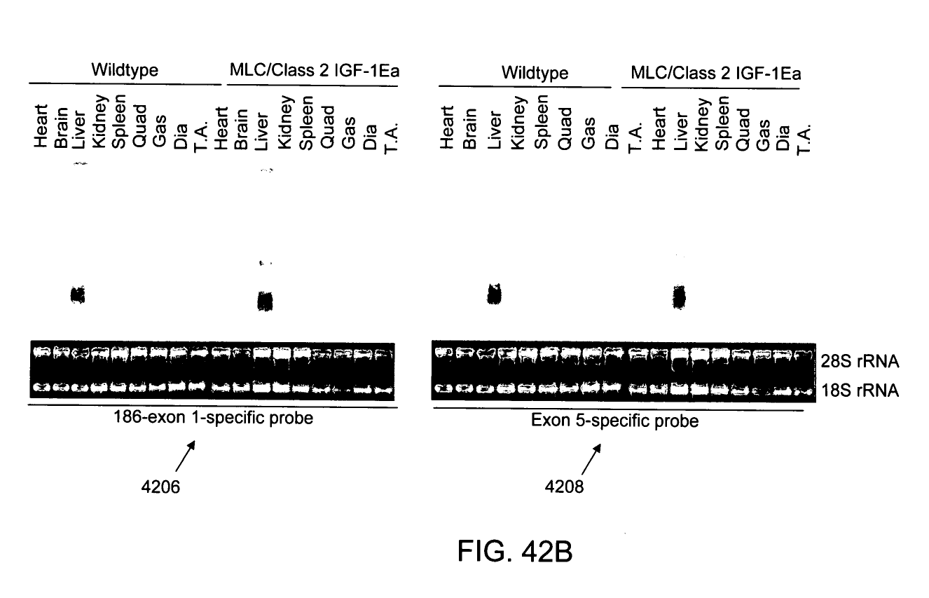

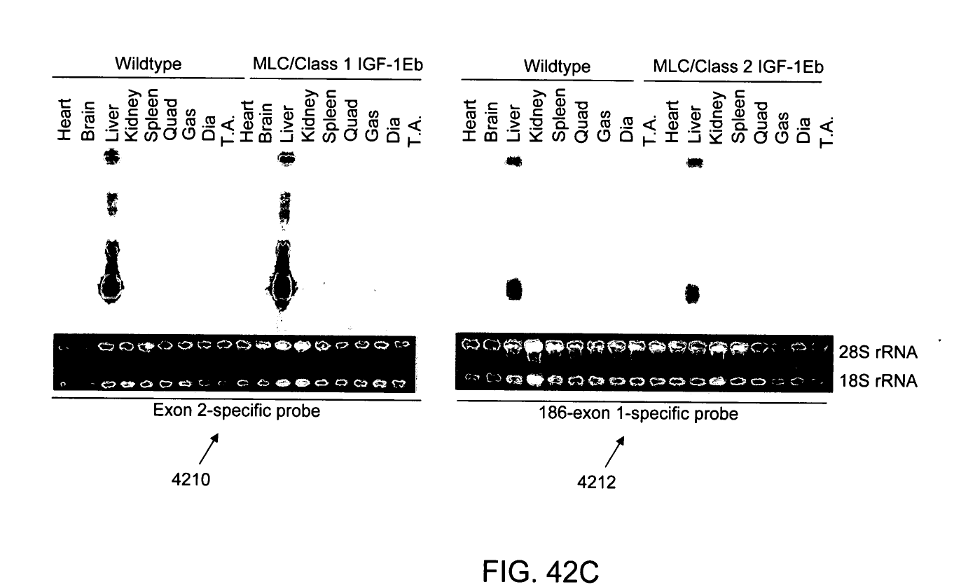

Results obtained with an exon 2-specific probe confirmed the slight skeletal muscle-specific up-regulation of a Class 2 isoform in response to the Class 1 IGF-1Ea (mIGF-1) transgene (FIG. 42A), while Northern blot analysis with an exon 5-specific probe to detect exon 4-5-6 splice variants giving rise to the Eb-peptide, confirmed no changes for Eb-encoding mRNAs (FIG. 42B). To monitor endogenous Class 1 isoform transcription in MLC/Class 2 IGF-1Ea animals, the 186 exon 1-, and the exon 5-specific probes were used. Both experiments confirmed no changes for those isoforms (FIG. 42C and D). Since the exon 4-6 splice variant that gives rise to the Ea-peptide cannot be specifically detected, Northern analysis for MLC/Class 1 IGF-1Eb was restricted to the exon 2-specific probe for Class 2 mRNA detection. Results shown in FIG. 42E confirm no changes for Class 2 IGF-1 transcripts. MLC/Class 2 IGF-1Eb analysis instead was restricted to detection of class 1 IGF-1 mRNAs and showed no changes in comparison to the WT samples (FIG. 42F), again confirming results obtained by quantitative RT-PCR.

Taken together, these results revealed that the Class 1 IGF-1Ea transgene present in MLC/mIGF-1 animals up-regulated endogenous Class 2 IGF-1Ea transcripts without affecting the expression of other endogenous isoforms. In contrast, the MLC/Class 1 IGF-1Eb transgene up-regulated endogenous Class 1 IGF-1Ea, even though this could not be confirmed by Northern blot analysis due to lack of a suitable probe. No effects on endogenous IGF-1 isoforms were seen in lines MLC/Class 2 IGF-1Ea and MLC/Class 2 IGF-1Eb.

General Phenotype of Selected Transgenic Lines—Effect IGF-1 Isoform on Body Weight and Weight of Visceral Organs

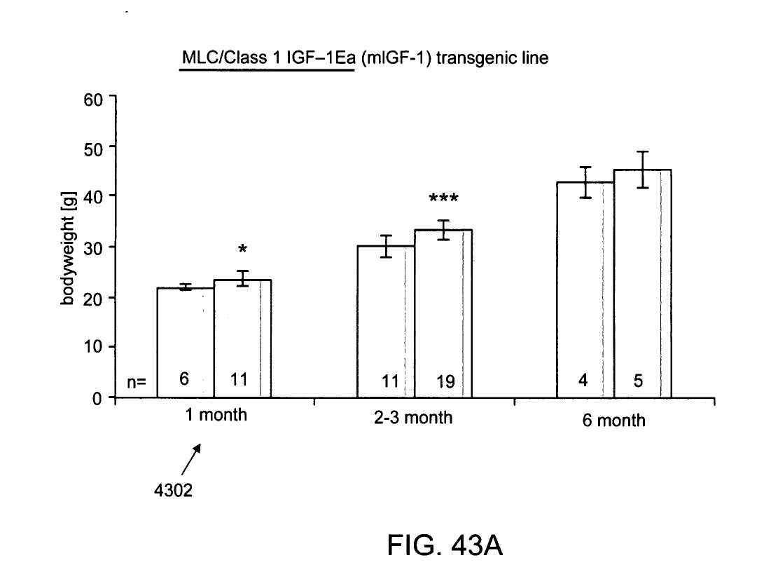

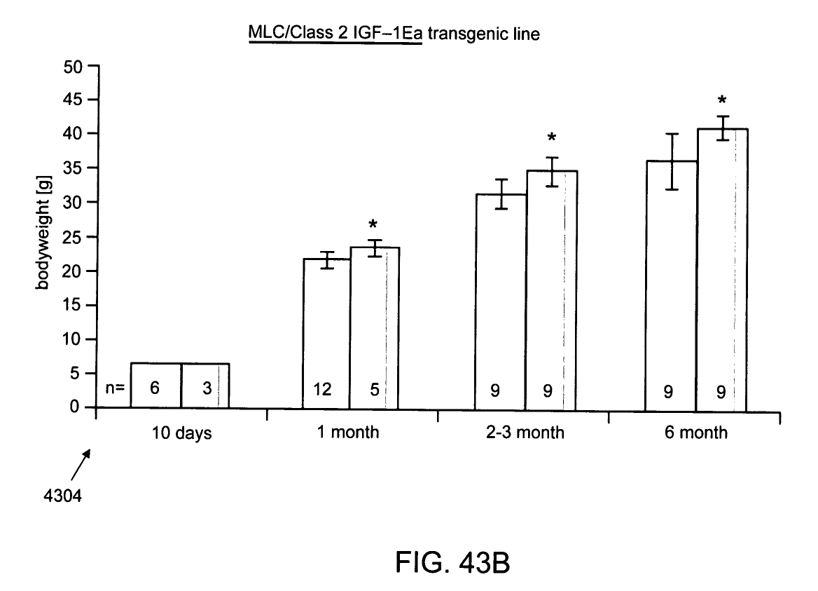

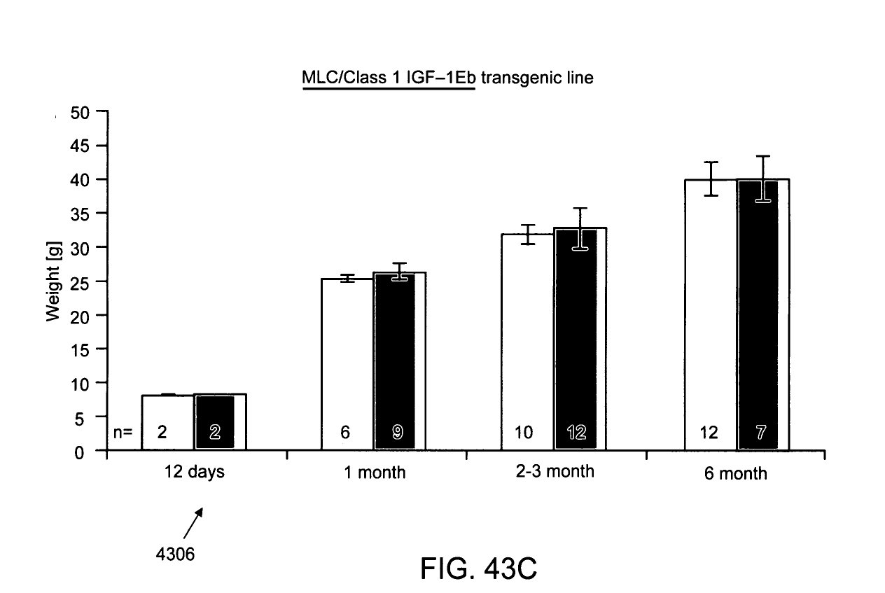

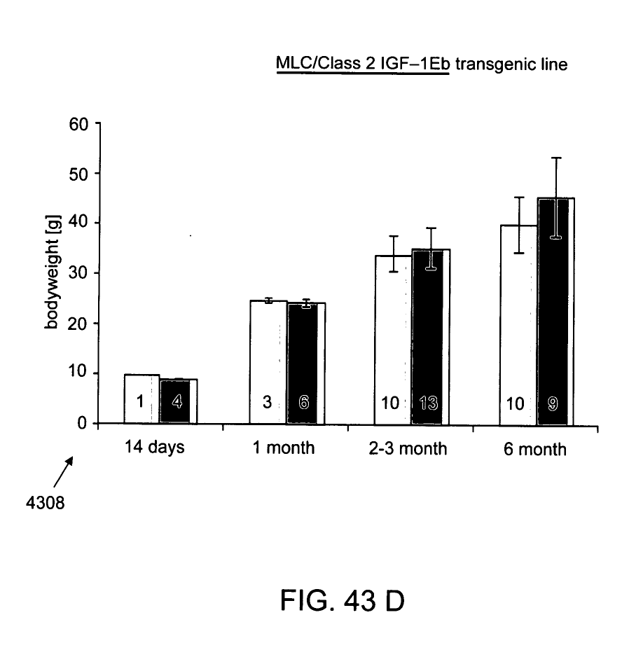

For further comparison to the previously well characterized transgenic line MLC/mIGF-1 (=Class 1 IGF-1A) (86), the three lines representing the remaining predominant isoforms: MLC/Class 1 IGF-1B, MLC/Class 2 IGF-1A, and MLC/Class 2 IGF-1B were focused on. On the basis of over-expressing the different IGF-1 isoforms under the same conditions, the effect of IGF-1 isoform over-expression on the over all body weight and the weight of visceral organs of the transgenic animals was monitored throughout life of the transgenic mice and their WT littermates. All selected transgenic lines were viable and appeared normal throughout development. Results shown in FIG. 43 demonstrate a significant (p<0.05) effect on the over all body weight for lines MLC/mIGF-1 and MLC/Class 2 IGF-1Ea. MLC/mIGF-1 body weight was increased by 8% at one month of age (p=0.0 1), 11% at 2-3 months (p=0.0001), and by 6% at six months of age, even though at six months, values no longer reached significance (p=0.3) (FIG. 43A). MLC/Class 2 IGF-1Ea animals showed a similar increase up to two-three months of age (9% at one month (p=0.01), 11% at two-three months (p=0.01)), but in contrast to MLC/mIGF-1 mice, the 14% increase measured at six months of age for MLC/Class 2 IGF-1Ea animals was still significant (p=0.01) (FIG. 43B). In contrast, MLC/Class 1 IGF-1Eb and MLC/Class 2 IGF-1Eb did not show any significant changes in body weight throughout all ages analyzed (p>0.05) (FIG. 43C and D).

As previously described (86), the MLC/mIGF-1 transgenic line showed skeletal muscle fiber hypertrophy, with an increase of muscle mass of over 50% and a decreased body fat content (86). Although these mice showed a pronounced increase in muscle mass, they did not change their total body weight.

MLC/Class 2 IGF-1B showed no difference in body weight up to an age of six months. Over-expression of MLC/Class 1 IGF-1B showed no effect on the total body weight at the age of one and three months, while by the age of six months, the body weight was significantly decreased. In contrast, MLC/Class 2 IGF-1A transgenic animals showed a significant increase in body weight already by the age of one and three months, which was maintained up to the age of six months.

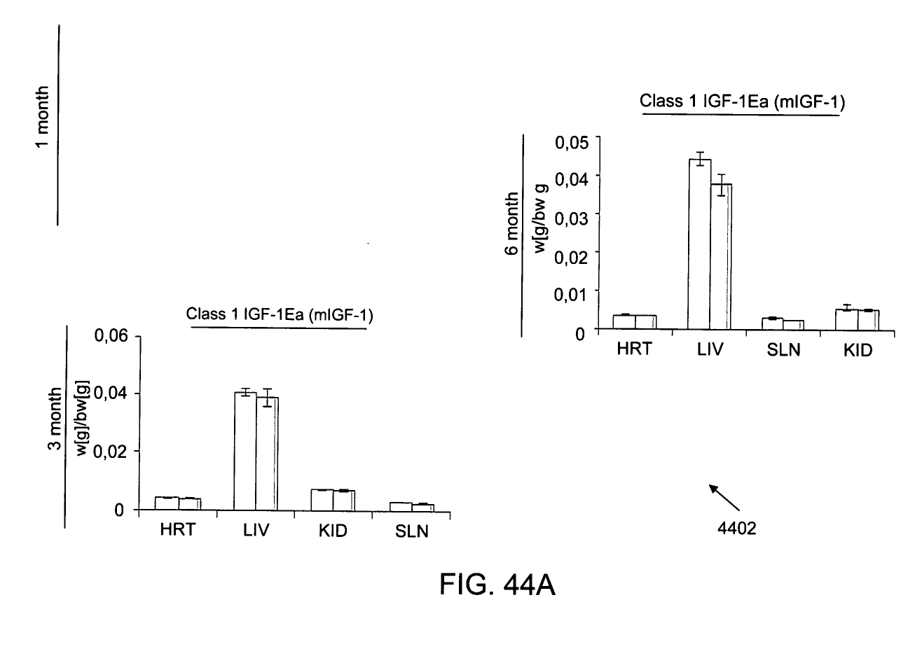

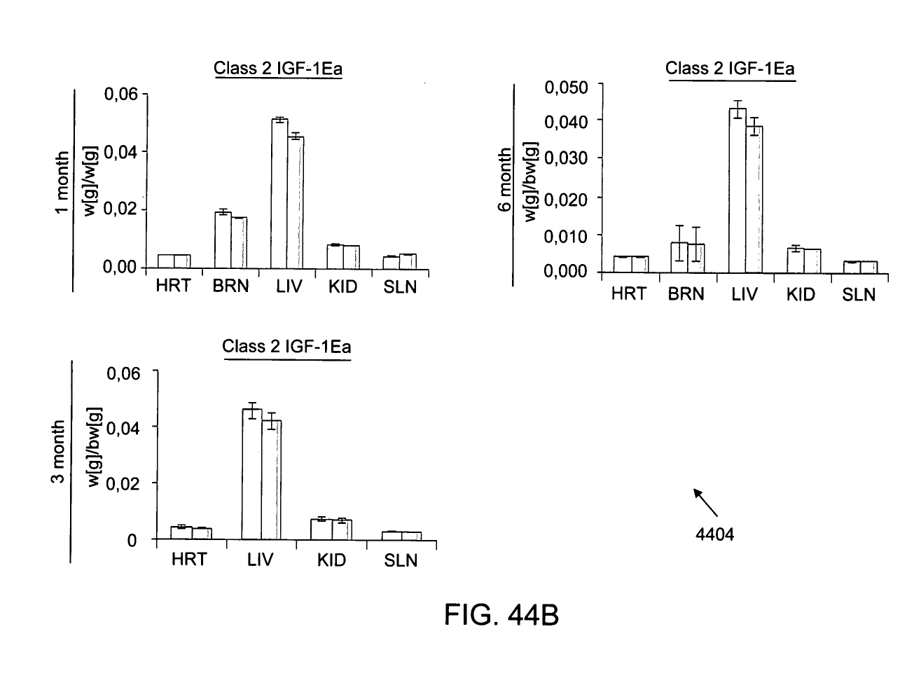

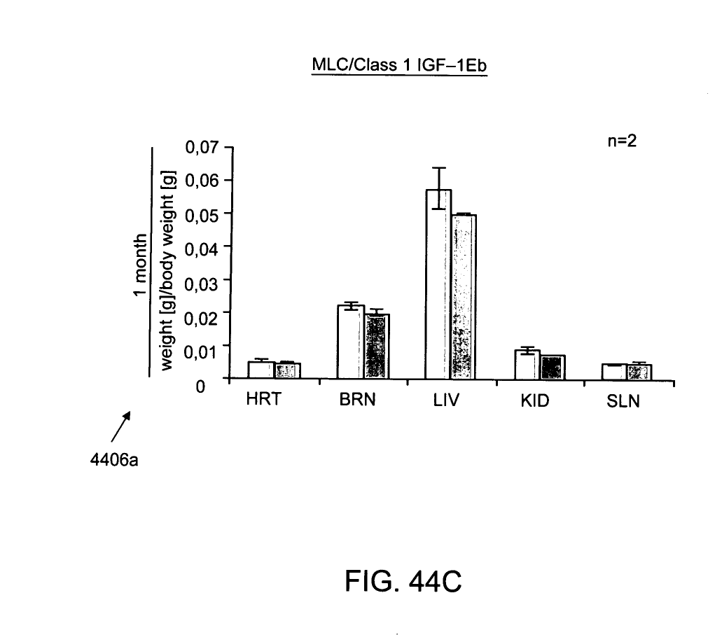

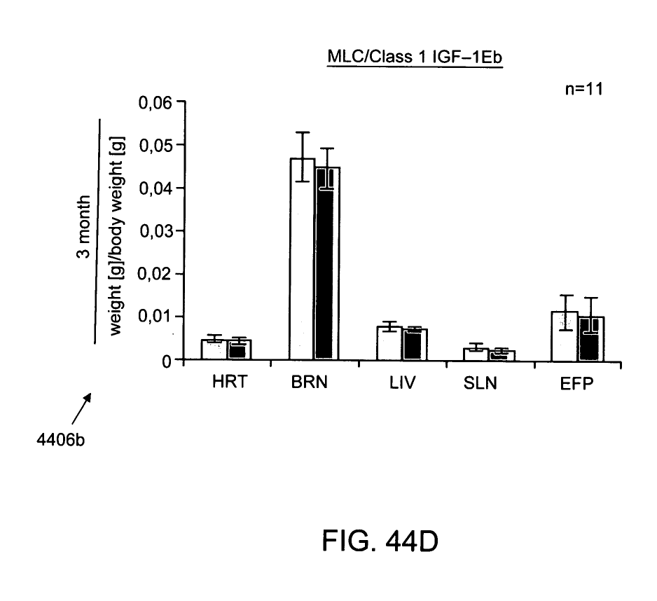

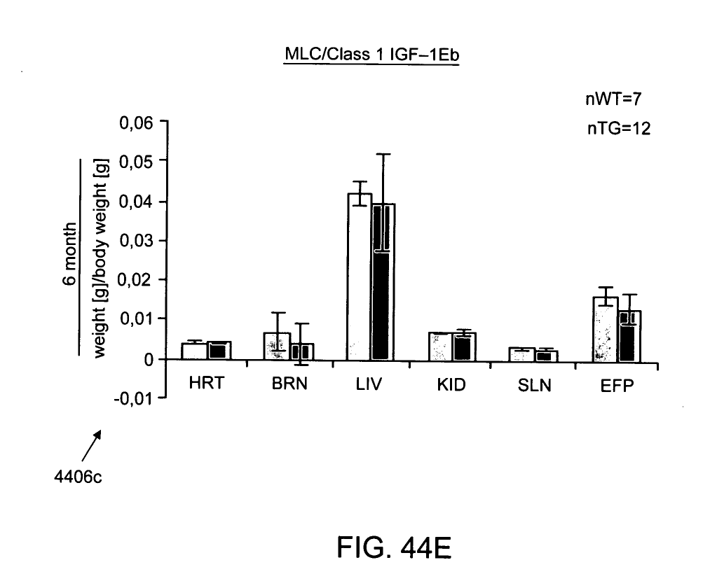

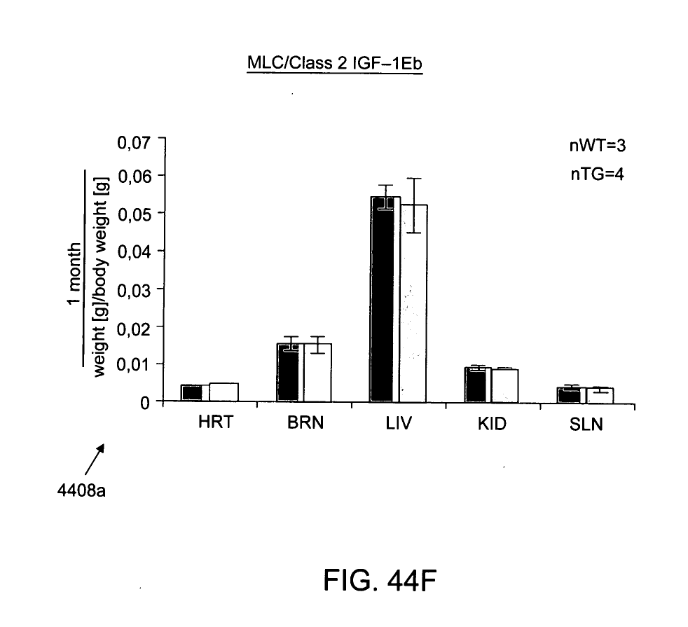

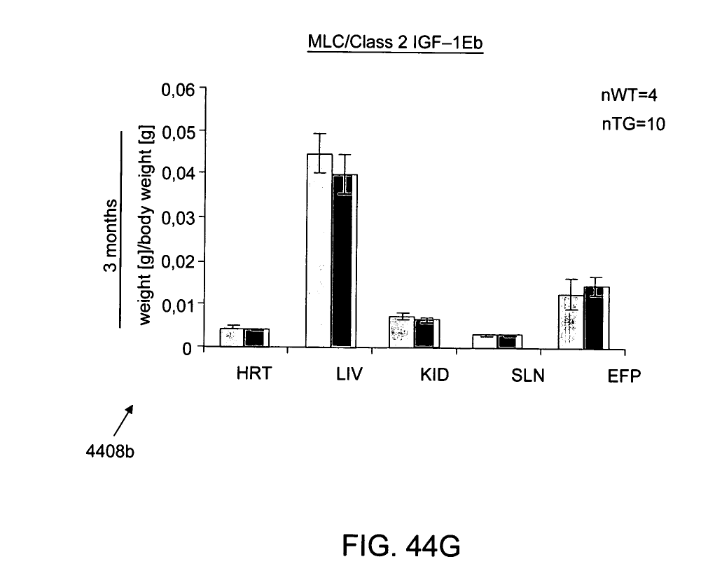

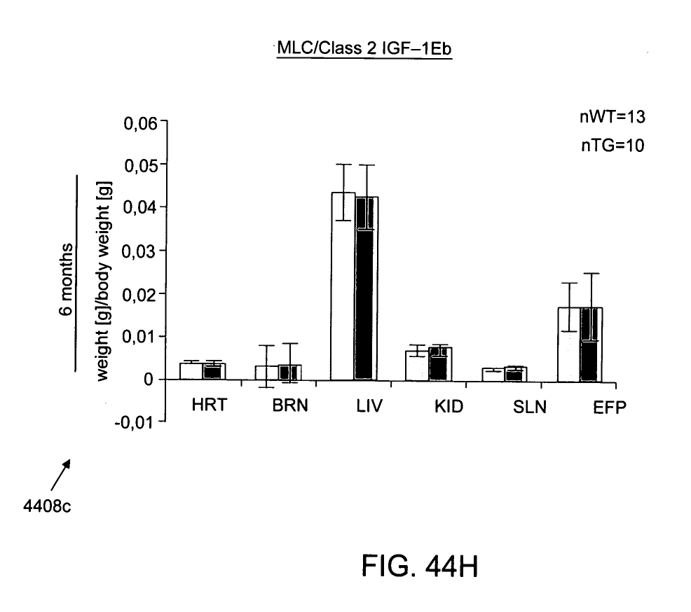

To exclude an effect of IGF-1 isoform over-expression on visceral organs, also the weight of visceral organs (heart, brain, liver, kidney, and spleen) was also monitored throughout life of the transgenic animals. All weight values were normalized for body weight and results are shown in FIG. 44A-D. None of the transgenic lines showed any influence on the weight of distal organs like heart, spleen, kidney, brain, or liver throughout the monitored ages (one, three, and six months) (p>0.05).

To determine whether IGF-1 isoform over-expression had an effect on fat deposition, as reported for male SIS2 mice, which deposited less fat between five and ten weeks of age, measurements of the epididymis fat pat (EFP) of male mice were included. The EFP is the only well defined fat pat in the organism and is therefore well suited for comparison. However, these measurements do not necessarily reflect the total fat content of the animal. Only MLC/mIGF-1 animals (over-expressing the same Class 1 IGF-1Ea isoform as the SIS2 mice, but of rat and not of human origin) showed significant changes, reflected in a 26% decrease (p=0.006) at three months of age and a 15% decrease (p=0.03) at six months of age. None of the other transgenic lines showed an influence on fat deposition in the EFP (p>0.05).

In summary, both IGF-1 isoforms that contain the Ea-peptide showed an effect on overall bodyweight, which was significant for MLC/mIGF-1 up until three months, while MLC/Class 2 IGF-1Ea showed a significant increase at all ages surveyed, while IGF-1 isoforms containing the Eb-peptide in contrast, did not show any changes in bodyweight. None of the transgenic lines showed an influence on the weight of visceral organs, and only MLC/mIGF-1 showed a significant decrease of fat deposition in the EFP.

Chances in Circulating IGF-1 Levels

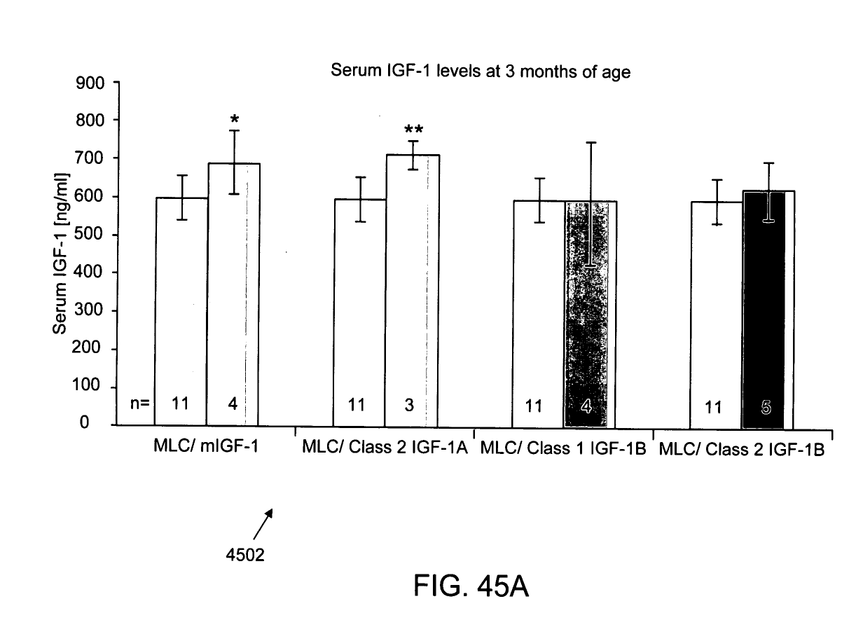

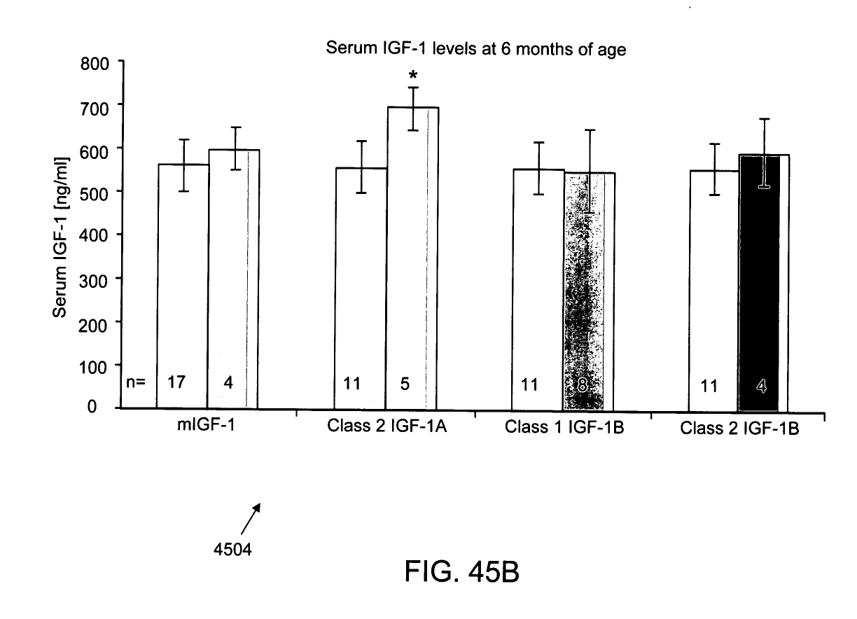

IGF-1 has been shown to act either as a circulating hormone or as a local growth factor. It is widely accepted that the circulating versus local distribution of IGF-1 isoforms is dependent on the specific signal peptide. Previously generated transgenic mice over-expressing IGF-1 have been reported to secrete IGF-1 transgenes to the circulation. The mechanism behind this distinction was tested using the different MLC/IGF-1 isoform transgenic animals, where all four isoforms were over-expressed specifically in skeletal muscle. All MLC/IGF-1 isoform transgenic lines were analyzed for changes in circulating IGF-1 levels. Since all transgenes are of mouse origin and cannot specifically be detected, total amounts of IGF-1 were monitored. Plasma was collected from one- and six-months-old animals and screened by ELISA for IGF-1(FIG. 45). By the age of one month, all lines showed elevated levels of total circulating IGF-1, which did not reach significance. At the age of three months, MLC/mIGF-1 and MLC/Class 2 IGF-1Ea mice showed modest, but still significantly elevated serum IGF-1 levels (16% increase in MLC/mIGF-1 serum samples (p=0,02) and 19% in MLC/Class 2 IGF-1Ea samples (p=0,005)). Total serum levels of IGF-1 were unchanged in lines MLC/Class 1 IGF-1Eb and MLC/Class 2 IGF-1Eb (FIG. 45A). At the age of six months MLC/mIGF-1 serum samples showed a 7.4% increase in total circulating IGF-1 concentrations, which is not significant (p>0.05), while total circulating IGF-1 levels of MLC/Class 2 IGF-1Ea mice showed a similar increase noticed in three-months-old animals (24% increase (p=0.0001))(FIG. 45B). MLC/Class 1 IGF-1Ea and MLC/Class 2 IGF-1Eb again showed no effect on serum IGF-1 levels (FIG. 45B).

Most surprisingly MLC/Class 1 IGF-1A and MLC/Class 1 IGF-1B showed a significant increase in total circulating IGF-1 by the age of six month, while MLC/Class 2 IGF-1A and MLC/Class 2 IGF-1B still showed elevated levels that did not reach significance, suggesting that at least when over-expressed in skeletal muscle, Class 1 rather then Class 2 isoforms influence total IGF-1 levels in the circulation. This effect becomes more pronounced with age, when endogenous IGF-1 levels actually decrease.

In summary, over-expression of both Eb-peptide containing versions of IGF-1 do not show any effect on total levels of IGF-1 in the blood serum, while the two isoforms including the Ea-peptide induce the increase of total serum IGF-1: MLC/Class 2 IGF-1Ea mice consistently show significant increases at three and six months of age, while MLC/mIGF-1 IGF-1 serum levels are only significantly increased by the age on three months, even though still elevated by six months of age. These results support the importance of the signal peptide choice in determining the fate of the processed peptide.

Skeletal Muscle Phenotype of Selected Transzenic Lines

The previously described transgenic line, over-expressing Class 1 IGF-1A (=mIGF-1), showed skeletal muscle fiber hypertrophy, first detectable at neonatal day 10, increasing into adulthood, when the transgenic mice have developed hypertrophic trunk and limb musculature with little or no body fat (86).

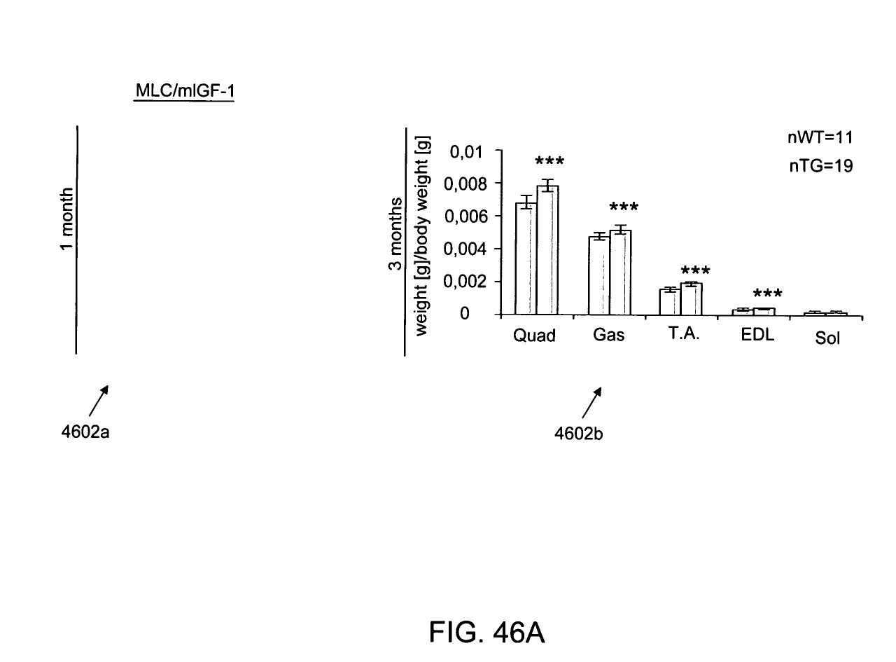

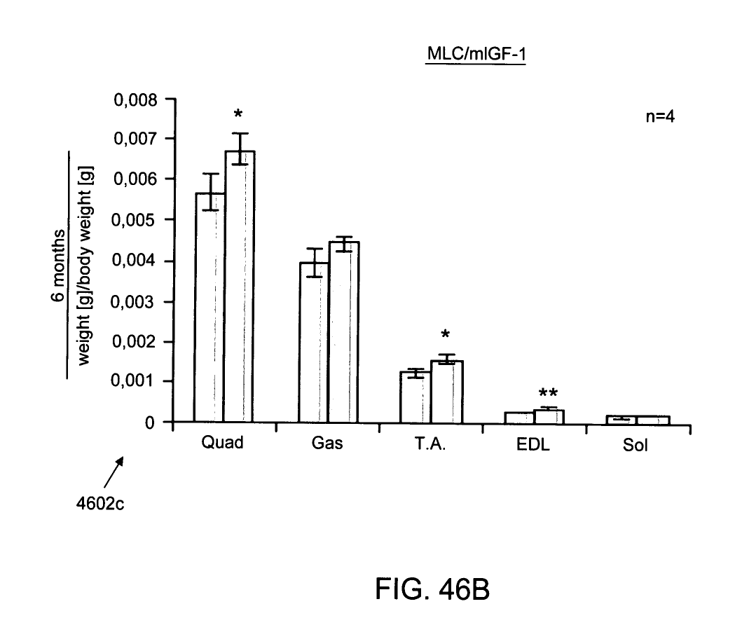

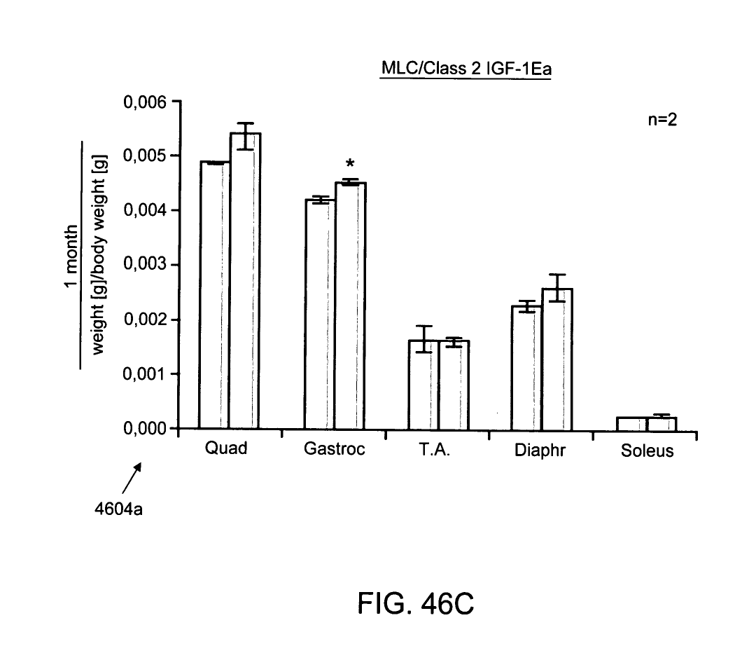

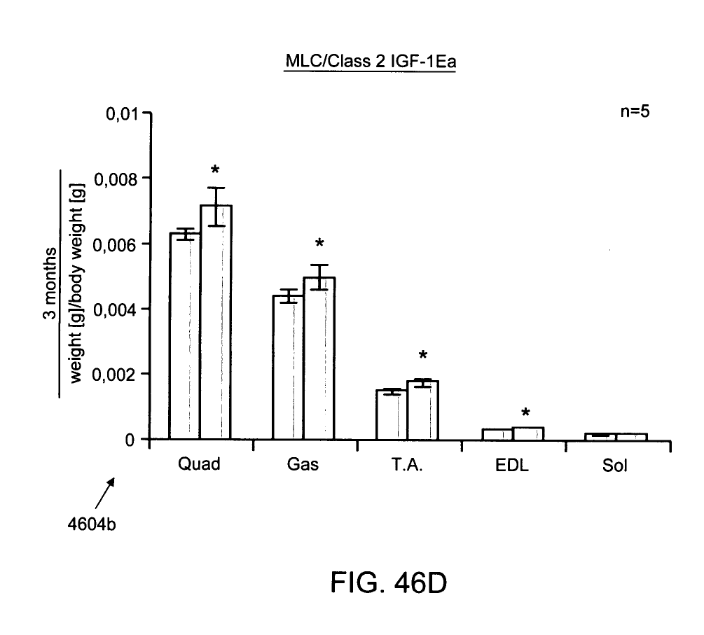

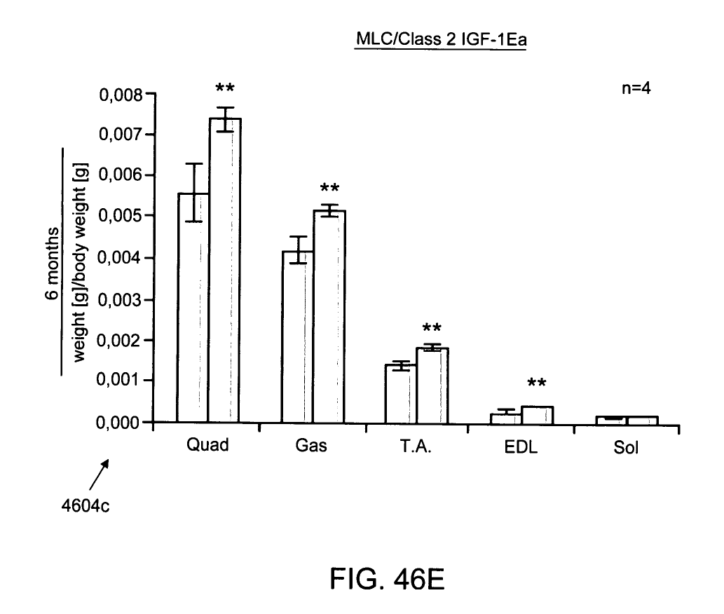

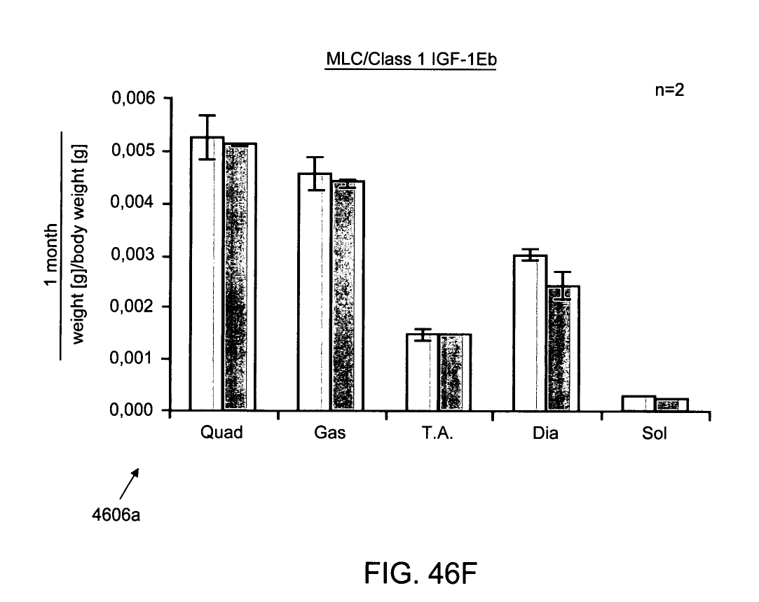

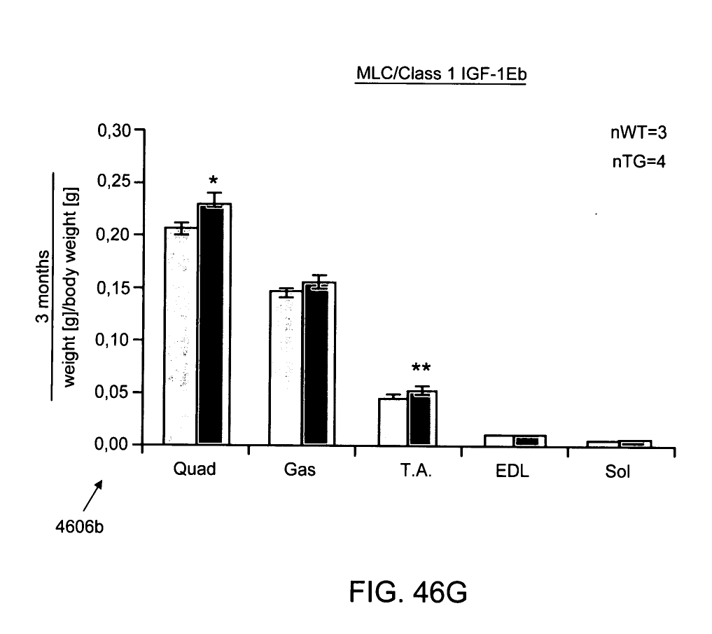

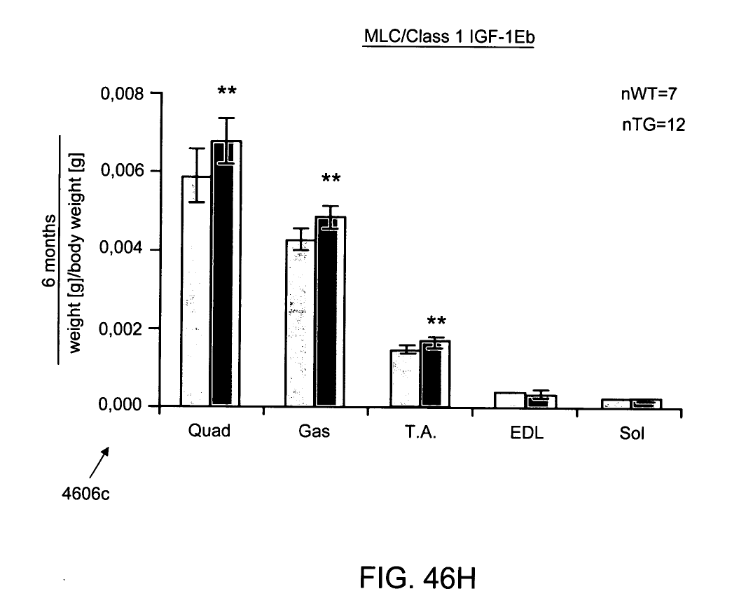

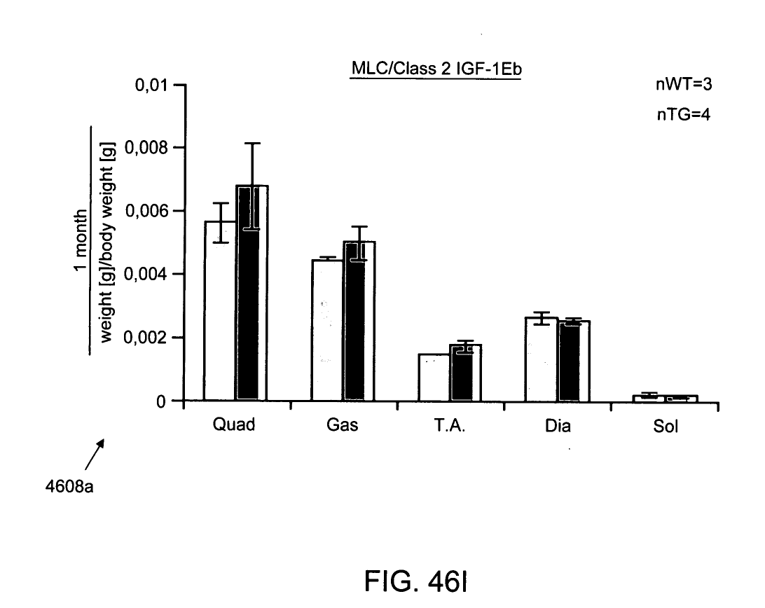

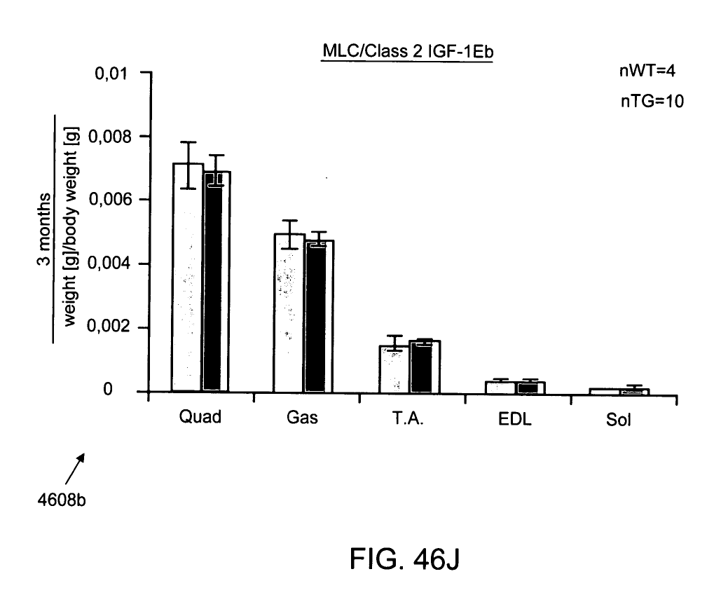

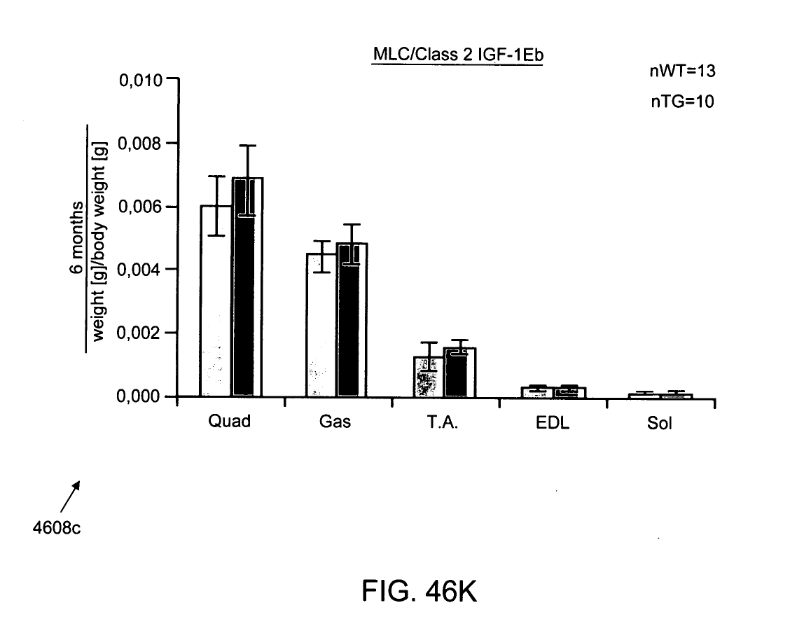

An elevated skeletal muscle weight was evident at 10 days of age for MLC/Class 2 IGF-1A and MLC/Class 2 IGF-1B, and increased significantly with age for MLC/Class 2 IGF-1A. MLC/Class 2 IGF-1B transgenic muscles showed a less pronounced, but still significant increase in skeletal muscle weight, which was maintained at comparable levels throughout the monitored ages. MLC/mIGF-1 animals showed a significant increase of all fast muscles at three months of age (quad 16%, gas 9%, TA 22%, EDL 30% (p<0.0002 for all)), which was maintained at similar levels at six months of age (quad 18%, gas 12%, TA 26%, EDL 32% (p<0.02 for all)), while the weight of the soleus, a slow muscle, was unchanged at all times (FIG. 46A). MLC/Class 2 IGF-1Ea mice displayed a modest increase of the fast muscles quadriceps (5%) and gastrocnemius (7%), which was only significant for the gastrocnemius muscle (p=0.03). The weight of TA, diaphragm, and soleus were not significantly changed (FIG. 3.17B). At three months of age the skeletal muscle weight was significantly increased in all fast muscle groups (quad 13%, gas 12%, TA 19%, EDL 24% (p<0.007 for all)), reaching even higher levels at six months of age (quad 32%, gas 23%, TA 32%, EDL 33% (p<0.007 for all)). The soleus muscle weight did not significantly change at any age, but showed a tendency for a modest weight gain (FIG. 3.17B).

In contrast to both of the Class 2 transgenic lines, MLC/Class 1 IGF-1B didn't show significant changes in skeletal muscle weight until six months of age, when skeletal muscle groups showed a very moderate, but significant increase in muscle mass. The increase of skeletal muscle weight in MLC/Class 1 IGF-1Eb animals was rather mild at all ages surveyed. At one month of age, no significant changes were found for the analyzed skeletal muscle groups (FIG. 46C). By the age of three months, quadriceps (11%) and TA (9%) weights are significant in their weight increase (p<0.03), while gastrocnemius (4%) and EDL (7%), values did not reach significance. The weight of the soleus muscle was unchanged (FIG. 46C). At six months of age, all fast skeletal muscle groups but the EDL showed a significant weight increase (quad 14%, gas 12%, and TA 12% (p<0.01). Weights of EDL and soleus were not significantly altered.

Results for the MLC/Class 2 IGF-1Eb transgenic animals revealed increased but not significant weights for fast skeletal muscle groups at one months of age (quad 20%, gas 11%, TA 15%), while measurements of three-months-old mice showed no significant differences for any of the muscles measured (FIG. 46D). By the age of six months, fast muscle weights were increased (quad 13%, gas 8%, TA 20%, and EDL 7%), but again did not reach significance. Soleus muscle weight was not influenced at any age. Taken together, lines MLC/mIGF-1 and MLC/Class 2 IGF-1Ea show the most prominent increase of skeletal muscle mass, which is consistently significant at three and six months of age. Lines MLC/Class 1 IGF-1Eb and MLC/Class 2 IGF-1Eb instead show only modest and highly variable changes in muscle weight, many which fall below the level of statistical significance.

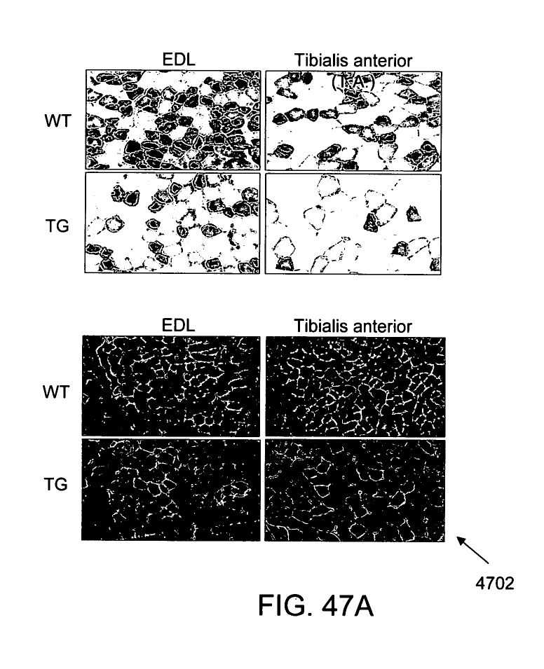

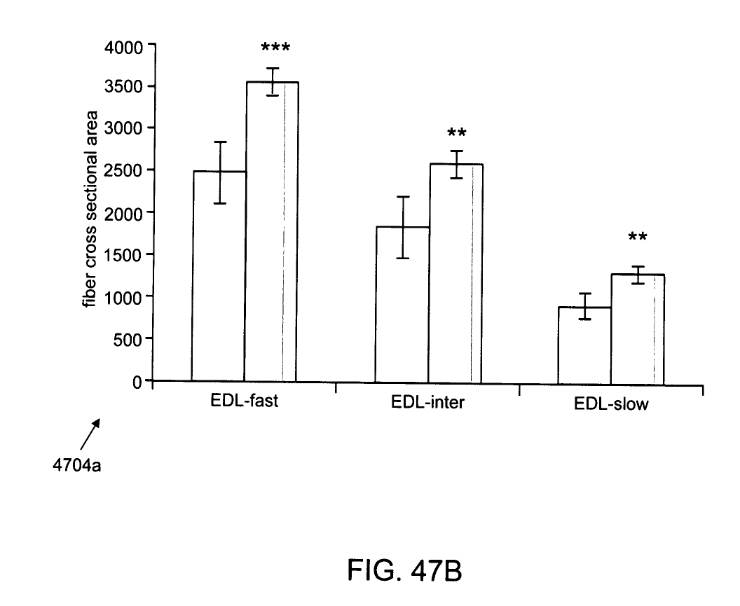

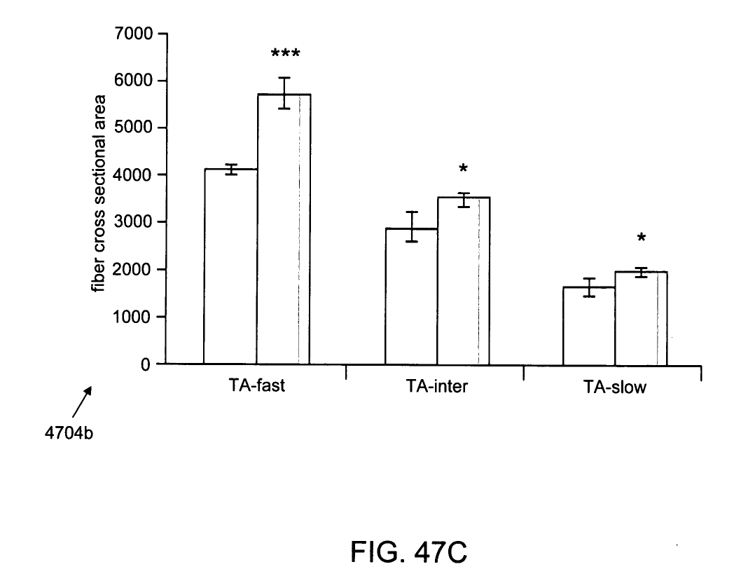

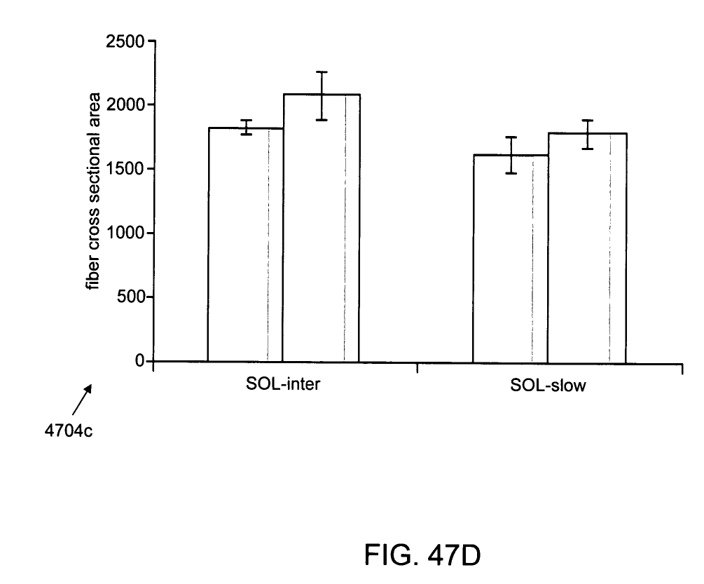

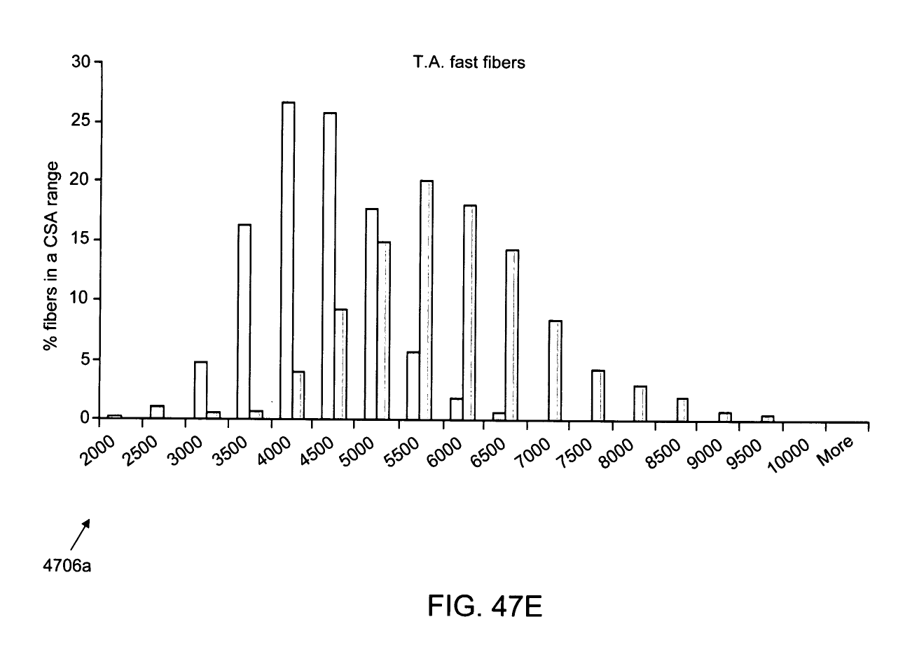

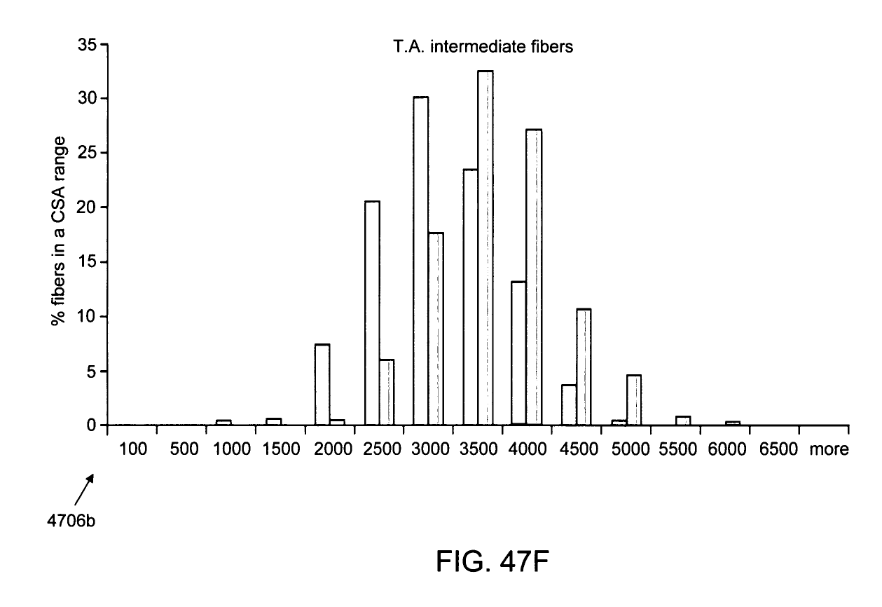

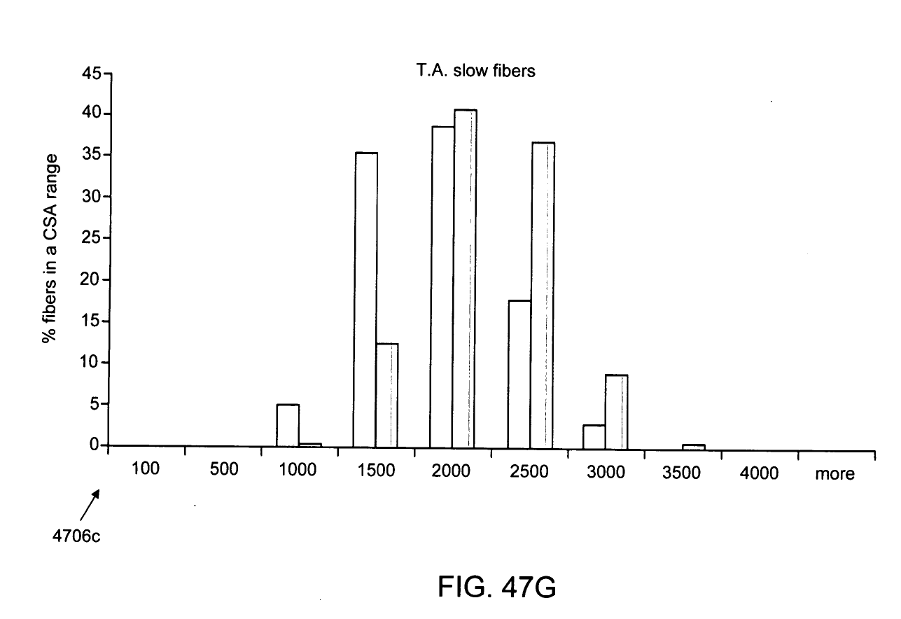

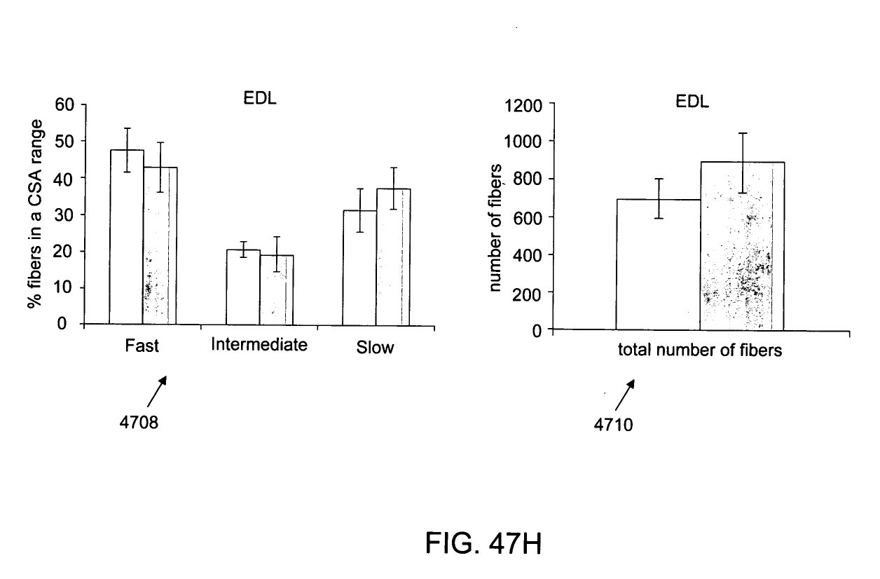

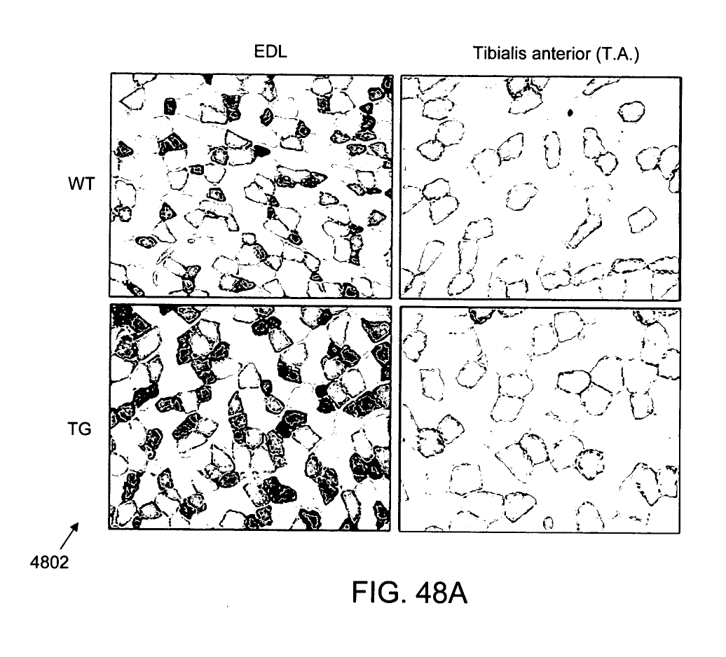

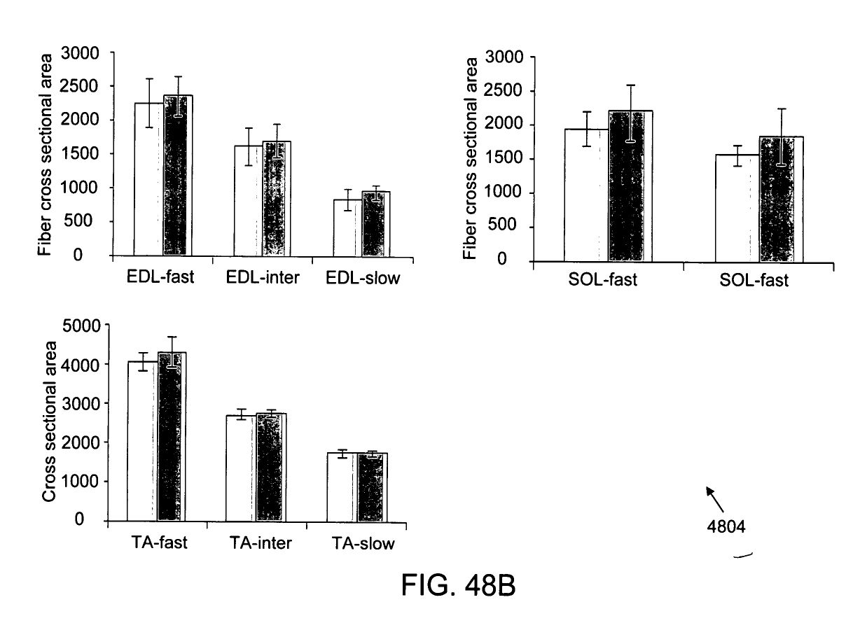

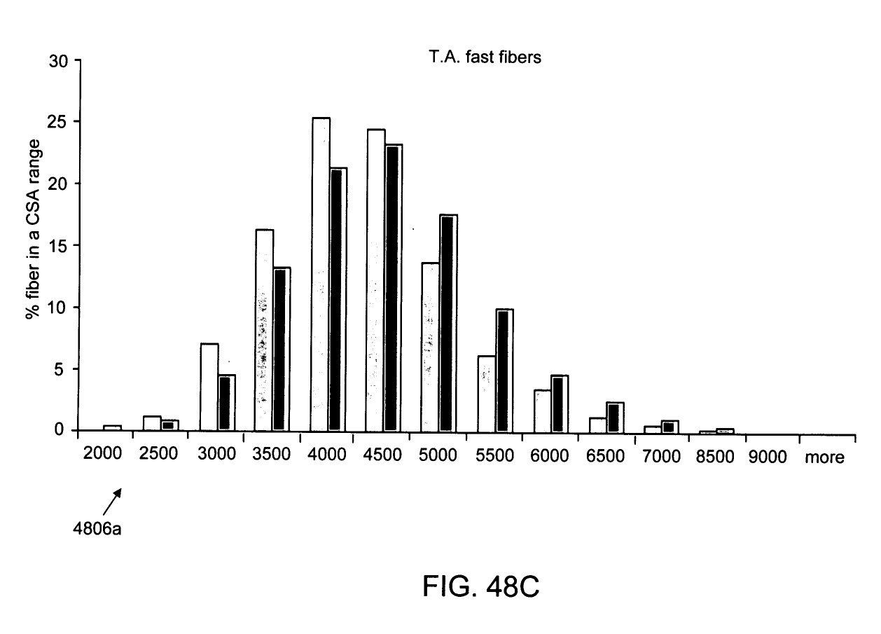

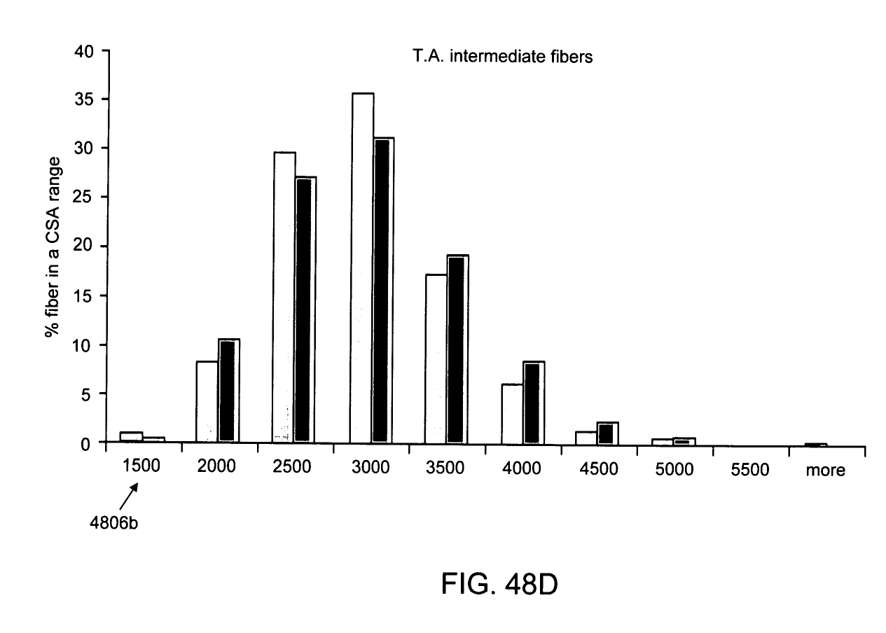

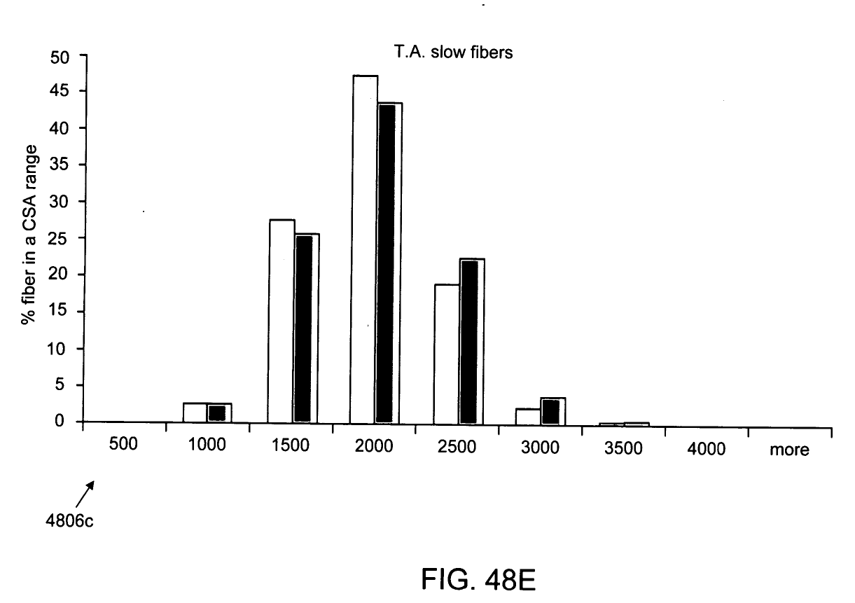

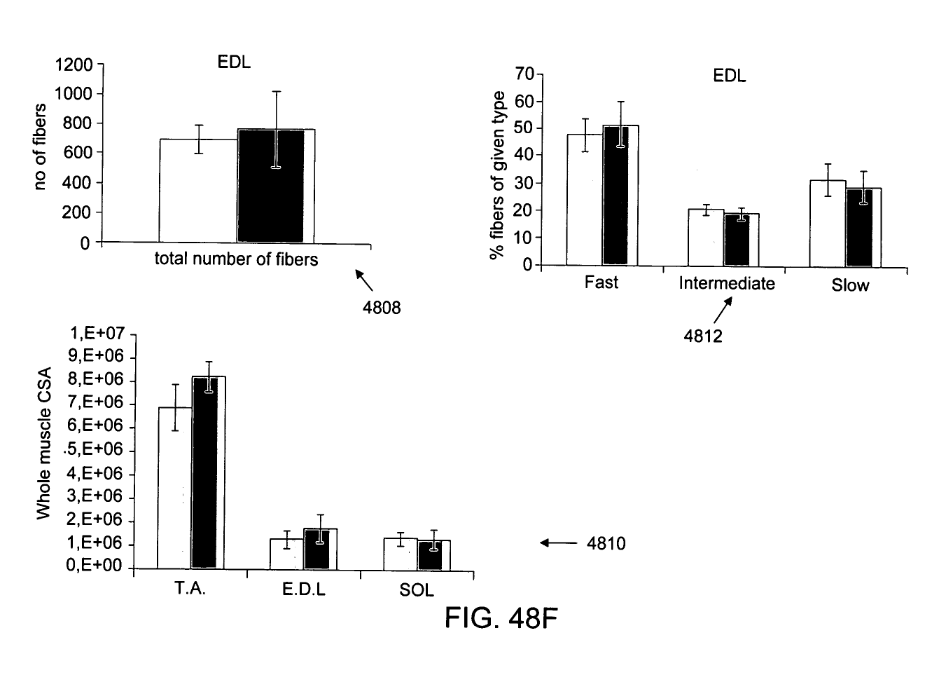

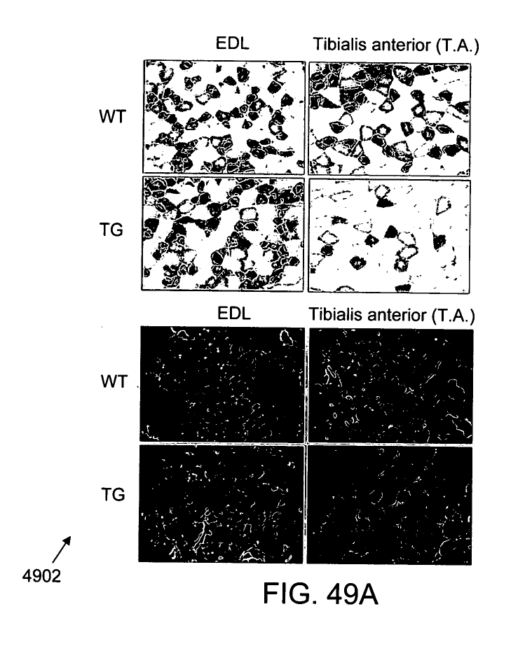

To determine whether the increase in skeletal muscle weight can be correlated to an increase of muscle fiber size, as reported for the MLC/mIGF-1 animals, six-months-old WT and transgenic mice of each line have been analyzed for differences in the single fiber cross sectional area (CSA) of the Tibialis Anterior (T.A.) and Extensor Digitorum Longus (E.D.L.) muscles. T.A. and E.D.L. both have a high content of fast type IIB fibers, which express MLC at the highest level. 12 μm frozen sections were stained for NADH-TR, which allows the differentiation between fast, intermediate, and slow fibers based on the intensity of the staining (slow fibers stain dark blue due to a higher content of mitochondria, fast fibers stain very light blue due to a low content of mitochondria, and intermediate fibers stain light blue due to a higher mitochondria content than fast fibers but a lower content than slow fibers) (FIGS. 47A, 48A, and 49A, upper panel). To confirm identity of the fast type IIB fibers, double-immunohistochemistry was performed with antibodies against type IIB myosin and laminin (FIGS. 47A and 48A, lower panel). MLC/Class 2 IGF-1 A and MLC/Class 2 IGF-1 B showed a significant increase of fast fiber CSA in T.A. fast: 39%, p=0.0002, intermediate: 20%, p=0.02, and slow: 20%, p=0.02), as well as E.D.L (fast: 44%, p=0.003, intermediate: 40%, p=0.004, and slow: 41%,.p=0.02). muscles. The slow soleus muscle showed a mild increase in CSA (13.5% for intermediate and 10% for slow fibers), which was not significant (FIG. 47B).In contrast, MLC/Class 1 IGF-1B didn't show a significant increase in the CSA of these muscles, despite the moderate but significant increase of muscle weight by the age of six months. However, when single fiber CSA was measured in six-month-old animals, a slight shift toward a higher percentage of bigger fibers was noticed in the MLC/Class 1 IGF-1B transgenic muscles, which might explain the weight increase of the different muscle groups. In these animals the CSA of the whole muscle was measured as well to determine if a higher percentage of bigger fibers was enough to increase the CSA of the whole muscle groups and therewith could account for an increased muscle mass. T.A. and E.D.L. showed an increase in CSA, which wasn't significant but still might explain the increased muscle mass of these animals.

Interestingly, MLC/Class 2 IGF-1A also showed a significant increase of the CSA of intermediate and slow fibers in T.A. and E.D.L. muscles, indicating that this isoform might be capable of functioning in a more paracrine way and thereby can act on adjacent intermediate and slow fibers to induce a hypertrophic response. Analyzing the data for the distribution of fibers of a certain CSA range as shown in FIG. 47C for the TA as an example, clearly shows that the normal size distribution of fibers in the muscle was maintained, but in presence of the MLC/Class 2 IGF-1Ea transgene, the curve was dramatically shifted towards bigger fibers. This effect was most pronounced in the fast fibers, where the transgene is predominantly expressed, but also visible for intermediate and slow fibers. The same shift was seen for the EDL muscle (data not shown). These findings indicate that the transgene effects not only the fast fibers, where the MLC promoter is most active, but also the intermediate and the slow fibers, where it is less active or not active at all.

To determine if the presence of the Class 2 IGF-1Ea transgene influenced the composition of fibers within the muscle, the number of fast, intermediate, and slow fibers was counted in the EDL muscle and did not show a significant change (fast fibers: 47±6% in the WT, 43±6% in the transgenic; intermediate fibers: 20±2% in the WT, 19±4% in the transgenic; slow fibers: 31±6% in the WT, 37±6% in the transgenic) (FIG. 47 D), indicating that the transgene did not induce any changes in fiber composition.

To rule out the possibility that the increase of skeletal muscle weight could also be due to an increase in fiber number (hyperplasia), the total number of fibers were counted in the EDL muscle of WT and transgenic animals. Results shown in FIG. 47E did not show a significant difference in the total number (695±101 in the WT and 892±156 in the transgenic EDL), providing the definite evidence that the increase of the wet weight in MLC/Class 2 IGF-1Ea transgenic animals was due to hypertrophy of the muscle fibers.

In contrast to line MLC/Class 2 IGF-1Ea, the CSA was not significantly changed in any of the muscles analyzed for line MLC/Class 1 IGF-1Eb (FIG. 48B). However, a modest increase was noted for all fiber types in the EDL muscle (fast: 4.5%, intermediate: 4.8%, and slow: 12%) and for the fast fibers in the TA muscle (6%). When analyzed for size distribution, the TA muscle (FIG. 48C), as well as the EDL muscle showed a modest shift towards a higher percentage of bigger fibers.

Since the analysis of wet skeletal muscle weight demonstrated a significant increase for both the EDL and TA muscle, the total number of fibers was counted in the EDL muscle to determine if a higher number of fibers could account for this weight increase. Results shown in FIG. 48D, demonstrate that there was no change between the total number of fibers between the WT and transgenic EDL muscle (771±101 in the WT and 694±260 in the transgenic). Thus, the weight increase could not be correlated to a higher amount of fibers in the muscle.

To determine whether the shift towards a higher percentage of big fibers in the EDL and TA was sufficient to account for the CSA increase of the whole muscle, this parameter was determined as well and showed a 19% increase for the TA CSA, a 36.5% increase for the EDL and no change for the soleus (FIG. 48E). None of these changes reached significance, but generally indicate that a small increase in individual fiber size can account for an increase of the whole muscle CSA, without affecting the mean values for fiber-specific CSA.

Finally, the analysis of fiber composition in the EDL of the MLC/Class 1 IGF-1Eb transgenic animals showed no changes (fast: 47±6% in the WT, 51±8% in the transgenic; intermediate: 20±2% in the WT, 19±2% in the transgenic; slow: 31±6% in the WT, and 29±6% in the transgenic) (FIG. 48F).

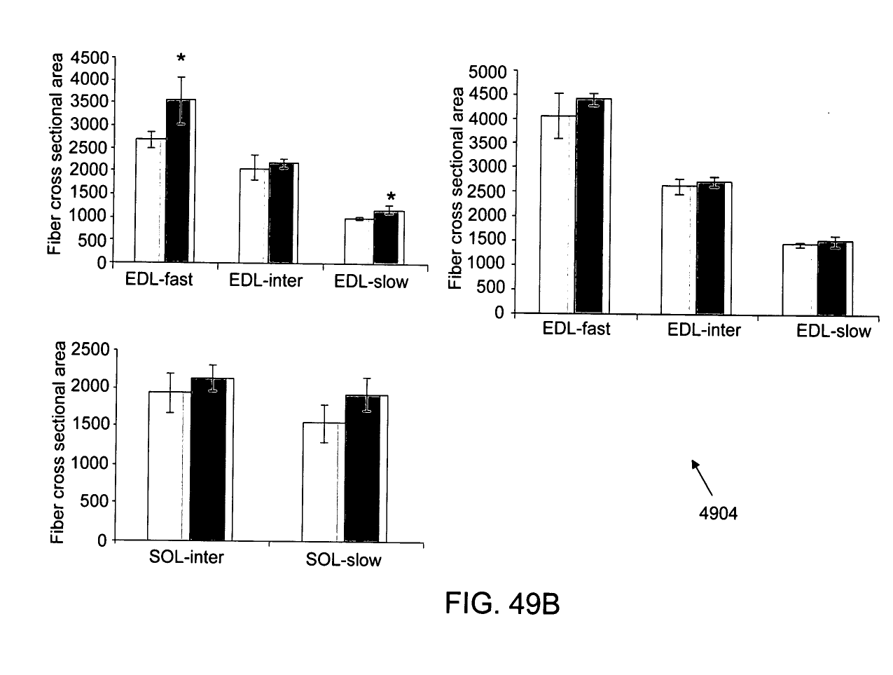

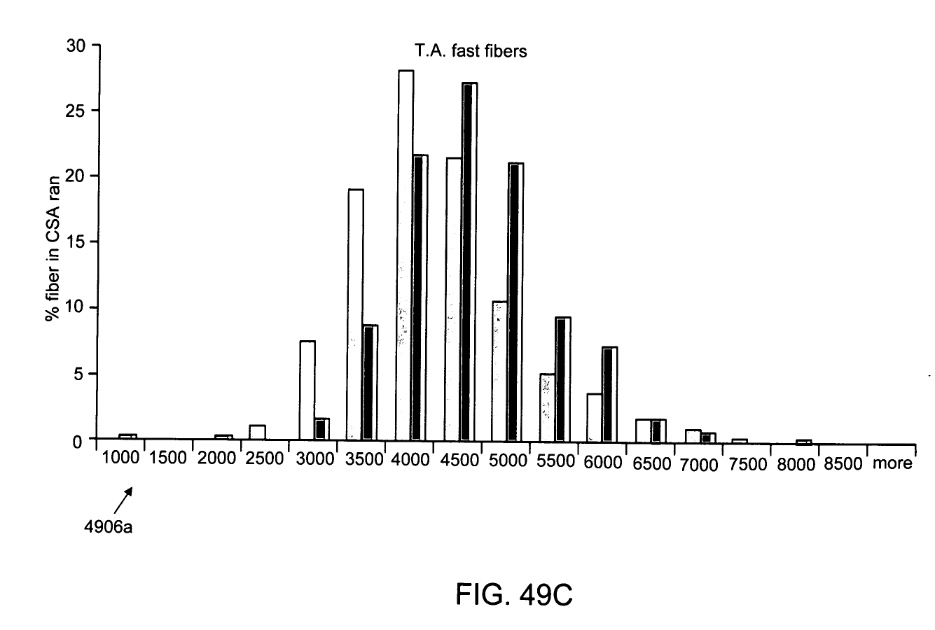

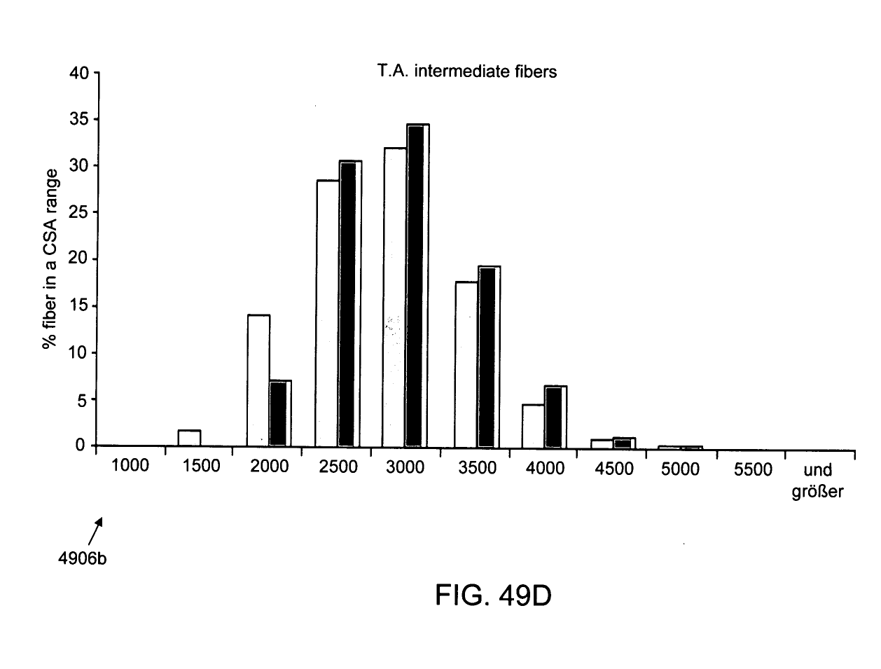

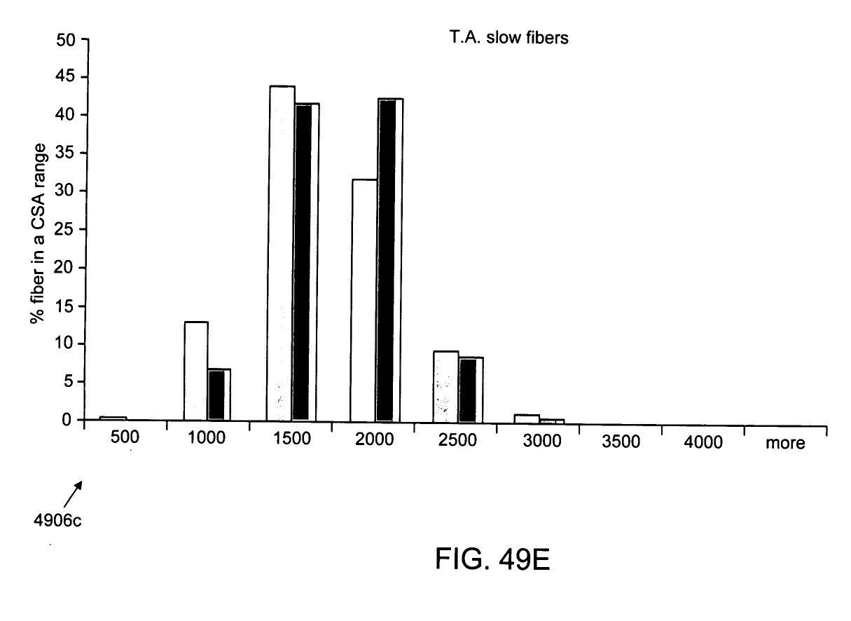

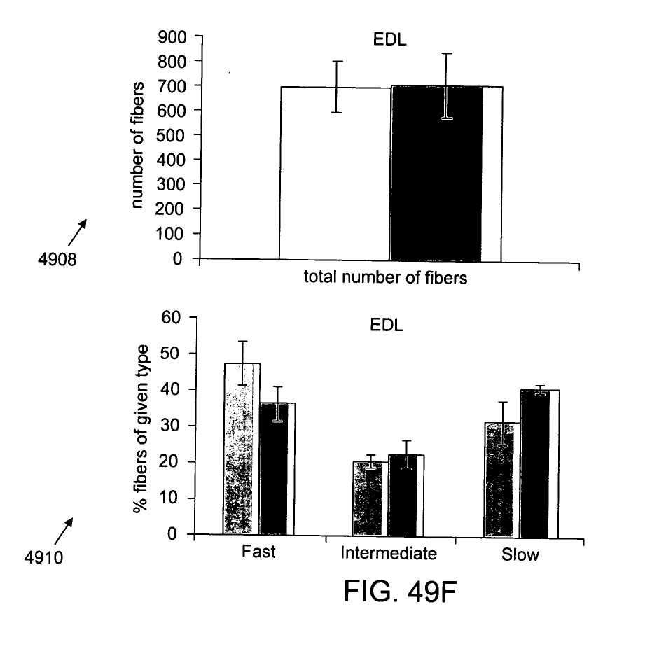

A similar analysis of the MLC/Class 2 IGF-1Eb transgenic muscles revealed that the CSA of EDL fast and slow fibers was significantly increased (fast 32%, p=0.04 and slow 19%, p=0.01), while intermediate fibers were not affected. The TA instead did not show a significant increase in any of the fiber types, even though a very moderate elevation was detectable (fast: 8.4%, intermediate: 4%, and slow: 4.4%) (FIG. 49B). A slight increase of the CSA was also seen in the soleus muscle, with intermediate fibers being 10% and slow fibers being 25% increased, but due to a high standard deviation, values did not reach significance (intermediate: 1933±264 μm² in the WT, 2129±178 μm² in the transgenic; slow: 1550±250 μm² in the WT and 1928±206 μm² in the transgenic) (FIG. 49B). The analysis of the fiber size distribution revealed that despite of the non-significant increase of fiber CSA in the TA muscle, a shift towards a higher percentage of bigger fibers was induced in presence of the Class 2 IGF-1Eb transgene (FIG. 49C). A similar shift was noted for fibers of the EDL muscle. The shift is much less pronounced than seen for MLC/Class 2 IGF-1Ea fibers and more comparable to line MLC/Class 1 IGF-1Eb.

Measurements of skeletal muscle weight of MLC/Class 2 IGF-1Eb mice did not show a significant increase of skeletal muscle wet weight, even though the TA and EDL muscle weights were elevated (20% TA and 7% EDL at six months of age).

To exclude a hyperplastic response to Class 2 IGF-1Eb over-expression, the total number of EDL fibers was determined and as seen for the other transgenic lines, did not show a significant change (695±101 in the WT and 703±129 in the transgenic) (FIG. 49D). Thus the increased amount of big fibers in the TA and EDL, which at least in the EDL muscle also resulted in a significant increase of CSA, accounts for the elevation of muscle weight. The investigation of changes in the distribution of fibers in the EDL muscle did not show any significant changes (fast: 47±6% in the WT, 36±5%, in the transgenic, intermediate: 20±2% in the WT, 22±4% in the transgenic, slow: 32±6% in the WT and 40±2% in the transgenic).

In summary the histological and morphometric analysis of three of the transgenic lines, showed a consistent increase in the CSA of all skeletal muscle fiber types in the EDL and TA muscle of MLC/Class 2 IGF-1Ea animals. The CSA of soleus fibers was unchanged. In contrast to these findings, MLC/Class 1 IGF-1Eb and MLC/Class 2 IGF-1Eb showed a milder phenotype, with a modest shift towards a higher percentage of bigger fibers, which did not reach significance in the MLC/Class 1 IGF-1Eb animals, and were only just significant for MLC/Class 2 IGF-1Eb EDL fast and slow muscle fibers. Single fiber CSA measurements of the soleus muscle, which is mainly comprised of slow and intermediate fibers and shows very low levels of MLC expression, revealed no differences to WT samples in all transgenic lines. Fiber type composition was unchanged in all transgenic lines and all analyzed muscles.

Skeletal Muscle Physiology of Selected IGF-1 Transgenic Lines

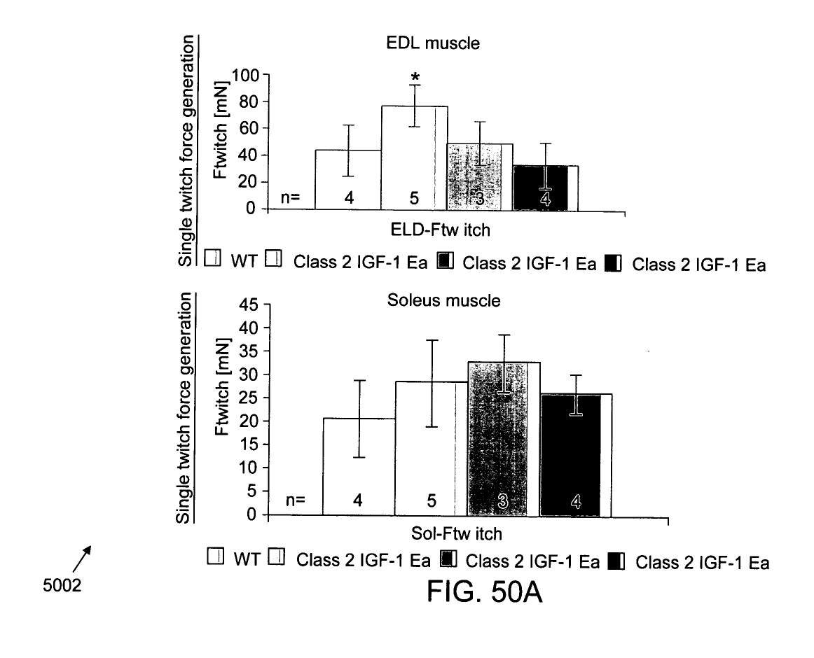

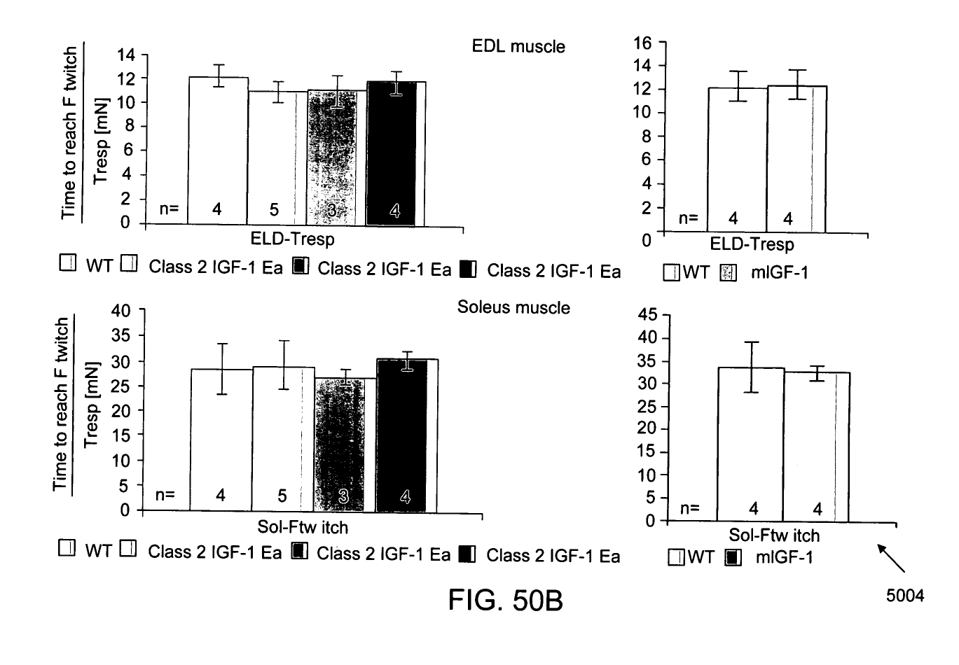

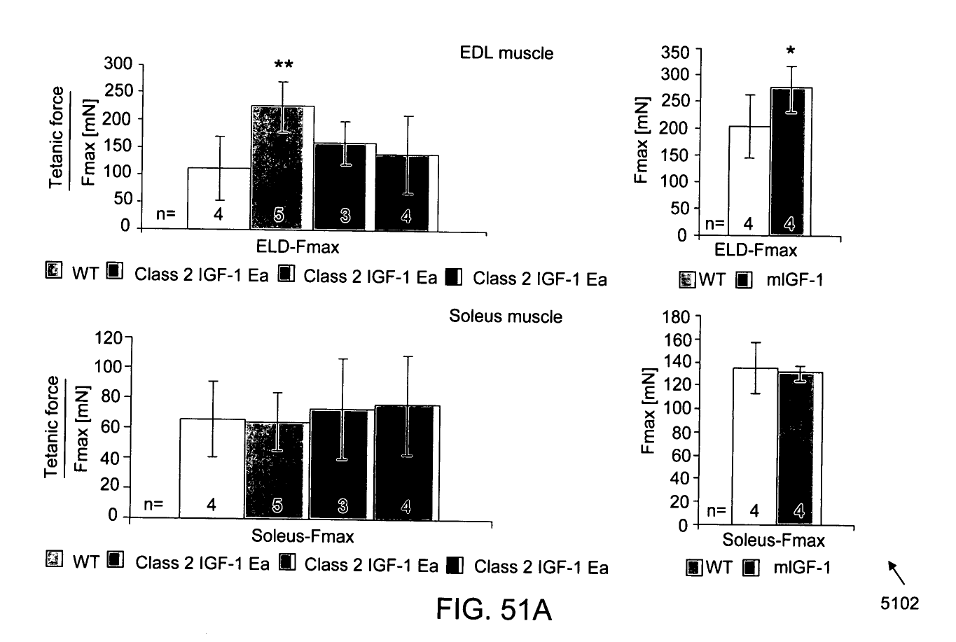

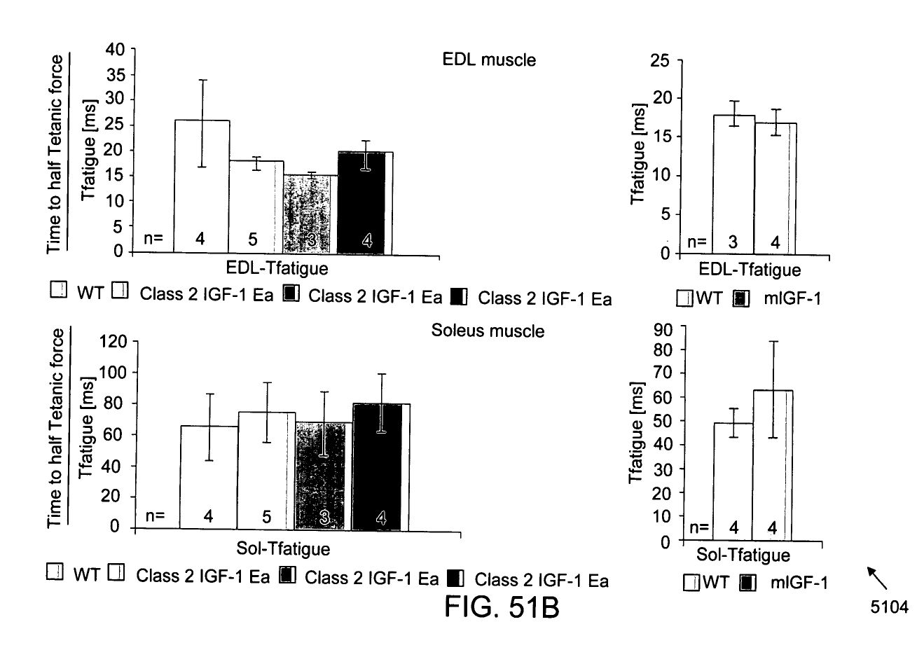

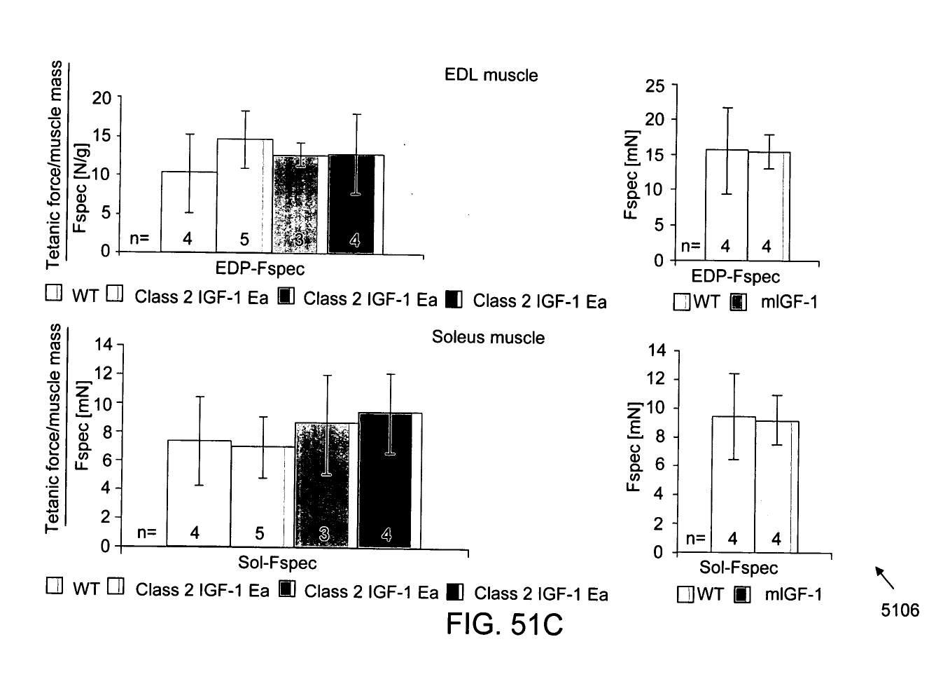

In order to translate the changes seen in the skeletal muscle phenotype of the different IGF-1 transgenic lines into functional performance of these muscles, the EDL and soleus muscle was analyzed for their physiological properties. Male WT animals (n=4) were compared to transgenic animals at 2.5 months of age (MLC/Class 2 IGF-1Ea n=5, MLC/Class 1 IGF-1Eb n=3, MLC/Class 2 IGF-1Eb n=4). Analysis of MLC/mIGF-1 animals (n=4 for WT and transgenic animals) was done separately on six-months-old mice. Measurement of the single twitch force (F_{twitch}), the tetanic force (Fₘₐₓ), and the specific force (F_{spec}) allow conclusions about the muscle strength and force generation. The measurements of the time the muscle needs to reach the peak of F_{twitch} (Tᵣₑₛₚₒₙₛₑ) reflects the contraction speed and is related to the muscle fiber composition, which also determines the time the muscle needs to reach half the titanic force (T_{fatigue}). The analysis of these parameters was analyzed in the EDL muscle as an example of a predominantly fast muscle, and in the soleus, as an example for a slow muscle. Analysis of the single twitch force FtWitch in the EDL muscle revealed a significant increase for MLC/Class 2 IGF-1Ea animals (76%, p=0.01), while MLC/Class 1 IGF-1Eb (12%) and MLC/Class 2 IGF-1Eb (23%) showed an increase that was not significant (FIG. 50A left panel). Data to determine this parameter for MLC/mIGF-1 was unfortunately not available. The EDL contraction speed (Tᵣₑₛₚₒₙₛₑ) was slightly decreased in MLC/Class 2 IGF-1Ea (9%) and Class 1 IGF-1Eb (9%) animals, while unchanged in MLC/mIGF-1 (12±1.2 ms in the WT and 12±1.2 ms in the transgenic) and MLC/Class 2 IGF-1Eb animals (12±0.9 ms in the WT and 12±0.9 ms in the transgenic) (FIG. 50B left panel). Measurements of the maximal force (tetanic force) Fₘₐₓ of the EDL muscle showed the most dramatic increase for line MLC/Class 2 IGF-1Ea (101% (p=0.003)). MLC/mIGF-1 transgenic EDL muscles also showed a significant increase of 54% (p=0.01), while values for MLC/Class 1 IGF-1Eb and MLC/Class 2 IGF-1Eb were not significant due to high variability of the measurements. However, the tetanic force was increased by 43% and 24% respectively (FIG. 51A left panel). The time the different muscles needed to reach half their EDL tetanic force Fₘₐₓ (T_{fatigue}) was decreased by 30% in MLC/Class 2 IGF-1Ea muscles, MLC/Class 1 IGF-1Eb T_{fatigue} was decreased by 39% and a decrease of 23% was measured in MLC/Class 2 IGF-1Eb EDL muscles. The high variability of the WT samples makes this decrease very unreliable and therefore none of the values were significant (FIG. 51B left panel). Values measured for line MLC/mIGF-1 were not changed at all (WT: 18.1±1.6 ms, transgene: 17.1±1.7 ms). Finally, the specific force F_{spec} for every muscle was calculated by dividing the tetanic force Fₘₐₓ by the weight of the muscle measured. EDL specific force was increased by 43% in MLC/Class 2 IGF-1Ea samples and both MLC/Class 1 IGF-1Eb and MLC/Class 2 IGF-1Eb showed an increase of 25%, without being significant in any of these animals. MLC/mIGF-1 did not show any changes in EDL specific force (WT: 15.5±6 N/g, transgenic: 15.4±2.4 N/g) (FIG. 51C left panel).

The same measurements were performed on the soleus to evaluate the effect of IGF-1 isoform over-expression on the overall physiological performance of a slow muscle. The single twitch force F_{twitch} of the soleus muscle of the different IGF-1 isoform transgenic lines showed a non-significant increase of 37% in Line MLC/Class 2 IGF-1Ea and 27% in MLC/Class 2 IGF-1Eb, while the 57% increase observed in MLC/Class 1 IGF-1Eb soleus was significant (p=0.04) (FIG. 50A right panel). The soleus contraction speed (Tᵣₑₛₚₒₙₛₑ) did not show any transgene-induced changes (MLC/mIGF-1: WT 34±5 ms, transgenic 33±1.6 ms; MLC/Class 2 IGF-1Ea: WT 28±5 ms transgenic 29±4 ms; MLC/Class 1 IGF-1B: WT 28±5 ms transgenic 27.1±1.4 ms; MLC/Class 2 IGF-lEb: WT 28±5 ms transgenic 30.6±1.9 ms) (FIG. 50B right panel). The assessment of the maximal force (tetanic force) Fₘₐₓ in the soleus muscle revealed no changes for MLC/Class 21GF1Ea (WT: 65±24 mN transgenic: 64±19 mN), MLC/Class 1 IGF-1Eb (WT: 65±24 mN transgenic: 73±33 mN), and MLC/Class 2 IGF-1Eb (WT: 65±24 mN transgenic: 75±33 mN). MLC/mIGF-1 samples instead showed a 40% increase, but due to a very high variability, this value did not reach significance (86±31 mN in the WT and 122±47 mN in the transgenic) (FIG. 51A right panel). The time the different muscles needed to reach half the tetanic force Fₘₐₓ (T_{fatigue}) was not significantly changed for MLC/Class 2 IGF-1Ea (WT: 65±21 ms, transgenic: 75±18 ms), MLC/Class 1 IGF-1Eb (WT: 65±21 ms, transgenic: 69±20 ms), and MLC/Class 2 IGF-1Eb (WT: 65±21 ms, transgenic: 81±18 ms). MLC/mIGF-1 animals showed a 27% increase, but the variability was too high to reach significance (FIG. 51B right panel). Finally, the specific force F_{spec}, calculated by dividing the tetanic force Fₘₐₓ by the weight of the muscle, did not show any differences in any of the transgenic mice analyzed (MLC/mIGF-1: WT 9.4±3 N/g, transgenic 69.2±1.8 N/g; MLC/Class 2 IGF-1Ea: WT 7±3 N/g, transgenic 6.9±2; MLC/Class 1 IGF-1Eb: WT 7±3 N/g, transgenic 8.5±3.5; MLC/Class 2 IGF-1Eb: WT 7±3 N/g, transgenic 9.4±2.8 N/g) (FIG. 51C right panel).

In summary, comparison of titanic force of Class 2 IGF-1A and Class 2 IGF-1B EDL muscles revealed a significant increase in strength of Class 2 IGF-1A over wildtype (60%) whereas Class 2 IGF-1B did not have significant increased in strength despite mild muscle hypertrophy. The electrophysiological analysis of the different IGF-1 isoform transgenic lines for the parameters related to force generation and strength (single twitch force (F_{twitch}), tetanic force (Fₘₐₓ), and specific force (F_{spec})), revealed a significant increase for the EDL muscle of MLC/Class 2 IGF-1Ea animals, and at least the maximal force generation was significantly increased in the EDL muscle of MLC/mIGF-1. These findings correlate to the increased muscle mass and fiber CSA of these two transgenic lines and establish a functional hypertrophy for MLC/Class 2 IGF-1 Ea, that is even more pronounced than previously described for line MLC/mIGF-1. In contrast, force-related parameters were not significantly changed in lines MLC/Class 1 IGF-1Eb and MLC/Class 2 IGF-1 Eb, also correlating the much milder increase in muscle mass and fiber CSA in these two transgenic lines. Parameters correlated to the fiber composition, Tresponse and T_{fatigue} in the EDL muscle were not significantly changed in any of the transgenic lines, confirming that the overall fiber composition was not affected by any of the transgenes. Analysis of these parameters in the soleus muscle of the IGF-1 isoform transgenic lines did not reveal any significant changes for none of the lines, but MLC/Class 1 IGF-1Eb, which showed a significant increase of the single twitch force F_{twitch}. Since the two Class 2 isoforms differ only by their E-peptide, these results provide further support for the inventors' proposal that the IGF-1 E peptides are responsible for controlling the different functions of the IGF-1 peptide.

Influence on Other Components of the IGF-System

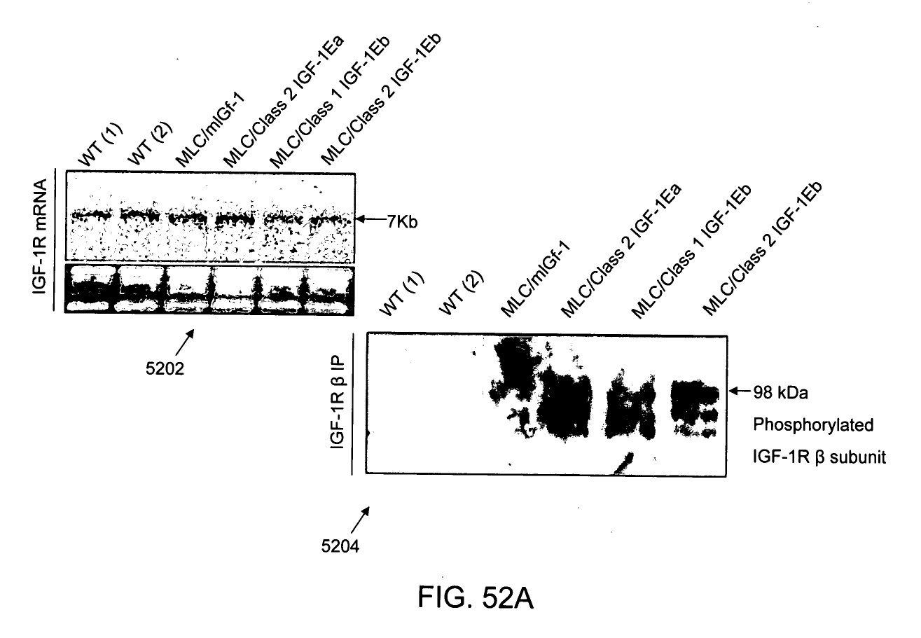

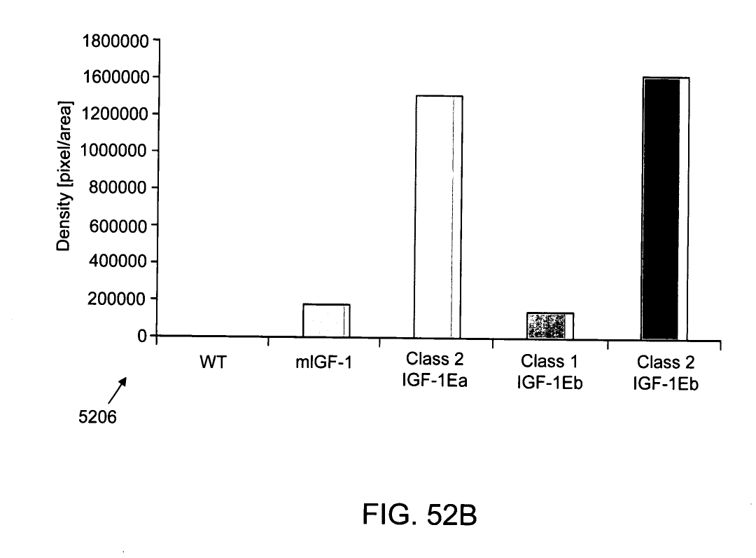

Even though two different IGF-1 receptors have been described, it is well established that IGF-1 function is exclusively mediated by the IGF-1 type 1 receptor (IGF-1R). Over-expression of IGF-1 could saturate the receptor and lead to down-regulation of transcriptional activity. To exclude the possibility that over-expression of different IGF-1 isoforms could interfere with IGF-1 receptor (IGF-1R) expression levels, Northern Blot analysis of IGF-1R mRNA levels isolated from the gastrocnemius muscle was carried out on one-month-old (FIG. 52A) and six-months-old WT and transgenic mice of each line. No differences could be detected, indicating that IGF-1R transcript levels are not influenced in the skeletal muscle of the transgenic animals and that transcriptional regulation was not affected by over-expression of the different IGF-1 isoforms.

To determine if the IGF-1R was regulated by phosphorylation, immunoprecipitations (IP) were performed with an antibody against the β-subunit of the IGF-R. Protein samples were prepared from six-months-old male mice of WT and transgenic background and 1.3 mg of protein were used for immunoprecipitations. For Western blot analysis the whole IP reaction was used for SDS-page and transfer and phosphorylation of the IGF-1R β-subunit was detected with an anti-phosphotyrosine antibody. Results presented in FIG. 52B clearly show the activation of the IGF-1R in all of the transgenic samples when compared to WT samples. Density analysis of the specific bands (Radames software) revealed that the strongest phosphorylation was seen in response to both Class 2 IGF-1 isoforms, Class 2 IGF1Ea and Class 2 IGF-1Eb (FIG. 52C).

Taken together, these results show that IGF-1R mRNA not influenced by over-expression of different IGF-1 isoforms, while activation of the IGF-1R β-subunit was seen in all IGF-1 isoform transgenic lines. These findings proof that over-expressed IGF-1 isoforms are all capable of activating the receptor, even though the extent of activation is variable, with Class 2 IGF-1 isoforms showing the strongest effect.

Other components of the IGF system are the seven different IGF-1 binding proteins (IGFBPs), which are able to either inhibit or potentiate IGF-1 action and thereby add another level to IGF-1 regulation. Affimetrix analysis of all transgenic lines at one month of age revealed an up-regulation of IGFBP-5 in MLC/Class 1 IGF-1B and was therefore the first candidate among the IGFBPs to be analyzed. IGFBP-5 can enhance IGF-1 action when bound to extracellular matrix, while it is cleaved to a biologically inactive fragment when it is soluble. Posttranslational glycosylation of IGFBP-5 has also been shown to modify the affinity to IGF-1 (106). In one-month-, as well as six-month-old animals, protein levels of non-modified IGFBP-5 were comparable to WT levels. However, MLC/Class 1 IGF-1A and MLC/Class 1 IGF-1B showed a slight induction of a higher molecular weight band, which represents a glycosylated form of IGFBP-5.

Signal Transduction Pathways

Different pathways have been implicated in IGF-1 mediated hypertrophy. While Rommel et al. (107) and Bodine at el. (63) have shown the P13 Kinase pathway and its downstream effectors Akt and GSK3 to be responsible for myocyte hypertrophy, Musaró et al. (86) have suggested the calcineurin pathway and the downstream effectors NF-ATc1 and Gata-2 as the mediator of skeletal muscle hypertrophy. To investigate whether similar activation of NF-AT and Gata-2 can be seen in the different transgenic lines, Northern and Western analysis has been performed. In contrast to the MLC/mIGF-1 muscles, Gata-2 expression was unchanged in the quadriceps of both, MLC/Class 2 IGF-1A and MLC/Class 2 IGF-1B animals, indicating that in the case of over-expressing Class 2 IGF-1 isoforms, other pathways must be implicated in the induction of hypertrophy. In MLC/Class 1 IGF-1B animals, which did not show hypertrophic muscle fibers, Gata-2 expression was expectedly not effected as well.

To further elucidate which pathway might be involved in mediating the effects of the different IGF-1 isoforms, quadriceps samples of one-month-old animals from all transgenic lines were screened for phosphorylation-mediated activation of a broad range of key kinases involved in downstream signaling of IGF-1.

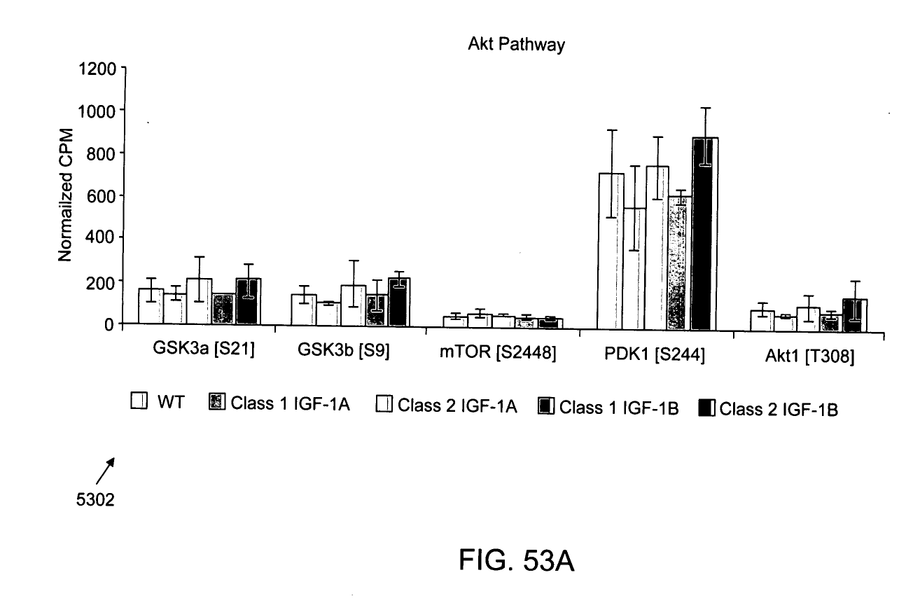

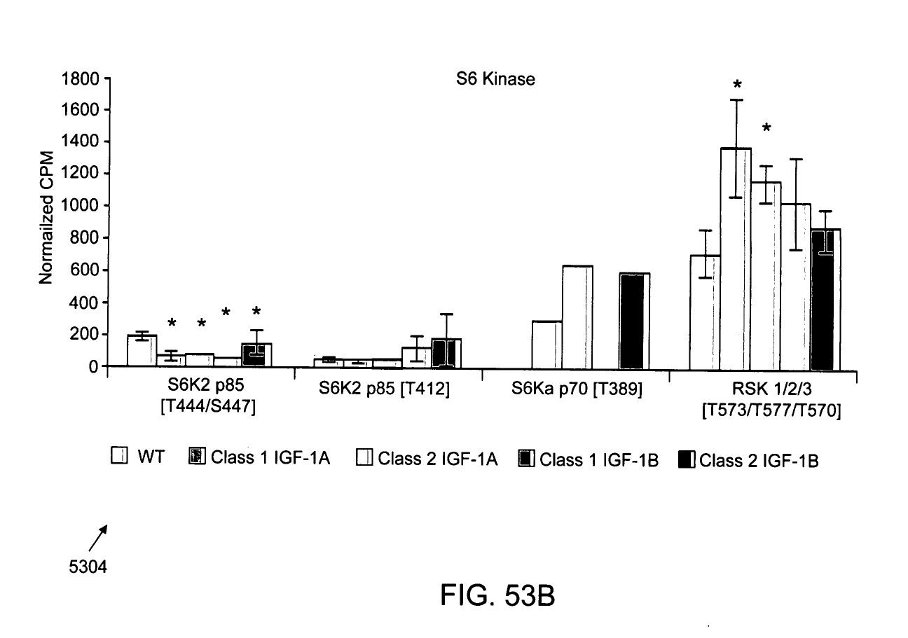

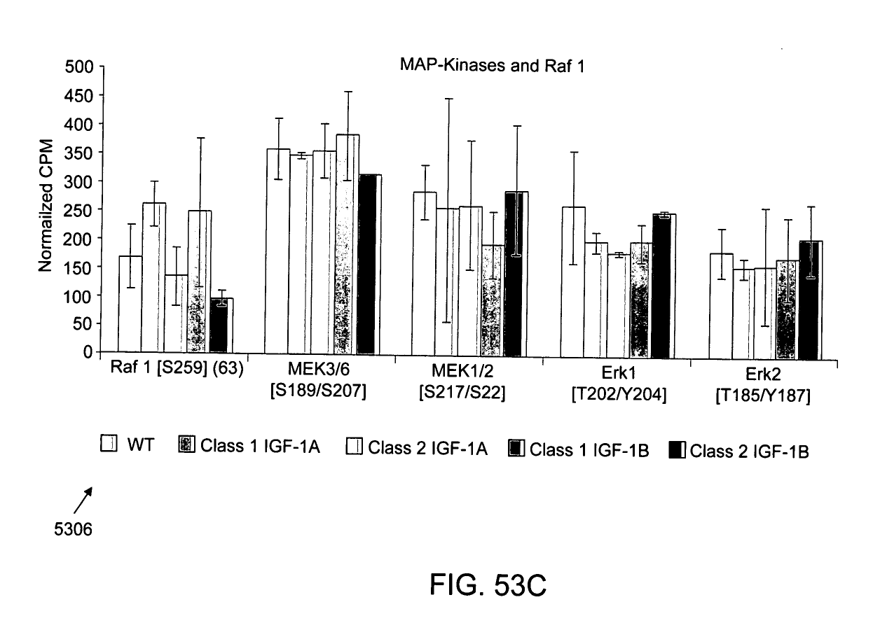

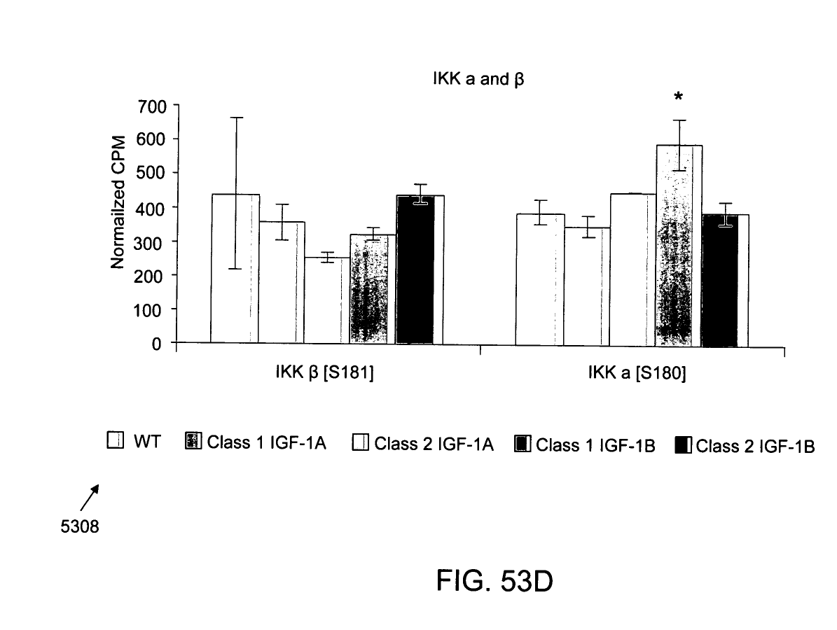

Results obtained by this high throughput screen are shown in FIG. 53A-D. The first pathway of interest was the PI(3)/Akt pathway (FIG. 53A), where GSK-3α and -3β, MTOR, PDK1, and Akt1 were analyzed for their phosphorylation state. In addition, members of the S6-kinase family, S6K p85, S6K p70 and ribosomal S6 protein-serine kinase (RSK1/2/3) were part of the screen (FIG. 53B). Notably, most of the values were not significantly changed, thus only tendencies can be described for this pathway. In the presence of both Class 1 isoforms protein phosphorylation was consistently down-regulated or did not change (MLC/mIGF-1: GSK-3α: −8%, GSK-3β: −29%, PDK1: −28%, Aktl: −26%; MLC/Class 1 IGF-1Eb: GSK-3α: −6%, GSK-3β: 2%, PDK1: −35%, Akt1: −19%). Class 2 isoforms in contrast up-regulated the phosphorylation of these protein-kinases (ML/Class 2 IGF1Ea: GSK-3α: 32%, GSK-3β: −35%, PDK1: 4%, Akt1: 12%; MLC/Class 2 IGF-1Eb: GSK-3α: 30%, GSK-3β: 55%, PDK1: 18%, Akt1: 50%). The only exception was mTOR phosphorylation, which was up-regulated by both Ea-containing isoforms (MLC/Class 1 IGF-1Ea: 32% and MLC/Class 2 IGF-1Ea: 18%), while unchanged in samples over-expressing the two Eb-containing isoforms (MLC/Class 1 IGF-1Eb: 2% and MLC/Class 2 IGF-1

Eb: 2%). These results point towards a preferential activation of the PI(3)-kinase/Akt pathway by isoforms containing a Class 2 signal peptide, while Class 1 isoforms do not seem to activate those molecules.

Interestingly this trend does not apply to the S6-kinases, which are also downstream of the PI(3)-kinase (FIG. 53B). S6-kinase p85 (S6K2) phosphorylation of residues T444 and S447 was significantly down-regulated by MLC/mIGF-1(−64%, p=0.01), MLC/Class 2 IGF-1Ea (−58%, p=0.01), and MLC/Class 1 IGF-1Eb (−71%, p=0.003), while the 17% decrease in MLC/Class 2 IGF-1Eb samples was not significant. Phosphorylation of residue T412 of S6K2 instead was unchanged in presence of MLC/Class 2 IGF-1Ea (−1%) and increased by the other three IGF-1 isoforms (MLC/mIGF-1: 30%, MLC/Class 1 IGF-1Eb: 121%, MLC/Class 2 IGF-1Eb: 218%). Our findings suggest a preferential phosphorylation of residue T412 in these latter three transgenic lines. Analysis of p₇₀56K (S6K1) phosphorylation showed a strong induction in presence of IGF-1 isoforms MLC/mIGF-1 (299%), MLC/Class 2 IGF-1Ea (635%), and MLC/Class 2 IGF-1Eb (606%), while p₇₀56K was not present in WT and MLC/Class 1 IGF-1Eb samples. The induction seen in the first three transgenic lines again did not reach significance, but allows speculations about a preferential activation of p₇₀56K over p₈₅56K. Finally, phosphorylation of RSK1/2/3 revealed a significant increase for both Ea-containing isoforms (MLC/mIGF-1: 91%, p=0.02 and MLC/Class 2 IGF-1Ea: 60%, p=0.02), while the induction by both Eb-containing isoforms was not significant (MLC/Class 1 IGF-1Eb: 42% and MLC/Class 2 IGF-1Ea: 21%). The significant results obtained for both Ea-containing isoforms do raise the possibility that these IGF-1 variants might transmit part of their hypertrophic signal via RSK1/2/3.

The other major pathway downstream of the IGF-1R is the MAP-kinase pathway. Raf-1, as the most upstream kinase, MAPK-Erk protein-serine kinase (MEK) 3/6 and MEK1/2, Erk1, and Erk2 were analyzed for their phosphorylation state by the Kinetworks™ Phospho-site screen (FIG. 53C). Phosphorylation of Raf1 was increased only in response to Class 1 IGF-1 isoforms (MLC/mIGF-1: 55% and MLC/Class 1 IGF-1Eb: 48%), while both Class 2 IGF-1Ea isoforms showed a decrease (MLC/Class 2 IGF-1Ea: −19% and MLC/Class 2 IGF-1Ea: −42%). MEK3/6 displayed minor differences (MLC/mIGF-1: −3%; MLC/Class 2 IGF-1Ea: −1%; MLC/Class 1 IGF-1Eb: 6%; MLC/Class 2 IGF-1Ea: −12%), MEKI/2 levels were highly variable and no pattern of Class or E-peptide-specificity could be noted (MLC/mIGF-1: −9%; MLC/Class 2 IGF-1Ea: −8%; MLC/Class 1 IGF-1Eb: −31%; MLC/Class 2 IGF-1Eb: 55%). Erk1 phosphorylation was down-regulated in all different IGF-1 isoform transgenic samples (MLC/mIGF-1: −23%; MLC/Class 2 IGF-1Ea: −31%; MLC/Class 1 IGF-1Ea: −23%; MLC/Class 2 IGF-1Eb: −3%), while Erk2 activation was decreased in MLC/mIGF-1 (−23%), MLC/Class 2 IGF-1Ea (−12%), and MLC/Class 1 IGF-1Eb (−5%) transgenic lines, but was slightly up-regulated in MLC/Class 2 IGF-1Eb (12%). The data exclude a pronounced up-regulation of Erk1 and Erk 2 in response to any of the IGF-1 isoforms.

Levels of Ikkα were moderately changed by MLC/mIGF-1 (−11%), MLC/Class 2 IGF-1Ea (14%), and MLC/Class 2 IGF-1Eb (−0.5%), while MLC/Class 1 IGF-1Eb significantly up-regulated Ikka-phosphorylation by 51% (FIG. 53D). IKKβ phosphorylation was not significantly affected by any of the IGF-1 isoform transgenes, but was slightly decreased (MLC/mIGF-1: −18%, MLC/Class 2 IGF-1Ea: −42%) or unchanged (MLC/Class 2 IGF-1Eb: 0.6%).

Downstream effectors of the PI3 kinase pathway, like Akt, PDK1, and GSK3α and β were up-regulated in both Class 2 IGF-1 isoforms, while not affected or down-regulated in both Class 1 IGF-1 isoforms. Down-regulation of Akt in Class 1 isoforms confirm recent findings of Song et al., (108) showing that Akt is not involved in mediating mIGF-1 (Class 1 IGF-1A) induced hypertrophy. On the other hand, the same group reported an increased phosphorylation of PDK1, mTOR, and p70S6K, which was not seen for either Class 1 IGF-1A, or Class 1 IGF-1B. These results imply a signal peptide-specific difference in the induction of signal transduction pathways.

In summary, results described above point towards Class 2 IGF-1 isoforms signaling through the classical PI(3)-kinase pathway, since they showed an increase in Akt1, PDK-1, and GSK-3α and -3β phosphorylation, while Class 1 IGF-1 isoforms did not. The analysis of the S6-kinases as further downstream targets of this pathway, revealed an induction of p70^{S6K}-phosphorylation in MLC/mIGF-1, MLC/Class 2 IGF-1Ea, and MLC/Class2 IGF-1Eb samples, while p70^{S6K} was absent in MLC/Class 1 IGF-1Eb samples. RSK1/2/3 S6-kinases-phosphorylation was increased in response to all IGF-1isoforms, but only Ea-peptide containing isoforms (MLC/mIGF-1 and MLC/Class 2 IGF-1Ea) values reached significance.

The MAP-Kinase Pathway was not Regulated to a Significant Extent, but showed Increased Levels of Raf1-phosphorylation for both Class 1 IGF-1 Isoform Transgenic Lines (MLC/mIGF-1 and MLC/Class 1 IGF-1Eb). Phosphorylation of Ikka was Significantly Increased in MLC/Class 1 IGF-1Eb Samples. Regeneration of Transgenic IGF-1 Isoform Muscles

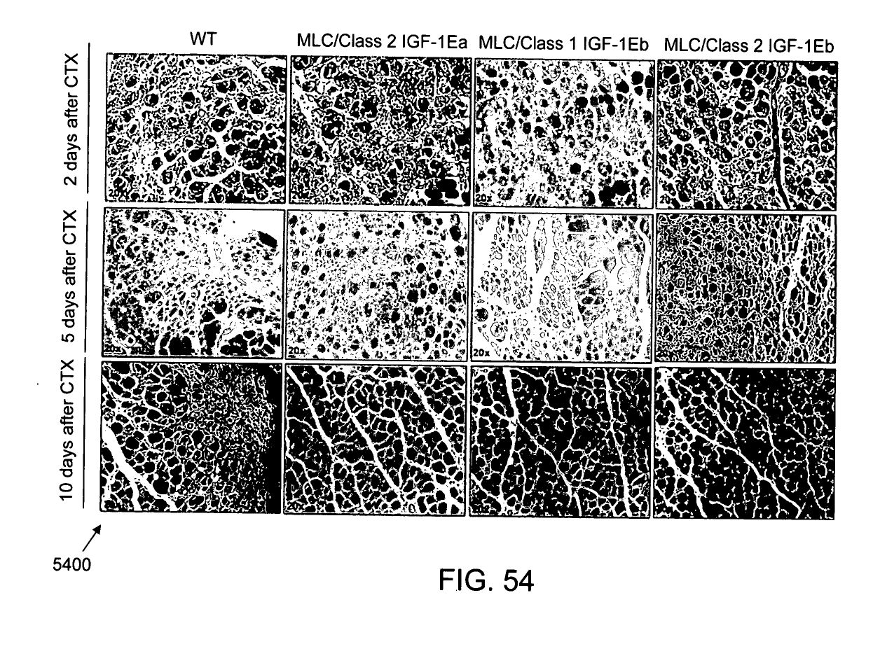

(MLC/mIGF-1 transgenic animals have been reported to show enhanced regeneration upon cardiotoxin-induced skeletal muscle injury (86). For analysis of regenerative capacity, MLC/Class 1 IGF-1B and MLC/Class 2 IGF-1A transgenic animals were focused on, since these two transgenic lines showed the most prominent phenotype in skeletal muscle. To evaluate changes in the regenerative capacity of the different IGF-1 isoform transgenic lines after CTX-induced injury, the TA muscle sections of WT and transgenic mice were stained with the Trichrome staining, which allows the visualization of skeletal muscle fibers, nuclei, as well as fibrotic tissue, by staining collagen. Representative pictures are shown in FIG. 54. Cardiotoxin was injected into the T.A. muscle and animals were analyzed at two, five, and ten days after the injections. MLC/Class 1 IGF-1B mice show a significantly enhanced regenerative response compared to wildtype mice. After two days, massive injury was seen in both, WT and transgenic muscles. Five days after injection, the WT muscle showed high levels of infiltrating mononuclear cells, indicating inflammatory processes. The proliferative response of muscle satellite cells was also initiated at this time point, characterized by small myofibers with centralized nuclei. In contrast to the WT, the MLC/Class 1 IGF-1B muscle showed a dramatic increase in the formation of new fibers, as well as a less severe inflammatory response. In addition, many newly formed fibers contained up to 3 nuclei already, while the majority of WT fibers only contained one central nucleus. This indicates that the regeneration process in MLC/Class 2 IGF-1Ea proceeded faster. Overall, the morphology of the regenerating muscle appeared to be improved by the presence of this IGF-1 isoform transgene. Similarly, MLC/Class 1 IGF-1Eb muscle sections displayed a more ordered morphology with more free intracellular space. In comparison to the WT and also to MLC/Class 2 IGF-1Ea samples, muscles of MLC/Class 1 IGF-1Eb animals had more smaller new fibers, rather than bigger new fibers as seen in MLC/Class 2 IGF-1Ea animals. The regenerating fibers did not have more than two nuclei and were only slightly increased in size when compared to WT controls. MLC/Class 2 IGF-1Eb samples instead had a similar or even slightly stronger inflammatory response when compared to the WT, but nevertheless displayed a visible increase in the amount of fibers present in the injured area. The size of the new myofibers showed a mild increase but did not reach the size of MLC/Class 2 IGF-1Ea fibers and the majority of those fibers contained only one central nucleus.

After 10 days the transgenic muscle had undergone almost complete regeneration. New fibers had reached normal size, no fibrotic tissue formation was detectable, and mononuclear cells were cleared, indicating that the inflammatory processes have been resolved. In the WT muscle the majority of newly formed fibers were still quite small, mononuclear cells were still visible and some fibrotic tissue formation was seen. The three IGF-1 isoform transgenic lines uniformly displayed a more normal morphology. These results imply that Class 1 IGF-1B enhances the regenerative process in the same way as mIGF-1, where an increased proliferation of satellite cells (86), an increased recruitment of bone marrow cells (27), and a down-regulation of the inflammatory response is seen. This is an important result as it demonstrates that an IGF-1 isoform, which does not induce skeletal muscle hypertrophy, is nevertheless capable of enhancing the regenerative response in response to traumatic injury. It shows that enhanced regeneration is not connected to the hypertrophic phenotype seen in MLC/mIGF-1 animals.

Taken together, this preliminary analysis of tissue morphology after CTX injury revealed an improved regeneration process in every transgenic line analyzed MLC/Class 2 IGF-1Ea displayed larder fibers containing between one and three nuclei at day five after injury, MLC/Class 1 IGF-1Eb and MLC/Class 2 IGF-1Eb showed more fibers when compared to WT and also to MLC/Class 2 IGF-1Ea. After ten days of regeneration, all transgenic lines displayed similar improvements in muscle morphology than the WT. Indicating that the presence of the different IGF-1 isoforms each resulted in enhanced regeneration. Changes in endogenous IGF-1 isoform expression in response to injury.

IGF-1 isoforms have been implicated in the regenerative response to exercise- and stretch-induced injury previously and have therefore been included in the present regeneration study for several reasons. First, this allowed the comprehensive analysis of WT IGF-1 isoform expression in response to muscle injury, which previously has been focused on Class 1 IGF-1Ea and Class 1 IGF-1Eb expression analysis. Second, endogenous IGF-1 levels of IGF-1 isoform transgenic lines were monitored and compared to the WT expression of the same isoform and time point after injury to evaluate if over-expression of certain IGF-1 isoforms had a feedback effect on the endogenous IGF-1 isoform expression pattern after CTX-injury.

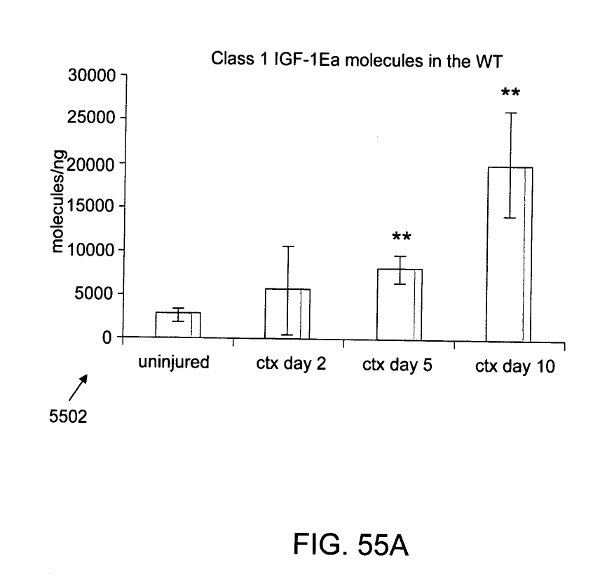

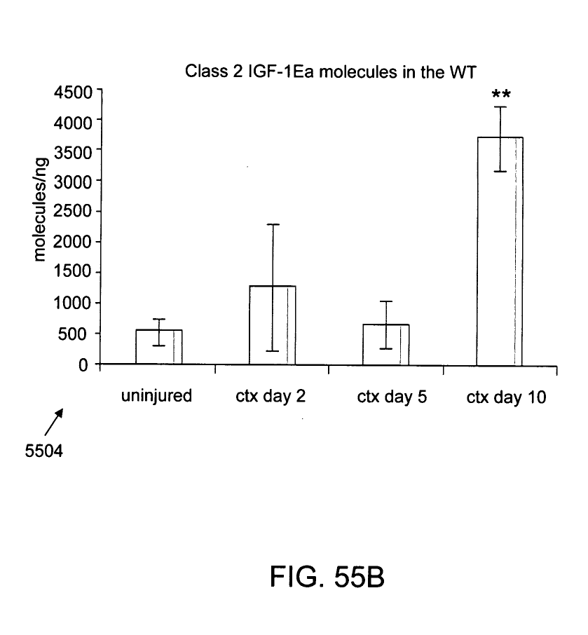

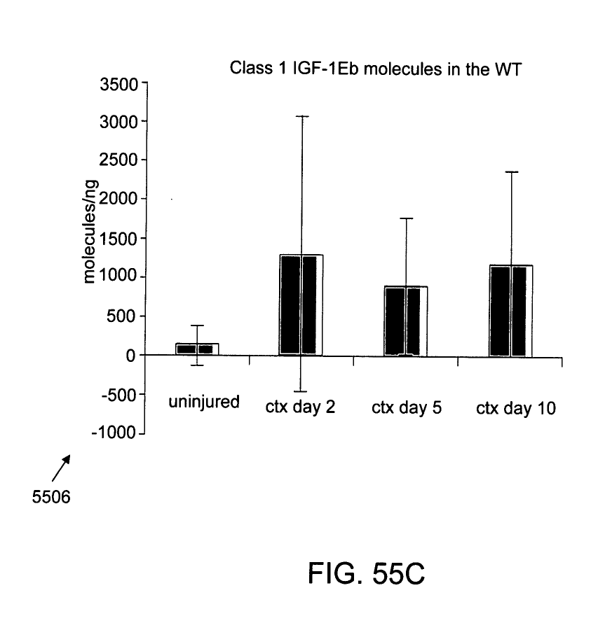

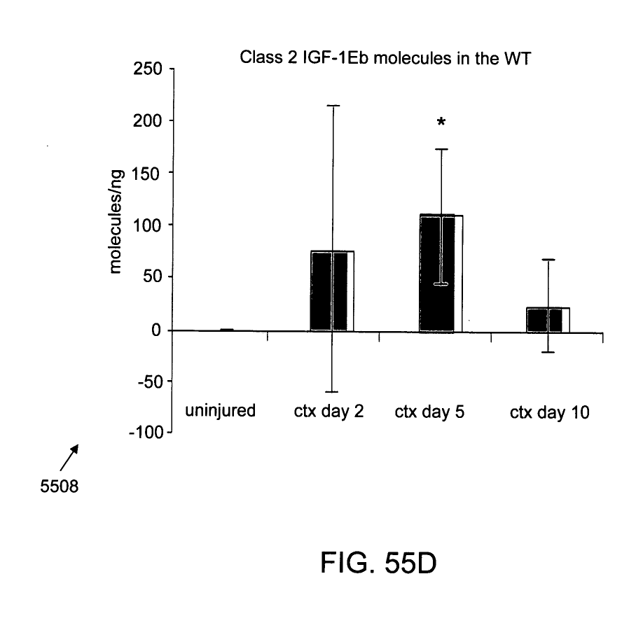

This analysis was performed by quantitative RT-PCR on RNA samples isolated from the quadriceps muscle of injured WT and transgenic animals (n=3 for all genotypes and time points). Three uninjured controls were included for every genotype. Analysis of endogenous Class 1 IGF1Ea in the WT background revealed a non-significant increase of 111% at day two after injury, and a significant and pronounced increase of this isoform at day five and day ten after injection (209% at day five (p=0.003) and 664% at day ten (p=0.001) when compared to uninjured controls (FIG. 55A). Class 2 IGF-1Ea molecules displayed a similar but less pronounced pattern of increased expression, culminating in a significant induction after ten days of regeneration (604%, p=0.005). Notable, expression of this isoform was generally lower than Class 1 IGF1Ea expression (FIG. 55B).

Class 1 IGF-1Eb and Class 2 IGF-1Eb isoforms instead showed very high variability with generally low expression profiles and were therefore not significantly changed throughout the process of regeneration. The only exception was a transient and significant up-regulation of Class 2 IGF-1Eb molecules at day five after injury (111%, p=0.02) (FIG. 55C and D). Despite the high variability, a mild induction of the two Eb-containing isoforms that was comparable throughout the different time points was observed.

In summary, the analysis of the WT expression pattern of IGF-1 isoforms upon injury revealed a pronounced up-regulation of both Ea-peptide-containing isoforms throughout the process of regeneration, reaching highest induction levels at day ten after injury. The two Eb-containing isoforms instead, showed mild induction upon injury without being significant. The only exception was a transient up-regulation of Class 2 IGF-1Eb at day five after injury.

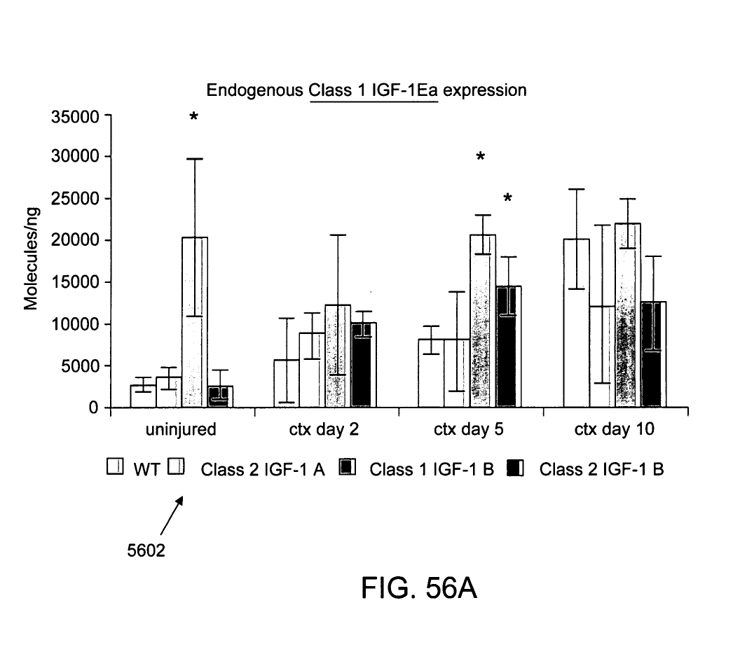

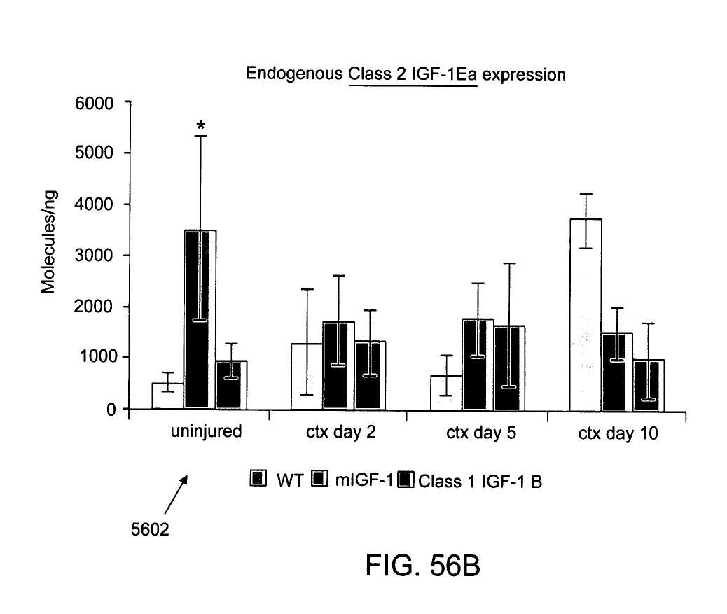

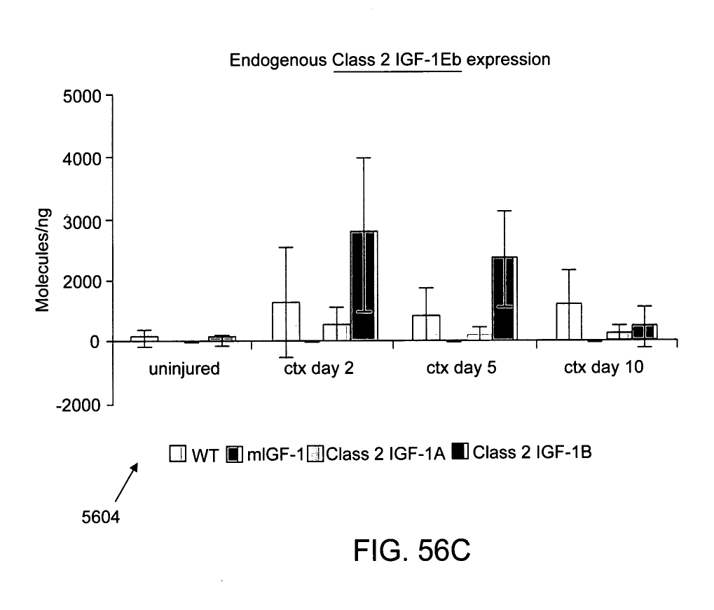

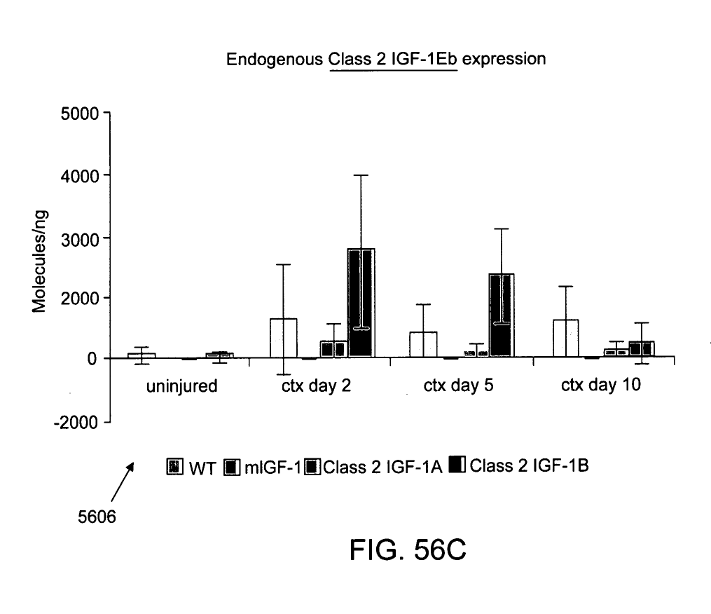

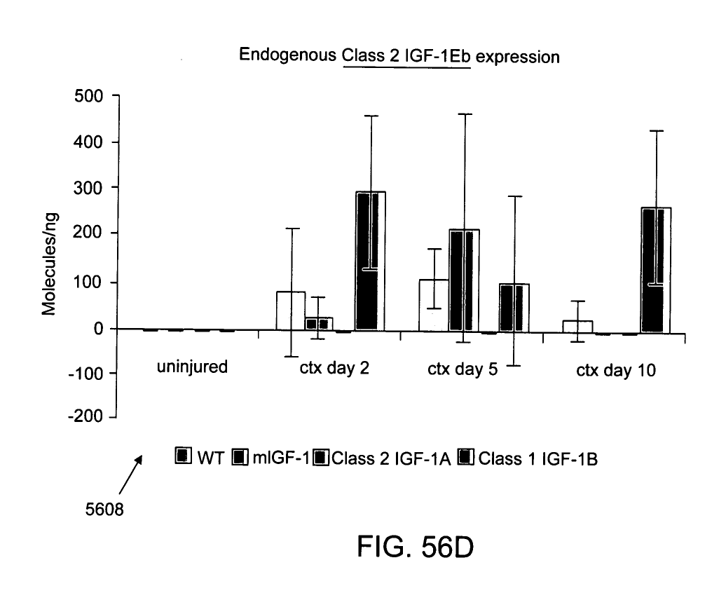

The WT expression pattern of IGF-1 isoforms was then compared to the expression pattern of endogenous isoforms in the different IGF-1 isoform transgenic animals. The line over-expressing the analyzed isoform of interest was not included in the analysis, as endogenous isoform levels were masked by the over-expression of the transgene. Results for endogenous Class 1 IGF-1Ea expression revealed no significant changes for line MLC/Class 2 IGF-1Ea throughout the regeneration process (FIG. 56A). Analysis of line MLC/Class 1 IGF-1Ea showed a significant (p=0.03) up-regulation of this isoform in uninjured MLC/Class 1 IGF-1Eb animals. At day two after injury, when many myofibers are destroyed this up-regulation is decreased, but an elevation of Class 1 IGF-1Ea mRNA was still visible (123%), but not significant anymore. Five days after injection, MLC/Class 1IGF-1Eb samples were again significantly increased by 154% (p=0.001), while at day ten the 9% increase of Class 1 IGF-1Ea mRNA was no longer significant (FIG. 56A), due to up-regulation of this isoform in the WT upon injury. Since values were compared to the WT expression of the same day, this increase is not significant. MLC/Class 2 IGF-1Eb samples showed no significant changes at day two and ten after injury, but significantly up-regulated Class 1 IGF-1Ea mRNA at day five when compared to the WT sample of the same time point (77%, p=0.04) (FIG. 56A). Taken together, endogenous Class 1 IGF-1Ea expression patterns observed in the WT were not influenced by over-expression of Class 2 IGF-1Ea, but showed a significant increase at day five after injury for Lines MLC/Class 1 IGF-1Eb and MLC/Class 2 IGF-1Eb.

The expression analysis of endogenous Class 2 IGF-1Ea revealed a significant increase in MLC/mIGF-1 animals. During the time course of regeneration neither line MLC/mIGF-1 nor line MLC/Class 1 IGF-1Eb had a significant influence of the endogenous expression pattern of Class 2 IGF-1Ea (FIG. 56B). However, an increase was noticed at day five post-injury (169% in MLC/mIGF-1 animals and 152% in MLC/Class 1 IGF-1Eb animals) and a decrease was observed for both transgenic lines at day ten, where MLC/mIGF-1 decreased Class 2 IGF-1Ea mRNA levels by 59% and MLC/Class 1 IGF-1Ea by 73%. MLC/Class 2 IGF-1Eb values displayed a standard deviation that was too high and therefore was not considered here. In summary, no significant alterations of endogenous Class 2 IGF-1Ea expression was observed in response to the MLC/mIGF-1 and MLC/Class 1 IGF-1Eb transgenes.

The evaluation of endogenous expression of Class 1 IGF-1Eb and Class 2 IGF-1Eb (FIG. 56C and D) showed no significant changes in response to the Class 1 IGF-1Ea transgene (mIGF-1) and the Class 2 IGF-1Ea transgene. In the background of MLC/Class 2 IGF-1Eb a non-significant increase of Class 1 IGF-1Eb RNA was observed throughout the regeneration process (FIG. 56C), while in the background of MLC/Class 1 IGF-1Eb endogenous Class 2 IGF-1Eb was elevated upon injury (FIG. 56D).

Affymetrix GeneChip Analysis of IGF-1 Isoform Transgenic Muscles

Gene array analysis represents a powerful tool for gaining an overview of changes in transcriptional regulation in response to a certain stimulus. Affymetrix GeneChip analysis was applied in this work in an attempt to understand IGF-1 isoform-induced alterations in RNA expression patterns.

Affymetrix analysis was performed on the quadriceps muscle of WT and transgenic mice for each IGF-1 transgenic line (n=2 for each) and a large set of data was obtained. A crucial aspect of analyzing the Affymetrix data is to find a reliable way of finding interesting candidate genes among the 22626 probe sets that returned a positive signal. To further decrease the number of genes to an acceptable amount of candidates we chose a cut off of 1.9-fold up- or down-regulated. This approach excludes genes that are only slightly affected and nevertheless might have an important role, but was necessary to evaluate the data in the manner described below.

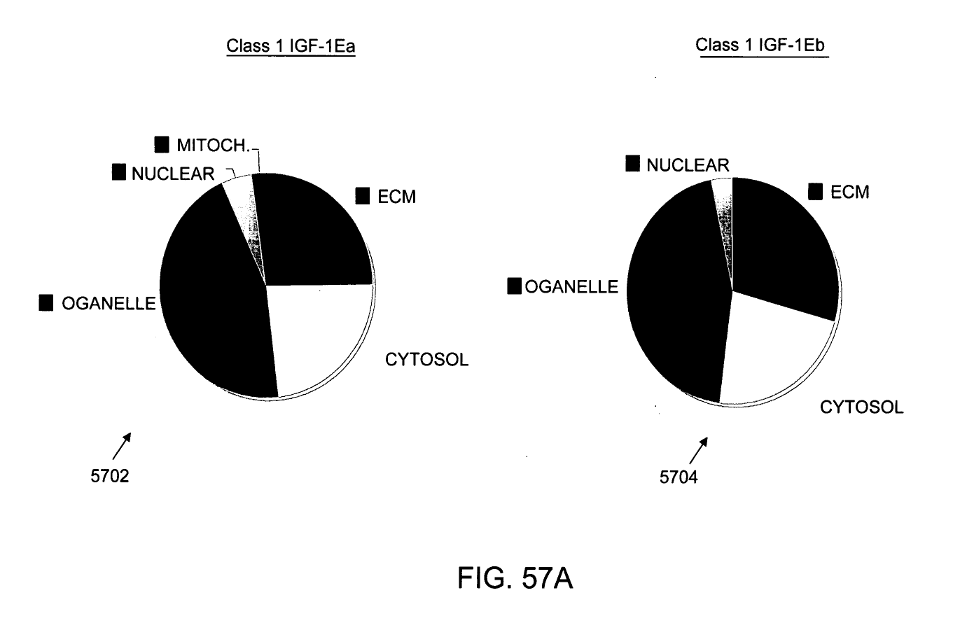

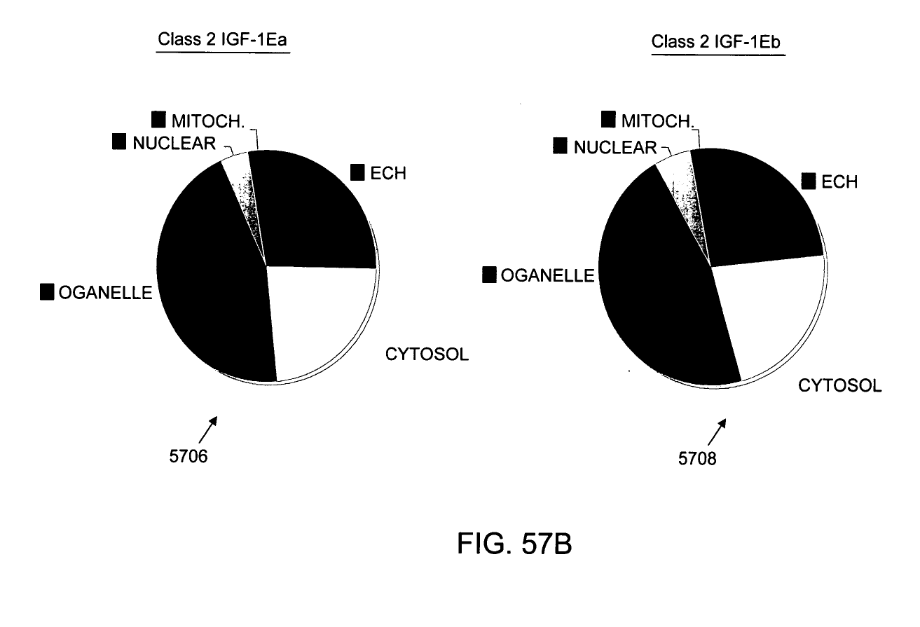

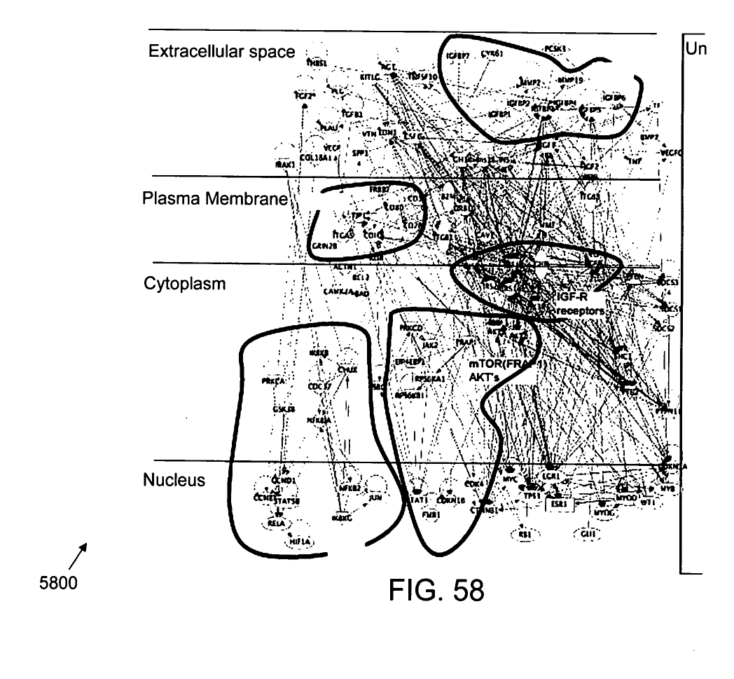

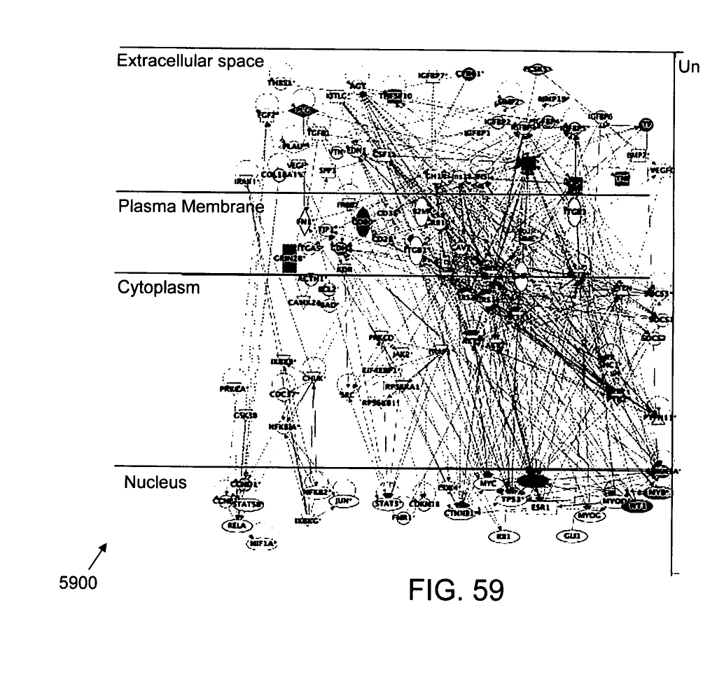

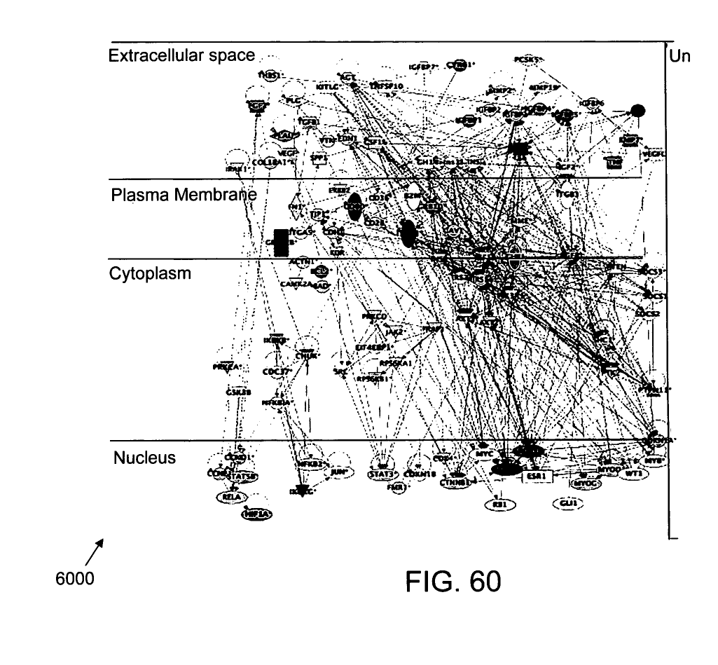

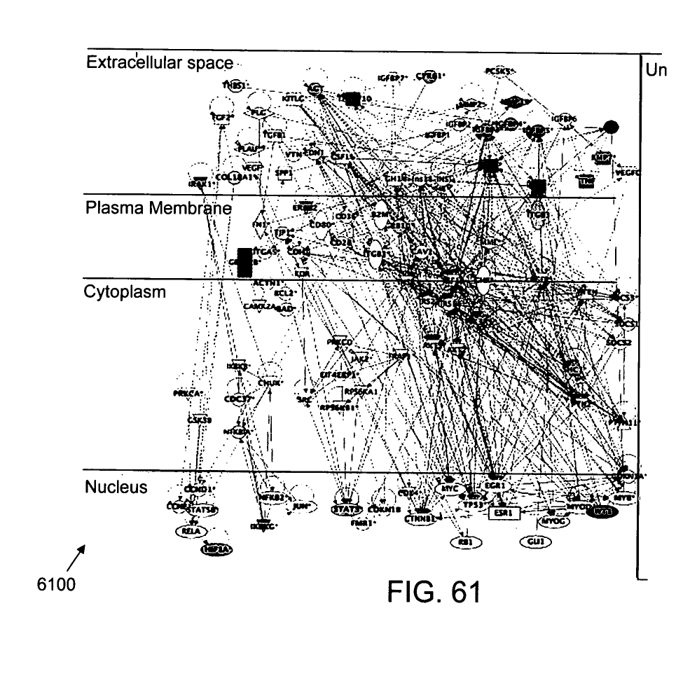

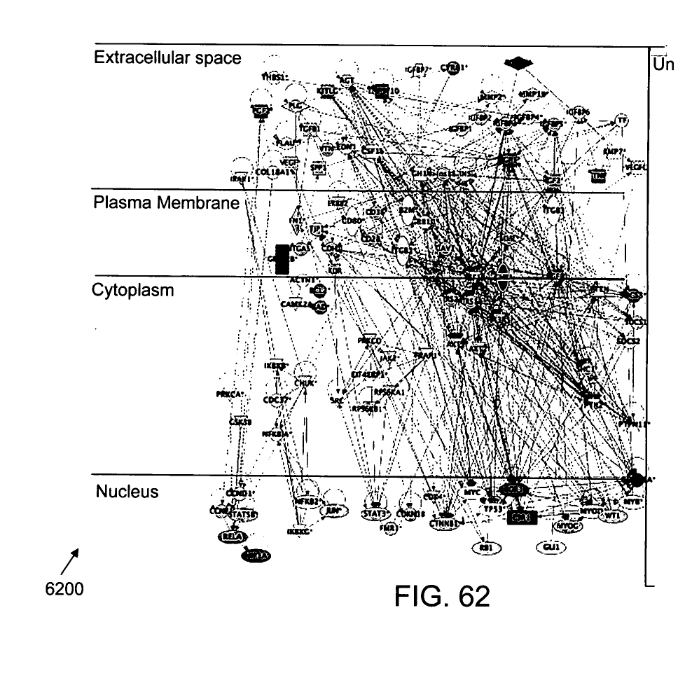

The filtered genes can be grouped under several criteria, such as sub-cellular localization, molecular function, involvement in disease and so on, depending on the interest of research. We chose to group by sub-cellular localization to gain an overview of regulated genes with a function in a certain compartment of the cell, and to provide an idea about the nature of IGF-1 isoform-mediated effects. FIG. 57 A-D shows the pooled up-or down-regulated genes for each IGF-1 isoform transgenic line. This approach demonstrates that almost half of the genes regulated by each IGF-1 isoform can be correlated to organelle function, while only a very small percentage can be connected to a function in the mitochondria. An almost equal amount of genes is related to a function in the extracellular matrix and the cytosol, while each IGF-1 isoform regulated only a very small percentage of genes with a nuclear function. Between the different isoforms, no major variations in cell compartment were seen. Tables 9-16 list a selection of genes that were up- or down-regulated in response to one specific isoform (highlighted with bold font), which might play an important role in the IGF-1 isoform-mediated response.

An interesting candidate transcript 2.8-fold down-regulated exclusively by the MLC/Class 1 IGF-1 Ea transgene is forkhead box 03a (FOXO3A), which is a downstream target of Insulin/IGF1-Akt pathway and has been shown to impede muscle size growth both in cardiac and skeletal muscle [109], [110]. Foxo3A can also stimulate expression of IGFBP1 under insulin signaling [111]. Another interesting candidate transcript 2.74-fold up-regulated in MLC/Class 1 IGF-1Eb animals is the matrix metalloproteinase 7 (MMP7), which has been associated with processing of an important regulator of IGF1 function, i.e. IGFBP5 [112]. In MLC/Class 2 IGF-1Ea animals the Bcl2-like 2 transcript, which was 2.05-fold down-regulated, represents an attractive candidate belonging to the family of Bcl-proteins that regulate cell death and that could play a key role in disease states [113], [114]. Although genes showing altered expression solely in the MLC/Class 2 IGF-1Eb were identified, they did not appear as solid candidates of interest. In summary, a stringent analysis of up- or down-regulated genes can offer the opportunity to explore molecular players previously ignored in the current view of IGF1 isoform function.

A general observation was that relatively more genes were down-regulated in the different IGF-1 transgenic samples, and that there was little overlap of differentially expressed genes among the different IGF-1 isoforms. To provide a preliminary overview of potentially common mediators of IGF1-isoform-specific functions, we lowered the cut off of the filtered genes and analyzed the lists of only those genes that presented opposing trends of expression in class 1 vs. Class 2, or in Ea vs. Eb isoforms. The list of such genes (see Table 7 and 8) is less stringent, but highlights a group of genes that in both IGF1Ea isoform transgenic models show a trend opposed to the IGF1Eb isoform transgenic models. These genes include putative candidates involved in regulating signal transduction, such as endothelin converting enzyme and proteins associated to Rho and G-protein activity, as well as proteins relevant for muscle function, such as cardiac myosin isoform, integrin subunits and cGMP-dependent protein kinase. These genes are potentially useful to elaborate a working hypothesis able to explain at least some of the phenotypes common to the IGF1Ea transgenic models.

The analysis described above, although only valid as initial assessment of the data, is useful to identify solid targets to confirm and possibly link to the different phenotypes. However, one could take an alternative approach. With increasing demand from researchers for tools that allow to analyze large sets of data (coming from gene expression analyses or other means of high throughput screenings), more sophisticated network analysis tools have been developed. These software platforms rely on an underlying knowledge database in which every node (gene of interest) is connected to any other node by either direct or indirect relationships (such as being a downstream/upstream regulator, a phosphorylating enzyme, a binding partner and so on). By screening the filtered array of probe sets through these network interaction repositories, it is possible to identify group of genes that are differentially regulated outside of specific stringency criteria, yet nevertheless belong to a coherent biochemical/molecular pathway. By identifying and grouping genes with altered expression in this way, we can gain a consistent overview of the significance of those candidate genes. Ingenuity Inc. (www.inpenuity.com) offers such a tool.

In order to validate our approach we selected a network related to IGF1R function, included additional elements (such as NF-kB related genes) and compared the results by overlapping the expression ratios relative to each of the 4 transgenic models. In this analysis, targets such as the IGFBPs, which are important regulators of IGF1 function and which were not highlighted in our previous analysis because of their lower level of expression changes (below 1.9-fold up- or down-regulated), were embedded in a coherent network and appeared to be selectively and differentially deregulated by the IGF1 isoforms. This method also highlights other genes with different levels of regulation, which could be interesting targets for further studies, such as IGF2 the GH or the IGFBP-related gene Cyr6l (FIGS. 58-62: Table 17).

### CONCLUSIONS

Effects of IGF-1 Isoforms in vitro

The initial in vitro experiments on the effect of IGF-1 isoforms on myoblast proliferation suggest that Ea-containing isoforms (Class 1 IGF-1A and Class 2 IGF-1A) are less efficient in stimulation of proliferation, as shown by a down-regulation of phospho Histone H3, a marker of mitosis. In contrast, Eb-containing isoforms (Class 1 IGF-1B and Class 2 IGF-1B) showed similar levels of phospho H3 when compared to the mock transfected cells. In addition, this conclusion was drawn from observations of the transfected cells upon shift from growth medium (GM) to differentiation medium (DM), where IGF-1 isoforms containing the Eb-peptide displayed a delayed exit from the cell cycle and underwent an additional round of replication as has been reported using recombinant, fully processed IGF-1.

Notably, none of the over-expressed prepro-variants of IGF-1 were processed to pro-IGF-1Ea/Eb or mature IGF-1 during proliferation. This result suggests that the entire prepro-peptide, including signal- and Eb-peptide, influenced proliferation of myoblasts in this system. The possibility that L6E9 cells are not equipped to process IGF-1 due to the lack of endogenous IGF-1 expression can be excluded, since processing was observed at later stages during differentiation.

These findings also shed light on the outstanding question of whether the IGF-1 isoforms are active or properly localized without cleavage of the signal or E peptides. The data presented here strongly indicate that the entire IGF-1 prepro-peptide does not need processing to be active, and raise the possibility that specificity of IGF-1 function might be determined by processing status, or by the presence of isolated E-peptide once the prepropeptide is cleaved.

The in vitro data described above revealed an enhancement of differentiation for cells transfected with Class 1 IGF-1Ea that culminated in the formation of hypertrophied myofibers. Interestingly, cells transfected with Class 2 IGF-1Ea showed normal differentiation kinetics but nevertheless showed an increased size of differentiated fibers after day three of differentiation. L6E9 cultures transfected with the Eb-peptide containing isoforms of IGF-1 showed a delay in the differentiation process and the formation of normally sized muscle fibers, suggesting that IGF-1 isoforms containing the Eb-peptide do not play a role in mediating IGF-1 induced hypertrophy. Moreover, cells transfected with 22-IGF1Ea revealed a third phenotype, since they showed fibers that were comparable to control fibers although an early initiation of the differentiation process was indicated by a stronger up-regulation of myogenin in comparison to the mock control cells and cells transfected with other IGF-1 isoforms after one day of differentiation. Notably, after two days of differentiation all isoforms uniformly up-regulated myogenin and MEF2C in comparison to mock controls, indicating that the myogenic program was efficiently induced by all of the IGF-1 isoforms.

These findings confirm earlier publications that demonstrated a hypertrophic response to the Class 1 IGF-1Ea isoforms. In the present study all the different isoforms containing the Eb-peptide up-regulated markers of myocyte maturation and differentiated into myotubes.

The in vitro findings presented here also show for the first time that Class 2 IGF-1Ea is also capable of inducing cell hypertrophy. In contrast, the three different IGF-1Eb isoforms analyzed here do not induce enlarged fiber sizes. These findings implicate that the Ea-peptide is involved in mediating a hypertrophic response, while the Eb-peptide is not involved in mediating such an effect. Clearly, context is critical, since the 22-IGF-1Ea isoform, with a truncated signal peptide, did not lead to a hypertrophic phenotype of transfected cells. It is possible that the 22 amino acid signal peptide directs this isoform to a different sub-cellular localization, which might result in less efficient secretion or differential binding of this isoform to the different IGFBPs.

Effects of IGF-1 Isoforms in vivo

Further in vivo evidence for a distinct role of the E-peptides in mediating IGF-1 function, arises from the analysis of transgenic animals, over-expressing the different IGF-1 isoforms in a post-mitotic manner. Six transgenic mouse lines over-expressing the six main IGF-1 isoforms were generated. Selected founders from different transgenic lines were chosen on the bases of high and comparable expression. High to moderate skeletal muscle-specific expression was seen for each transgene, depending on the specific skeletal muscle group analyzed: fast IIb fibers showed higher transgene expression levels due to the fast fiber restriction of the MLC regulatory elements. For further analysis, priority was given to the main IGF-1 isoforms expressed in mouse liver: MLC/Class 2 IGF-1Ea, MLC/Class 1 IGF-1Eb, MLC/Class 2 IGF-1Eb. Further, comparative expression analysis between the different transgenic lines revealed variable differences in IGF-1 transgene expression on the transcriptional level, which were nevertheless translated into comparable protein levels. This observation implies a threshold for IGF-1 protein translation that is not dependent on absolute transcript levels.

Pronounced skeletal muscle hypertrophy, as seen in the MLC/Class 1 IGF-1 (MLC/mIGF-1) animals previously described (86), was also seen in MLC/Class 2 IGF-1A animals. In correlation with the in vitro findings, detailed analysis of skeletal muscle phenotype and physiological performance revealed striking muscle hypertrophy in line MLC/Class 2 IGF-1Ea. This was reflected by an increase in CSA of fast, intermediate, and slow fibers in the TA and EDL muscle. The increase of skeletal muscle fiber CSA was accompanied by a significant increase of skeletal muscle wet weight and a pronounced increase in force generation of fast but not slow skeletal muscle groups, confirming functional hypertrophy. The transgenic MLC/Class 2 IGF-1Ea animals also showed increased body weight at all ages studied, which can be correlated to the pronounced increase of fast skeletal muscle wet weight, since distal organs were not affected in their weight. Interestingly, the phenotype observed in MLC/Class 2 IGF-1Ea animals was more pronounced than in either the Class 1 IGF-1Ea (mouse) or the MLC/mIGF-1 (rat) line, reflected in a stronger increase of skeletal muscle weight and physiological performance, as well as a consistent, but small relative increase in overall bodyweight. These results highlight a strong connection between IGF-1Ea isoforms and induction of hypertrophy in skeletal muscle.

The fact that the MLC/Class 1 IGF- I Ea transgene increased endogenous levels of Class 2 IGF-1Ea raises the possibility that hypertrophy in these animals is predominately due to the Class 2 IGF1Ea isoform. This is supported by the fact that the increase in endogenous Class 1 IGF-1Ea induced by the MLC/Class 1 IGF-1Eb transgene did not result in a hypertrophic response. It also appears that only Class 1 isoforms can influence endogenous IGF-1 expression, since endogenous IGF-1 levels were unaffected in transgenic lines over-expressing Class 2 IGF-1 isoforms. Thus the crosstalk between exogenous and endogenous IGF-1 isoforms must be taken into consideration when interpreting the results of any over-expression studies.

The two lines over-expressing the Ea-peptide containing isoforms Class 1 IGF-1Ea (mIGF-1) and Class 2 IGF-1Ea also showed a modest, but significant up-regulation of total circulating levels of IGF-1, which was not the case for transgenic lines over-expressing IGF-1 isoforms containing the Eb-peptide. Notably, the increase of total serum IGF-1 remained significant in MLC/Class 2 IGF-1Ea mice at the age of six months, while levels were still elevated, but no longer significant in MLC/mIGF-1 mice. In either case, the total IGF-1 elevation did not lead to an increase in the weight of visceral organs, such as heart, brain, liver, kidney or spleen at all ages analyzed for both lines, indicating that the mild increase of serum IGF-1 did not disturb other organ systems. The assay used for these experiments included a step to strip off IGF-binding proteins and therefore does not reflect levels of free, bio-available IGF-1.

Relative changes in the levels of IGF-1 isoforms suggest that IGF-1 isoforms containing the Ea-peptide could enter the circulation, since this has been observed for the SIS2, the MLC/Class 2 IGF-1Ea, and the MLC/mIGF-1 mice, at least in the present study. Since no correlation with the signal peptide was seen (Class 1 versus Class 2), the Ea-peptide might therefore determine if an isoform is secreted to the circulation or not. This could be mediated by the binding to certain IGFBPs.

By contrast, Eb-containing isoforms show either mild (MLC/Class 2 IGF-1B) or no significant hypertrophy (MLC/Class 1 IGF-1B) accompanied by a small shift towards a higher proportion of larger fibers in the fast muscles that did not result in a consistent increase of fiber CSA. These modest changes did not result in physiological improvements of muscle performance and did not affect the overall bodyweight of these animals. The in vitro and in vivo results presented for both Eb-peptide containing isoforms demonstrate for the first time that these versions of IGF-1 do not play a role in mediating IGF-1 induced functional hypertrophy of skeletal muscle and imply that the nature of the E-peptide plays an important role in determination of IGF-1 isoform function.

As with the original MLC/mIGF-1 animals, Class 2 IGF-1A animals are significantly stronger compared to wildtype, and compared to Class 2 IGF-1B animals.

Apart from the presence of different E-peptides, IGF-1 isoforms also differ by the signal peptide (=Class), providing another means of influencing IGF-1 isoform function (115). As described previously (116-119), Class 2 isoforms are considered to be the endocrine version of IGF-1, while Class 1 isoforms have been thought to have a local role. The animal models presented in this work, where IGF-1 expression is restricted to skeletal muscle, allowed us to address which of the IGF-1 isoforms stays in the tissue of origin. Measuring total IGF-1 levels in the circulation revealed an up-regulation of circulating IGF-1 in response to over-expressing Class 1, rather than Class 2 isoforms. These results contradict the current technical literature and may suggest a peculiarity to skeletal muscle or imply the involvement of other tissue-specific factors, such as IGFBPs, in the determination of the fate of IGF-1. The results certainly confirm that the signal peptide plays an important role in determining IGF-1 function.

Further support for the importance of the signal peptide comes from differences seen in the activation of signal transduction pathways, where Class 2 IGF-1 isoforms show up-regulation of certain pathways that are not affected by Class 1 isoforms.

These studies further suggest that some IGF-1 functions are mediated by the presence of a certain E-peptide, whilst others are correlated to the presence of a certain signal peptide.

### Example 6

the effect of mIGF-1 on Inflammatory Response during Muscle Regeneration and in Muscular Dystrophy

Inflammation is a critical component of muscle regeneration and is an important phase necessary to activate the stem cell compartment and therefore regeneration. Nevertheless, the inflammatory response must be resolved to proceed towards muscle repair. In fact, muscle regeneration fails when muscle injury is associated with altered spatial distribution of inflammatory cells, altered identity of the inflammatory infiltrate and altered temporal pattern.

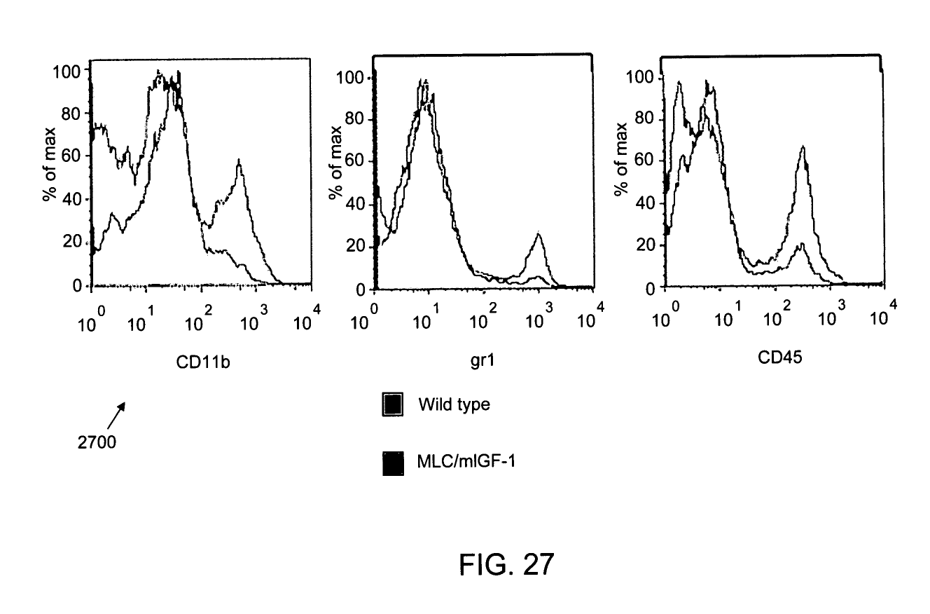

We accumulated evidence demonstrating that mIGF-1 accelerates the timing of regeneration and reduced the amount of mononucleated infiltrating cells at five days post-injury, while the regenerative capacity of injured wild type muscle was substantially delayed (FIG. 27).

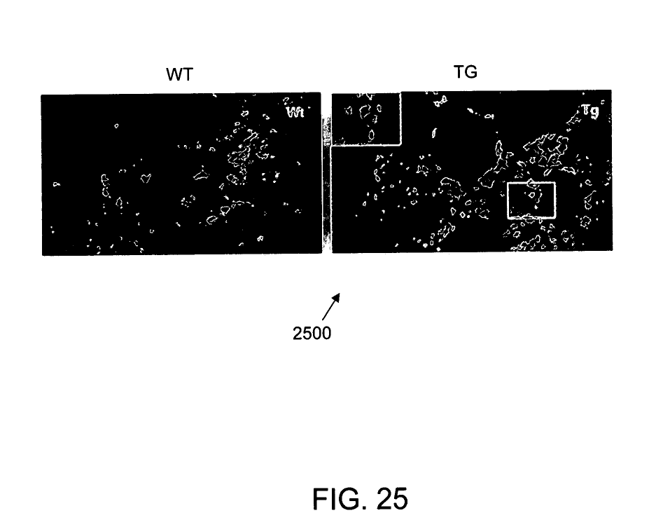

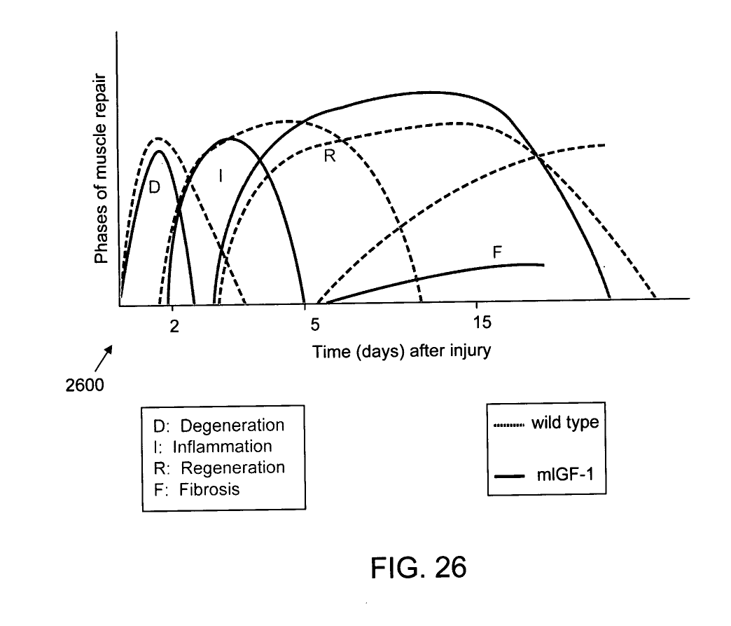

Our results demonstrated that the local expression of mIGF-1 improves the regenerative phase increasing the pool of satellite cells (FIG. 25) and modulating the inflammatory response of injured skeletal muscle (FIG. 26).

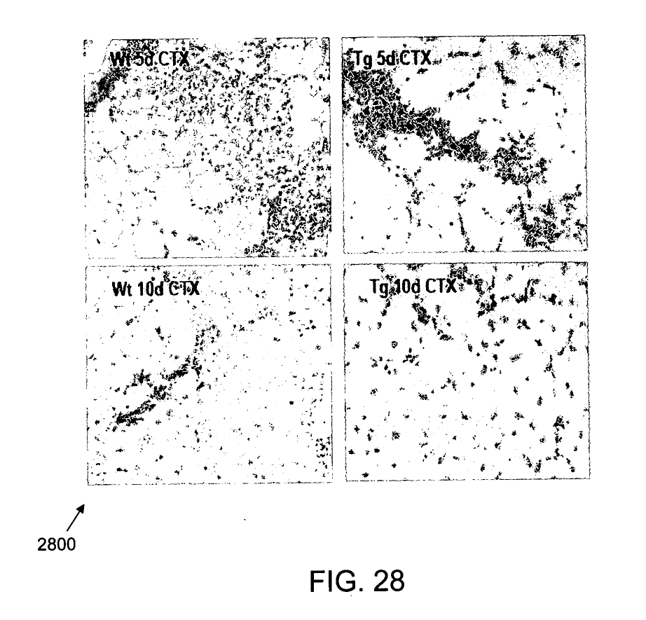

In particular, quantitative RT-PCR and proteomic analysis demonstrated that mIGF-1 modulates inflammatory cytokines, such as MCP1, MCP2, MIP-1 α, and MIP-1β at early stages, stimulating a qualitative environment for a complete functional recovery. Indeed, the muscle architecture of MLC/mIGF-1 injured mice was rapidly and almost completely restored compared to wild type muscle (FIG. 28).

These results suggest that mIGF-1 modulates inflammatory cytokines at early stages, stimulating a qualitative environment for a complete functional recovery. This was confirmed by analyzing the effect of mIGF-1 in the mdx, dystrophic mouse model.

It has been reported that that hematopoietic stem cell (HCS) migration into sites of injury may be a mechanism by which damaged tissues are repaired. However, this is a rare event and presents limitations for efficient tissue repair. It has been reported that the poor recruitment of HSC into the dystrophic muscle of the mdx mouse is the major obstacle for muscle regeneration and therefore for the rescue of the genetic disease.

It is proposed that the recruitment and mobilisation of stem cells is not the only limitation for stem cell-mediated muscle regeneration in dystrophic muscle. Among potential parameters that have impeded the generation of satisfactory protocols for stem cell therapy is the activation of deleterious signal transduction pathways by dystrophic milieu. In this context, our working hypothesis was that dystrophic microenvironment renders unproductive the stem cell-mediated therapy.

FACS analysis revealed that dystrophic muscle was able to recruit a large population of cells expressing markers of the hematopoietic stem cell, such as Sca1, c-kit and CD45. In this context, the recruitment of stem cells is not the critical parameter for the success of stem cell therapy. Preliminary evidence suggests that the microenvironment plays a pivotal role limiting the stem cell mediated therapy.

RT-PCR analysis revealed that inflammation was modulated by mIGF-1 expression in mdx/mIGF-1 transgenic mice. This opened the question whether the mIGF-1 expression, that improves the dystrophic environment, also stimulates the regenerative capacity of stem cells.

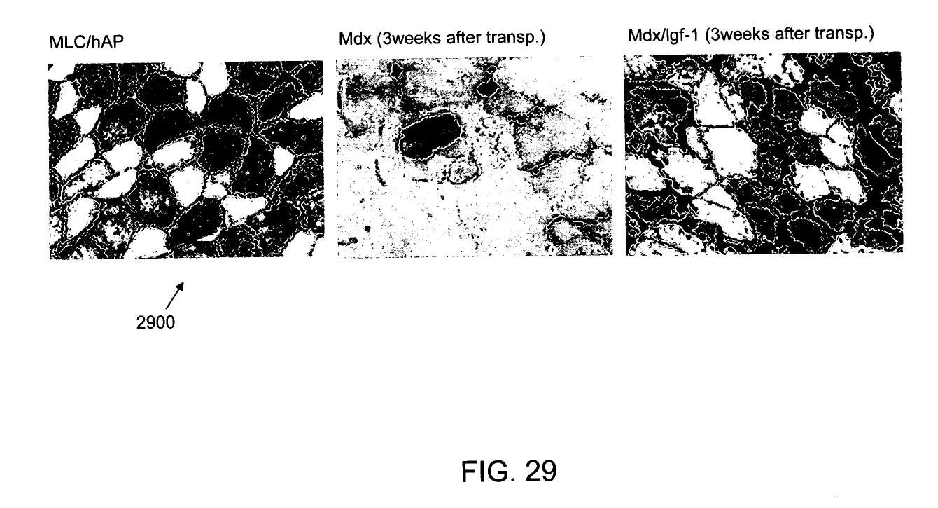

Stem cells isolated from the bone marrow of MLC/hAP mouse were transplanted into the mdx and mdx/mlgf-1 dystrophic muscle to investigate whether the mIGF-1 expression, that improves the dystrophic environment, also stimulates the regenerative capacity of stem cells. The MLC/hAP mouse is a good model to follow the differentiative fate of bone marrow stem cells, since these stem cells will activate the transgene hAP only when transdifferentiated into skeletal muscle. Histological analysis revealed that transplanted stem cells massively participated in muscle regeneration only in mdx/mIGF-1 dystrophic mice (FIG. 29).

These data confirm the hypothesis that mIGF-1 promotes a qualitative environment for an efficient muscle regeneration.

### Example 7

Identification of a Rodent IGF-1 Isoform Containing a Third E-peptide.

In rodents, two 3′ splice variants have been described, encoding different E-peptides. The rodent and human Ea-peptides share extensive homology, whereas the second rodent E-peptide (Eb) corresponds to the Ec peptide in human nomenclature. The possible existence of an exon 4-5 splice variant, which encodes a third E-peptide in humans (confusingly named Eb-peptide), was also predicted in rats [10], but since it was not detected in rat liver using Northern blot analysis, it was assumed that the human Eb splice variant does not exist in rodents. This section describes the cloning of a mouse Class 2 IGF-1 isoform containing the exon 4-5 splice variant, which we term En-peptide.

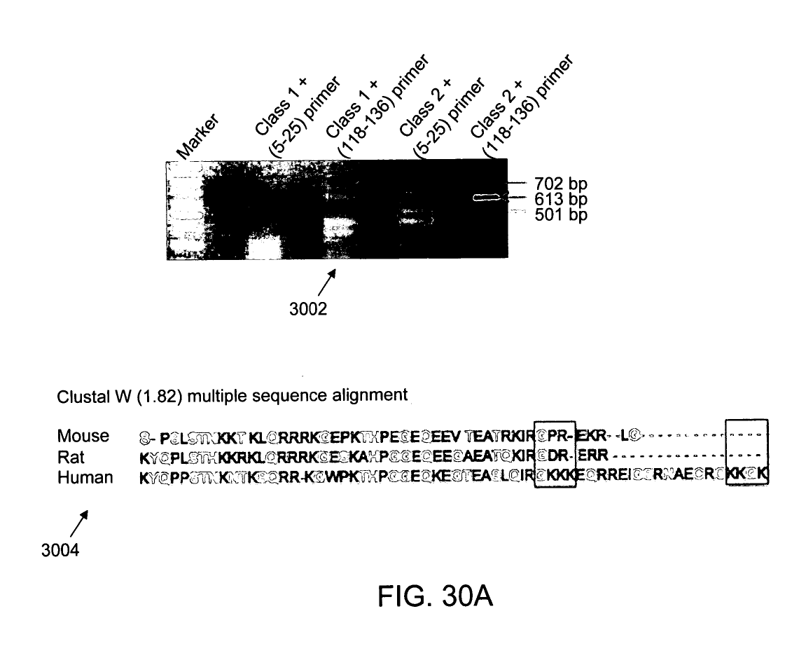

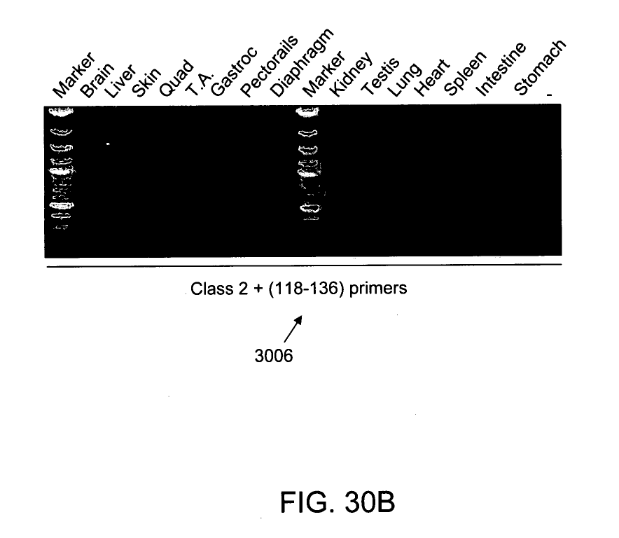

Forward primers were designed to bind to exon 1 or 2, thereby amplifying Class 1 or Class 2 isoforms, and reverse primers were designed to bind to two different regions in the intron sequence downstream of 52 bp region (bp 5-25 and 118-136) that until now was considered to represent rodent exon 5. By choosing such primer pairs, Class 1 and Class 2 isoforms containing the exon 4-5 splice variant were expected to be amplified. Since liver is the one tissue in the body that is known to express all IGF-1 isoforms, total RNA isolated from three-months-old mouse liver was chosen for RT-PCR (FIG. 30A) RT-PCR with the primer pair Class 1 and (5-25) did not yield any fragments, while primer pairs Class 1 and (118+136) amplified four fragments, but only showed the expected size (702 bp). RT-PCR performed with primers for Class 2 and (5-25) yielded two fragments with one being of the expected size (501 bp), and primers for Class 2 and (118-136) amplified one 613 bp fragment (FIG. 30A). DNA fragments of expected sizes were sub-cloned and sequenced. Analysis of the sub-cloned sequences revealed that the 702 bp fragment obtained from RT-PCR with Class 1 and (5-25) and the 501 bp fragment from RT-PCR with Class 2 and (5-25) were unspecific, while the 613 bp fragment amplified with Class 2 and (118-136) primers represented a Class 2 IGF-1 isoform encoding the third E-peptide described in humans (human Eb-peptide). Since the Eb-peptide in rodents corresponds to the Ec-peptide in humans, and to avoid more confusion in the nomenclature of IGF-1 isoforms an E-peptides, we termed this third rodent version Enew-peptide (En-peptide). The En-peptide could only be amplified together with the Class 2-specific primers, suggesting that no Class 1 IGF-1En isoforms are expressed in the liver.

Analysis of the mRNA sequence of the En-peptide revealed a TAG stop codon 141 bp downstream of the exon 5 start as well as four AATATA polyadenylation signals (458, 569, 2888, and 5178 bp downstream of the exon 5 start respectively). No consensus AATAAA polyadenylation signals could be predicted. Further characterization is needed to identify the functional poly (A) addition sites of the exon 5-encoded 3′-UTR for this splice variant.

The mouse exon 4-5 splice variant encodes a 63 amino acid En-peptide sequence (16 aa encoded by exon 4 and 47 aa encoded by exon 5), while the human counterpart (Eb-peptide in humans) encodes a 77 amino acid E-peptide (16 aa from exon 4 and 61 aa from exon 5) and there is only 57% homology between the common N-terminal 63 amino acids of those two sequences. Most importantly, the predicted amino acid sequence encoded by the mouse exon 4-5 splice variant does not contain a GKKK signal for proteolytic cleavage and peptidyl C-terminal amidation, which has been shown to be used in human Eb-peptides to generate the IGF-1B(103-124) E₁amide (IBE₁) [120]. Synthetic IBE₁ has been shown to have mitogenic effects on normal and malignant human bronchial epithelial cells and to bind to specific high-affinity receptors [120]. In addition, the mouse exon 4-5 splice variant lacks a nuclear and nucleolar localization signal (KKGK), which has also been described for the human Eb-peptide and has been shown to direct this human IGF-1 isoform to the nucleolus [121] (FIG. 30B). Further analysis will therefore be necessary to determine whether the rodent En variant is functionally equivalent to the longer human Eb variant.

To evaluate the expression of the mouse Class 2 IGF-1En isoform, RT-PCR was performed (FIG. 30C). Total RNA was isolated form mouse brain, liver, skin, kidney, testis, lung, heart, spleen, intestine, and stomach, as well as from the skeletal muscles quadriceps, gastrocnemius, pectoralis, and the diaphragm. Using this approach, no specific signals could be detected in any of the tissues analysed for expression. In contrast to the RT-PCR performed for initial amplification of this isoform, were mRNA was used, expression analysis was performed with total RNA. Even in liver mRNA, expression of the Class 2 IGF-1En isoform was very weak, which might be the reason why amplification from total RNA gave a negative result.

Until now the rodent exon 5 has been considered to be a 52 bp cassette exon (in contrast to a terminal exon giving rise to a functional 3′-UTR), while the human exon 5 represents a terminal exon, encoding a stop codon and a 3′-UTR. With the description of the Ec-peptide in humans, where exon 5 is spliced to exon 4 and 6, it became clear that also the human exon 5 can become a cassette exon, if a cryptic 5′-donor splice site is used [118]. With the cloning of the exon 4-5 splice variant in mice, this model will need to be revised for rodents as well.

In humans, splicing of the exon 4-5-6 variant (encoding the Ec-peptide in humans) occurs by use of a cryptic IGF₆₃₃ donor splice site located 49 bp downstream from the 5′ end of exon 5. The sequence of this donor splice site deviates from the vertebrate 5′-donor splice site consensus and failure to use this cryptic IGF₆₃₃ donor splice site results in the exon 4-5 splice variant (corresponds to the human Eb-peptide) [118]. A possible reason for the low expression of the exon 4-5 splice variants in rodents might be the strength of the 5′-splice donor site. When comparing the 3′ exon5:5′ intron boundary to the vertebrate 5′-donor splice site consensus AG:AGTAAGT, the rat sequence matches by five out of six bases, the mouse sequence matches by four out of six bases, and the human sequence matches only by three out of six bases. These polymorphisms might alter the strength of the donor splice site and influence the splicing machinery. Because the rat donor splice site shows the highest match to the vertebrate 5′-donor splice site consensus, it is less likely to be overseen by the splicing machinery. In addition, the rat intron sequence following the 52 bp cassette exon contains 4 purine-rich repeats (GGAAG) within 300 bp downstream of the 5′-donor splice site, which have been shown to enhance splicing in the bovine GH gene. Finally, the sequence downstream of the 5′-donor splice site contains only one AATATA polyadenylation signal, which might not be strong enough to compete with the stronger 5′-donor splice site.

The observation that En-peptide variant could only be detected in Class 2 IGF-1 mRNAs and showed weak expression only in mouse liver suggests a specialized, possibly endocrine function for this isoform, which might need specific stimuli to be induced. However, since the rodent En-peptide lacks sequences that have been demonstrated to be important for human Eb-peptide function [120-121], further analysis will be necessary to evaluate the function of this splice variant in rodents.

TABLE 9
MLC/Class 1 IGF-1Ea list of up-and down-regulated genes. A selection of the
genes that were either up- or down-regulated by 1.9 fold is shown. Genes in bold are
unique for the IGF1-1Ea transgenics, i.e. they scored an increase or decrease of 1.9 fold in
the IGF1-1Ea transgenic while at the same time scoring less than 1.3 up- or down
regulation in the other three transgenic models. The presence of a star on the right of each
shown value indicates a higher variability of signal intensity between the two RNA
samples analyzed for each model.

| GENE NAME | 1EA | 1EB | 2EA | 2EB |
|---|---|---|---|---|
| insulin-like growth factor 1 | 25.52* | 3.32 | 15.69 | 1.80 |
| zinc finger protein, subfamily 1A, 1 (Ikaros) | 2.91* | 2.79* | 2.07* | 2.55* |
| DIX domain containing 1 | 2.82 | 2.00* | 2.29 | 3.29 |
| TGFB-induced factor 2 | 2.34 | 1.46 | 1.44* | 1.69 |
| solute carrier family 16 (monocarboxylic acid transporters), | 2.30 | 1.32* | −1.22* | −1.40 |
| member 11 | | | | |
| sarcolipin | 2.15* | 1.11 | −1.49* | 1.19* |
| Bcl2-interacting killer-like | 2.14 | 1.26 | 1.08* | 1.64 |
| colony stimulating factor 3 receptor (granulocyte) | 2.11 | 1.44 | 12.39 | −1.13* |
| RIKEN cDNA 9130019O22 gene | 1.94 | 1.69 | 1.05* | −2.50 |
| putative zinc-finger containing protein | | | | |
| hyaluronan mediated motility receptor (RHAMM) | 1.91 | 1.20 | 1.05 | 1.35 |
| pterin 4 alpha carbinolamine dehydratase/dimerization cofactor | −1.91* | 1.07* | −1.09* | 1.58 |
| of hepatocyte nuclear factor 1 alpha (TCF1) 1 | | | | |
| Sjogren syndrome antigen B | −1.98 | −1.11 | −1.44 | −1.10* |
| src homology 2 domain-containing transforming protein C1 | −2.01 | 1.04 | 1.37 | 1.14 |
| elongation factor Tu GTP binding domain containing 2 | −2.02 | 1.12 | −1.47 | −1.08 |
| synaptotagmin III | −2.04* | 1.02* | −1.77 | 1.14 |
| RNA binding motif protein, X chromosome, mRNA | −2.06 | −1.85 | −1.16* | 1.17* |
| Expressed sequence AW538196 (A W538196), mRNA | −2.08 | 1.12* | 1.20 | −1.13* |
| RIKEN cDNA 5730466C23 gene | −2.11 | 1.27 | −1.02 | 1.40 |
| phenylalanine-tRNA synthetase-like, alpha subunit | −2.12 | 1.17 | 1.20 | −1.74 |
| — | −2.12 | −1.18 | −1.18 | −1.93 |
| RIKEN cDNA 1810057C19 gene | −2.17 | −1.72 | 1.07 | −1.50* |
| melanoma antigen, family A, 7 | −2.26 | −2.04 | 1.12 | 1.75 |
| plakophilin 3 | −2.27 | −1.52 | −1.38* | −1.25* |
| solute carrier organic anion transporter family, member 1c1 | −2.28 | −1.33 | 1.53 | −1.36* |
| 3-hydroxybutyrate dehydrogenase (heart, mitochondrial) | −2.36 | −1.50 | −1.04 | 1.55 |
| contactin associated protein-like 2 | −2.36 | 1.17 | 1.10 | −1.13 |
| forkhead box N4 | −2.51 | −2.09 | −1.43 | −1.87 |
| forkhead box O3a | −2.83 | −1.19* | 1.12* | −1.26 |
| reproductive homeobox on X chromosome, 9 | −2.89 | 1.15 | −1.00 | −1.66* |
| serine (or cysteine) peptidase inhibitor, clade D, member 1 | −2.96 | −1.00 | 1.08 | −2.92* |
| lysyl oxidase-like 4 | −3.04 | 1.25 | 1.02 | −1.08 |
| CD28 antigen | −3.10 | 1.01 | −1.54 | 1.14 |
| solute carrier family 35 (UDP-galactose transporter), member 2 | −3.35 | 1.67 | 1.35 | −1.25 |
| aldo-keto reductase family 1, member C18 | −3.67 | −1.76* | −1.39* | 1.48 |
| CD28 antigen | −6.46* | 1.18 | −1.14 | 1.02 |
| solute carrier family 35, member A5 | −6.63 | −1.84 | −1.25 | −1.23 |

TABLE 10
MLC/Class 1 IGF-1Eb list of up-and down-regulated genes. A selection of
the genes that were either up- or down-regulated by 1.9 fold is shown. Genes in bold are
unique for the IGF1-1Ea transgenics, i.e. they scored an increase or decrease of 1.9 fold in
the IGF1-1Ea transgenic while at the same time scoring less than 1.3 up- or down
regulation in the other three transgenic models. The presence of a star on the right of each

| GENE NAME | 1 EA | 1 EB | 2 EA | 2 EB |
|---|---|---|---|---|
| insulin-like growth factor 1 | 25.52* | 3.32 | 15.69 | 1.80 |
| zinc finger protein, subfamily 1A, 1 (Ikaros) | 2.91* | 2.79* | 2.07* | 2.55* |
| matrix metallopeptidase 7 | 1.01 | 2.74* | −1.14 | −1.54 |
| bromodomain adjacent to zinc finger domain, 1B | 2.14* | 2.68 | −1.21 | 1.65* |
| Monoclonal antiidiotypic antibody IgK (hypervariable region) | 1.54* | 2.01 | 1.14* | 1.39 |
| mRNA | | | | |
| DIX domain containing 1 | 2.82 | 2.00* | 2.29 | 3.29 |
| CEA-related cell adhesion molecule 1 | 1.11 | 1.96 | 1.46 | 1.79 |
| N-terminal Asn amidase, mRNA (cDNA clone MGC: 29106 | 1.53 | 1.95 | 1.22 | 1.78 |
| IMAGE: 5037501) | | | | |
| T-cell receptor beta, variable 13 | 1.64 | 1.94 | 1.25 | 2.03 |
| protein tyrosine phosphatase, non-receptor type 13 | −1.41 | −1.90 | −1.18 | 1.13 |
| Kruppel-like factor 4 (gut) | −1.11 | −1.93 | −1.63 | −1.07 |
| CD3 antigen, zeta polypeptide | 1.05 | −1.93 | −1.20 | 1.02 |
| cytidine monophospho-N-acetylneuraminic acid hydroxylase | −1.11* | −1.93 | −1.80 | −1.02 |
| ecotropic viral integration site 5 | 1.12 | −1.94 | −2.29* | −2.40* |
| uroplakin 1A | −1.08* | −1.96 | 1.21 | 1.19 |
| RNA binding motif protein, X chromosome, mRNA | 1.07 | −1.96 | −1.14 | −1.03 |
| presenilin 2 | −1.22 | −1.97 | −1.05 | 1.32 |
| heat shock protein 1B | −1.02 | −1.98 | 1.16* | −1.98 |
| fascin homolog 1, actin bundling protein | 1.15 | −1.99 | 1.18 | 1.09 |
| deoxyuridine triphosphatase | 1.29 | −2.03 | −1.10 | 1.31 |
| sodium channel, nonvoltage-gated 1 beta | −1.04* | −2.03 | −1.07 | −1.00 |
| melanoma antigen, family A, 7 | −2.26 | −2.04 | 1.12 | 1.75 |
| Ras association (RalGDS/AF-6) domain family 7 | −1.81* | −2.05* | −1.21 | −1.15 |
| peroxisome biogenesis factor 16 | 1.38 | −2.07 | −1.56* | −1.88 |
| forkhead box N4 | −2.51 | −2.09 | −1.43 | −1.87 |
| RIKEN cDNA 1200007D18 gene | 1.14* | −2.11 | −1.15 | −1.18 |
| SH3 multiple domains 1 | 1.05* | −2.11 | 1.22 | −1.10 |
| membrane metallo endopeptidase | 1.01 | −2.11* | 1.08 | −1.14 |
| neuron specific gene family member 2 | 1.31 | −2.12 | 1.09 | −1.21 |
| Ros1 proto-oncogene | 1.37* | −2.13 | 1.04 | 1.25 |
| glutamate receptor, ionotropic, NMDA2D (epsilon 4) | 1.26 | −2.23 | −1.58 | −1.18 |
| heat shock protein 1A | 1.02 | −2.25 | 1.37 | −1.88 |
| nuclear factor of kappa light polypeptide gene enhancer in B-cells | −1.28* | −2.46 | −2.35 | −2.09 |
| 2, p49/p100 | | | | |
| DNA segment, Chr 7, ERATO Doi 462, expressed | −1.09* | −2.61 | −1.53* | −1.06 |
| immunoglobulin superfamily member 4B | 1.00 | −2.82 | 1.47 | −1.23* |
| target of mybl-like 1 chicken | 1.42 | −2.98* | −1.56* | −1.02 |
| myo-inositol oxygenase | −1.35* | −3.17 | −1.06 | −1.23* |
| RIKEN cDNA 5730507H05 gene | −1.21* | −4.58 | −4.22 | −2.61* |

shown value indicates a higher variability of signal intensity between the two RNA samples analyzed for each model.

TABLE 11
MLC/Class 2 I GF-1Ea list of up-and down-regulated genes. A selection of
the genes that were either up- or down-regulated by 1.9 fold is shown. Genes in bold are
unique for the IGF1-1Ea transgenics, i.e. they scored an increase or decrease of 1.9 fold in
the IGF1-1Ea transgenic while at the same time scoring less than 1.3 up- or down
regulation in the other three transgenic models. The presence of a star on the right of each
shown value indicates a higher variability of signal intensity between the two RNA
samples analyzed for each model.

| GENE NAME | 1 EA | 1 EB | 2 EA | 2 EB |
|---|---|---|---|---|
| insulin-like growth factor 1 | 25.52* | 3.32 | 15.69 | 1.80 |
| colony stimulating factor 3 receptor (granulocyte) | 2.11 | 1.44 | 2.39 | −1.13* |
| gap junction membrane channel protein alpha 1 | −1.03 | 1.13 | 2.33 | −1.07 |
| adaptor-related protein complex AP-4, mu 1 | 1.39 | 1.89* | 2.29 | 3.29 |
| DIX domain containing 1 | 2.82 | 2.00* | 2.29 | 3.29 |
| beta-site APP-cleaving enzyme 2 | 1.89 | −1.32 | 2.27 | −1.16* |
| shroom | −1.07* | −1.10* | 2.09 | −2.63* |
| zinc finger protein, subfamily 1A, 1 (Ikaros) | 2.91* | 2.79* | 2.07* | 2.55* |
| serine (or cysteine) peptidase inhibitor, clade A, member 3A | 1.23* | 1.30 | 1.97 | 1.11 |
| carbonic anhydrase 5a, mitochondrial | 1.82 | 1.31* | 1.94 | −1.02* |
| FK506 binding protein 5 (immunophilin) | 1.03* | 1.18* | 1.93 | 1.18 |
| deltex 2 homolog (Drosophila) | 1.09* | −1.01 | 1.91 | 1.81 |
| transmembrane protein 54 | 1.08 | −1.18 | −1.94 | 1.24 |
| Slit-like 2 (Drosophila) | −1.30 | −1.09 | −1.95 | −1.20 |
| RNA binding motif protein, X chromosome, mRNA | −1.07 | −1.16 | −1.95 | 1.18 |
| chloride channel CLIC-like 1 | −1.14* | −1.12 | −1.99 | −1.16 |
| cytochrome P450, family 2, subfamily b, polypeptide 10 | −1.03 | −1.27* | −2.02 | −1.07 |
| Bcl2-like 2 | −1.30 | 1.00 | −2.05* | 1.21 |
| RIKEN cDNA 2310043L02 gene | 1.25 | 1.39 | −2.09 | 1.10 |
| splicing factor proline/glutamine rich | −1.14 | −1.42 | −2.09 | −1.18 |
| phosphatidylserine synthase 2 | −1.47* | −1.02* | −2.12 | 1.09 |
| tRNA nucleotidyl transferase, CCA-adding, 1 | −1.55 | 1.14 | −2.14 | 1.36* |
| C79248: similar to platelet-activated factor acetylhydrolase (PAF-AH) | 1.50 | −1.53 | −2.15 | −1.43 |
| DnaJ (Hsp40) homolog, subfamily C, member 18 | −1.34 | −1.23 | −2.16 | −1.02 |
| calcium regulated heat stable protein 1 | 1.20 | 1.09* | −2.23 | 1.44 |
| B-cell CLL/lymphoma 6, member B | 1.20 | −1.32* | −2.25 | 1.36* |
| cerebellin 1 precursor protein | −1.20 | −1.34* | −2.27 | −1.64 |
| CEA-related cell adhesion molecule 2 | 1.75* | −1.58 | −2.31 | 2.11* |
| proteasome (prosome, macropain) 26S subunit, ATPase, 6 | −1.41 | 1.12 | −2.31 | 1.18 |
| suppressor of cytokine signaling 3 | −1.06 | −1.25* | −2.31 | 1.29* |
| NF-kB 2, p49/p100 | −1.28* | −2.46 | −2.35 | −2.09 |
| cell division cycle associated 3 | 1.30 | −1.18* | −2.42 | 1.14 |
| tissue factor pathway inhibitor | 1.18 | −1.03* | −2.46 | 1.21* |
| cyclic AMP-regulated phosphoprotein, 21 | 1.52 | −1.01 | −2.52* | −1.36 |
| mutY homolog (E. coli) | 1.15 | −1.13* | −2.54 | −1.46 |
| apoptosis antagonizing transcription factor | −1.04 | 1.36 | −2.56* | −1.08* |
| integrin alpha 5 (fibronectin receptor alpha) | −1.81* | −1.04 | −2.66 | 1.18 |
| calsyntenin 3 | 1.15 | −1.76* | −2.71 | 1.20 |
| SRY-box containing gene 4 (Sox4), mRNA | 1.68 | −1.41* | −2.85 | 1.21 |
| coagulation factor VII | 1.31 | 1.11* | −2.86 | 1.09 |
| aldehyde dehydrogenase 2, mitochondrial | −1.71* | −1.12 | −3.06 | −1.51 |
| tetraspan 1 | −1.71 | 1.14 | −3.12 | −2.03* |
| myelin basic protein expression factor 2, repressor | −1.85* | 1.03 | −3.36* | −1.06 |
| Ubiquitin specific peptidase 7 (Usp7), mRNA | −1.31 | −2.27* | −3.39 | −1.48* |
| Notch-regulated ankyrin repeat protein | −1.18 | 1.14 | −3.51* | −1.11* |
| scaffold attachment factor B2 | −1.41* | −1.65* | −3.78 | −2.41 |
| zinc finger protein 503 | 1.12 | −1.65* | −3.94 | −2.09* |
| per-pentamer repeat gene | 1.35 | −1.06* | −4.03 | −1.27 |
| thyroid stimulating hormone, beta subunit | 1.07 | 1.17* | −4.03* | 1.12 |
| contactin associated protein-like 2 | 1.71 | 1.39 | −5.11 | 1.37 |
| splA/ryanodine receptor domain and SOCS box containing 4 | 1.45 | 1.40 | −7.87* | 1.58 |

TABLE 12
MLC/Class 2 IGF-1Eb list of up-and down-regulated genes. A selection of
the genes that were either up- or down-regulated by 1.9 fold is shown. Genes in bold are
unique for the IGF1-1Ea transgenics, i.e. they scored an increase or decrease of 1.9 fold in
the IGF1-1Ea transgenic while at the same time scoring less than 1.3 up- or down
regulation in the other three transgenic models. The presence of a star on the right of each
shown value indicates a higher variability of signal intensity between the two RNA
samples analyzed for each model.

| GENE NAME | 1 EA | 1 EB | 2 EA | 2 EB |
|---|---|---|---|---|
| DIX domain containing 1 | 2.82 | 2.00* | 2.29 | 3.29 |
| adaptor-related protein complex AP-4, mu 1 | 1.39 | 1.89* | 2.29 | 3.29 |
| zinc finger protein, subfamily 1A, 1 (Ikaros) | 2.91* | 2.79* | 2.07* | 2.55* |
| homeodomain interacting protein kinase 2 | 1.35 | 1.33 | 1.43 | 2.34 |
| WAP four-disulfide core domain 2 | 1.23* | −1.05* | −1.02* | 2.18 |
| CEA-related cell adhesion molecule 2 | 1.75* | −1.58 | −2.31 | 2.11* |
| ferritin mitochondrial | 1.63 | 1.58 | 1.18 | 2.07 |
| T-cell receptor beta, variable 13 | 1.64 | 1.94 | 1.25 | 2.03 |
| EH-domain containing 1 | 1.14 | −1.26 | 1.33 | −1.93 |
| angiopoietin-like 1 | −1.29 | 1.03 | 1.09 | −1.93* |
| heat shock protein 1B | −1.02 | −1.98 | 1.16* | −1.98 |
| homeo box C5 | 1.02 | 1.17 | 1.42 | −1.99 |
| tetraspan 1 | −1.71 | 1.14 | −3.12 | −2.03* |
| Rho GTPase activating protein 4 | 1.10* | −1.80 | 1.32 | −2.04 |
| nuclear factor of kappa light polypeptide gene enhancer in B-cells 2, | −1.28* | −2.46 | −2.35 | −2.09 |
| p49/p100 | | | | |
| RNA binding motif protein, X chromosome, mRNA | 1.40 | −1.20 | 1.01* | −2.09 |
| ephrin B3 | −1.13 | 1.00* | 1.29 | −2.09 |
| transmembrane protein 45b | −1.18 | −1.04 | −1.09 | −2.13 |
| erythroid associated factor | 1.07 | −1.51* | 1.47 | −2.18 |
| procollagen, type II, alpha 1 | −1.16 | 1.05 | −1.59 | −2.23* |
| sphingomyelin phosphodiesterase 2, neutral | −1.13* | −1.64* | 1.22 | −2.23 |
| PKD2 interactor, golgi and endoplasmic reticulum associated 1 | −1.00 | −1.03* | 1.22 | −2.30 |
| scaffold attachment factor B2 | −1.41* | −1.65* | −3.78 | −2.41 |
| phosphoenolpyruvate carboxykinase 1, cytosolic | −1.17* | −1.00 | 1.15 | −2.49 |
| RIKEN cDNA 9130019O22, putative zinc finger protein | 1.94 | 1.69 | 1.05* | −2.50 |
| mast cell protease 5 | 1.30 | −1.34 | 1.13 | −2.61* |
| Shroom | −1.07* | −1.10* | 2.09 | −2.63* |
| endothelin 1 | −1.08* | 1.02 | −1.10 | −2.67* |
| serine (or cysteine) peptidase inhibitor, clade D, member 1 | −2.96 | −1.00 | 1.08 | −2.92* |
| potassium channel, subfamily K, member 7 | 1.29 | −1.23 | −1.07 | −3.12 |
| myosin, light polypeptide 4 | −1.20* | −1.12 | −1.83* | −3.21 |
| major urinary protein 1 and 3 | −1.13* | 1.05* | −1.01 | −3.38 |
| cofactor required for Sp1 transcriptional activation, subunit 6 | −1.92* | −1.20 | −1.71* | −3.58 |
| RIKEN cDNA 5730410I19 gene | 1.05 | −1.32 | −1.18 | −3.96 |
| Sulfiredoxin 1 homolog (S. cerevisiae) (Srxn1), mRNA | 1.01 | −1.45 | 1.45 | −4.44 |

TABLE 13
List of up-and down-regulated genes only in response to Class 1 isoforms. A
selection of the genes that resulted either up- or down-regulated by 1.3 fold only in the
Class 1 expressing transgenic samples. The presence of a star on the right of each shown
value indicates a higher variability of signal intensity between the two RNA samples
analyzed for each model.

| GENE NAME | 1 Ea | 1 Eb | 2 Ea | 2 Eb |
|---|---|---|---|---|
| melanoma antigen, family A, 7 | −2.26 | −2.04 | 1.12 | 1.75 |
| DNA segment, Chr 2, ERATO Doi 63, expressed | −2.05* | −1.58 | 1.01 | 1.61 |
| transmembrane channel-like gene family 1 | −1.89* | −1.31 | 1.33 | 1.17 |
| cofactor required for Sp1 transcriptional activation, subunit 2 | −1.49* | −1.84* | 1.01 | 1.19 |
| potassium voltage-gated channel, shaker-related subfamily, member 1 | −1.63* | −1.56* | 1.06 | 1.14 |
| potassium voltage-gated channel, shaker-related subfamily, beta member 1 | −1.53 | −1.35 | 1.04 | 1.25 |
| melanophilin | −1.45 | −1.46* | 1.12 | 1.09 |
| growth differentiation factor 11 | −1.47 | −1.33 | 1.04 | 1.03 |
| 3-hydroxybutyrate dehydrogenase (heart, mitochondrial) | −2.36 | −1.50 | −1.04 | 1.55 |
| small EDRK-rich factor 1 | −2.06 | −1.85 | −1.16* | 1.17* |
| 6-phosphofructo-2-kinase/fructose-2,6-biphosphatase 3 | −1.60 | −1.74* | −1.17* | 1.08 |
| PHD finger protein 2 | −1.74 | −1.30 | 1.22 | −1.14 |
| protein kinase C, gamma | −1.34 | −1.32 | −1.21* | 1.46 |
| Ras association (RalGDS/AF-6) domain family 7 | −1.81* | −2.05* | −1.21 | −1.15 |
| toll-like receptor 9 | 1.43 | 1.59 | 1.11* | −1.02 |
| ubiquitination factor E4B, UFD2 homolog (S. cerevisiae) | 1.62 | 1.53 | −1.03 | 1.08* |
| syndecan 3 | 1.57* | 1.53 | −1.37 | 1.10 |
| Immunoglobulin kappa light variable region (IgKV gene) | 1.90 | 1.36 | 1.02 | −1.64* |
| protocadherin beta 8 | 1.35 | 1.31 | −1.30* | −1.00 |
| apolipoprotein B editing complex 3 | 1.41 | 1.41 | −1.06* | −1.10 |
| Syntaxin 5A, mRNA (cDNA clone MGC: 25518 IMAGE: 3487476) | 1.40 | 1.48 | −1.12 | −1.03 |
| RIKEN cDNA 9130019O22 gene | 1.94 | 1.69 | 1.05* | −2.50 |
| antigen identified by monoclonal antibody Ki 67 | 1.46 | 1.32 | −1.11 | −1.23 |
| bleomycin hydrolase | 1.36 | 1.32 | −1.35 | −1.29 |
| keratin associated protein 5-1 | 1.52* | 1.52* | −1.07 | −1.38 |
| zinc finger protein 30 | 1.45 | 1.52 | −1.41* | −1.38 |
| hyaluronidase 3 | 1.42* | 2.14* | −1.15 | −1.23 |
| midasin homolog (yeast) | 1.72 | 1.30* | −1.67* | −1.32* |
| solute carrier family 16 (monocarboxylic acid transporters), member 11 | 2.30 | 1.32* | −1.22* | −1.40 |

TABLE 14
List of up-and down-regulated genes only in response to Class 2 isoforms. A
selection of the genes that resulted either up- or down-regulated by 1.3 fold only in the
Class 1 expressing transgenic samples. The presence of a star on the right of each shown
value indicates a higher variability of signal intensity between the two RNA samples
analyzed for each model.

| GENE NAME | 1 Ea | 1 Eb | 2 Ea | 2 Eb |
|---|---|---|---|---|
| cyclin D1 | 1.31 | 1.04 | −2.13* | −1.89* |
| interleukin 4 receptor, alpha | 1.47* | 1.26 | −1.56 | −1.83 |
| phosphatidylinositol glycan, class O | 1.19* | 1.47 | −1.58 | −1.80* |
| Prkr interacting protein 1 (IL11 inducible) | 1.37* | 1.20* | −1.35* | −1.41* |
| ADP-ribosylation factor 3 | 1.21 | 1.08 | −1.32* | −1.68 |
| protein tyrosine phosphatase, receptor type, C | 1.47 | 1.12 | −1.31 | −1.36 |
| REX4, RNA exonuclease 4 homolog (S. cerevisiae) | 1.10 | 1.05 | −1.53 | −1.31 |
| DEAD (Asp-Glu-Ala-Asp) box polypeptide 39 | 1.20 | 1.00 | −1.35 | −1.33 |
| A kinase (PRKA) anchor protein 10 | 1.02 | 1.14* | −1.38 | −1.31* |
| cyclic AMP-regulated phosphoprotein, 21 | 1.52 | −1.01 | −2.52* | −1.36 |
| TSC22 domain family, member 1 | −1.04 | 1.12* | −2.20* | −1.45* |
| procollagen, type II, alpha 1 | −1.16 | 1.05 | −1.59 | −2.23* |
| neurobeachin-like 2 | 1.16 | −1.05 | −1.53 | −1.84* |
| lactate dehydrogenase 3, C chain, sperm specific | 1.11 | −1.18 | −1.50 | −1.85* |
| progesterone receptor membrane component 2 | −1.14 | 1.35 | −1.31* | −1.71* |
| isopentenyl-diphosphate delta isomerase | 1.02 | −1.07 | −1.54 | −1.71 |
| deiodinase, iodothyronine, type II | 1.02 | −1.01 | −1.40 | −1.77 |
| tumor protein D52-like 1 | 1.06 | −1.28 | −1.50 | −1.82 |
| cDNA sequence BC002059 | 1.01 | −1.08 | −1.73 | −1.41 |
| annexin A2 | −1.01 | 1.22 | −1.39 | −1.45* |
| dystrobrevin alpha | 1.18 | −1.10 | −1.53 | −1.42 |
| ring finger and FYVE like domain containing protein | −1.07 | 1.09 | −1.49 | −1.47 |
| myosin light polypeptide 4 | −1.20* | −1.12 | −1.83* | −3.21 |
| poly (A) polymerase alpha | −1.02 | −1.19 | −1.69 | −1.60* |
| DNA segment, Chr 6, ERATO Doi 47, expressed | −1.04 | −1.29 | −1.88 | −1.46* |
| leucine-zipper-like transcriptional regulator, 1 | −1.15 | −1.24 | −1.69 | −1.51 |
| HIV-1 Rev binding protein-like | −1.12 | −1.15 | −1.52 | −1.52 |
| hypoxia inducible factor 1, alpha subunit | 1.21* | 1.07 | 1.36 | 1.76* |
| alcohol dehydrogenase 1 (class I) | 1.05 | 1.05 | 1.61 | 1.34 |
| mitogen-activated protein kinase kinase kinase kinase 5 | 1.15* | 1.02* | 1.44 | 1.63* |
| phosphatidylinositol-3-phosphate/phosphatidylinositol 5-kinase, type III | 1.14* | −1.15* | 1.33 | 1.62 |
| X-linked myotubular myopathy gene 1 | −1.41* | 1.10 | 1.55* | 1.37 |
| deltex 2 homolog (Drosophila) | 1.09* | −1.01 | 1.91 | 1.81 |
| decapping enzyme, scavenger | −1.29 | −1.41 | 1.39 | 1.38 |

TABLE 15
List of up-and down-regulated genes only in response to Ea isoforms. A
selection of the genes that resulted either up- or down-regulated by 1.3 fold only in the
Class 1 expressing transgenic samples. The presence of a star on the right of each shown
value indicates a higher variability of signal intensity between the two RNA samples
analyzed for each model.

| GENE NAME | 1 Ea | 2 Ea | 1 Eb | 2 Eb |
|---|---|---|---|---|
| CD28 antigen | −3.10 | −1.54 | 1.01 | 1.14 |
| proteasome (prosome, macropain) 26S subunit, ATPase, 6 | −1.41 | −2.31 | 1.12 | 1.18 |
| synaptotagmin III | −2.04* | −1.77 | 1.02* | 1.14 |
| src homology 2 domain-containing transforming protein C3 | −1.61 | −1.66* | 1.17 | 1.07 |
| myelin basic protein expression factor 2, repressor | −1.85* | −3.36* | 1.03 | −1.06 |
| asparagine-linked glycosylation 1 homolog | −1.36 | −1.31 | 1.01 | 1.30 |
| myosln, heavy polypeptide 6, cardiac muscle, alpha | −1.37 | −1.32 | 1.06 | 1.21* |
| integrin alpha 5 (fibronectin receptor alpha) | −1.81* | −2.66 | −1.04 | 1.18 |
| NADH dehydrogenase (ubiquinone) 1 alpha subcomplex 10 | −1.42* | −3.14* | −1.10* | 1.12 |
| phosphatidylserine synthase 2 | −1.47* | −2.12 | −1.02* | 1.09 |
| elongation factor Tu GTP binding domain containing 2 | −2.02 | −1.47 | 1.12 | −1.08 |
| RecQ protein-like | −1.70* | −1.66 | −1.09 | 1.19* |
| regulator of G-protein signaling 5 | −1.37 | −1.83 | −1.03 | 1.27 |
| zinc finger, FYVE domain containing 19 | −1.40* | −1.67 | 1.19* | −1.06 |
| Rho GTPase activating protein 21 | −1.46 | −1.50* | −1.04* | 1.02 |
| protein kinase, cGMP-dependent, type I | −1.47 | −2.32* | −1.23* | −1.17 |
| DnaJ (Hsp40) homolog, subfamily C, member 18 | −1.34 | −2.16 | −1.23 | −1.02 |
| Sjogren syndrome antigen B | −1.98 | −1.44 | −1.11 | −1.10* |
| ring finger protein 11 | −1.50 | −1.69 | −1.06 | −1.11 |
| neutral sphingomyelinase (N-SMase) activation associated factor | −1.33 | −1.74 | −1.01 | −1.05 |
| tumor necrosis factor receptor superfamily, member 19 | −1.37 | −1.78 | −1.10 | −1.06 |
| enhancer of yellow 2 homolog (Drosophila) | −1.49 | −1.61 | −1.11 | −1.11 |
| ATPase, H+ transporting, lysosomal accessory protein 2 | −1.45 | −1.38 | −1.02 | −1.02 |
| CDC42 effector protein (Rho GTPase binding) 3 | −1.42* | −1.46 | −1.10 | −1.02 |
| methylenetetrahydrofolate dehydrogenase (NAD+ dependent) | −1.31 | −1.47 | −1.04 | −1.04 |
| GTP binding protein 4 | −1.49 | −1.37 | −1.14 | −1.12 |
| ADP-ribosylation factor-like 6 interacting protein 6 | −1.42* | −1.44 | −1.09 | −1.29 |
| fibroblast growth factor 11 | −1.36 | −1.50 | −1.23 | −1.28 |
| RAS related protein 1b | −1.33 | −1.36 | −1.23 | −1.11 |
| Rho GTPase activating protein 5 | 1.65 | 1.30 | 1.11 | 1.20 |
| upregulated during skeletal muscle growth 1 | 1.40 | 1.47 | 1.17 | 1.04* |
| sushi domain containing 4 | 1.45 | 1.43 | 1.16 | 1.04 |
| growth factor receptor bound protein 2-associated protein 3 | 1.48 | 1.42 | 1.02 | 1.08* |
| SH3-domain GRB2-like 2 | 1.32 | 1.76* | 1.22* | 1.02 |
| trace amine-associated receptor 1 | 1.32 | 1.77 | 1.10 | 1.15 |
| DNA segment, Chr 9, ERATO Doi 720, expressed | 1.53 | 1.65 | 1.05 | 1.29 |
| keratin complex 1, acidic, gene 24 | 1.88 | 1.56 | 1.05* | 1.27 |
| leucine rich repeat containing G protein coupled receptor 5 | 1.91* | 1.58* | 1.24 | 1.02 |
| adaptor protein with pleckstrin homology and src | 1.38 | 1.57 | −1.03 | 1.08 |
| RASD family, member 2 | 1.66 | 1.40 | 1.16* | −1.01 |
| synaptotagmin XII | 1.31 | 1.62 | 1.09 | −1.09 |
| laminin, beta 3 | 1.37 | 1.56 | 1.17 | −1.33* |
| midasin homolog (yeast) | 1.58 | 1.56* | 1.01 | −1.11 |
| ankyrin repeat and SOCS box-containing protein 6 | 1.53* | 1.70* | −1.04 | 1.02 |
| CEA-related cell adhesion molecule 2 | 1.32 | 1.85 | −1.54 | 1.06 |
| Rho GDP dissociation inhibitor (GDI) gamma | 1.32 | 1.32 | −1.27 | −1.06 |
| mbt domain containing 1 | 1.32 | 1.35 | −1.31 | −1.02* |
| endothelin converting enzyme 2 | 1.38 | 1.52 | −1.23* | −1.06 |
| expressed in non-metastatic cells 6, protein | 1.64 | 1.50 | −1.18* | −1.01 |
| phosphatidylinositol membrane-associated 1 | 1.53 | 1.61* | −1.21* | −1.05* |
| lin 7 homolog b (C. elegans) | 1.34 | 1.41 | −1.60 | −1.10 |
| beta-site APP-cleaving enyme 2 | 1.89 | 2.27 | −1.32 | −1.16* |
| proline rich protein HaeIII subfamily 1 | 3.83* | 2.99* | −1.42 | −1.09 |

TABLE 16
List of up-and down-regulated genes only in response to Eb isoforms. A
selection of the genes that resulted either up- or down-regulated by 1.3 fold only in the
Class 1 expressing transgenic samples. The presence of a star on the right of each shown
value indicates a higher variability of signal intensity between the two RNA samples
analyzed for each model.

| GENE NAME | 1 Ea | 2 Ea | 1 Eb | 2 Eb |
|---|---|---|---|---|
| Sulfiredoxin 1 homolog (S. cerevisiae) (Srxn1), mRNA | 1.01 | 1.45 | −1.45 | −4.44 |
| heat shock protein 1A | 1.02 | 1.37 | −2.25 | −1.88 |
| mast cell protease 5 | 1.30 | 1.13 | −1.34 | −2.61* |
| CD52 antigen | 1.35* | −1.13* | −2.13* | −3.99 |
| Rho GTPase activating protein 4 | 1.10* | 1.32 | −1.80 | −2.04 |
| lactalbumin, alpha | 1.45 | 1.26* | −1.63* | −1.91* |
| erythroid associated factor | 1.07 | 1.47 | −1.51* | −2.18 |
| activating transcription factor 3 | 1.01 | 1.34 | −1.83 | −1.41 |
| leucine-rich repeat kinase 1 | 1.30 | 1.02 | −1.48 | −1.71* |
| Immunoglobulin A heavy chain variable region (IGHV gene), | 1.34* | 1.04 | −1.50* | −1.63* |
| clone WJ17 | | | | |
| heat shock protein 1B | −1.02 | 1.16* | −1.98 | −1.98 |
| sphingomyelin phosphodiesterase 2, neutral | −1.13* | 1.22 | −1.64* | −2.23 |
| early B-cell factor 3 | 1.01 | −1.09 | −1.39 | −1.69 |
| transient receptor potential cation channel, subfamily M, member 7 | −1.06 | 1.00 | −1.55 | −1.49* |
| thyrotroph embryonic factor | −1.13 | 1.07 | −1.30 | −1.67 |
| DNA segment, Chr 16, ERATO Doi 472, expressed | 1.07 | −1.04 | −1.31 | −1.34 |
| solute carrier family 5 (choline transporter), member 7 | −1.15 | −1.05 | −1.57 | −1.69 |
| chemokine (C—C motif) ligand 6 | −1.04 | −1.07 | −1.66* | −1.39 |
| cholinergic receptor, nicotinic, alpha polypeptide 4 | −1.26* | −1.08 | −1.77* | −1.50 |
| ribosomal protein S9 | −1.24 | −1.15 | −1.36 | −1.76 |
| RIKEN cDNA 4631403P03 gene | −1.05 | −1.09 | −1.31 | −1.36* |
| EGF-like-domain, multiple 9 | −1.24 | −1.20 | −1.35 | −1.55 |
| RER1 retention in endoplasmic reticulum 1 homolog (S. cerevisiae) | 1.01 | −1.03 | 1.32 | 1.61 |
| scratch homolog 1, zinc finger protein (Drosophila) | −1.01* | 1.19* | 1.48 | 1.76 |
| eukaryotic translation initiation factor 3, subunit 6 interacting | 1.02* | −1.35 | 1.31 | 1.85 |
| protein | | | | |

TABLE 17
List of Ingenuity gene abbreviations

| | |
|---|---|
| ACTN1 | actinin, alpha 1 |
| AGT | angiotensinogen |
| AKT1 | thymoma viral proto-oncogene 1 |
| AKT2 | thymoma viral proto-oncogene 2 |
| B2M | beta-2 microglobulin |
| BAD | Bcl-associated death promoter |
| BCL2 | B-cell leukemia/lymphoma 2 |
| BMP7 | bone morphogenetic protein 7 |
| CAMK2A | calcium/calmodulin-dependent protein kinase II |
| | alpha |
| CAV1 | caveolin, caveolae protein 1 |
| CCND1 | cyclin D1 |
| CCNE1 | cyclin E1 |
| CD28 | CD28 antigen |
| CD36 | CD36 antigen |
| CD80 | CD80 antigen |
| CDC37 | cell division cycle 37 homolog |
| CDH1 | cadherin 1 |
| CDK4 | cyclin-dependent kinase 4 |
| CDKN1A | cyclin-dependent kinase inhibitor 1A (P21) |
| CDKN1B | cyclin-dependent kinase inhibitor 1B (P27) |
| CHUK | conserved helix-loop-helix ubiquitous kinase |
| COL18A1 | procollagen, type XVIII, alpha 1 |
| CSF1 | colony stimulating factor 1 (macrophage) |
| CTNNB1 | beta-catenin |
| CYR61 | cysteine rich protein 61 (IGFBP10) |
| EDN1 | endothelin 1 |
| EGFR | epidermal growth factor receptor |
| EGR1 | early growth response 1 |
| EIF4EBP1 | eukaryotic translation initiation factor 4E binding |
| | protein 1 |
| ERBB2 | v-erb-b2 erythroblastic leukemia viral oncogene |
| | homolog 2 |
| ESR1 | estrogen receptor 1 (alpha) |
| FGF2 | fibroblast growth factor 2 |
| FMR1 | fragile X mental retardation syndrome 1 homolog |
| FN1 | fibronectin 1 |
| FRAP1 | mTOR |
| GH1 | growth hormone |
| GHR | growth hormone receptor |
| GLI1 | GLI-Kruppel family member GUI1 |
| GRB10 | growth factor receptor bound protein 10 |
| GRIN2B | glutamate receptor, ionotropic, NMDA2B |
| GSK3B | glycogen synthase kinase 3 beta |
| HIF1A | hypoxia inducible factor 1, alpha subunit |
| IGF1 | insulin-like growth factor 1 |
| IGF1R | insulin-like growth factor I receptor |
| IGF2 | insulin-like growth factor 2 |
| IGFBP1 | insulin-like growth factor binding protein 1 |
| IGFBP2 | insulin-like growth factor binding protein 2 |
| IGFBP3 | insulin-like growth factor binding protein 3 |
| IGFBP4 | insulin-like growth factor binding protein 4 |
| IGFBP5 | insulin-like growth factor binding protein 5 |
| IGFBP6 | insulin-like growth factor binding protein 6 |
| IGFBP7 | insulin-like growth factor binding protein 7 |
| IKBKB | inhibitor of kappaB kinase beta |
| IKBKG | inhibitor of kappaB kinase gamma |
| INS | insulin II |
| Ins1 | insulin I |
| INSR | insulin receptor |
| IRAK1 | interleukin-1 receptor-associated kinase 1 |
| IRS1 | insulin receptor substrate 1 |
| IRS2 | insulin receptor substrate 2 |
| ITGA5 | integrin alpha 5 |
| ITGB1 | integrin beta 1 |
| ITGB3 | integrin beta 3 |
| JAK2 | Janus kinase 2 |
| JUN | Jun oncogene |
| KDR | kinase insert domain protein receptor |
| KITLG | kit ligand |
| MME | membrane metallo endopeptidase |
| MMP19 | matrix metallopeptidase 19 |
| MMP2 | matrix metallopeptidase 2 |
| MYB | MYB (myeloblastosis oncogene) |
| MYC | c-myc |
| MYOD1 | MyoD |
| MYOG | myogenin |
| NFKB2 | nuclear factor of kappa light polypeptide gene |
| | enhancer in B-cells 2, p49/p100 |
| NFKBIA | nuclear factor of kappa light chain gene enhancer in |
| | B-cells inhibitor, alpha |
| PCSK5 | proprotein convertase subtilisin/kexin type 5 |
| PIK3R1 | phosphatidylinositol 3-kinase, regulatory subunit |
| PLAU | plasminogen activator, urokinase |
| PLG | plasminogen |
| PRKCA | protein kinase C, alpha |
| PRKCD | protein kinase C, delta |
| PTEN | phosphatase and tensin homolog |
| PTK2 | Focal Adhesion Kinase |
| PTPN11 | protein tyrosine phosphatase, non-receptor type 11 |
| RB1 | retinoblastoma 1 |
| RELA | v-rel reticuloendotheliosis viral oncogene homolog A |
| RPS6KA1 | ribosomal protein S6 kinase polypeptide 1 |
| RPS6KB1 | ribosomal protein S6 kinase, polypeptide 1 |
| SHC1 | src homology 2 domain-containing transforming |
| | protein C1 |
| SOCS1 | suppressor of cytokine signaling 1 |
| SOCS2 | suppressor of cytokine signaling 2 |
| SOCS3 | suppressor of cytokine signaling 3 |
| SPP1 | secreted phosphoprotein 1 |
| SRC | src |
| STAT3 | STAT 3 |
| STAT5B | STAT 5B |
| TF | transferrin |
| TGFB1 | transforming growth factor, beta 1 |
| THBS1 | thrombospondin 1 |
| TJP1 | tight junction protein 1 |
| TNF | tumor necrosis factor |
| TNFSF10 | tumor necrosis factor (ligand) superfamily |
| TP53 | transformation related protein 53 |
| VEGF | vascular endothelial growth factor A |
| VEGFC | vascular endothelial growth factor C |
| VTN | vitronectin |
| WT1 | Wilms tumor homolog |

### REFERENCES

{1} Blundell, T. L. and Humbel, R. E., 1980, Nature 287:781-787
{2} Ebberink, R. H. M. et al., 1989, Biol. Bull 177:176-182
{3} Smit, A. B., et al., 1989, Nature 331:535-538
{4} Froesh, E. R., in Insulin-like Growth Factors/Somatomediams, de Gruyter, S. M. (ed.), New York, pp. 18-29 (1983)
{5} Lowe, M. W. Jr., in Insulin-likeGrowth Factors:Molecular and Cellular Aspects, LeRoith, D. (ed.), Boca Raton, Fla., CRC Press, pp 49-80 (1991)
{6} Adamo M. L., Ben-Hur H., Roberts C. T., Jr., LeRoith D., (1991) Regulation of start site usage in the leader exons of the rat insulin-like growth factor-I gene by development, fasting, and diabetes, Mol Endocrinol 5:1677-1686
{7} Simmons J G, Van Wyk J J, Hoyt E C, Lund P K. (1993) Multiple transcription start sites in the rat insulin-like growth factor-I gene give rise to IGF-I mRNAs that encode different IGF-I precursors and are processed differently in vitro.Growth Factors. 9(3):205-21
{8} Yang H., Adamo M. L., Koval A. P. et al., (1995) Alternative leader sequences in insulin-like growth factor I mRNAs modulate translational efficiency and encode multiple signal peptides, Mol Endocrinol 9:1380-1395
{9} Bell G. I., Stempien M. M., Fong N. M., (1986) Rall L. B., Sequences of liver cDNAs encoding two different mouse insulin-like growth factor I precursors, Nucleic Acid Research 14:7873-7882
{10} Shimatsu A., Rotwein P., (1987) Mosaic evolution of the insulin-like growth factors. Organization, sequence, and expression of the rat insulin-like growth factor I gene, J Biol Chem 262:7894-7900
{11} Roberts C. T. Jr., Lasky S. R., Lowe W. L., Jr., Seaman W. T., LeRoith D., (1987) Molecular cloning of rat insulin-like growth factor I complementary deoxyribonucleic acids: Differential messenger ribonucleic acid processing and regulation by growth hormone in extrahepatic tissues, Mol Endocrinol 1:243-248.
{12} Le Bouc Y., Dreyer D., Jaeger F., Binoux M., Sondermeyer P., (1986) Complete characterization of the human IGF-I nucleotide sequence isolated from a newly constructed adult liver cDNA library, FEBS Lett 196:108-112.
{13} Lund P. K., Hoyt E. C., Van Wyk J. J., (1989) The size heterogeneity of rat insulin-like growth factor-I mRNAs is due primarily to differences in the length of 31-untranslated sequence, Mol Endocrinol 3:2054-2061.
{14} Ito H, Hiroe M, Hirata Y, Tsujino M, Adachi S, Shichiri M, Koike A, Nogami A, Marumo F. (1993) Insulin-like growth factor-I induces hypertrophy with enhanced expression of muscle specific genes in cultured rat cardiomyocytes, Circulation 87:1715-21.
{15} Kajstura J, Cheng W, Reiss K, Anversa P. (1994) The IGF-1-IGF-1 receptor system modulates myocyte proliferation but not myocyte cellular hypertrophy in vitro. Exp Cell Res. 215:273-83.
{16} Bark T H, McNurlan M A, Lang C H, Garlick P J. (1998) Increased protein synthesis after acute IGF-I or insulin infusion is localized to muscle in mice. Am J Physiol. 275:E118-23.
{17} Cittadini A, Stromer H, Katz S E, Clark R, Moses A C, Morgan J P, Douglas P S. (1996) Differential cardiac effects of growth hormone and insulin-like growth factor-1 in the rat. A combined in vivo and in vitro evaluation. Circulation. 93:800-9.
{18} Reiss K, Cheng W, Ferber A, Kajstura J, Li P, Li B, Olivetti G, Homcy C J, Baserga R, Anversa P. (1996) Overexpression of insulin-like growth factor-1 in the heart is coupled with myocyte proliferation in transgenic mice. Proc Natl Acad Sci U S A. 93:8630-5.
{19} Delaughter M C, Taffet G E, Fiorotto M L, Entman M L, Schwartz R J. 1999 Local insulin-like growth factor I expression induces physiologic, then pathologic, cardiac hypertrophy in transgenic mice. FASEB J. 13:1923-9.
{20} Barton-Davis E R, Shoturma D I, Musaro A, Rosenthal N, Sweeney H L. 1998 Viral mediated expression of insulin-like growth factor I blocks the aging-related loss of skeletal muscle function. Proc Natl Acad Sci U S A. 95:15603-7.
{21} Musaro A, Giacinti C, Borsellino G, Dobrowolny G, Pelosi L, Cairns L, Ottolenghi S, Cossu G, Bernardi G, Battistini L, Molinaro M, Rosenthal N. (2004) Stem cell-mediated muscle regeneration is enhanced by local isoform of insulin-like growth factor 1. Proc Natl Acad Sci U S A. 101:1206-10.
{22} Paul AC, Rosenthal N. (2002) Different modes of hypertrophy in skeletal muscle fibers. J Cell Biol. 156(4):751-60.
{23} Barton E R, Morris L, Musaro A, Rosenthal N, Sweeney H L. (2002) Muscle-specific expression of insulin-like growth factor I counters muscle decline in mdx mice. J Cell Biol. 157:137-48.
{24} Yang S, Alnaqeeb M, Simpson H, Goldspink G Cloning and characterization of an IGF-1 isoform expressed in skeletal muscle subjected to stretch. J Muscle Res Cell Motil. 1996 Aug;17(4):487-95.
{25} Rotwein P, Pollock K M, Didier D K, Krivi G G. Organization and sequence of the human insulin-like growth factor I gene. Alternative RNA processing produces two insulin-like growth factor I precursor peptides. J Biol Chem. 1986 Apr 15;261(11):4828-32.
{26} Chew S L, Lavender P, Clark A J, Ross R J. An alternatively spliced human insulin-like growth factor-I transcript with hepatic tissue expression that diverts away from the mitogenic IBE1 peptide.Endocrinology. 1995 May;136(5):1939-44.
{27} Musaro A, Giacinti C, Borsellino G, Dobrowolny G, Pelosi L, Cairns L, Ottolenghi S, Cossu G, Bernardi G, Battistini L, Molinaro M, Rosenthal N. (2004) Stem cell-mediated muscle regeneration is enhanced by local isoform of insulin-like growth factor l.Proc Natl Acad Sci USA.101:1206-10.
{28} Ibba (1996) Biotechnol Genet Eng Rev 13:197-216 (291 Sambrook (1989) Molecular Cloning; A Laboratory Manual ISBN: 0879695773 (301 Fernandez et al. (1998) eds Gene expression systems. Using nature for the art of expression ISBN: 0122538404
{31} Summers et al. (1987) Texas Agricultural Experiment Station Bulletin No. 1555
{32} U.S. Pat. No. 5,693,506
{33} U.S. Pat. No. 5,659,122
{34} U.S. Pat. No. 5,608,143
{35} Zenk (1991) Phytochemistry 30:3861-3863
{36} Bodanszky (1993) Principles of Peptide Synthesis ISBN: 0387564314
{37} Fields et al (1997) Methods in Enzymology 289: Solid-Phase Peptide Synthesis ISBN: 0121821900
{38} Chan & White (2000) Fmoc Solid Phase Peptide Synthesis ISBN: 0199637245
{39} Kullmann (1987) Enzymatic Peptide Synthesis ISBN: 0849368413
{40} Gennaro (2000) Remington: The Science and Practice of Pharmacy 20th ed, ISBN: 0683306472
{41} WO 02/36161
{42} Almeida & Alpar (1996) J Drug Targeting 3:455-467
{43} Merly F, Lescaudron L, Rouaud T, Crossin F, Gardahaut M F (1999) Macrophages enhance muscle satellite cell proliferation and delay their differentiation. Muscle Nerve. 22:724-32.
{44} Camargo F D, Green R, Capetanaki Y, Jackson K A, Goodell M A, Capetenaki Y.
(2003) Single hematopoietic stem cells generate skeletal muscle through myeloid intermediates.Nat Med. 9(12):1520-7.
{45} Berkner, K.L., Curr. Top. Microbiol. Immunol., 158, 39-66 (1992)
{46} Muzyczka, N., Curr. Top. Microbiol. Immunol., 158, 97-129 (1992)
{47} U.S. Patent No. 5,252,479
{48} Chapter 20, Gene Therapy and other Molecular Genetic-based Therapeutic Approaches, (and references cited therein) in Human Molecular Genetics (1996), T Strachan and A P Read, BIOS Scientific Publishers Ltd
{49} Current Protocols in Molecular Biology (FM Ausubel et al, eds. 1987) Supplement 30
{50} Smith & Waterman (1981) Adv Appl Math 2: 482-489
{51} Hambrecht R, Schulze P C, Gielen S, Linke A, Mobius-Winkler S, et al. (2002) Reduction of insulin-like growth factor-I expression in the skeletal muscle of noncachectic patients with chronic heart failure. J Am Coll Cardiol 39(7): 1175-1181.
{52} Schulze PC, Gielen S, Adams V, Linke A, Mobius-Winkler S, et al. (2003) Muscular levels of proinflammatory cytokines correlate with a reduced expression of insulin-like growth factor-I in chronic heart failure. Basic Res Cardiol 98(4): 267-274.
{53} Musaro A, McCullagh K, Paul A, Houghton L, Dobrowolny G, et al. (2001) Localized IGF-1 transgene expression sustains hypertrophy and regeneration in senescent skeletal muscle. Nat Genet 27(2): 195-200.
{54} Paul A C, Rosenthal N (2002) Different modes of hypertrophy in skeletal muscle fibers. J Cell Biol 156(4): 751-760.
{55} Sandri M, Sandri C, Gilbert A, Skurk C, Calabria E, et al. (2004) Foxo transcription factors induce the atrophy-related ubiquitin ligase atrogin-1 and cause skeletal muscle atrophy. Cell 117(3): 399-412.
{56} Florini J, Ewton D, Coolican S (1996) Growth hormone and the insulin-like growth factor system in myogenesis. Endocrine Rev 17: 481-517.
{57} Fiorotto M L, Schwartz R J, Delaughter M C (2003) Persistent IGF-I overexpression in skeletal muscle transiently enhances DNA accretion and growth. Faseb J 17(1): 59-60.
{58} Barton E R, Morris L, Musaro A, Rosenthal N, Sweeney H L (2002) Muscle-specific expression of insulin-like growth factor I counters muscle decline in mdx mice. J Cell Biol 157(1): 137-148.
{59} Subramaniam, A. et al. Tissue-specific regulation of the alpha-myosin heavy chain gene promoter in transgenic mice. J Biol Chem 266, 24613-20 (1991).
{60} Musaro, A. & Rosenthal, N. Maturation of the myogenic program is induced by postmitotic expression of insulin-like growth factor I. Mol Cell Biol 19, 3115-24 (1999).
{b 61} Orlic, D. et al. Mobilized bone marrow cells repair the infarcted heart, improving function and survival. Proc Natl Acad Sci U S A 98, 10344-9 (2001).
{62} M. a. M. a. a. a. s. o. m. o. S. Online.
{63} S. C. Bodine et al., Nat Cell Biol 3, 1014-9 (November, 2001).
{64} D. J. Glass, Nat Cell Biol 5, 87-90 (Febuary, 2003).
{65} J. Montagne et al., Science 285, 2126-9 (Sep 24, 1999).
{66} R. Bohni et al., Cell 97, 865-75 (Jun 25, 1999).
{67} H. Shima et al., Embo J 17, 6649-59 (Nov 16, 1998).
{68} D. J. Glass, Trends Mol Med 9, 344-50 (Aug, 2003).
{69} N. Pullen et al., Science 279, 707-10 (Jan 30, 1998).
{70} Hertweck, M., Gobel, C. & Baumeister, R. C. elegans SGK-1 is the critical component in the Akt/PKB kinase complex to control stress response and life span. Dev Cell 6, 577-88 (2004).
{71} Paradis, S. & Ruvkun, G. Caenorhabditis elegans Akt/PKB transduces insulin receptor-like signals from AGE-1 PI3 kinase to the DAF-16 transcription factor. Genes Dev 12, 2488-98 (1998).
{72} Frodin, M., Jensen, C. J., Merienne, K. & Gammeltoft, S. A phosphoserine-regulated docking site in the protein kinase RSK2 that recruits and activates PDK1. Embo J 19, 2924-34 (2000).
{73} G. Ren, 0. Dewald, N. G. Frangogiannis, Curr Drug Targets Inflamm Allergy 2, 242-56 (September, 2003).
{74} K. W. Liechty, H. B. Kim, N. S. Adzick, T. M. Crombleholme, J Pediatr Surg 35, 866-72; discussion 872-3 (Jun, 2000).
{75} G. P. Dotto, Biochim Biophys Acta 1471, M43-56 (Jul 31, 2000).
{76} S. A. Murray, H. Zheng, L. Gu, Z. X. Jim Xiao, Oncogene 22, 1703-11 (Mar 20, 2003).
{77} Y. Nonomura, H. Kohsaka, K. Nagasaka, N. Miyasaka, J Immunol 171, 4913-9 (Nov 1, 2003).
{78} H. Perlman et al., J Immunol 170, 838-45 (Jan 15, 2003).
{79} J. P. Brockes, A. Kumar, C. P. Velloso, J Anat 199, 3-11 (Jul-Aug, 2001).
{80} K. D. Poss, L. G. Wilson, M. T. Keating, Science 298, 2188-90 (Dec 13, 2002).
{81} J. 0. Oberpriller, J. C. Oberpriller, D. G. Matz, M. H. Soonpaa, Ann N Y Acad Sci 752, 30-46 (Mar 27, 1995).
{82} M. T. Longaker, Z. M. Peled, J. Chang, T. M. Krummel, Surgery 130, 785-7 (November, 2001).
{83} B. Nadal-Ginard, J. Kajstura, P. Anversa, A. Leri, J Clin Invest 111, 1457-9 (May, 2003).
{84} B. Nadal-Ginard, J. Kajstura, A. Leri, P. Anversa, Circ Res 92, 139-50 (Feb 7, 2003).
{85} A. Musaro et al., Proc Natl Acad Sci U S A 101, 1206-10 (Feb 3, 2004).
{86} A. Musaro et al., Nat Genet 27, 195-200 (Febuary, 2001).
{87} Rossig, L., Badorff, C., Holzmann, Y., Zeiher, A.M. & Dimmeler, S. Glycogen synthase kinase-3 couples AKT-dependent signaling to the regulation of p21Cipl degradation. J Biol Chem 277, 9684-9 (2002).
{88} Urbanek, K. et al. Cardiac stem cells possess growth factor-receptor systems that after activation regenerate the infarcted myocardium, improving ventricular function and long-term survival. Circ Res 97, 663-73 (2005).
{89} Musaro A, K. McCullagh, A. Paul, L. Houghton, G. Dobrowolny, M. Molinaro, E. R. Barton, H. L. Sweeney, and N. Rosenthal. 2001. Localized IGF-1 transgene expression sustains hypertrophy and regeneration in senescent skeletal muscle. Nat Genet. 27: 195-200.
{90} Musaro, A. and N. Rosenthal. 2002.The role of local Insulin-like Growth Factor-1 isoforms in the pathophysiology of skeletal muscle. Current Genomics 3: 149-162.
{91} Barton, E. R., L. Morris, A. Musaro, N. Rosenthal, and H.L. Sweeney. 2002. Muscle-specific expression of insulin-like growth factor I counters muscle decline in mdx mice. J Cell Biol. 157: 137-48.
{92} Musar6, A., C. Giacinti, G. Borsellino, G. Dobrowolny, L. Pelosi, L. Cairns, S. Ottolenghi, G. Bernardi, G. Cossu, L. Battistini, M. Molinaro, and N. Rosenthal. 2004. Muscle restricted expression of mIGF-l enhances the recruitment of stem cells during muscle regeneration. Proc Natl Acad Sci U S A. 101:1206-1210.
{93} Barton-Davis, E.R., D. I. Shoturma, A. Musaro, N. Rosenthal, and H. L. Sweeney. 1998. Viral mediated expression of insulin-like growth factor I blocks the aging-related loss of skeletal muscle function. Proc Natl Acad Sci U S A. 95: 15603-7.
{94} Ferri A., R. Gabbianelli, A. Casciati, E. Paolucci, G. Rotilio, M. T. Carri. 2000. Calcineurin activity is regulated both by redox compounds and by mutant familial amyotrophic lateral sclerosissuperoxide dismutase. J Neurochem. 75: 606-13.
{95} Musaro, A., K. J. McCullagh, F. J. Naya, E. N. Olson, and N. Rosenthal. 1999. IGF-1 induces skeletal myocyte hypertrophy through calcineurin in association with GATA-2 and NF-ATcl. Nature. 400: 581-585.
{96} Chin E. R., E. N. Olson, J. A. Richardson, Q. Yang, C. Humphries, J. M. Shelton, H. Wu, W. Zhu, R. Bassel-Duby, and R. S. Williams. 1998. A calcineurin-dependent transcriptional pathway controls skeletal muscle fiber type. Genes Dev. 12: 2499-509.
{97} Sakuma K., J. Nishikawa, R. Nakao, K. Watanabe, T. Totsuka, H. Nakano, M. Sano, and M. (2003)
{98} McConville, J. and A. Vincent. 2002. Diseases of the neuromuscular junction. Curr Opin Pharmacol.2: 296-301.
{99} Hall, E. D., J. A. Oostveen, and M. E. Gurney. 1998. Relationship of microglial and astrocytic activation to disease onset and progression in a transgenic model of familial ALS. Glia 23: 249-256.
{100} Elliott J L. 2001 Cytokine upregulation in a murine model of familial amyotrophic lateral sclerosis. Brain Res Mol Brain Res. 95: 172-178.
{101} Ferri A., M. Nencini, A. Casciati, M. Cozzolino, D. F. Angelini, P. Longone, A. Spalloni, G. Rotilio, and M. T Carri.2004. Cell death in amyotrophic lateral sclerosis: interplay between neuronal and glial cells. FASEB J.
{102} Kaspar, B. K., J. Llado, N. Sherkat, J. D. Rothstein, and F.H. Gage. 2003. Retrograde viral delivery of IGF-1 prolongs survival in a mouse ALS model. Science. 301: 839-842.
{103} Gurney M. E., H. Pu, A.Y. Chiu, M. C. Dal Canto, C. Y. Polchow, D. D. Alexander, J. Caliendo, A. Hentati, Y. W. Kwon, H. X. and Deng. 1994. Motor neuron degeneration in mice that express a human Cu,Zn superoxide dismutase mutation. Science 264: 1772-1775.
{104} Gossen, M. and H. Bujard (1992). “Tight control of gene expression in mammalian cells by tetracycline-responsive promoters.” Proc Natl Acad Sci U S A 89(12): 5547-51.
{105} Baron U, Bujard H. (2000) Tet repressor-based system for regulated gene expression in eukaryotic cells: principles and advances. Methods Enzymol. 327:401-21.
{106} Jones, J. I., Clemmons, D. R. (1995), Insulin-like growth factors and their binding proteins: biological actions. Endocr Rev 16(1): 3-34
{107} Rommel, C. et al., Mediation of IGF-1-induced skeletal myotube hypertrophy by PI(3)K/Akt/GSK3 pathways. Nat Cell Biol, 2001,3(11):p.1009-13
{108} Song, Y. H. et al. (2005). “Insulin-like growth factor-1-mediated skeletal muscle hypertrophy is characterized by increased mTOR/p70S6K signaling without increased Akt phosphorylation.” J Investig Med. 53(3):135-142
{109} Sandri M, Sandri C, Gilbert A, Skurk C, Calabria E, Picard A, Walsh K, Schiaffino S, Lecker S H, Goldberg A L. Foxo transcription factors induce the atrophy-related ubiquitin ligase atrogin-1 and cause skeletal muscle atrophy. Cell. 2004 Apr 30;117(3):399-412
{110} Skurk C, Izumiya Y, Maatz H, Razeghi P, Shiojima I, Sandri M, Sato K, Zeng L, Schiekofer S, Pimentel D, Lecker S, Taegtmeyer H, Goldberg A L, Walsh K. The FOXO3a transcription factor regulates cardiac myocyte size downstream of AKT signaling. J Biol Chem. 2005 May 27;280(21):20814-23. Epub 2005 Mar 21. {111} Hall R K, Yamasaki T, Kucera T, Waltner-Law M, O'Brien R, Granner D K. Regulation of phosphoenolpyruvate carboxykinase and insulin-like growth factor-binding protein-1 gene expression by insulin. The role of winged helix/forkhead proteins. J Biol Chem. 2000 Sep 29;275(39):30169-75. Erratum in: J Biol Chem 2001 Jun 22;276(25):23212.
{112} Hemers E, Duval C, McCaig C, Handley M, Dockray G J, Varro A. Insulin-like growth factor binding protein-5 is a target of matrix metalloproteinase-7: implications for epithelial-mesenchymal signaling. Cancer Res. 2005 Aug 15;65(16):7363-9.
{113} Dominov J A, Houlihan-Kawamoto C A, Swap C J, Miller J B. Pro- and anti-apoptotic members of the Bcl-2 family in skeletal muscle: a distinct role for Bcl-2 in later stages of myogenesis. Dev Dyn. 2001 Jan;220(1):18-26.
{114} Dominov J A, Kravetz A J, Ardelt M, Kostek C A, Beermann ML, Miller J B. Muscle-specific BCL2 expression ameliorates muscle disease in laminin {alpha}2-deficient, but not in dystrophin-deficient, mice. Hum Mol Genet. 2005 Apr 15;14(8):1029-40. Epub 2005 Mar 9.
{115} Shavlakadze, T., Winn, N. Rosenthal,N., Grounds, M. (2005). “Reconciling data from transgenic mice that overexpress IGF-1 specifically in skeletal muscle.” Growth Hormone & IGF Res 15:4-18
{116} Lowe, W. L., Jr., C. T. Roberts, Jr., S. R. Lasky and D. LeRoith (1987). “Differential expression of alternative 5′untranslated regions in mRNAs encoding rat insulin-like growth factor I.” Proc Natl Acad Sci U S A 84(24): 8946-50.
{117} Hoyt, E., Van Wyk, J J., Lund, P K. (1988). “Tissue and development specific regulation of a complex family of rat insulin-like growth factor I messenger ribonucleic acids.” Mol Endocrinol 2: 1077-1086.
{118} Lowe, W. L., Jr., S. R. Lasky, D. LeRoith and C. T. Roberts, Jr. (1988). “Distribution and regulation of rat insulin-like growth factor I messenger ribonucleic acids encoding alternative carboxyterminal E- peptides: evidence for differential processing and regulation in liver.” Mol Endocrinol 2(6): 528-35.
{119} Adamo, M. L., H. Ben-Hur, C. T. Roberts, Jr. and D. LeRoith (1991). “Regulation of start site usage in the leader exons of the rat insulin- like growth factor-I gene by development, fasting, and diabetes.” Mol Endocrinol 5(11): 1677-86.dhl
{120} Siegfried, J. M., P. G. Kasprzyk, et al. (1992). “A mitogenic peptide amide encoded within the E peptide domain of the insulin-like growth factor IB prohormone.” Proc Natl Acad Sci U S A 89(17): 8107-11.
{121} Tan, D. S., A. Cook, et al. (2002). “Nucleolar localization of an isoform of the IGF-I precursor.” BMC Cell Biol 3: 17.

1. A peptide having the formula NH₂-A-B-C-D-COOH, wherein: -A- is an optional N-terminus amino acid sequence consisting of a amino acids; -B- is an optional amino acid sequence consisting of b amino acids; -C- is a sequence derived from an IGF-1 Ea or Eb peptide; and -D- is an optional C-terminus amino acid sequence consisting of d amino acids.

2. A peptide according to claim 1, wherein -C- is a sequence derived from an IGF-1 Ea peptide.

3. A peptide according to claim 1, wherein -C- is a sequence derived from an IGF-1 Eb peptide.

4. A peptide according to any of claims 1-3, wherein the value of a, b and d is 0.

5. A peptide according to any of claims 1-3 or 5, -wherein -B- is a sequence derived from the mature processed IGF-1 peptide.

6. A peptide according to claim 5, wherein the value of a and/or d is 0.

7. A peptide according to any of claims 1-3 or 5, wherein -A- is a Class 1 IGF-1 signal peptide.

8. A peptide according to any of claims 1-3 or 5, wherein -A- is a Class 2 IGF-1 signal peptide.

9. A peptide according to any of claims 1-3 or 5, wherein -A- is a Class 3 IGF-1 signal peptide.

10. A peptide according to any of claims 7-9, wherein the value of d is 0.

11. A peptide according to any of the preceding claims, wherein -C- comprises the amino acid sequence as recited in SEQ ID NO:2.

12. A peptide according to any of the preceding claims, wherein -C- comprises the amino acid sequence as recited in SEQ ID NO:4.

13. A peptide according to any of claims 1-3, 5, or 7-12, wherein -A- comprises the amino acid sequence as recited in SEQ ID NO:6.

14. A peptide according to any of claims 1-3, 5, or 7-12, wherein -A- comprises the amino acid sequence as recited in SEQ ID NO:8.

15. A peptide according to any of claims 1-3, 5, or 7-12, wherein -A- comprises the amino acid sequence as recited in SEQ ID NO: 10.

16. A peptide according to any of claims 1-3, 5, or 7-12, wherein -B- comprises the amino acid sequence as recited in SEQ ID NO:12.

17. A purified nucleic acid molecule which encodes a polypeptide according to any one of the preceding claims.

18. A purified nucleic acid molecule according to claim 17, which comprises the nucleic acid sequence as recited in SEQ ID NO:1, SEQ ID NO:3, SEQ ID NO:5, SEQ ID NO:7, SEQ ID NO:9 or SEQ ID NO: 11, or is a redundant equivalent or fragment thereof.

19. A purified nucleic acid molecule according to claim 18, which consists of the nucleic acid sequence as recited in SEQ ID NO:1, SEQ ID NO:3, SEQ ID NO:5, SEQ ID NO:7, SEQ ID NO:9 or SEQ ID NO: 11, or is a redundant equivalent or fragment thereof.

20. A vector comprising a nucleic acid molecule as recited in any one of claims 17 to 19.

21. A host cell transformed with a vector according to claim 20.

22. Use of a peptide according to any one of claims 1-16, a nucleic acid molecule according to any one of claims 17-19, a vector according to claim 20 or a host cell according to claim 21, for the regulation of cellular growth or differentiation.

23. Use according to claim 22 wherein said regulation is of muscle tissue; nervous tissue; adipose tissue; cartilage; bone; hepatic tissue; kidney tissue; or skin.

24. Use according to claim 23 wherein said muscle tissue is skeletal, smooth or cardiac muscle.

25. Use of a peptide according to any of claims 1-16, a nucleic acid molecule according to any one of claims 17-19, a vector according to claim 19 or a host cell according to claim 21 in therapy.

26. Use of a peptide according to any of claims 1-16 , a nucleic acid molecule according to any one of claims 17-19, a vector according to claim 20 or a host cell according to claim 21, in the manufacture of a medicament for the prevention or limitation of cellular trauma.

27. Use according to claim 26, wherein said cellular trauma is to muscle tissue; nervous tissue; adipose tissue; cartilage; bone; hepatic tissue; kidney tissue; or skin.

28. Use according to claim 26 or 27, wherein the damage is caused by crush injury; surgical damage; muscle tear injury; nerve damage; surgical damage;
ischemia bums; bone fractures or UV damage.

29. Use according to claim 27 or 28, wherein said trauma to muscle tissue is to the myocardium.

30. Use according to any of claims 26-29 wherein said medicament is administered prior to said cellular trauma occurring in an attempt to prevent and reduce the damage.

31. Use according to any of claims 26-29 wherein said medicament is administered after said cellular trauma has occurred.

32. Use according to any of claims 26-31 wherein said peptide has the ability to induce a hypertrophic phenotype in said cells.

33. Use of a peptide according to any of claims 1-16, a nucleic acid molecule according to any one of claims 17-19, a vector according to claim 20 or a host cell according to claim 21, in the manufacture of a medicament for reducing muscular atrophy.

34. Use according to claim 33 wherein said muscular atrophy is caused by a degenerative disorder (cachexia); disuse of the muscle; sarcopenia; congestive heart failure or a stroke.

35. Use according to claim 34 wherein said disuse is caused by a restriction to normal muscle movement for a prolonged period of time or muscular paralysis.

36. Use according to claim 34 wherein the degenerative disorder is a neuromuscular disorder; a neurodegenerative disorder or a muscular dystrophy.

37. Use according to claim 34 wherein congestive heart failure includes cardiomyopathies; atherosclerosis; acute insult including myocarditis or myocardial infarction.

38. Use according to any of claims 34-37 wherein said medicament is administered as a preventative measure to slow said muscular atrophy or a therapeutic measure to both slow and reverse said muscular atrophy.

39. Use of a peptide according to any of claims 1-16, a nucleic acid molecule according to any one of claims 17-19, a vector according to claim 20 or a host cell according to claim 21, in the manufacture of a medicament for increasing muscular hypertrophy.

40. Use according to claim 39 wherein said medicament is administered to livestock to increase edible volume of the livestock.

41. Use according to claim 39 wherein said medicament is administered to a patient as an aid to physical therapy.

42. Use of a peptide of any one of claims 1-16, a nucleic acid molecule according to any one of claims 17-19, a vector according to claim 20 or a host cell according to claim 21, in the manufacture of a medicament for increasing adipose tissue.

43. Use according to claim 39 or 42 wherein said medicament is provided to help a patient gain weight after a severe illness, injury or continuing infection.

44. Use according to claim 42 wherein said medicament is used to treat Anorexia nervosa or Bulimia nervosa.

45. Use according to any of claims 22-44 wherein said medicament is administered orally, intravenously, intramuscularly, intra-arterially, intramedullary, intrathecally, intraventricularly, transdermally, subcutaneously, intraperitoneally, intranasally, enterally, topically, sublingually, intravaginally or rectally.

46. Use of claim 45 wherein said medicament is co-administered with thrombin.

47. Use according to claim 45 or 46 wherein said medicament is intended to act either at the site of administration or systemically.

48. A transgenic non-human animal that has been transformed to express higher levels of a polypeptide according to any one of claims 1 to 16.

49. A kit comprising one or more of: a peptide of any one of claims 1-16, a nucleic acid molecule according to any one of claims 17-19, a vector according to claim 20 or a host cell according to claim 21.

50. A kit according to claim 49, additionally comprising thrombin.


SEQUENCE LISTING (58 sequences)

SEQ ID NO: 1 - DNA, 105 bp, Mus musculus
cgctctatccgtgcccagcgccacactgacatgcccaagactcagaaggaagtacatttgaagaacacaagtagaggaagtgcaggaaacaagacctacagaatg

SEQ ID NO: 2 - protein, 35 aa, Mus musculus
RSIRAQRHTDMPKTQKEVHLKNTSRGSAGNKTYRM

SEQ ID NO: 3 - DNA, 123 bp, Mus musculus
cgctctatccgtgcccagcgccacactgacatgcccaagactcagaagtccccgtccctatcgacaaacaagaaaacgaagctgcaaaggagaaggaaaggaagtacatttgaagaacacaag

SEQ ID NO: 4 - protein, 41 aa, Mus musculus
RSIRAQRHTDMPKTQKSPSLSTNKKTKLQRRRKGSTFEEHK

SEQ ID NO: 5 - DNA, 138 bp, Mus musculus
atggggaaaatcagcagccttccaactcaattatttaagatctgcctctgtgacttcttgaagataaagatacacatcatgtcgtcttcacacctcttctacctggcgctctgcttgctcaccttcaccagctccacc

SEQ ID NO: 6 - protein, 46 aa, Mus musculus
MGKISSLPTQLFKICLCDFLKIKIHIMSSSHLFYLALCLLTFTSST

SEQ ID NO: 7 - DNA, 90 bp, Mus musculus
atgaccgcacctgcaataaagatacacatcatgtcgtcttcacacctcttctacctggcgctctgcttgctcaccttcaccagctccacc

SEQ ID NO: 8 - protein, 30 aa, Mus musculus
MTAPAIKIHIMSSSHLFYLALCLLTFTSST

SEQ ID NO: 9 - DNA, 60 bp, Mus musculus
atgtcgtcttcacacctcttctacctggcgctctgcttgctcaccttcaccagctccacc

SEQ ID NO: 10 - protein, 20 aa, Mus musculus
MSSSHLFYLALCLLTFTSST

SEQ ID NO: 11 - DNA, 216 bp, Mus musculus
acagctggaccagagaccctttgcggggctgagctggtggatgctcttcagttcgtgtgtggaccgaggggcttttacttcaacaagcccacaggctatggctccagcattcggagggcacctcagacaggcattgtggatgagtgttgcttccggagctgtgatctgaggagactggagatgtactgtgccccactgaagcctacaaaagcagcc

SEQ ID NO: 12 - protein, 72 aa, Mus musculus
TAGPETLCGAELVDALQFVCGPRGFYFNKPTGYGSSIRRAPQTGIVDECCFRSCDLRRLEMYCAPLKPTKAA

SEQ ID NO: 13 - DNA, 144 bp, Homo sapiens
atgggaaaaatcagcagtcttccaacccaattatttaagtgctgcttttgtgatttcttgaaggtgaagatgcacaccatgtcctcctcgcatctcttctacctggcgctgtgcctgctcaccttcaccagctctgccacggct

SEQ ID NO: 14 - protein, 48 aa, Homo sapiens
MGKISSLPTQLFKCCFCDFLKVKMHTMSSSHLFYLALCLLTFTSSATA

SEQ ID NO: 15 - DNA, 144 bp, Mus musculus
atggggaaaatcagcagccttccaactcaattatttaagatctgcctctgtgacttcttgaagataaagatacacatcatgtcgtcttcacacctcttctacctggcgctctgcttgctcaccttcaccagctccaccacagct

SEQ ID NO: 16 - protein, 48 aa, Mus musculus
MGKISSLPTQLFKICLCDFLKIKIHIMSSSHLFYLALCLLTFTSSTTA

SEQ ID NO: 17 - DNA, 96 bp, Homo sapiens
atgattacacctacagtgaagatgcacaccatgtcctcctcgcatctcttctacctggcgctgtgcctgctcaccttcaccagctctgccacggct

SEQ ID NO: 18 - protein, 32 aa, Homo sapiens
MITPTVKMHTMSSSHLFYLALCLLTFTSSATA

SEQ ID NO: 19 - DNA, 96 bp, Mus musculus
atgaccgcacctgcaataaagatacacatcatgtcgtcttcacacctcttctacctggcgctctgcttgctcaccttcaccagctccaccacagct

SEQ ID NO: 20 - protein, 32 aa, Mus musculus
MTAPAIKIHIMSSSHLFYLALCLLTFTSSTTA

SEQ ID NO: 21 - DNA, 105 bp, Homo sapiens
cgctctgtccgtgcccagcgccacaccgacatgcccaagacccagaaggaagtacatttgaagaacgcaagtagagggagtgcaggaaacaagaactacaggatg

SEQ ID NO: 22 - protein, 35 aa, Homo sapiens
RSVRAQRHTDMPKTQKEVHLKNASRGSAGNKNYRM

SEQ ID NO: 23 - DNA, 120 bp, Homo sapiens
cgctctgtccgtgcccagcgccacaccgacatgcccaagacccagaagtatcagcccccatctaccaacaagaacacgaagtctcagagaaggaaaggaagtacatttgaagaacgcaag

SEQ ID NO: 24 - protein, 40 aa, Homo sapiens
RSVRAQRHTDMPKTQKYQPPSTNKNTKSQRRKGSTFEERK

SEQ ID NO: 25 - DNA, 231 bp, Homo sapiens
cgctctgtccgtgcccagcgccacaccgacatgcccaagacccagaagtatcagcccccatctaccaacaagaacacgaagtctcagagaaggaaaggttggccaaagacacatccaggaggggaacagaaggaggggacagaagcaagtctgcagatcagaggaaagaagaaagagcagaggagggagattggaagtagaaatgctgaatgcagaggcaaaaaaggaaaa

SEQ ID NO: 26 - protein, 77 aa, Homo sapiens
RSVRAQRHTDMPKTQKYQPPSTNKNTKSQRRKGWPKTHPGGEQKEGTEASLQIRGKKKEQRREIGSRNAECRGKKGK

SEQ ID NO: 27 - DNA, 189 bp, Mus musculus
cgctctatccgtgcccagcgccacactgacatgcccaagactcagaagtccccgtccctatcgacaaacaagaaaacgaagctgcaaaggagaaggaaaggtgagccaaagacacacccagaaggggaacaggaggaggtaacggaggcaactcggaaaatcagaggtcccagagaaaaaagactgggc

SEQ ID NO: 28 - protein, 63 aa, Mus musculus
RSIRAQRHTDMPKTQKSPSLSTNKKTKLQRRRKGEPKTHPEGEQEEVTEATRKIRGPREKRLG

SEQ ID NO: 29 - DNA, 210 bp, Homo sapiens
ggaccggagacgctctgcggggctgagctggtggatgctcttcagttcgtgtgtggagacaggggcttttatttcaacaagcccacagggtatggctccagcagtcggagggcgcctcagacaggcatcgtggatgagtgctgcttccggagctgtgatctaaggaggctggagatgtattgcgcacccctcaagcctgccaagtcagct

SEQ ID NO: 30 - protein, 70 aa, Homo sapiens
GPETLCGAELVDALQFVCGDRGFYFNKPTGYGSSSRRAPQTGIVDECCFRSCDLRRLEMYCAPLKPAKSA

SEQ ID NO: 31 - DNA, 210 bp, Mus musculus
ggaccagagaccctttgcggggctgagctggtggatgctcttcagttcgtgtgtggaccgaggggcttttacttcaacaagcccacaggctatggctccagcattcggagggcacctcagacaggcattgtggatgagtgttgcttccggagctgtgatctgaggagactggagatgtactgtgccccactgaagcctacaaaagcagcc

SEQ ID NO: 32 - protein, 70 aa, Mus musculus
GPETLCGAELVDALQFVCGPRGFYFNKPTGYGSSIRRAPQTGIVDECCFRSCDLRRLEMYCAPLKPTKAA

SEQ ID NO: 33 - DNA, 25 bp, Artificial Sequence
  oligonucleotide
acatcaacaagagcttgacccaggc

SEQ ID NO: 34 - DNA, 24 bp, Artificial Sequence
  oligonucleotide
agctcatatggtccgacagcacga

SEQ ID NO: 35 - DNA, 24 bp, Artificial Sequence
  oligonucleotide
aggataccactcccaacagacgtg

SEQ ID NO: 36 - DNA, 24 bp, Artificial Sequence
  oligonucleotide
gtagctatggtactccagaagacc

SEQ ID NO: 37 - DNA, 19 bp, Artificial Sequence
  oligonucleotide
ccaagccttatcggaaatg

SEQ ID NO: 38 - DNA, 16 bp, Artificial Sequence
  oligonucleotide
tggccttgtagacacc

SEQ ID NO: 39 - DNA, 15 bp, Artificial Sequence
  oligonucleotide
catcggcattttgaa

SEQ ID NO: 40 - DNA, 20 bp, Artificial Sequence
  oligonucleotide
cgtttggcacatccatctcc

SEQ ID NO: 41 - DNA, 17 bp, Artificial Sequence
  oligonucleotide
tgggtgtgaaccacgaa

SEQ ID NO: 42 - DNA, 16 bp, Artificial Sequence
  oligonucleotide
acagctttccagaggg

SEQ ID NO: 43 - DNA, 24 bp, Artificial Sequence
  oligonucleotide
atgggctccttctccatcaccctg

SEQ ID NO: 44 - DNA, 23 bp, Artificial Sequence
  oligonucleotide
tcggtaccggaagctgttgcagc

SEQ ID NO: 45 - DNA, 24 bp, Artificial Sequence
  oligonucleotide
atggatctcctgaaggtgctgtcc

SEQ ID NO: 46 - DNA, 24 bp, Artificial Sequence
  oligonucleotide
gcgttacagcccaaacgactgacg

SEQ ID NO: 47 - DNA, 27 bp, Artificial Sequence
  oligonucleotide
ctgagcagaagcgcaatgcagagtcgg

SEQ ID NO: 48 - DNA, 28 bp, Artificial Sequence
  oligonucleotide
ctcctcattcaggcccttggcaccaatg

SEQ ID NO: 49 - DNA, 24 bp, Artificial Sequence
  oligonucleotide
atgtgcgacgaagacgagaccacc

SEQ ID NO: 50 - DNA, 24 bp, Artificial Sequence
  oligonucleotide
gccacatacatggcaggcacgttg

SEQ ID NO: 51 - DNA, 24 bp, Artificial Sequence
  oligonucleotide
gatcccagcagcaagaaggtgacg

SEQ ID NO: 52 - DNA, 24 bp, Artificial Sequence
  oligonucleotide
tggagaagcccataagcacagcag

SEQ ID NO: 53 - DNA, 25 bp, Artificial Sequence
  oligonucleotide
taaaacgcagctcagtaacagtccg

SEQ ID NO: 54 - DNA, 25 bp, Artificial Sequence
  oligonucleotide
tggaatcctgtggcatccatgaaac

SEQ ID NO: 55 - DNA, 23 bp, Artificial Sequence
  oligonucleotide
cccagaccctcacacactcagat

SEQ ID NO: 56 - DNA, 21 bp, Artificial Sequence
  oligonucleotide
ttgtcccttgaagagaacctg

SEQ ID NO: 57 - DNA, 20 bp, Artificial Sequence
  oligonucleotide
gtgggccgctctaggcacaa

SEQ ID NO: 58 - DNA, 24 bp, Artificial Sequence
  oligonucleotide
ctctttgatgtcacgcacgatttc